(19) United States
(12) Patent Application Publication
(10) Pub. No.: US 2008/0241166 A1
(43) Pub. Date: Oct. 2, 2008

(54) Ligands that bind a receptor

(75) Inventors: Ian M. Tomlinson, Great Shelford (GB); Gregory P. Winter, Cambridge (GB); Olga Ignatovich, Cambridge (GB); Philip C. Jones, Cambridge (GB); Lucy J. Holt, Cambridge (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Domantis Limited, Cambridge (GB)

(21) Appl. No.: 12/006,933

(22) Filed: Jan. 7, 2008

Related U.S. Application Data
(63) Continuation-in-part of application No. 11/331,415, filed on Jan. 11, 2006.
Application No. 11/331,415 is a continuation-in-part of application No. 11/098,758, filed on Apr. 4, 2005.
Application No. 11/098,758 is a continuation-in-part of application No. 10/925,366, filed on Aug. 24, 2004.
Application No. 10/925,366 is a continuation-in-part of application No. 11/664,542, filed on Sep. 6, 2007.
Application No. 11/664,542 is a continuation-in-part of application No. 10/985,847, filed on Nov. 10, 2004.
Application No. 10/985,847 is a continuation-in-part of application No. PCT/GB04/04253, filed on Oct. 8, 2004.
Application No. PCT/GB04/04253 is a continuation-in-part of application No. PCT/GB03/05646, filed on Dec. 24, 2003.
Application No. PCT/GB03/05646 is a continuation-in-part of application No. PCT/GB03/02804, filed on Jun. 30, 2003.
Application No. PCT/GB03/02804 is a continuation-in-part of application No. PCT/GB02/03014, filed on Jun. 28, 2002.

(30) Foreign Application Priority Data
Dec. 27, 2002 (GB) 0230202.4
Nov. 28, 2003 (GB) 0327706.8
Dec. 5, 2006 (GB) PCT/GB2006/004559

(51) Int. Cl. A61K 39/395; C07K 16/00; C07H 21/00; C12N 15/63; C12N 5/00; A61P 35/00

(52) U.S. Cl. 424/172.1; 530/387.1; 536/23.53; 435/320.1; 435/325; 435/375

(57) ABSTRACT

The invention relates to ligands, such as immunoglobulin single variable domains, that have binding specificity for a receptor. Preferably the receptor is a cell surface receptor and/or the ligand inhibits the activity of the receptor.

### BACKGROUND OF THE INVENTION

Expression or overexpression of certain receptors on the surfaces of cells has been correlated with a number of different diseases and disorders (e.g., inflammatory conditions, cancer). Thus, agents that specifically bind receptors, such as antibodies, are potentially useful therapeutic and diagnostic agents. However, approaches for treating such conditions using conventional therapeutic antibodies can have limited success. One factor limiting the use of conventional antibodies is that antibodies often induce receptor cross-linking upon binding to a receptor, resulting in the activation of the receptor. In this situation, the underlying disease or disorder may be exacerbated by the therapy.

Certain cancer cells express or overexpress certain cellular components such as receptors or membrane bound (e.g., cell surface) proteins in comparison to normal cells. One approach to improve conventional approaches to cancer therapy (e.g., surgical and chemotherapeutic approaches) involves targeting tumor cells directly, for example using antibodies or antibody fragments that bind to proteins that are expressed or overexpressed on tumor cells. A number of such target proteins have been identified. Among such proteins is the epidermal growth factor receptor (EGFR).

EGFR is a member of the ErbB1 family and transduces signals that lead to cellular proliferation and survival, and the elaboration of motility, growth and angiogenic factors upon binding epidermal growth factor (EGF) and/or transforming growth factor alpha (TGF alpha). EGFR has been demonstrated to be involved in tumor growth, metastasis and angiogenesis. Many cancers express EGFR, such as bladder cancer, ovarian cancer, colorectal cancer, breast cancer, lung cancer (e.g., non-small cell lung carcinoma), gastric cancer, pancreatic cancer, prostate cancer, head and neck cancer, renal cancer and gall bladder cancer. ERBITUX® (cetuximab; Imclone Systems Inc.), a chimeric mouse/human antibody that binds human EGFR, has been approved for treating certain EGFR-expressing cancers in combination with irinotecan. Targeting EGFR with currently available therapeutics is not effective in all patients, or for all cancers (e.g., EGFR-expressing cancers).

Other receptors, such as CD38, CD138, carcinoembryonic antigen (CEA) and CD56 are expressed or overexpressed on certain cancer cells. CD38 is a novel multifunctional ectoenzyme widely expressed in cells and tissues especially in leukocytes. CD38 also functions in cell adhesion, signal transduction and calcium signaling. Ferrero E, J. Leukoc. Biol. 65:151 (1999). CD56 is a receptor that mediates homophilic adhesion in certain cell types. Thiery J P et al., Proc Natl Acad Sci USA 79:6737 (1982). CD138 is also known as syndecan-1. The syndicans mediate cell binding, cell signaling, and cytoskeletal organization and syndecan receptors are required for internalization of the HIV-1 tat protein. J Biol Regul Homeost Agents. April-June; 16(2): 152-5 (2002) CEA is a complex immunoreactive glycoprotein that can mediated cell adhesion, and is a widely used tumor marker. Duffy, M. J., Clin Chem. April; 47(4):624-30 (2001).

TNFR1 is a transmembrane receptor containing an extracellular region that binds ligand and an intracellular domain that lacks intrinsic signal transduction activity but can associate with signal transduction molecules. The complex of TNFR1 with bound TNF contains three TNFR1 chains and three TNF chains. (Banner et al., Cell, 73(3) 431-445 (1993).) The TNF ligand is present as a trimer, which is bound by three TNFR1 chains. (Id.) The three TNFR1 chains are clustered closely together in the receptor-ligand complex, and this clustering is a prerequisite to TNFR1-mediated signal transduction. In fact, multivalent agents that bind TNFR1, such as anti-TNFR1 antibodies, can induce TNFR1 clustering and signal transduction in the absence of TNF and are commonly used as TNFR1 agonists. (See, e.g., Belka et al., EMBO, 14(6):1156-1165 (1995); Mandik-Nayak et al., J Immunol, 167:1920-1928 (2001).) Accordingly, multivalent agents that bind TNFR1, are generally not effective antagonists of TNFR1 even if they block the binding of TNFα to TNFR1.

Interleukin 1 binds to two receptors Interleukin 1 Receptor type 1 (IL-1R1, CD121a, p80) which transduce signal into cells upon binding IL-1, and Interleukin 1 Receptor type 2 (IL-1R1, CDw121b) which does not transduce signals upon binding IL-1 and acts as an endogenous regulator of IL-1. Signals transduced through IL-1R1 upon binding IL-1 (e.g., IL-1α or IL-1β) induce a wide spectrum of biological activities that can contribute to or exacerbate pathological states. For example, signals transduced through IL-1R1 upon binding of IL-1 can lead to local or systemic inflammation, the elaboration of additional inflammatory mediators (e.g., IL-6, Il-8, TNF), fever, activate immune cells (e.g., lymphocytes, neutrophils), anorexia, hypotension, leucopenia, and thrombocytopenia. Signals transduced through IL-1R1 upon binding of IL-1 also have effects on non-immune cells, such as stimulating chondrocytes to release collagenase and other enzymes that degrade cartilage, and stimulates the differentiation of osteoclast progenitor cells into mature osteoclasts which leads to resorption of bone. (See, e.g., Hallegua and Weisman, Ann. Theum. Dis. 61:960-967 (2002).) The interaction of IL-1 with IL-1R1 has been implicated in the pathogenesis of several diseases such as arthritis (e.g., rheumatoid arthritis, osteoarthritis) and inflammatory bowel disease. Certain agents that bind Interleukin 1 Receptor Type 1 (IL-1R1) and neutralize its activity (e.g., IL-1ra) have proven to be effective therapeutic agents for certain inflammatory conditions, such as moderately to severely active rheumatoid arthritis. However, other agents that bind IL-1R1, such as the anti-IL-1R1 antibody AMG 108 (Amgen) have failed to meet primary endpoints in clinical studies.

### SUMMARY OF THE INVENTION

The invention relates to a ligand (e.g., an isolated domain antibody (dAb)) that has binding specificity for a receptor (e.g., human EGFR). As described herein, the ligands of the invention (e.g., isolated domain antibody monomers) are promising therapeutic agents for the treatment of a variety of conditions, such as conditions associated with the expression, overexpression or activity of a receptor (e.g., inflammatory conditions, cancer). Unlike conventional antibodies, which can cause receptor clustering or cross-linking and activation upon binding of the antibody to the receptor, domain antibodies (e.g., dAb monomers) and certain ligands that comprise domain antibodies (e.g., monovalent ligands) do not substantially induce receptor clustering or cross-linking, and therefore, can antagonize a receptor without substantially agonizing the receptor

In some embodiments, the ligand is an isolated domain antibody (dAb) (e.g., a dAb monomer) that has binding specificity for a receptor, preferably a human receptor. The isolated domain antibody can be from any desired species such as a mouse or rat. Preferably, the isolated dAb is a human dAb.

Preferred isolated dAbs inhibit the receptor to which they bind. For example, the isolated dAb can inhibit the bind of a cognate ligand to the receptor, inhibit receptor clustering, and/or inhibits receptor signalling.

The isolated dAb can have binding specificity for any desired receptor, such as a receptor that forms dimers, trimers or multimers upon binding of cognate ligand (e.g. dimeric receptors, trimeric receptors, multimeric receptors). In particular embodiments, the receptor binds a cognate ligand that is a growth factor of cytokine, i.e., the receptor is a growth factor receptor or a cytokine receptor.

In some embodiments, the isolated dAb comprises a heavy chain variable domain from the V_{H}3-family. For example, the isolated dAb can comprise a variable domain encoded by the DP47, DP45 or DP38 V_{H} gene segment. In one embodiment, the isolated dAb comprises or further comprises a heavy chain hypervariable loop having the canonical structure of the H3 loop of DP47 and JH4b. Alternatively or in addition, the isolated dAb can comprise a hypervariable loop having the canonical structure of the H2 loop of DP47.

In some embodiments, the isolated dAb is an antagonist of the receptor to which it binds. The isolated dAb can bind receptor with high affinity, such as an affinity of 300 nM to 5 pM.

In some embodiments, the ligand is a dual specific ligand comprising a dAb monomer that has binding specificity for a receptor, and further comprises a second dAb. The second dAb can have binding specificity for the same or different receptor or for a polypeptide that increases in vivo serum half life (e.g., human serum albumin). In some embodiments, the second dAb binds mouse serum albumin and human serum albumin. In other embodiments, the second dAb binds human serum albumin, and further binds rat serum albumin, pig serum albumin or cynomolgus monkey serum albumin.

In some embodiments, the ligand comprises a dAb monomer and further comprises an antibody constant domain, such as CH1, hinge, CH2, CH3, Cλ, Cκ. In some embodiments, the ligand is an IgG-like format. For example, the ligand can comprise an antibody Fc region (e.g., comprising one or both of C_{H}2 and C_{H}3 domains, and optionally a hinge region).

In some embodiments, the ligand further comprises a toxin. Alternatively or in addition, the ligand can comprise a half-life extending moiety.

In certain embodiments, the ligand comprises at least one immunoglobulin single variable domain that has binding specificity for a receptor, and at least one immunoglobulin single variable domain that has binding specificity for an antigen or epitope which increases the half-life of said ligand. For example, the ligand can comprise two immunoglobulin single variable domains that have binding specificity for a receptor, and one immunoglobulin single variable domain that has binding specificity for an antigen or epitope which increases the half-life of said ligand (e.g., human serum albumin). The ligand can bind a dimeric receptor or multimeric receptor, such as a dimeric or multimeric cytokine receptor or dimeric or multimeric growth factor receptor.

The invention also relates to a pharmaceutical composition comprising a ligand of the invention and a pharmacologically acceptable carrier. In a certain embodiment, the pharmaceutical composition further comprises a cytotoxic agent.

The invention further relates to isolated or recombinant nucleic acids encoding a ligand of the invention (e.g., a ligand that comprises a dAb monomer), recombinant vectors comprising such recombinant nucleic acids, and host cells that comprise such recombinant vectors. The invention also relates to a method of making a ligand of the invention comprising maintaining a host cell that contains a recombinant nucleic acid encoding the ligand under conditions suitable for expression and production of the ligand. In some embodiments, the method further comprises isolating the ligand.

The invention also relates to a method for antagonizing a receptor in a patient without substantially agonizing the receptor, comprising administering to a patient in need thereof a therapeutically effective dose of a ligand that binds said receptor.

The invention also relates to a method for antagonizing a receptor without substantially agonizing the receptor, comprising combining a cell that expresses the receptor with a ligand that binds said receptor under conditions suitable for binding of said ligand to said receptor.

The invention also relates to use of a ligand that binds receptor in therapy or diagnosis, and to the use of a ligand that binds a receptor for the manufacture of a medicament for antagonizing a receptor without substantially agonizing the receptor or for treating a disease described herein.

### BRIEF DESCRIPTION OF THE DRAWINGS

FIG. 1 shows the diversification of V_{H}/HSA at positions H50, H52, H52a, H53, H55, H56, H58, H95, H96, H97, H98 (DVT or NNK encoded respectively) which are in the antigen binding site of V_{H} HSA. (SEQ ID NO:1, nucleotide sequence; SEQ ID NO:2, amino acid sequence.) The sequence of V_{K} is diversified at positions L50, L53.

FIG. 2 is a schematic showing the structure of the plasmid pIT1/pIT2 used to prepare single chain Fv (scFv) libraries, and shows the nucleotide sequence of the plasmid across the expression control and cloning regions (SEQ ID NO:3) and the encoded amino acid sequence (SEQ ID NO:4). The plasmid was used to prepare

Library 1: Germline V_{K}/DVT V_{H},

Library 2: Germline V_{K}/NNK V_{H},

Library 3: Germline V_{H}/DVT V_{K}, and

Library 4: Germline V_{H}/NNK V_{K} in phage display/ScFv format.

These libraries were pre-selected for binding to generic ligands protein A and protein L so that the majority of the clones and selected libraries are functional. Libraries were selected on HSA (first round) and β-gal (second round) or HSA β-gal selection or on β-gal (first round) and HSA (second round) β-gal HSA selection. Soluble scFv from these clones of PCR are amplified in the sequence. One clone encoding a dual specific antibody K8 was chosen for further work.

FIG. 3 shows an alignment of V_{H} chains (V_{H} dummy (SEQ ID NO:5), K8 (SEQ ID NO:6), V_{H}2 (SEQ ID NO:7), V_{H}4 (SEQ ID NO:8), VHC11 (SEQ ID NO:9), VHA10sd (SEQ ID NO:10), VHA1sd (SEQ ID NO:11), VHA5sd (SEQ ID NO:12), VHC5sd (SEQ ID NO:13), VHC11sd (SEQ ID NO:14), and V_{κ} chains (Vk dummy (SEQ ID NO:15), K8 (SEQ ID NO:16), E5sc (SEQ ID NO:17), C3 (SEQ ID NO:18)).

FIG. 4 shows the characterisation of the binding properties of the K8 antibody, the binding properties of the K8 antibody characterised by monoclonal phage ELISA, the dual specific K8 antibody was found to bind HSA and β-gal and displayed on the surface of the phage with absorbant signals greater than 1.0. No cross reactivity with other proteins was detected.

FIG. 5 shows soluble scFv ELISA performed using known concentrations of the K8 antibody fragment. A 96-well plate was coated with 100 μg of HSA, BSA and β-gal at 10 μg/ml and 100 μg/ml of Protein A at 1 μg/ml concentration. 50 μg of the serial dilutions of the K8 scFv was applied and the bound antibody fragments were detected with Protein L-HRP. ELISA results confirm the dual specific nature of the K8 antibody.

FIG. 6 shows the binding characteristics of the clone K8V_{κ}/dummy V_{H} analysed using soluble scFv ELISA. Production of the soluble scFv fragments was induced by IPTG as described by Harrison et al, Methods Enzymol. 1996; 267:83-109 and the supernatant containing scFv assayed directly. Soluble scFv ELISA is performed as described in example 1 and the bound scFvs were detected with Protein L-HRP. The ELISA results revealed that this clone was still able to bind β-gal, whereas binding BSA was abolished.

FIG. 7 shows the sequence (SEQ ID NO:2 and SEQ ID NO:3) of variable domain vectors 1 and 2.

FIG. 8 is a map of the C_{H} vector used to construct a V_{H}1/V_{H}2 multispecific ligand.

FIG. 9 is a map of the C_{κ} vector used to construct a V_{κ}1/V_{κ}2 multispecific ligand.

FIG. 10 shows the results of a TNF receptor assay comparing TAR1-5 dimer 4, TAR1-5-19 dimer 4 and TAR1-5-19 monomer.

FIG. 11 shows the results of a TNF receptor assay comparing TAR1-5 dimers 1-6. All dimers were FPLC purified and the results for the optimal dimeric species are shown.

FIG. 12 shows the results of a TNF receptor assay of TAR1-5 19 homodimers in different formats: dAb-linker-dAb format with 3U, 5U or 7U linker, Fab format and cysteine hinge linker format.

FIG. 13 shows Dummy V_{H} sequence for library 1. (amino acid sequence ((SEQ ID NO:5; nucleotide sequences: coding strand (SEQ ID NO:19), noncoding strand (SEQ ID NO:20) The sequence of the V_{H} framework based on germline sequence DP47-JH4b. Positions where NNK randomisation (N=A or T or C or G nucleotides; K=G or T nucleotides) has been incorporated into library 1 are indicated in bold underlined text.

FIG. 14 shows Dummy V_{H} sequence for library 2. (amino acid sequence (SEQ ID NO:21); nucleotide sequences: coding strand (SEQ ID NO:22), noncoding strand (SEQ ID NO:23)). The sequence of the V_{H} framework based on germline sequence DP47-JH4b. Positions where NNK randomisation (N=A or T or C or G nucleotides; K=G or T nucleotides) has been incorporated into library 2 are indicated in bold underlined text.

FIG. 15 shows Dummy V_{κ} sequence for library 3. (amino acid sequence ((SEQ ID NO:15; nucleotide sequences: coding strand (SEQ ID NO:24), noncoding strand (SEQ ID NO:25) The sequence of the V_{κ} framework based on germline sequence DP_{K}9-J_{K}1. Positions where NNK randomisation (N=A or T or C or G nucleotides; K=G or T nucleotides) has been incorporated into library 3 are indicated in bold underlined text.

FIG. 16 shows nucleotide and amino acid sequence of anti MSA dAbs MSA 16 (nucleotide sequence (SEQ ID NO:26), amino acid sequence (SEQ ID NO:27) and MSA 26 (nucleotide sequence (SEQ ID NO:28), amino acid sequence ((SEQ ID NO:29).

FIGS. 17A and 17B show inhibition biacore of MSA 16 and 26, respectively. Purified dAbs MSA16 and MSA26 were analysed by inhibition biacore to determine K_{d}. Briefly, the dAbs were tested to determine the concentration of dAb required to achieve 200RUs of response on a biacore CM5 chip coated with a high density of MSA. Once the required concentrations of dAb had been determined, MSA antigen at a range of concentrations around the expected K_{d} was premixed with the dAb and incubated overnight. Binding to the MSA coated biacore chip of dAb in each of the premixes was then measured at a high flow-rate of 30 μl/minute.

FIG. 18 shows serum levels of MSA16 following injection. Serum half life of the dAb MSA16 was determined in mouse. MSA16 was dosed as single i.v. injections at approx 1.5 mg/kg into CD1 mice. Modelling with a 2 compartment model showed MSA16 had a t1/2α of 0.98 hr, a t1/2β of 36.5 hr and an AUC of 913 hr.mg/ml. MSA16 had a considerably lengthened half life compared with HEL4 (an anti-hen egg white lysozyme dAb) which had a t1/2α of 0.06 hr and a t1/2β of 0.34 hr.

FIG. 19(a) shows an ELISA showing inhibition of TNF binding with a Fab-like fragment comprising MSA26Ck and TAR1-5-19CH. Addition of MSA with the Fab-like fragment reduces the level of inhibition. An ELISA plate coated with 1 μg/ml TNFα was probed with dual specific VκC_{H} and VκCκ Fab like fragment and also with a control TNFα binding dAb at a concentration calculated to give a similar signal on the ELISA. Both the dual specific and control dAb were used to probe the ELISA plate in the presence and in the absence of 2 mg/ml MSA. The signal in the dual specific well was reduced by more than 50% but the signal in the dAb well was not reduced at all. These data demonstrate that binding of MSA to the dual specific is competitive with binding to TNFα. FIG. 19(b) shows a schematic illustrating the TNF receptor assay. FIG. 19(c) shows a TNF receptor assay showing inhibition of TNF binding with a Fab-like fragment comprising MSA26Ck and TAR1-5-19CH. Addition of MSA with the Fab-like fragment reduces the level of inhibition. The dual specific VκC_{H} and VκCκ Fab like fragment used in FIG. 19(a) was also put into the receptor assay with and without MSA and competition by MSA was also shown. These data also demonstrate that binding of MSA to the dual specific is competitive with binding to TNFα.

FIG. 20 shows a TNF receptor assay showing inhibition of TNF binding with a disulphide bonded heterodimer of TAR1-5-19 dAb and MSA16 dAb. Addition of MSA with the dimer reduces the level of inhibition in a dose dependant manner. The TNF receptor assay (FIG. 19 (b)) was conducted in the presence of a constant concentration of heterodimer (18 nM) and a dilution series of MSA and HSA. The presence of HSA at a range of concentrations (up to 2 mg/ml) did not cause a reduction in the ability of the dimer to inhibit TNFα. However, the addition of MSA caused a dose dependant reduction in the ability of the dimer to inhibit TNFα (FIG. 19a). This demonstrates that MSA and TNFα compete for binding to the cys bonded TAR1-5-19, MSA16 dimer. MSA and HSA alone did not have an effect on the TNF binding level in the assay.

FIG. 21 shows the vectors used for Fab construction according to the invention.

FIG. 22 shows the binding of Fab comprising TAR1/TAR2Dabs to TNF and TNFR1 via an ELISA assay.

FIG. 23 shows the results of sandwich ELISA to test the ability of TAR1/TAR2 Fab to bind to both TNF and TNFR antigens simultaneously, that is to test whether the Fab is of open or closed conformation.

FIG. 24 shows the results of competition ELISA to test the ability of TAR1/TAR2 Fab to bind to both antigens simultaneously, that is to test whether the Fab is of open or closed conformation.

FIG. 25 shows the results of cell based assays using dAbs and a Fab dual specific ligand according to the invention.

FIG. 26 shows murine TNF cytoxicity on murine cells with soluble human TNFR1 and increasing concentrations of mutant TNF (competition on cells).

FIG. 27 shows the construction of IgG vectors which express IgG1 heavy chain constant region and light chain kappa constant region respectively.

FIG. 28 shows the binding of TAR1/TAR2 IgG to TNF and TNFR1 in ELISA assay.

FIG. 29 shows the analysis of TAR1/TAR2 IgG properties in cell assays.
(a) Human TNF cytotoxicity on murine cells.
(b) Murine TNF cytotoxicity assay on murine cells with human soluble TNF receptor.
(c) Human TNF induced IL-8 release from human cells.
(d) Murine TNF induced IL-8 secretion from human cells.

FIG. 30 shows Human TNF induced IL-8 secretion on human cells

FIG. 31 shows the amino acid sequence of TAR2h-10-27 and the nucleotide sequence of a nucleic acid that encodes TAR2h-10-27.

FIG. 32 is a graph showing that anti-TNFR1 dAb formats do not substantially agonize TNFR1 in an L929 assay. L929 cells were cultured in media that contained a range of concentrations of anti-TNFR1 dAb monomer (TAR2m-21-23), TAR2m-21-23 monomer cross-linked by a commercially available anti-myc antibody (9E10), dual specific anti-TNFR1 dAb/anti-SA dAb (TAR2m-21-23 3U TAR7m-16), or pegylated anti-TNFR1 dAb monomer (TAR2m-21-23 40K PEG). In the case of TAR2m-21-23 monomer cross-linked by the anti-myc antibody, the dAb and antibody were mixed in a 2:1 ratio and pre-incubated for one hour at room-temperature to simulate the effects of in vivo immune cross-linking prior to culture. (The TAR2m-21-23 monomer includes a myc epitope.) TAR2m-21-23 monomer was incubated with the L929 cells at a concentration of 3,000 nM. TAR2m-21-23 monomer and anti-Myc antibody were incubated at a dAb concentration of 3,000 nM. TAR2m-21-23 3U TAR7m-16 was incubated with the cells at 25 nM, 83.3 nM, 250 nM, 833 nM and 2,500 nM concentrations. TAR2m-21-23 40K PEG was incubated with the cells at 158.25 nM, 527.5 nM, 1582.5 nM, 5,275 nM and 15,825 nM concentrations. After incubation overnight, cell viability was assessed. The results revealed that incubation of L929 cells with 10 nM, 1 nM or 0.1 nM of a commercially-available anti-TNFR1 IgG antibody that crosslinks and agonizes TNFR1 (Catalog No. AF-425-PB; R&D Systems, Minneapolis, Minn.) resulted in a dose-dependent increase in non-viable cells, thereby demonstrating the sensitivity of these cells to agonists of TNFR1. In contrast, incubation with various amounts of anti-TNFR1 formats did not antagonize TNFR1 and did not result in an increase in the number of non-viable cells in the cultures, even when used at more than 1000 times the concentration of the commercially-available anti-TNFR1 IgG antibody.

### DETAILED DESCRIPTION OF THE INVENTION

### Definitions

As used herein, two immunoglobulin domains are “complementary” where they belong to families of structures which form cognate pairs or groups or are derived from such families and retain this feature. For example, a V_{H} domain and a V_{L} domain of an antibody are complementary; two V_{H} domains are not complementary, and two V_{L} domains are not complementary. Complementary domains may be found in other members of the immunoglobulin superfamily, such as the V_{α} and V_{β} (or γ and δ) domains of the T-cell receptor. In the context of the present invention, complementary domains do not bind a target molecule co-operatively, but act independently on different target epitopes which may be on the same or different molecules.

The term “immunoglobulin” refers to a family of polypeptides which retain the immunoglobulin fold characteristic of antibody molecules, which contains two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention is applicable to all immunoglobulin superfamily molecules which possess complementary domains. Preferably, the present invention relates to antibodies.

A “domain” is a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. By single antibody variable domain is meant a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

The term “repertoire” refers to a collection of diverse variants, for example polypeptide variants which differ in their primary sequence. A library used in the present invention will encompass a repertoire of polypeptides comprising at least 1000 members.

The term “library” refers to a mixture of heterogeneous polypeptides or nucleic acids. The library is composed of members, which have a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members. Advantageously, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein, the term “ligand” refers to a compound that comprises at least one peptide, polypeptide, protein moiety that has a binding site with binding specificity for a desired target. For example, the ligand can comprise a (e.g., at least as one) protein moiety (e.g., a dAb) that has a binding site with-binding specificity for a receptor.

A “dual-specific ligand” is a ligand comprising a first immunoglobulin single variable domain and a second immunoglobulin single variable domain as herein defined, wherein the variable domains are capable of binding to two different antigens or two epitopes on the same antigen which are not normally bound by a monospecific immunoglobulin. For example, the two epitopes may be on the same hapten, but are not the same epitope or sufficiently adjacent to be bound by a monospecific ligand. The dual specific ligands according to the invention may comprise mutually complementary variable domain pairs which have different specificities, and do not contain mutually complementary variable domain pairs which have the same specificity.

The term “antigen” refers to a molecule that is bound by a ligand that binds to a small fraction of the members of a repertoire according to the present invention. It may be a polypeptide, protein, nucleic acid or other molecule. Generally, the ligands according to the invention are selected for target specificity against a particular antigen. In the case of conventional antibodies and fragments thereof, the antibody binding site defined by the variable loops (L1, L2, L3 and H1, H2, H3) is capable of binding to the antigen.

The term “epitope” refers to a unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation.

A “generic ligand” is a ligand that binds to all members of a repertoire. Generally, not bound through the antigen binding site as defined above. Examples include protein A and protein L.

As used herein, the term “selecting” means derived by screening, or derived by a Darwinian selection process, in which binding interactions are made between a domain and the antigen or epitope or between an antibody and an antigen or epitope. Thus a first variable domain may be selected for binding to an antigen or epitope in the presence or in the absence of a complementary variable domain.

A “universal framework” as used herein refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196: 910-917, The invention provides for the use of a single framework, or a set of such frameworks, which has been found to permit the derivation of virtually any binding specificity though variation in the hypervariable regions alone.

The phrase “immunoglobulin single variable domain” refers to an antibody variable domain (V_{H}, V_{HH}, V_{L}) that specifically binds a target, antigen or epitope independently of other V domains. An immunoglobulin single variable domain can be present in a format (e.g., hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” as the term is used herein. An “immunoglobulin single variable domain polypeptide”, as used herein refers to a mammalian immunoglobulin single variable domain, preferably human, but also rodent (for example, as disclosed in WO 00/29004, the contents of which are incorporated herein by reference in their entirety) or camelid V_{HH} dAbs.

“Camelid dAbs” are immunoglobulin single variable domain polypeptides which are derived from species including camel, llama, alpaca, dromedary, and guanaco, and comprise heavy chain antibodies naturally devoid of light chain (V_{HH}). Similar dAbs, can be obtained for single chain antibodies from other species, such as nurse shark.

As used herein, a “humanized” immunoglobulin single variable domain (dAb) comprises a framework region (e.g., FR1, FR2, FR3 and/or FR4) that is encoded by a human germline immunoglobulin gene segment but not a germline immunoglobulin gene segment of another species (e.g., a Camelid). The amino acid sequence of one or more of the complementarity determining regions (CDRs) of a humanized immunoglobulin single variable domain is the same as the amino acid sequence of the corresponding CDR of a non-human immunoglobulin variable domain (e.g., Camelid, murine) that has the same binding specificity as the humanized immunoglobulin single variable domain.

“Affinity” and “avidity” are terms of art that describe the strength of a binding interaction. With respect to the ligands of the invention, avidity refers to the overall strength of binding between the target(s) (e.g., first receptor and second receptor) on the cell and the ligand. Avidity is more than the sum of the individual affinities for the individual targets.

As used herein, “toxin moiety” refers to a moiety that comprises a toxin. A toxin is an agent that has deleterious effects on or alters cellular physiology (e.g., causes cellular necrosis, apoptosis or inhibits cellular division).

As used herein, the term “dose” refers to the quantity of ligand administered to a subject all at one time (unit dose), or in two or more administrations over a defined time interval. For example, dose can refer to the quantity of ligand administered to a subject over the course of one day (24 hours) (daily dose), two days, one week, two weeks, three weeks or one or more months (e.g., by a single administration, or by two or more administrations). The interval between doses can be any desired amount of time.

As used herein “receptor” refers to naturally occurring or endogenous proteins that are associated with the cell membrane (e.g., membrane bound proteins, integral membrane proteins, transmembrane proteins, glycophosphatidylinosital anchored proteins) and have binding specificity for a cognate ligand, and to proteins having an amino acid sequence which is the same as that of a naturally occurring or endogenous receptor protein (e.g., recombinant proteins, synthetic proteins (i.e., produced using the methods of synthetic organic chemistry)). Accordingly, as defined herein, the term includes mature receptor protein, naturally occurring polymorphic or allelic variants, and other naturally occurring isoforms of a receptor (e.g., produced by alternative splicing or other cellular processes), and modified (e.g. post-translational modifications, lipidated, glycosylated) or unmodified forms of the foregoing. Alternative splicing of RNA encoding a receptor may yield several isoforms of the receptor that differ in the number of amino acids in the protein sequence. These isoforms and other naturally occurring isoforms are expressly encompassed by the term “receptor”. Naturally occurring or endogenous receptors can be recovered or isolated from a source which naturally produces the receptor, for example. These proteins and proteins having the same amino acid sequence as a naturally occurring or endogenous corresponding receptor, are referred to by the name of the corresponding mammal. For example, where the corresponding mammal is a human, the protein is designated as a human receptor.

As used herein, the term “cognate ligand” refers to a naturally occurring endogenous ligand (e.g., protein, polypeptide) that binds to the ligand-binding site of a receptor, affects (e.g., induce, inhibits) receptor activity (e.g., signalling, adhesion) and is a component of the normal biochemical pathways that controls receptor activity. Examples of cognate ligands include growth factors (e.g., epidermal growth factor is a cognate ligand for epidermal growth factor receptor), cytokines, adhesion molecules and other soluble, cellular and matix proteins that bind receptors.

As used herein, “antibody” refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from serum, B-cells, hybridomas, transfectomas, yeast or bacteria.

The phrase, “half-life,” refers to the time taken for the serum concentration of the ligand to reduce by 50%, in vivo, for example due to degradation of the ligand and/or clearance or sequestration of the dual-specific ligand by natural mechanisms. The ligands of the invention are stabilized in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. Thus a ligand specific for HSA and a target molecule is compared with the same ligand wherein the specificity for HSA is not present, that is does not bind HSA but binds another molecule. For example, it may bind a second epitope on the target molecule. Typically, the half life is increased by 10%, 20%, 30%, 40%, 50% or more. Increases in the range of 2×, 3×, 4×, 5×, 10×, 20×, 30×, 40×, 50× or more of the half life are possible. Alternatively, or in addition, increases in the range of up to 30×, 40×, 50×, 60×, 70×, 80×, 90×, 100×, 150× of the half life are possible.

As referred to herein, the term “competes” means that the binding of a first binding agent (e.g., cognate ligand) to its binding site on a target (e.g., receptor) is inhibited when a second binding agent (e.g., dAb) is bound to its binding site on the target. For example, binding of the first binding agent may be inhibited sterically, for example by physical blocking of the binding site by the second binding agent, or by alteration of the structure or environment of a binding site caused by binding of the second binding agent, such that the affinity or avidity of the first agent for the target is reduced.

As used herein, the terms “low stringency,” “medium stringency,” “high stringency,” or “very high stringency conditions” describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and preferably (4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

Sequences similar or homologous (e.g., at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., very high stringency hybridization conditions), to the complement of the strand. The nucleic acids may be present in whole cells, in a cell lysate, or in a partially purified or substantially pure form.

Calculations of “homology” or “sequence identity” or “similarity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Amino acid and nucleotide sequence alignments and homology, similarity or identity, as defined herein are preferably prepared and determined using the algorithm BLAST 2 Sequences, using default parameters (Tatusova, T. A. et al., FEMS Microbiol Lett, 174:187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

### DETAILED DESCRIPTION

The invention relates to a ligand (e.g., an isolated domain antibody) that has binding specificity for a receptor (e.g., growth factor receptor, cytokine receptor, adhesion receptor, G protein-coupled receptor, receptor tyrosine kinase). The ligands generally comprise a polypeptide domain that has a binding site with binding specificity for a receptor. Preferred ligands are receptor inhibitors. Many receptors bind cognate ligands and cluster, i.e., form dimers, trimers or multimers, upon binding their cognate ligands (dimeric or multimeric receptor). For example, the PDGF receptor and TNF receptor superfamily members form dimers and trimers upon ligand binding, respectively. Cognate ligand-induced clustering (e.g., dimerization, multimerization) induces signal transduction through the receptor. Accordingly, the ligands of the invention can inhibit a receptor by, for example, inhibiting binding of cognate ligand, by inhibiting receptor clustering (e.g., dimerization, trimerization, multimerization) with or without also inhibiting cognate ligand binding, and/or inhibit receptor signalling.

The ligands of the invention (e.g., isolated domain antibody monomers) are promising therapeutic agents for the treatment of a variety of conditions, such as conditions associated with the expression, overexpression or activity of a receptor (e.g., inflammatory conditions, cancer). Unlike conventional antibodies, which can cause receptor clustering or cross-linking and activation upon binding of the antibody to the receptor, domain antibodies (e.g., dAb monomers) and certain ligands that comprise domain antibodies (e.g., monovalent ligands) do not substantially induce receptor clustering or cross-linking, and therefore, can antagonize a receptor without substantially agonizing the receptor.

For example, a ligand that binds receptor (e.g., EGFR, TNFR1) can be monovalent (e.g., a dAb monomer) and contain one binding site that binds receptor. Monovalent antagonists bind one chain of a receptor (e.g., dimeric receptor, trimeric receptor, multimeric receptor) and do not induce cross-linking or clustering of receptor chains on the surface of cells, which can lead to activation of the receptor and signal transduction. A ligand that inhibits receptor can also be multivalent and contain two or more copies of a particular binding site (e.g., dAb) for a receptor or contain two or more different binding sites that bind the receptor. For example, the ligand can be a dimer, trimer or multimer comprising two or more copies of a particular dAb that binds a receptor (e.g., TNFR1), or two or more different dAbs that bind receptor. Preferably, a multivalent antagonist of receptor does not substantially agonize the receptor (act as an agonist of the receptor by, for example, inducing receptor clustering and/or signalling) in a standard cell assay (i.e., when present at a concentration of 1 nM, 10 nM, 100 nM, 1 μM, 10 μM, 100 μM, 1000 μM or 5,000 μM (i.e., 5 mM), results in ≦5% of the receptor (e.g., TNFR1-mediated activity induced by cognate ligand (e.g., TNFα (100 pg/ml)) in the assay). Exemplary cell assays for TNFR1, EGFR and IL-1R are disclosed herein, and suitable assays for other receptors are known in the art.

The ligands of the invention provide several advantages in addition to not substantially clustering or cross-linking receptors. For example, dAbs are much smaller than conventional antibodies, and can be administered to achieve better tissue penetration than conventional antibodies. Thus, dAbs and ligands that comprise a dAb provide advantages over conventional antibodies when administered to treat cancer, for example by targeting solid tumors. Further, as described herein, a ligand of the invention can be tailored to have a desired in vivo serum half-life. Thus, the ligands can be used to control, reduce or eliminate general toxicity of therapeutic agents, such as a cytotoxin used to treat cancer.

Generally, the ligand comprises a polypeptide domain that has a binding site with binding specificity for a receptor, such as a binding domain based on an suitable scaffold, as described herein. Preferred ligands comprise or consist of a domain antibody (dAb) that has binding specificity for a receptor (e.g., a dAb monomer). In some embodiments, the ligand has binding specificity for a receptor and comprises two or more immunoglobulin single variable domain (dAb) with binding specificity for the receptor.

The ligand of the invention can be formatted as described herein. For example, the ligand can be formatted to tailor in vivo serum half-life. In particular embodiments, the ligand comprises an Fc portion of an antibody (e.g., an Fc portion of an IgG1, IgG2, IgG3 or IgG4) and/or is PEGylated.

The ligand of the invention (e.g., an isolated dAb) can have binding specificity for any desired receptor, such as a receptor tyrosine kinase, a receptor serine kinase, a G protein-coupled receptor, an adhesion receptor, or other receptor that is expressed on the surface of a cell (e.g., membrane bound). Generally the ligand (e.g., dAb) has binding specificity for a receptor that binds a cognate ligand. Preferably, the ligand inhibits the receptor, i.e., is an antagonist. For example, the ligand (e.g., dAb) can have binding specificity for a receptor included in the following list, or for a receptor that binds a cognate ligand included in the following list: ApoE, Apo-SAA, BDNF, Cardiotrophin-1, EGF, EGF receptor, ENA-78, Eotaxin, Eotaxin-2, Exodus-2, FGF-acidic, FGF-basic, fibroblast growth factor-10, FLT3 ligand, Fractalkine (CX3C), GDNF, G-CSF, GM-CSF, GF-β1, insulin, IFN-γ, IGF-I, IGF-II, IL-1α, IL-1β, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin α, Inhibin β, IP-10, keratinocyte growth factor-2 (KGF-2), KGF, Leptin, LIF, Lymphotactin, Mullerian inhibitory substance, monocyte colony inhibitory factor, monocyte attractant protein, M-CSF, MCP-1 (MCAF), MCP-2, MCP-3, MCP-4, MDC (67 a.a.), MDC (69 a.a.), MIG, MIP-1α, MIP-1β, MIP-3α, MIP-3β, MIP-4, myeloid progenitor inhibitor factor-1 (MPIF-1), NAP-2, Neurturin, Nerve growth factor, β-NGF, NT-3, NT-4, Oncostatin M, PDGF-AA, PDGF-AB, PDGF-BB, PF-4, RANTES, SDF1α, SDF1β, SCF, SCGF, stem cell factor (SCF), TARC, TGF-α, TGF-β, TGF-β2, TGF-β3, tumour necrosis factor (TNF), TNF-α, TNF-β, TNF receptor I, TNF receptor II, TNIL-1, TPO, VEGF, VEGF receptor 1, VEGF receptor 2, VEGF receptor 3, GCP-2, GRO/MGSA, GRO-β, GRO-γ, HCC1, 1-309, HER1, HER2, HER3, and HER4.

Additional receptors that the ligand (e.g., domain antibody) can have binding specificity for include the receptors in the following list, or a receptor that binds a cognate ligand included in the following list: EpoR, TACE recognition site, TNF BP-I, TNF BP-II, IL-1R1, IL-6R, IL-10R, IL-18R, IL-1, IL-19, IL-20, IL-21, IL-23, IL-24, IL-25, IL-27, IFN-γ, IFN-α/β, CD4, CD89, CD19, HLA-DR, CD38, CD138, CD33, CD56, CEA, and VEGF receptor.

Further receptors that the ligand (e.g., domain antibody) can have binding specificity for include gastrin releasing peptide receptor, neurotensin receptor, adrenomedullin receptor, H2 histamine receptor, HCG receptor, MET receptor, sphingosine 1-phosphate receptor, CD126, CD213a1, and KDR, among others.

The ligand can have binding specificity for a receptor that dimerizes upon binding to a cognate ligand (a dimeric receptor), or a receptor that forms multimers, such as trimers, upon binding to a cognate ligand (a multimeric receptor). Many cytokine receptors and growth factor receptors, such as members of the TNF receptor superfamily (e.g., TNFR1, TNFR2) and members of the receptor tyrosine kinase family (e.g., EGFR, PDGFR, M-CSF receptor (c-Fms)) form dimers or multimers upon binding their cognate ligands. The TNF receptor superfamily is an art recognized group of proteins that includes TNFR1 (p55, CD120a, p60, TNF receptor superfamily member 1A, TNFRSF1A), TNFR2 (p75, p80, CD120b, TNF receptor superfamily member 1B, TNFRSF1B), CD (TNFRSF3, LTβR, TNFR2-RP, TNFR-RP, TNFCR, TNF-R-III), OX40 (TNFRSF4, ACT35, TXGP1L), CD40 (TNFRSF5, p50, Bp50), Fas (CD95, TNFRSF6, APO-1, APTI), DcR3 (TNFRSF6B), CD27 (TNFRSF7, Tp55, S152), CD30 (TNFRSF8, Ki-1, D1S166E), CD137 (TNFRSF9, 4-1BB, ILA), TRAILR-1 (TNFRSF10A, DR4, Apo2), TRAIL-R2 (TNFRSF10B, DR5, KILLER, TRICK2A, TRICKB), TRAILR3 (TNFRSF10C, DcR1, LIT, TRID), TRAILR4 (TNFRSF10D, DcR2, TRUNDD), RANK (TNFRSF11A), OPG (TNFRSF11B, OCIF, TR1), DR3 (TNFRSF12, TRAMP, WSL-1, LARD, WSL-LR, DDR3, TR3, APO-3), DR3L (TNFRSF12L), TAC1 (TNFRSF13B), BAFFR (TNFRSF13C), HVEM (TNFRSF14, ATAR, TR2, LIGHTR, HVEA), NGFR (TNFRSF16), BCMA (TNFRSF17, BCM), AITR (TNFRSF18, GITR), TNFRSF19, FLJ14993 (TNFRSF19L, RELT), DR6 (TNFRSF21), SOBa (TNFRSF22, Tnfrh2, 2810028K06Rik), and mSOB (THFRSF23, Tnfrh1). The receptor tyrosine kinase family is an art recognized group of proteins that includes EGFR (ERBB1, HER1), PDGFR, c-Fms, FGFR1, FGFR2, FGFR3, FGFR4, Insulin receptor, and Insulin-like growth factor receptors (IGF1R, IGF2R). See, Grassot et al., Nucleic Acids Research, 31(1):353-358 (2003).

Preferably, the ligand binds a dimeric or multimeric receptor and inhibits the receptor. For example, the ligand can inhibit binding of cognate ligand to receptor, inhibit clustering (e.g., dimerization, trimerization or multimerization) of receptor upon ligand binding, and/or inhibit receptor signaling. In some embodiments, the ligand is monovalent for the target receptor (e.g., contains one binding site for the receptor) and does not induce receptor clustering or cross-linking. As described herein, such a ligand can contain additional binding sites, such as a binding site for a polypeptide that increases in vivo serum half life (e.g., serum albumin) or a different receptor if desired. In one example, the ligand is an isolated dAb monomer that binds receptor (e.g., EGFR, TNFR1, IL-1R1) and does not induce receptor clustering.

For illustrative purposes, the binding specificity and activities of exemplary ligands of the inventions are further described below with reference to embodiments that bind TNFR1. It should be understood that the binding specificities and biological functions of the described embodiments are generally applicable to ligands (e.g., dAbs) that bind a desired receptor, in particular, ligands that bind other members of the TNF receptor super family.

The extracellular region of TNFR1 comprises a thirteen amino acid amino-terminal segment (amino acids 1-13 of SEQ ID NO:30 (human); amino acids 1-13 of SEQ ID NO:31 (mouse)), Domain 1 (amino acids 14-53 of SEQ ID NO:30 (human); amino acids 14-53 of SEQ ID NO:31 (mouse)), Domain 2 (amino acids 54-97 of SEQ ID NO:30 (human); amino acids 54-97 of SEQ ID NO:31 (mouse)), Domain 3 (amino acids 98-138 of SEQ ID NO:30 (human); amino acid 98-138 of SEQ ID NO:31 (mouse)), and Domain 4 (amino acids 139-167 of SEQ ID NO:30 (human); amino acids 139-167 of SEQ ID NO:31 (mouse)) which is followed by a membrane-proximal region (amino acids 168-182 of SEQ ID NO:30 (human); amino acids 168-183 SEQ ID NO:31 (mouse)). (See, Banner et al., Cell 73(3) 431-445 (1993) and Loetscher et al., Cell 61(2) 351-359 (1990).) Domains 2 and 3 make contact with bound ligand (TNFβ, TNFα). (Banner et al., Cell, 73(3) 431-445 (1993).) The extracellular region of TNFR1 also contains a region referred to as the pre-ligand binding assembly domain or PLAD domain (amino acids 1-53 of SEQ ID NO:30 (human); amino acids 1-53 of SEQ ID NO:31 (mouse)). See, e.g., WO 01/58953; Deng et al., Nature Medicine, doi: 10.1038/nm1304 (2005)).

In some embodiments, the ligand (e.g., dAb monomer) binds TNFR1 but does not compete with TNF for binding to TNFR1. For example, the ligand (e.g., dAb monomer) can bind Domain 1 of TNFR1 or Domain 4 of TNFR1. Such ligands (e.g., dAb) provide advantages as diagnostic agents, and can be used to bind and detect, quantify or measure TNFR1 in a sample but will not compete with TNF in the sample for binding to TNFR1. Accordingly, an accurate determination of whether TNFR1 is present in the sample or how much TNFR1 is in the sample can be made.

In certain embodiments, the ligand is multivalent and contains two or more binding sites (e.g, dAbs) for a desired epitope or domain of TNFR1. For example, the multivalent ligand can comprise two or more binding sites that bind the same epitope in Domain 1 of TNFR1. In other embodiments, the multivalent ligand contains two or more binding sites that bind to different epitopes or domains of TNFR1. In one example, the multivalent ligand comprises a first binding site (e.g., a first dAb) that binds a first epitope in Domain 1 of TNFR1, and a second binding site (e.g., a second dAb) that binds a second different epitope in Domain 1. In other examples, the multivalent ligand comprises binding sites (e.g., dAbs) for Domains 1 and 2, Domains 1 and 3, Domains 1 and 4, Domains 2 and 3, Domains 2 and 4, or Domains 3 and 4 of TNFR1. In additional examples, the multivalent ligand comprises binding sites (e.g., dAbs) for Domains 1, 2, and 3, binding sites (e.g., dAbs) for Domains 1, 2 and 4, or binding sites (e.g., dAbs) for Domains 1, 3 and 4 of TNFR1. Preferably, such multivalent antagonists do not agonize TNFR1 when present at a concentration of about 1 nM, or about 10 nM, or about 100 nM, or about 1 μM, or about 10 μM, in a standard L929 cytotoxicity assay or a standard HeLa IL-8 assay as described herein.

In some embodiments, the ligand (e.g., dAb monomer) binds Domain 2 and/or Domain 3 of TNFR1. This type of ligand inhibits binding of TNFα to TNFR1. In particular embodiments, the antagonist competes with TAR2h-10-27, TAR2h-131-8, TAR2h-15-8, TAR2h-35-4, TAR2h-154-7, TAR2h-154-10 or TAR2h-185-25 for binding to TNFR1.

In other embodiments, the ligand (e.g., dAb monomer) binds Domain 1 and/or Domain 4 of TNFR1. This type of ligand does not inhibit binding of TNFα to TNFR1. This type of ligand can be used to inhibit clustering and/or signal transduction mediated through TNFR1, but not inhibit binding of TNFα to TNFR1.

In other embodiments, the ligand (e.g., dAb monomer) binds TNFR1, but does not bind in Domain 4 (e.g., binds in Domain 1, 2, 3 or the PLAD Domain). Such antagonists inhibit ligand-binding, clustering and/or signalling through TNFR1, but do not inhibit shedding of soluble TNFR1. Accordingly, administering such an antagonist to a mammal in need thereof can complement the endogenous regulatory pathways that inhibit the activity TNFα and the activity of TNFR1 in vivo.

In particular embodiments, the ligand inhibits clustering (e.g., ligand-induced dimerization or multimerization) of receptor, but does not inhibit binding of cognate ligand. For example, the ligand (e.g., dAb monomer) can bind the PLAD domain of a receptor that is a member of the TNF receptor superfamily (e.g., TNFR1) but not inhibit binding of ligand (e.g., TNFα) to the receptor.

As described above with respect to embodiments that bind TNFR1, the ligands (e.g., dAbs) of the invention can bind any desired region of a desired target receptor and can inhibit the receptor by inhibiting binding of cognate ligand, inhibiting receptor clustering, and/or inhibiting receptor signalling. For example, ligands that bind receptor tyrosine kinases can bind extracellular Ig-like domains, L domains, furin-like repeats, cystein-rich domains, fibronectin type III domains, ephrin binding domains or other domains that are well-known components of the extracellular portion of receptor tyrosine kinases. See, e.g., Grassot et al., Nucleic Acids Research, 31(1):353-358 (2003). For example, ligands that bind EGFR (e.g. an isolated dAb monomer) can bind an L domain or furin-like repeat in the extracellular portion of EGFR, and can inhibit binding of cognate ligand (e.g., EGF) to EGFR, inhibit EGFR clustering and/or inhibit EGFR signalling.

In particular embodiments, the ligand has binding specificity for a cytokine receptor or a growth factor receptor (i.e., the cognate ligand is a cytokine or growth factor). Preferably, the ligand inhibits the cytokine receptor or growth factor receptor. Suitable cytokine receptors and growth factor receptors include the receptors disclosed herein and receptors for the cytokines and growth factors disclosed herein. For example, in some embodiments, the ligand has binding specificity for a cytokine receptor or growth factor receptor selected from the group consisting of HER1 (EGF receptor), HER2, HER3, HER4, TNF receptor I, TNF receptor II, VEGF receptor 1, VEGF receptor 2, and VEGF receptor 3. In other embodiments, the ligand has binding specificity for the receptor of a cytokine or growth factor selected from the group consisting of EGF, TNFα, TNFβ, IGF-I, IGF-II, IL-1α, IL-1β, MCS-F, TGF-α, TGF-β, TGF-β2, and TGF-β3. In other embodiments, the ligand has binding specificity for a cytokine receptor or growth factor receptor selected from the group consisting of EpoR, TACE recognition site, TNF BP-I, TNF BP-II, IL-1R1, IL-6R, IL-10R, and IL-18R.

A ligand (e.g., an isolated dAb) that has binding specificity for a receptor can antagonize the receptor. For example, certain ligands antagonize the receptor without substantially agonizing the receptor. A ligand dose not substantially agonize a receptor when the ligand, at a concentration of 1 nM, 10 nM, 100 nM, 1 μM, 10 μM, 100 μM, 1000 μM or 5,000 μM, induces no more than about 5% of the receptor-mediated activity that is induced by the cognate ligand of the receptor (e.g., at about 100 pg/ml) in a standard receptor activity assay, such as a cell-based assay. Suitable receptor-specific assays for assessing the activity of receptor are known in the art and include, for example, the EGFR, TNFR1 and IL-1R1 activity and binding assays disclosed herein.

Some ligands comprise a polypeptide domain that has a binding site with binding specificity for a receptor that is provided by an antibody fragment, such as an immunoglobulin single variable domain with binding specificity for the receptor (dAb). The dAb can comprise an immunoglobulin heavy chain V region, such as a Camelid V_{HH} or a human V_{H}, or an immunoglobulin light chain V region, such as a Vλ or Vκ. In particular embodiments, the ligand does not comprise a Camelid V_{HH}.

In some embodiments, the ligand comprises a humanized immunoglobulin single variable domain with binding specificity for receptor, preferably a human receptor. In preferred embodiments, the ligand comprises a human immunoglobulin single variable domain with binding specificity for receptor, preferably a human receptor.

As described herein, the dAb can be an immunoglobulin heavy chain V region of the VH3 family, such as the DP-47 V_{H} segment with JH4b J_{H} segment. Other suitable immunoglobulin heavy chain V regions can comprise the DP45 or DP38 V_{H} segment. As described herein, preferred immunoglobulin heavy chain V regions comprise a hypervariable loop (e.g., H3) that has the canonical structure of the H3 loop of DP47 and JH4b. Optionally, such an immunoglobulin heavy chain V region further comprises another hypervariable loop (e.g., H1 and/or H2) that has the canonical structure of the corresponding hypervariable loop of DP47. In particular examples, the immunoglobulin heavy chain V regions comprise an H3 loop that has the canonical structure of the H3 loop of DP47 and JH4b, and H1 and H2 loops that have the canonical structure of the H1 and H2 loops of DP47.

The ligand that has binding specificity for a receptor (e.g., EGFR) can comprise a universal framework. For example, the universal framework can comprise a DPK9 V_{L} framework, or a V_{H} framework selected from the group consisting of DP47, DP45 and DP38.

The ligand can comprise an antibody variable domain having one or more framework regions comprising an amino acid sequence that is the same as the amino acid sequence of a corresponding framework region encoded by a human germline antibody gene segment, or the amino acid sequences of one or more of said framework regions collectively comprise up to 5 amino acid differences relative to the amino acid sequence of said corresponding framework region encoded by a human germline antibody gene segment.

In some embodiments, the ligand comprises an antibody variable domain, wherein the amino acid sequences of FW1, FW2, FW3 and FW4 are the same as the amino acid sequences of corresponding framework regions encoded by a human germline antibody gene segment, or the amino acid sequences of FW1, FW2, FW3 and FW4 collectively contain up to 10 amino acid differences relative to the amino acid sequences of corresponding framework regions encoded by said human germline antibody gene segment. In other embodiments, the ligand comprises an antibody variable domain comprising FW1, FW2 and FW3 regions, and the and the amino acid sequence of said FW1, FW2 and FW3 are the same as the amino acid sequences of corresponding framework regions encoded by human germline antibody gene segments. In particular embodiments, the human germline antibody gene segment is selected from the group consisting from the group consisting of DP38, DP47, DP45, DP48 and DPK9. The ligands of the invention can also include a terminal Cys residue and/or an antibody Fc region.

The ligands of the invention (e.g., dAbs) can comprise a polypeptide domain that has a binding site with binding specificity for a receptor (e.g., a dAb) that binds the receptor with high affinity. In preferred embodiments, the binding domain (e.g., dAb) binds to a receptor with an affinity (KD; KD=K_{off}(kd)/Kₒₙ(ka)) of 300 nM to 1 pM (i.e., 3×10⁻⁷ to 1×10⁻¹²M), preferably 300 nM to 5 pM or 50 nM to 1 pM, more preferably 5 nM to 1 pM and most preferably 1 nM to 1 pM, for example and a K_{D} of 1×10⁻⁷ M or less, preferably 1×10⁻⁸ M or less, more preferably 1×10⁻⁹ M or less, advantageously 1×10⁻¹⁰ M or less, and most preferably 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance.

### Ligand Formats

The ligand of the invention (e.g., a dAb monomer) can be formatted as a monospecific, dual specific or multispecific ligand, and/or as monovalent, bivalent or multivalent ligand as described herein. The ligand can be dual specific or multispecific, but be monovalent for a target receptor. For example, this type of ligand can comprise one binding site for the target receptor and one or more additional binding sites that bind, for example, a polypeptide the increases in vivo serum half life. As described herein, ligands that are monovalent for a target receptor (e.g., a dAb monomer) do not induce receptor clustering.

In particular embodiments, the ligands of the invention are dual specific ligands. Such dual specific ligands comprise immunoglobulin single variable domains that have different binding specificities. For example, a dual specific ligand can have binding specificity for a receptor and an antigen or epitope (e.g., from a half-life extending moiety) which increases the half-life of said ligand (e.g., serum albumin). Dual specific ligands can comprise combinations of heavy and light chain domains. For example, the dual specific ligand may comprise a V_{H} domain and a V_{L} domain, which may be linked together in the form of an scFv (e.g., using a suitable linker such as Gly₄Ser), or formatted into a bispecific antibody or antigen-binding fragment thereof (e.g. F(ab′)₂ fragment). The dual specific ligands do not comprise complementary V_{H}/V_{L} pairs which form a conventional two chain antibody antigen-binding site that binds antigen or epitope co-operatively. Instead, the dual specific ligands comprise two single V domains that have different binding specificities.

In addition, the dual specific ligands may comprise one or more C_{H} or C_{L} domains if desired. A hinge region domain may also be included if desired. Such combinations of domains may, for example, mimic natural antibodies, such as IgG or IgM, or fragments thereof, such as Fv, scFv, Fab or F(ab′)₂ molecules. Other structures, such as a single arm of an IgG molecule comprising V_{H}, V_{L}, C_{H}1 and C_{L} domains, are envisaged. In some embodiments, the dual specific ligand of the invention comprises only two variable domains although several such ligands may be incorporated together into the same protein, for example two such ligands can be incorporated into an IgG or a multimeric immunoglobulin, such as IgM. In another embodiment, a plurality of ligands (e.g., dual specific ligands) are combined to form a multimer. For example, two or more different mono-, dual-, or multi-specific ligands can be combined to create a multispecific (e.g., tri-specific, tetra-specific) molecule. It will be appreciated by one skilled in the art that the light and heavy variable regions of a mono-, dual-, or multi-specific ligands produced according to the method of the present invention may be on the same polypeptide chain, or alternatively, on different polypeptide chains. In the case that the variable regions are on different polypeptide chains, then they may be linked via a linker, generally a flexible linker (such as a polypeptide chain), a chemical linking group, or any other method known in the art.

Ligands can be formatted as mono-, bi- or multispecific (or mono-, bi- or multivalent) antibodies or antibody fragments or into mono-, bi- or multispecific (or mono-, bi- or multivalent) non-antibody structures. Suitable formats include, any suitable polypeptide structure in which an antibody variable domain or one or more of the CDRs thereof can be incorporated so as to confer binding specificity for antigen on the structure. A variety of suitable antibody formats are known in the art, such as, mono-, bi- or multi-specific IgG-like formats (e.g., chimeric antibodies, humanized antibodies, human antibodies, single chain antibodies, heterodimers of antibody heavy chains and/or light chains, antigen-binding fragments of any of the foregoing (e.g., a Fv fragment (e.g., single chain Fv (scFv), a disulfide bonded Fv), a Fab fragment, a Fab′ fragment, a F(ab′)₂ fragment), a dAb or single variable domain (e.g., V_{H}, V_{L}, V_{HH}), and modified versions of any of the foregoing (e.g., modified by the covalent attachment of polyalkylene glycol (e.g., polyethylene glycol, polypropylene glycol, polybutylene glycol) or other suitable polymer). See, PCT/GB03/002804, filed Jun. 30, 2003, which designated the United States, (WO 2004/081026) regarding PEGylated single variable domains and dAbs, suitable methods for preparing same, increased in vivo half life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teachings of PCT/GB03/002804 (WO 2004/081026), including the portions referred to above, are incorporated herein by reference.

The ligand can be formatted using a suitable linker such as (Gly₄Ser)ₙ, where n is from 1 to 8, e.g., 2, 3, 4, 5, 6 or 7. If desired, ligands, including dAb monomers, dimers and trimers, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides.

Ligands can be combined and/or formatted into non-antibody multi-ligand structures to form multivalent complexes, which provide superior avidity. For example natural bacterial receptors such as SpA can been used as scaffolds for the grafting of CDRs to generate ligands which bind specifically to one or more epitopes. Details of this procedure are described in U.S. Pat. No. 5,831,012. Other suitable scaffolds include those based on fibronectin and affibodies. Details of suitable procedures are described in WO 98/58965. Other suitable scaffolds include lipocallin and CTLA4, as described in van den Beuken et al., J. Mol. Biol. 310:591-601 (2001), and scaffolds such as those described in WO 00/69907 (Medical Research Council), which are based for example on the ring structure of bacterial GroEL or other chaperone polypeptides. Protein scaffolds may be combined; for example, CDRs may be grafted on to a CTLA4 scaffold and used together with immunoglobulin V_{H} or V_{L} domains to form a ligand. Likewise, fibronectin, lipocallin and other scaffolds (e.g., immunoglobulin domains, those based on fibronectin, those based on affibodies, those based on CTLA4, those based on chaperones such as GroEL, those based on lipocallin and those based on the bacterial Fc receptors SpA and SpD, an SpA scaffold, an LDL receptor class A domain, an EGF domain, and avimer (see, e.g., U.S. Patent Application Publication Nos. 2005/0053973, 2005/0089932, 2005/0164301)) can be combined.

A variety of suitable methods for preparing any desired format are known in the art. For example, antibody chains and formats (e.g., mono-, bi- or multi-specific IgG-like formats, chimeric antibodies, humanized antibodies, human antibodies, single chain antibodies, homodimers and heterodimers of antibody heavy chains and/or light chains) can be prepared by expression of suitable expression constructs and/or culture of suitable cells (e.g., hybridomas, heterohybridomas, recombinant host cells containing recombinant constructs encoding the format). Other formats, such as antigen-binding fragments of antibodies or antibody chains (e.g., bispecific binding fragments, such as a Fv fragment (e.g., single chain Fv (scFv), a disulfide bonded Fv), a Fab fragment, a Fab′ fragment, a F(ab′)₂ fragment), can be prepared by expression of suitable expression constructs or by enzymatic digestion of antibodies, for example using papain or pepsin.

The ligand can be formatted as a multispecific ligand, for example as described in WO 03/002609, the entire teachings of which are incorporated herein by reference. Such multispecific ligands possess more than one epitope binding specificity. Generally, the multi-specific ligand comprises two or more epitope binding domains, such as dAbs or non-antibody protein domain comprising a binding site for an epitope, e.g., an affibody, an SpA domain, an LDL receptor class A domain, an EGF domain, an avimer. Multispecific ligands can be formatted further as described herein.

In some embodiments, the ligand is an IgG-like format. Such formats have the conventional four chain structure of an IgG molecule (2 heavy chains and two light chains), in which one or more of the variable domains (V_{H} and or V_{L}) have been replaced with a dAb or immunoglobulin single variable domain of a desired specificity. Preferably, each of the variable domains (2 V_{H} regions and 2 V_{L} regions) is replaced with a dAb or immunoglobulin single variable domain. The dAb(s) or immunoglobulin single variable domain(s) that are included in an IgG-like format can have the same specificity or different specificities. In some embodiments, the IgG-like format is tetravalent and can have one, two, three or four specificities. For example, the IgG-like format can be monospecific and comprise 4 dAbs that have the same specificity; bispecific and comprise 3 dAbs that have the same specificity and another dAb that has a different specificity; bispecific and comprise two first dAbs that have the same specificity and two second dAbs that have a common but different specificity; trispecific and comprises first and second dAbs that have the same specificity, a third dAbs with a different specificity and a fourth dAb with a different specificity from the first, second and third dAbs; or tetraspecific and comprise four dAbs that each have a different specificity. In a particular example, the IgG-like format is monospecific and tetravalent, and comprises four copies of a dAb or immunoglobulin single variable domain that binds a receptor (e.g., EGFR, TNFR1)

Antigen-binding fragments of IgG-like formats (e.g., Fab, F(ab′)₂, Fab′, Fv, scFv) can be prepared. In addition, a particular constant region or Fc portion (e.g., of an IgG, such as IgG1), variant or portion thereof can be selected in order to tailor effector function. For example, if complement activation and/or antibody dependent cellular cytotoxicity (ADCC) function is desired, the ligand can be an IgG1-like format. If desired, the IgG-like format can comprise a mutated constant region (variant IgG heavy chain constant region) to minimize binding to Fc receptors and/or ability to fix complement (see e.g Winter et al, GB 2,209,757 B; Morrison et al., WO 89/07142; Morgan et al., WO 94/29351, Dec. 22, 1994).

The ligands of the invention can be formatted as a fusion protein that contains a first dAb or immunoglobulin single variable domain that is fused directly to a second dAb or immunoglobulin single variable domain. If desired such a format can further comprise a half life extending moiety. For example, the ligand can comprise three dAbs or immunoglobulin single variable domains that are directly fused to form a fusion protein, wherein the first dAb or immunoglobulin single variable domain binds a receptor, the second dAb or immunoglobulin single variable domain that binds the same or different receptor, and the third dAb or immunoglobulin single variable domain binds serum albumin. In one example, the ligand comprises two copies of a dAb or immunoglobulin single variable domain that binds a receptor (e.g., EGFR, TNFR1) and a dAb or immunoglobulin single variable domain that binds serum albumin. If desired, suitable peptide linkers can be incorporated between the dAbs of a fusion protein.

Generally the orientation of the binding domains (e.g., dAbs) that have a binding site with binding specificity receptor and/or the binding domains (e.g., dAbs) that have a binding site with binding specificity polypeptide that enhances half-life in vivo, and whether the ligand comprises a linker, is a matter of design choice. However, some orientations, with or without linkers, may provide better binding characteristics than other orientations. All orientations (e.g., dAb1-linker-dAb2; dAb2-linker-dAb1) are encompassed by the invention. Ligands that contain an orientation that provides desired binding characteristics can be easily identified by screening.

Ligands according to the invention, including isolated dAb monomers, can be provided as dimers (e.g., dAb dimers), trimers or other multimers (e.g., polymers). For example, variable domains may be linked together to form multivalent ligands by, for example, provision of a hinge region at the C-terminus of each V domain and disulphide bonding between cysteines in the hinge regions; or provision of dAbs each with a cysteine at the C-terminus of the domain, the cysteines being disulphide bonded together; or production of V-CH & V-CL to produce a Fab format; or use of peptide linkers (for example Gly₄Ser linkers discussed herein) to produce dimers, trimers and further multimers. For example, such ligands can be linked to an antibody Fc region comprising one or both of C_{H}2 and CH3 domains, and optionally a hinge region. For example, vectors encoding ligands linked as a single nucleotide sequence to an Fc region may be used to prepare such ligands (e.g., by expression).

Ligand formats specific for multiple copies of the same epitope, or adjacent epitopes, on the same target (known as chelating dAbs) may also be trimeric or polymeric (tertrameric or more) ligands comprising three, four or more non-complementary binding domains. For example, ligands may be constructed comprising three or four V_{H} domains or V_{L} domains.

Ligand formats can comprise multiple binding domains which bind to multisubunit receptors, wherein each binding domain is specific for a subunit of said receptor. Such ligands may be dimeric, trimeric or polymeric.

If desired, any of the ligands described herein can further comprise a toxin. In particular embodiments, the toxin is a surface active toxin (e.g., a surface active toxin comprising a free radical generator, a surface active toxin comprising a radionuclide). Suitable toxins include, but are not limited to a cytotoxin, free radical generator, antimetabolite, protein, polypeptide, peptide, photoactive agent, antisense compound, chemotherapeutic, radionuclide or intrabody (e.g., an intracellular antibody).

The invention also relates to ligands that comprise a toxin moiety or toxin. Suitable toxin moieties comprise a toxin (e.g., surface active toxin, cytotoxin). The toxin moiety or toxin can be linked or conjugated to the ligand using any suitable method. For example, the toxin moiety or toxin can be covalently bonded to the ligand directly or through a suitable linker. Suitable linkers can include noncleavable or cleavable linkers, for example, pH cleavable linkers that comprise a cleavage site for a cellular enzyme (e.g., cellular esterases, cellular proteases such as cathepsin B). Such cleavable linkers can be used to prepare a ligand that can release a toxin moiety or toxin after the ligand is internalized.

A variety of methods for linking or conjugating a toxin moiety or toxin to a ligand can be used. The particular method selected will depend on the toxin moiety or toxin and ligand to be linked or conjugated. If desired, linkers that contain terminal functional groups can be used to link the ligand and toxin moiety or toxin. Generally, conjugation is accomplished by reacting toxin moiety or toxin that contains a reactive functional group (or is modified to contain a reactive functional group) with a linker or directly with a ligand. Covalent bonds formed by reacting an toxin moiety or toxin that contains (or is modified to contain) a chemical moiety or functional group that can, under appropriate conditions, react with a second chemical group thereby forming a covalent bond. If desired, a suitable reactive chemical group can be added to ligand or to a linker using any suitable method. (See, e.g., Hermanson, G. T., Bioconjugate Techniques, Academic Press: San Diego, Calif. (1996).) Many suitable reactive chemical group combinations are known in the art, for example an amine group can react with an electrophilic group such as tosylate, mesylate, halo (chloro, bromo, fluoro, iodo), N-hydroxysuccinimidyl ester (NHS), and the like. Thiols can react with maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

Suitable toxin moieties and toxins include, for example, a maytansinoid (e.g., maytansinol, e.g., DM1, DM4), a taxane, a calicheamicin, a duocarmycin, or derivatives thereof. The maytansinoid can be, for example, maytansinol or a maytansinol analogue. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U.S. Pat. Nos. 5,208,020 and 6,333,410, the contents of which is incorporated herein by reference. Maytansinol can be coupled to antibodies and antibody fragments using, e.g., an N-succinimidyl 3-(2-pyridyldithio)proprionate (also known as N-succinimidyl 4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2 iminothiolane, or S-acetylsuccinic anhydride. The taxane can be, for example, a taxol, taxotere, or novel taxane (see, e.g., WO 01/38318). The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include I1-BR, I2-BR, I3-BR, I4-BR, J1-BR, J2-BR and K1-BR. Iodo-complex calicheamicins include I1-I, I2-I, I3-I, J1-I, J2-I, L1-I and K1-BR. Calicheamicin and mutants, analogs and mimics thereof are described, for example, in U.S. Pat. Nos. 4,970,198; 5,264,586; 5,550,246; 5,712,374, and 5,714,586, the contents of each of which are incorporated herein by reference. Duocarmycin analogs (e.g., KW-2189, DC88, DC89 CBI-TMI, and derivatives thereof are described, for example, in U.S. Pat. No. 5,070,092, U.S. Pat. No. 5,187,186, U.S. Pat. No. 5,641,780, U.S. Pat. No. 5,641,780, U.S. Pat. No. 4,923,990, and U.S. Pat. No. 5,101,038, the contents of each of which are incorporated herein by reference.

Examples of other toxins include, but are not limited to antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065 (see U.S. Pat. Nos. 5,475,092, 5,585,499, 5,846,545), melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, mitomycin, puromycin anthramycin (AMC)), duocarmycin and analogs or derivatives thereof, and anti-mitotic agents (e.g., vincristine, vinblastine, taxol, auristatins (e.g., auristatin E) and maytansinoids, and analogs or homologs thereof.

The toxin can also be a surface active toxin, such as a toxin that is a free radical generator (e.g. selenium containing toxin moieties), or radionuclide containing moiety. Suitable radionuclide containing moieties, include for example, moieties that contain radioactive iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

The toxin can be a protein, polypeptide or peptide, from bacterial sources, e.g., diphtheria toxin, pseudomonas exotoxin (PE) and plant proteins, e.g., the A chain of ricin (RTA), the ribosome inactivating proteins (RIPs) gelonin, pokeweed antiviral protein, saporin, and dodecandron are contemplated for use as toxins.

Antisense compounds of nucleic acids designed to bind, disable, promote degradation or prevent the production of the mRNA responsible for generating a particular target protein can also be used as a toxin. Antisense compounds include antisense RNA or DNA, single or double stranded, oligonucleotides, or their analogs, which can hybridize specifically to individual mRNA species and prevent transcription and/or RNA processing of the mRNA species and/or translation of the encoded polypeptide and thereby effect a reduction in the amount of the respective encoded polypeptide. Ching, et al., Proc. Natl. Acad. Sci. U.S.A. 86: 10006-10010 (1989); Broder, et al., Ann. Int. Med. 113: 604-618 (1990); Loreau, et al., FEBS Letters 274: 53-56 (1990); Useful antisense therapeutics include for example: Veglin™ (VasGene) and OGX-011 (Oncogenix).

Toxins can also be photoactive agents. Suitable photoactive agents include porphyrin-based materials such as porfimer sodium, the green porphyrins, chlorin E6, hematoporphyrin derivative itself, phthalocyanines, etiopurpurins, texaphrin, and the like.

The toxin can be an antibody or antibody fragment that binds an intracellular target, such as a dAb that binds an intracellular target (an intrabody). Such antibodies or antibody fragments (dAbs) can be directed to defined subcellular compartments or targets. For example, the antibodies or antibody fragments (dAbs) can bind an intracellular target selected from erbB2, EGFR, BCR-ABL, p21Ras, Caspase3, Caspase7, Bcl-2, p53, Cyclin E, ATF-1/CREB, HPV16 E7, HP1, Type IV collagenases, cathepsin L as well as others described in Kontermann, R. E., Methods, 34:163-170 (2004), incorporated herein by reference in its entirety.

The ligands of the invention can comprise a half-life extending moiety, such as a polyalkylene glycol moiety, serum albumin or a fragment thereof, transferrin receptor or a transferrin-binding portion thereof. In other embodiments, the ligands of the invention contain a moiety comprising a binding site for a polypeptide that enhances half-life in vivo (e.g., an affibody, an SpA domain, an LDL receptor class A domain, an EGF domain, an avimer, polyethylene glycol, serum albumin, neonatal Fc receptor). Suitable types of moieties that comprise a binding site for a polypeptide that enhances half-life in vivo include, but are not limited to, antibody fragments, dAbs and immunoglobulin single variable domains.

### Half-life Extended Formats

The ligands disclosed herein can be formatted to extend its in vivo serum half life. Increased in vivo half-life is useful in in vivo applications, especially in vivo applications of antibodies and most especially antibody fragments of small size. Such fragments (Fvs, disulphide bonded Fvs, Fabs, scFvs, dAbs) are rapidly cleared from the body, which can limit clinical use.

A ligand can be formatted as a larger antigen-binding fragment of an antibody or as an antibody (e.g., formatted as a Fab, Fab′, F(ab)₂, F(ab′)₂, IgG, scFv) that has larger hydrodynamic size. Ligands can also be formatted to have a larger hydrodynamic size, for example, by attachment of a polyalkyleneglycol group (e.g. polyethyleneglycol (PEG) group, polypropylene glycol, polybutylene glycol), serum albumin, transferrin, transferrin receptor or at least the transferrin-binding portion thereof, an antibody Fc region, or by conjugation to an antibody domain. In some embodiments, the ligand (e.g., dAb) is PEGylated. Preferably the PEGylated ligand (e.g., dAb) binds a receptor (e.g., EGFR) with substantially the same affinity or avidity as the same ligand that is not PEGylated. For example, the ligand can be a PEGylated ligand comprising a dAb that binds a receptor with an affinity or avidity that differs from the affinity or avidity of ligand in unPEGylated form by no more than a factor of about 1000, preferably no more than a factor of about 100, more preferably no more than a factor of about 10, or with affinity or avidity substantially unchanged relative to the unPEGylated form. See, PCT/GB03/002804, filed Jun. 30, 2003, which designated the United States, (WO 2004/081026) regarding PEGylated single variable domains and dAbs, suitable methods for preparing same, increased in vivo half life of the PEGylated single variable domains and dAb monomers and multimers, suitable PEGs, preferred hydrodynamic sizes of PEGs, and preferred hydrodynamic sizes of PEGylated single variable domains and dAb monomers and multimers. The entire teachings of PCT/GB03/002804 (WO 2004/081026), including the portions referred to above, are incorporated herein by reference.

Hydrodynamic size of the ligands (e.g., dAb monomers and multimers) of the invention may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

The size of a ligand format (e.g., the size of a PEG moiety attached to a dAb monomer), can be varied depending on the desired application. For example, where ligand is intended to leave the circulation and enter into peripheral tissues, it is desirable to keep the hydrodynamic size of the ligand low to facilitate extravazation from the blood stream. When it is desired that the ligand remain in the systemic circulation for a longer period of time, the size of the ligand can be increased, for example by formatting as and Ig-like protein or by addition of a 30 to 60 kDa PEG moiety (e.g., linear or branched 30 to 40 kDa PEG, such as addition of two 20 kDa PEG moieties.) The size of the ligand format can be tailored to achieve a desired in vivo serum half life, for example to control exposure to a toxin and/or to reduce side effects of toxic agents.

Examples of suitable albumin, albumin fragments or albumin variants for use in a ligand according to the invention are described in WO 2005/077042A2, which is incorporated herein by reference in its entirety. In particular, the following albumin, albumin fragments or albumin variants can be used in the present invention:
SEQ ID NO:32 (disclosed as SEQ ID NO:365 in WO 2005/077042A2, this sequence being explicitly incorporated into the present disclosure by reference);
a Albumin fragment or variant comprising or consisting of amino acids 1-387 of SEQ ID NO:32;
Albumin, or fragment or variant thereof, comprising an amino acid sequence selected from the group consisting of: (a) amino acids 54 to 61 of SEQ ID NO:32; (b) amino acids 76 to 89 of SEQ ID NO:32; (c) amino acids 92 to 100 of SEQ ID NO:32; (d) amino acids 170 to 176 of SEQ ID NO:32; (e) amino acids 247 to 252 of SEQ ID NO:32; (f) amino acids 266 to 277 of SEQ ID NO:32; (g) amino acids 280 to 288 of SEQ ID NO:32; (h) amino acids 362 to 368 of SEQ ID NO:32; (i) amino acids 439 to 447 of SEQ ID NO:32; (j) amino acids 462 to 475 of SEQ ID NO:32; (k) amino acids 478 to 486 of SEQ ID NO:32; and (l) amino acids 560 to 566 of SEQ ID NO:32.

Further examples of suitable albumin, fragments and analogs for use in a ligand according to the invention are described in WO 03/076567A2, which is incorporated herein by reference in its entirety. In particular, the following albumin, fragments or variants can be used in the present invention:
Human serum albumin as described in WO 03/076567A2, e.g., in FIG. 3 (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, e.g., HA(1-373., HA(1-388), HA(1-389), HA(1-369), and HA(1-419) and fragments between 1-369 and 1-419;
An albumin fragment or variant as described in EP 399666, e.g., HA(1-177) and HA(1-200) and fragments between HA(1-X), where X is any number from 178 to 199.

When a (one or more) half-life extending moiety (eg, albumin, transferrin and fragments and analogues thereof) is used in the ligands of the invention, it can be conjugated to the ligand using any suitable method, such as, by direct fusion to the receptor-binding moiety (e.g., dAb or antibody fragment), for example by using a single nucleotide construct that encodes a fusion protein, wherein the fusion protein is encoded as a single polypeptide chain with the half-life extending moiety located N- or C-terminally to the receptor target binding moieties. Conjugation can also be achieved by using a peptide linker between moieties, e.g., a peptide linker as described in WO 03/076567A2 or WO 2004/003019 (these linker disclosures being incorporated by reference in the present disclosure to provide examples for use in the present invention).

The hydrodynaminc size of ligand (e.g., dAb monomer) and its serum half-life can also be increased by conjugating or linking the ligand to a binding domain (e.g., antibody or antibody fragment) that binds an antigen or epitope that increases half-live in vivo, as described herein. For example, the ligand (e.g., dAb monomer) can be conjugated or linked to an anti-serum albumin or anti-neonatal Fc receptor antibody or antibody fragment, e.g an anti-SA or anti-neonatal Fc receptor dAb, Fab, Fab′ or scFv, or to an anti-SA affibody or anti-neonatal Fc receptor affibody.

Typically, a polypeptide that enhances serum half-life in vivo is a polypeptide which occurs naturally in vivo and which resists degradation or removal by endogenous mechanisms which remove unwanted material from the organism (e.g., human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fc transport.

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g., soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-I glycoprotein (orosomucoid; AAG), alpha-1 antichymotrypsin (ACT), alpha-1 microglobulin (protein HC; AIM), antithrombin III (AT III), apolipoprotein A-I (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g. type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g., fibrin, α-2 macroglobulin, serum albumin, fibrinogen (e.g., fibrinogen A, fibrinogen B), serum amyloid protein A, haptoglobin, profilin, ubiquitin, uteroglobulin and β-2-microglobulin), enzymes and enzyme inhibitors (e.g., plasminogen, lysozyme, cystatin C, alpha-1-antitrypsin and pancreatic trypsin inhibitor), proteins of the immune system, such as immunoglobulin proteins (e.g., IgA, IgD, IgE, IgG, IgM, immunoglobulin light chains (kappa/lambda)), transport proteins (e.g., retinol binding protein, α-1 microglobulin), defensins (e.g., beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhances serum half-life in vivo also include proteins localized to the kidney (e.g., polycystin, type IV collagen, organic anion transporter K1, Heymann's antigen), proteins localized to the liver (e.g., alcohol dehydrogenase, G250), proteins localized to the lung (e.g., secretory component, which binds IgA), proteins localized to the heart (e.g., HSP 27, which is associated with dilated cardiomyopathy), proteins localized to the skin (e.g., keratin), bone specific proteins such as morphogenic proteins (BMPs), which are a subset of the transforming growth factor β superfamily of proteins that demonstrate osteogenic activity (e.g., BMP-2, BMP-4, BMP-5, BMP-6, BMP-7, BMP-8), tumor specific proteins (e.g., trophoblast antigen, herceptin receptor, oestrogen receptor, cathepsins (e.g., cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetc analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

Polypeptide Domains that Bind EGFR

The invention provides ligands (e.g., isolated dAbs) that have a binding domain (e.g., a domain comprising a binding site) with binding specificity for EGFR. In preferred embodiments, the ligand (e.g., dAb) binds to EGFR with an affinity (KD; KD=K_{off}(kd)/Kₒₙ(ka)) of 300 nM to 1 pM (i.e., 3×10⁻⁷ to 1×10⁻¹²M), preferably 300 nM to 5 pM or 100 nM to 1 pM, or 50 nM to 10 pM, more preferably 10 nM to 100 pM and most preferably about 1 nM, for example and KD of 1×10⁻⁷ M or less, preferably 1×10⁻⁸ M or less, more preferably about 1×10⁻⁹ M or less, 1×10⁻¹⁰ M or less or 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance.

The ligand can be a monospecific ligand, such as a dAb monomer, a dual specific ligand or a multispecific ligand, that is monovalent, bivalent or multivalent as described herein. Preferably, the ligand comprises an immunoglobulin single variable domain (dAb) that has binding specificity for EGFR (HER1). The ligand can comprise any suitable immunoglobulin single variable domain that has binding specificity for EGFR, such a an immunoglobulin heavy chain single variable domain (e.g., V_{H}, Camelid V_{HH}) or immunoglobulin light chain single variable domain (e.g., V_{λ}, V_{κ}). Preferably, the immunoglobulin single variable domain is a heavy chain single variable domain, such as a V_{H} (e.g., a human V_{H}) or a Camelid V_{HH}. Preferably, the immunoglobulin single variable domain binds EGFR (e.g, human EGFR) with high affinity, and inhibits EGFR (e.g., is an EGFR antagonist) as described herein. Preferred ligands, generally comprises a human immunoglobulin single variable domain, or an immunoglobulin single variable domain that comprises human framework regions. In certain embodiments, the ligand comprises a human immunoglobulin single variable domain that comprises a universal framework, as described herein.

In some embodiments, the ligand comprises a humanized immunoglobulin single variable domain with binding specificity for EGFR, preferably human EGFR. In preferred embodiments, the ligand comprises a human immunoglobulin single variable domain with binding specificity for human EGFR.

In some embodiment, the ligand is a dual specific ligand that comprises at least one first polypeptide domain that has a binding site with binding specificity for EGFR, and at least one second polypeptide binding domain that has a binding site with binding specificity for another protein. For example, the second polypeptide domain can have a binding site with binding specificity for a receptor disclosed herein, for a receptor for a cytokine or growth factor as disclosed herein, or for a polypeptide that enhances serum half life in vivo (e.g., serum albumin). In particular embodiments, both the first polypeptide domain and the second polypeptide domain are immunoglobulin single variable domains, preferably single heavy chain variable domains, such as V_{H} (e.g., a human V_{H}) or a Camelid V_{HH}.

Examples of such dual specific ligands include ligands that comprise an immunoglobulin single variable domain that has binding specificity for EGFR, and an immunoglobulin single variable domain that has binding specificity for a polypeptide selected from the group consisting of HER2, HER3, HER4, CSF1R, IGF1R, gastrin releasing peptide receptor, neurotensin receptor, adrenomedullin receptor, H2 histamine receptor, HCG receptor, Met receptor, sphignosine 1-phosphate receptor, carcinoembryonic antigen (CEA), neural cell adhesion molecule (NCAM), carcinoembryonic antigen-related cell adhesion molecule (CEACAM), fibroblast activation protein (FAP), IL-8, CD126 and CD213a1.

In another example of a dual specific ligand that has binding specificity for EGFR, the ligand comprise at least one immunoglobulin single variable domain (dAb) that has binding specificity for EGFR (e.g., human EGFR) and at least one immunoglobulin single variable domain that has binding specificity for serum albumin (e.g., human serum albumin). In particular embodiments, the immunoglobulin single variable domains are heavy chain variable domains (e.g., V_{H}, V_{HH}). For example, the ligand can contain two immunoglobulin single heavy chain variable domains (e.g., V_{H}, V_{HH}) that have binding specificity for EGFR and an immunoglobulin single heavy chain variable domain (e.g., V_{H}, V_{HH}) that has binding specificity for serum albumin. The immunoglobulin single variable domains that have binding specificity for EGFR can bind to the same or different epitopes on EGFR as desired. Additionally, the ligand can contain two or more copies of an immunoglobulin single variable domain that has binding specificity for EGFR, or can contain two or more different immunoglobulin single variable domains that each have binding specificity for EGFR.

In some embodiments, the ligand (e.g., isolated dAb) that inhibits binding of EGF and/or TGF alpha to EGFR in the EGFR binding assay or EGFR kinase assay described herein with an IC50 of about 1 μM or less, about 500 nM or less, about 100 nM or less, about 75 nM or less, about 50 nM or less, about 10 nM or less or about 1 nM or less. In other embodiments, the ligand (e.g., isolated dAb) inhibits kinase activity of EGFR in the EGFR kinase assay described herein with an IC50 of about 1 μM or less, about 500 nM or less, about 100 nM or less, about 75 nM or less, about 50 nM or less, about 10 nM or less or about 1 nM or less.

In particular embodiments, the ligand that has a binding site with binding specificity for EGFR competes for binding to EGFR with a dAb that has binding specificity for EGFR such as any one of the anti-EGFR dAbs disclosed in International Application No. PCT/GB2007/000049, filed on Jan. 10, 2007, which designates the United States, the entire contents of which are incorporated herein by reference. For example, in some embodiments, the ligand has a binding site with binding specificity for EGFR competes for binding to EGFR with an anti-EGFR dAb selected from the group consisting of DOM16-39 (SEQ ID NO:33), DOM16-39-87 (SEQ ID NO:34), DOM16-39-100 (SEQ ID NO:35), DOM16-39-107 (SEQ ID NO:36), DOM16-39-109 (SEQ ID NO:37), DOM16-39-115 (SEQ ID NO:38), and DOM16-39-200 (SEQ ID NO:39).

In particular exemplary embodiments, the ligand that has a binding site with binding specificity for EGFR comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence of DOM16-39 (SEQ ID NO:33). For example, the ligand that has a binding site with binding specificity for EGFR can comprise the amino acid sequence of DOM16-39-87 (SEQ ID NO:34), DOM16-39-100 (SEQ ID NO:35), DOM16-39-107 (SEQ ID NO:36), DOM16-39-109 (SEQ ID NO:37), DOM16-39-115 (SEQ ID NO:38), or DOM16-39-200 (SEQ ID NO:39).

In other embodiments, the ligand that has a binding site with binding specificity for EGFR competes for binding to EGFR with a dAb that has binding specificity for EGFR such as any one of the anti-EGFR dAbs disclosed in International Publication No. WO 2005/044858 (PCT/BE2003/000189) or WO 2004/041867 (PCT/BE2003/000190) the entire contents of each of the foregoing are incorporated herein by reference. In particular exemplary embodiments, the ligand that has a binding site with binding specificity for EGFR comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence of any one of the anti-EGFR dAbs disclosed in International Publication No. WO 2005/044858 (PCT/BE2003/000189) or WO 2004/041867 (PCT/BE2003/000190). For example, the ligand that has a binding site with binding specificity for EGFR can comprise the amino acid sequence of any one of the anti-EGFR dAbs disclosed in International Publication No. WO 2005/044858 (PCT/BE2003/000189) or WO 2004/041867 (PCT/BE2003/000190).

Polypeptide Domains that Bind Bind TNFR1

The invention also provides ligands (e.g., isolated dAbs) that have a binding domain (e.g., a domain comprising a binding site) with binding specificity for TNF Receptor I (TNFR1). In preferred embodiments, the ligand binds to TNF Receptor I with an affinity of 300 nM to 5 pM (ie, 3×10⁻⁷ to 5×10⁻¹²M) or 300 nM to 1 pM, preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance.

The ligand can be a monospecific ligand, such as a dAb monomer, a dual specific ligand or a multispecific ligand, that is monovalent, bivalent or multivalent as described herein. Preferably, the ligand comprises an immunoglobulin single variable domain (dAb) that has binding specificity for TNFR1. The ligand can comprise any suitable immunoglobulin single variable domain that has binding specificity for TNFR1, such a an immunoglobulin heavy chain single variable domain (e.g., V_{H}, Camelid V_{HH}) or immunoglobulin light chain single variable domain (e.g., V_{λ}, V_{κ}). Preferably, the immunoglobulin single variable domain is a heavy chain single variable domain, such as a V_{H} (e.g., a human V_{H}) or a Camelid V_{HH}. Preferably, the immunoglobulin single variable domain binds TNFR1 (e.g., human TNFR1) with high affinity, and inhibits the activity of TNFR1 (e.g., is a TNFR1 antagonist) as described herein. Preferred ligands, generally comprises a human immunoglobulin single variable domain, or an immunoglobulin single variable domain that comprises human framework regions. In certain embodiments, the ligand comprises a human immunoglobulin single variable domain that comprises a universal framework, as described herein.

In some embodiments, the ligand comprises a humanized immunoglobulin single variable domain with binding specificity for TNFR1, preferably human TNFR1. In preferred embodiments, the ligand comprises a human immunoglobulin single variable domain with binding specificity for human TNFR1.

In some embodiment, the ligand is a dual specific ligand that comprises at least one first polypeptide domain that has a binding site with binding specificity for TNFR1, and at least one second polypeptide binding domain that has a binding site with binding specificity for another protein. For example, the second polypeptide domain can have a binding site with binding specificity for a receptor disclosed herein, for a receptor for a cytokine or growth factor as disclosed herein, or for a polypeptide that enhances serum half life in vivo (e.g., serum albumin). In particular embodiments, both the first polypeptide domain and the second polypeptide domain are immunoglobulin single variable domains, preferably single heavy chain variable domains, such as V_{H} (e.g., a human V_{H}) or a Camelid V_{HH}.

In one example of a dual specific ligand that has binding specificity for TNFR1, the ligand comprise at least one immunoglobulin single variable domain (dAb) that has binding specificity for TNFR1 (e.g., human TNFR1) and at least one immunoglobulin single variable domain that has binding specificity for serum albumin (e.g., human serum albumin). In particular embodiments, the immunoglobulin single variable domains are heavy chain variable domains (e.g., V_{H}, V_{HH}). For example, the ligand can contain two immunoglobulin single heavy chain variable domains (e.g., V_{H}, V_{HH}) that have binding specificity for TNFR1 and an immunoglobulin single heavy chain variable domain (e.g., V_{H}, V_{HH}) that has binding specificity for serum albumin. The immunoglobulin single variable domains that have binding specificity for TNFR1 can bind to the same or different epitopes on TNFR1 as desired. Additionally, the ligand can contain two or more copies of an immunoglobulin single variable domain that has binding specificity for TNFR1, or can contain two or more different immunoglobulin single variable domains that each have binding specificity for TNFR1.

Preferably, the ligand inhibits binding of TNF alpha to TNF alpha Receptor I (p55 receptor) with an inhibitory concentration 50 (IC50) of 500 nM to 50 pM, preferably 100 nM to 50 pM, more preferably 10 nM to 100 pM, advantageously 1 nM to 100 pM; for example 50 nM or less, preferably 5 nM or less, more preferably 500 pM or less, advantageously 200 pM or less, and most preferably 100 pM or less.

Preferably, the ligand binds human TNFR1 and inhibits binding of human TNF alpha to human TNFR1, or inhibits clustering and/or signaling through TNFR1 in response to TNF alpha binding. For example, in certain embodiments, a ligand can bind TNFR1 and inhibit TNFR-1-mediated signaling, but does not substantially inhibit binding of TNFα to TNFR1. In some embodiments, the ligand inhibits TNFα-induced crosslinking or clustering of TNFR1 on the surface of a cell. Such ligands (e.g., ligands that comprise the dAb, TAR2m-21-23, described herein) are advantageous because they can antagonize cell surface TNFR1 but do not substantially reduce the inhibitory activity of endogenous soluble TNFR1. For example, the ligand can bind TNFR1, but inhibit binding of TNFα to TNFR1 in a receptor binding assay by no more that about 10%, no more that about 5%, no more than about 4%, no more than about 3%, no more than about 2%, or no more than about 1%. Also, in these embodiments, the ligand inhibits TNFα-induced crosslinking of TNFR1 and/or TNFR1-mediated signaling in a standard cell assay by at least about 10%, at least about 20%, at least about 30%, at least about 40%, at least about 50%, at least about 60%, at least about 70%, at least about 80%, at least about 90%, at least about 95%, or at least about 99%.

Preferably, the ligand neutralizes (inhibits the activity of) TNFα or TNFR1 in a standard assay (e.g., the standard L929 or standard HeLa IL-8 assays described herein) with a neutralizing dose 50 (ND50) of 500 nM to 50 pM, preferably 100 nM to 50 pM, more preferably 10 nM to 100 pM, advantageously 1 mM to 100 pM; for example 50 pM or less, preferably 5 nM or less, more preferably 500 pM or less, advantageously 200 pM or less, and most preferably 100 pM or less.

In certain embodiments, the ligand specifically binds human Tumor Necrosis Factor Receptor 1 (TNFR1; p55), and dissociates from human TNFR1 with a dissociation constant (K_{d}) of 50 nM to 20 pM, and a K_{off} rate constant of 5×10⁻¹ s⁻¹ to 1×10⁻⁷ s⁻¹, as determined by surface plasmon resonance.

In other embodiments, the ligand binds TNFR1 and antagonizes the activity of the TNFR1 in a standard cell assay with an ND₅₀ of ≦100 nM, and at a concentration of ≦10 μM the dAb agonizes the activity of the TNFR1 by ≦5% in the assay.

In particular embodiments, ligand does not substantially agonize TNFR1 (act as an agonist of TNFR1) in a standard cell assay (i.e., when present at a concentration of 1 nM, 10 nM, 100 nM, 1 μM, 10 μM, 100 μM, 1000 μM or 5,000 μM, results in no more than about 5% of the TNFR1-mediated activity induced by TNFα (100 pg/ml) in the assay).

In particular embodiments, the ligand that has a binding site with binding specificity for TNFR1 competes for binding to TNFR1 with a dAb that has binding specificity for TNFR1 such as any one of the anti-TNFR1 dAbs disclosed in U.S. patent application Ser. No. 10/985,847, filed Nov. 10, 2004, or U.S. patent application Ser. No. 11/664,542, filed Apr. 2, 2007, the entire contents of each of the foregoing U.S. applications are incorporated herein by reference. For example, in some embodiments, the ligand has a binding site with binding specificity for TNFR1 competes for binding to TNFR1 with an anti-TNFR1 dAb selected from the group consisting of TAR2-5 (SEQ ID NO:40), and TAR2-10 (SEQ ID NO:41). In other embodiments, the ligand has a binding site with binding specificity for TNFR1 competes for binding to TNFR1 with the anti-TNFR1 dAb TAR2h-10-27 (SEQ ID NO:42).

In particular exemplary embodiments, the ligand that has a binding site with binding specificity for TNFR1 comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence of TAR2-5 (SEQ ID NO:40) or TAR2-10 (SEQ ID NO:41). In other embodiments, the ligand that has a binding site with binding specificity for TNFR1 comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence of TAR2h-10-27 (SEQ ID NO:42).

In other embodiments, the ligand has a binding site with binding specificity for TNFR1 competes for binding to TNFR1 with an anti-TNFR1 dAb selected from the group consisting of TAR2h-131-8 (SEQ ID NO:43), TAR2h-15-8 (SEQ ID NO:44), TAR2h-35-4 (SEQ ID NO:45), TAR2h-154-7 (SEQ ID NO:46), TAR2h-154-10 (SEQ ID NO:47) and TAR2h-185-25 (SEQ ID NO:48). In particular embodiments, the ligand that has a binding site with binding specificity for TNFR1 comprises an amino acid sequence that has at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of TAR2h-131-8 (SEQ ID NO:43), TAR2h-15-8 (SEQ ID NO:44), TAR2h-35-4 (SEQ ID NO:45), TAR2h-154-7 (SEQ ID NO:46), TAR2h-154-10 (SEQ ID NO:47) and TAR2h-185-25 (SEQ ID NO:48).

Polypeptide Domains that Bind IL-1R1

The invention also provides ligands (e.g., isolated dAbs) that have a binding domain (e.g., a domain comprising a binding site) with binding specificity for Interleukin 1 Receptor Type 1 (IL-1R1). In preferred embodiments, the ligand binds to IL-1R1 with an affinity of 300 nM to 5 pM (ie, 3×10⁻⁷ to 5×10⁻¹²M) or 300 nM to 1 pM, preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance.

The ligand can be a monospecific ligand, such as a dAb monomer, a dual specific ligand or a multispecific ligand, that is monovalent, bivalent or multivalent as described herein. Preferably the ligand comprises an immunoglobulin single variable domain (dAb) that has binding specificity for IL-1R1. The ligand can comprise any suitable immunoglobulin single variable domain that has binding specificity for IL-1R1, such a an immunoglobulin heavy chain single variable domain (e.g., V_{H}, Camelid V_{HH}) or immunoglobulin light chain single variable domain (e.g., V_{λ}, V_{κ}). Preferably, the immunoglobulin single variable domain is a heavy chain single variable domain, such as a V_{H} (e.g., a human V_{H}) or a Camelid V_{HH}. Preferably, the immunoglobulin single variable domain binds IL-1R1 (e.g. human IL-1R1) with high affinity, and inhibits the activity of IL-1R1 (e.g., is a IL-1R1antagonist) as described herein. Preferred ligands, generally comprises a human immunoglobulin single variable domain, or an immunoglobulin single variable domain that comprises human framework regions. In certain embodiments, the ligand comprises a human immunoglobulin single variable domain that comprises a universal framework, as described herein.

In some embodiments, the ligand comprises a humanized immunoglobulin single variable domain with binding specificity for IL-1R1, preferably a human IL-1R1. In preferred embodiments, the ligand comprises a human immunoglobulin single variable domain with binding specificity for human IL-1R1.

In some embodiment, the ligand is a dual specific ligand that comprises at least one first polypeptide domain that has a binding site with binding specificity for IL-1R1, and at least one second polypeptide binding domain that has a binding site with binding specificity for another protein. For example, the second polypeptide domain can have a binding site with binding specificity for a receptor disclosed herein, for a receptor for a cytokine or growth factor as disclosed herein, or for a polypeptide that enhances serum half life in vivo (e.g., serum albumin). In particular embodiments, both the first polypeptide domain and the second polypeptide domain are immunoglobulin single variable domains, preferably single heavy chain variable domains, such as V_{H} (e.g., a human V_{H}) or a Camelid V_{HH}.

In one example of a dual specific ligand that has binding specificity for IL-1R1, the ligand comprise at least one immunoglobulin single variable domain (dAb) that has binding specificity for IL-1R1 (e.g., human IL-1R1) and at least one immunoglobulin single variable domain that has binding specificity for serum albumin (e.g., human serum albumin). In particular embodiments, the immunoglobulin single variable domains are heavy chain variable domains (e.g., V_{H}, V_{HH}). For example, the ligand can contain two immunoglobulin single heavy chain variable domains (e.g., V_{H}, V_{HH}) that have binding specificity for IL-1R1 and an immunoglobulin single heavy chain variable domain (e.g., V_{H}, V_{HH}) that has binding specificity for serum albumin. The immunoglobulin single variable domains that have binding specificity for IL-1R1 can bind to the same or different epitopes on IL-1R1 as desired. Additionally, the ligand can contain two or more copies of an immunoglobulin single variable domain that has binding specificity for IL-1R1, or can contain two or more different immunoglobulin single variable domains that each have binding specificity for IL-1R1.

Preferably, the ligand (e.g., isolated dAb monomer) inhibits binding of L-1 (e.g., IL-1α and/or IL-1β) to IL-1R1, for example in a receptor binding assay, with an inhibitory concentration 50 (IC50) that is equal to or less than about 1 μM, for example an IC50 of about 500 nM to about 50 pM, preferably about 100 nM to about 50 pM, more preferably about 10 nM to about 100 pM, advantageously about 1 nM to about 100 pM; for example about 50 nM or less, preferably about 5 nM or less, more preferably about 500 pM or less, advantageously about 200 pM or less, and most preferably about 100 pM or less.

Preferably, the ligand (e.g., isolated dAb monomer) binds human IL-1R1 and inhibits binding of human IL-1 (e.g., IL-1α and/or IL-1β) to human IL-1R1 and inhibits clustering and/or signaling through human IL-1R1 in response to IL-1 binding.

Preferably, the ligand (e.g., isolated dAb monomer) neutralizes (inhibits the activity of) IL-1 or IL-1R1 in a standard assay (e.g., IL-1-induced release of Interleukin-8 by MRC-5 cells, IL-1-induced release of Interleukin-6 by whole blood cells) with a neutralizing dose 50 (ND50) that is less than or equal to about 1 pM, for example an ND50 of about 500 mM to about 50 pM, preferably about 100 nM to about 50 pM, more preferably about 10 nM to about 100 pM, advantageously about 1 nM to about 100 pM; for example about 50 nM or less, preferably about 5 nM or less, more preferably about 500 pM or less, advantageously about 200 pM or less, and most preferably about 100 pM or less. For example, the ligand (e.g., isolated dAb monomer) inhibits IL-1-induced (e.g., IL-1α- or IL-1β-induced) release of Interleukin-8 by MRC-5 cells (ATCC Accession No. CCL-171) in an in vitro assay with a ND50 that is ≦10 μM, ≦1 μM, ≦100 nM, ≦10 nM, ≦1 nM, ≦500 pM, ≦300 pM, ≦100 pM, or ≦10 pM. In another example, the ligand (e.g., isolated dAb monomer) inhibits IL-1-induced (e.g., IL-1α- or IL-1β-induced) release of Interleukin-6 in an in vitro whole blood assay with a ND50 that is ≦10 μM, ≦1 μM, ≦100 nM, ≦10 nM, ≦nM, ≦500 pM, ≦300 pM, ≦100 pM, or ≦10 pM.

Preferably, the ligand (e.g., a multivalent ligand) does not substantially agonize IL-1R1 (act as an agonist of IL-1R1) in a standard cell assay (i.e., when present at a concentration of 1 nM, 10 nM, 100 nM, 1 μM, 10 μM, 100 μM, 1000 μM or 5,000 μM, results in no more than about 5% of the IL-1R1-mediated activity induced by IL-1 (100 pg/ml) in the assay).

In particular embodiments, the ligand that has a binding site with binding specificity for IL-1R1 competes for binding to IL-1R1 with a dAb that has binding specificity for IL-1R1 such as any one of the anti-IL-1R1 dAbs disclosed in International Application No. PCT/GB2006/004471, filed Nov. 30, 2006, which designates the United States, or International Application No. PCT/GB2006/004477, filed Nov. 30, 2006, filed Nov. 30, 2006, which designates the United States, the entire contents of each of the foregoing international applications are incorporated herein by reference. In some embodiments the ligand comprises a dAb (e.g, human dAb) that competes for binding to IL-1R1 with a dAb selected from the group consisting of DOM4-122-23 (SEQ ID NO:49), DOM4-122-24 (SEQ ID NO:50), DOM4-122 (SEQ ID NO:51), DOM4-122-1 (SEQ ID NO:52), DOM4-122-2 (SEQ ID NO:53), DOM4-122-3 (SEQ ID NO:54), DOM4-122-4 (SEQ ID NO:55), DOM4-122-5 (SEQ ID NO:56), DOM4-122-6 (SEQ ID NO:57), DOM4-122-7 (SEQ ID NO:58), DOM4-122-8 (SEQ ID NO:59), DOM4-122-9 (SEQ ID NO:60), DOM4-122-10 (SEQ ID NO:61), DOM4-122-11 (SEQ ID NO:62), DOM4-122-12 (SEQ ID NO:63), DOM4-122-13 (SEQ ID NO:64), DOM4-122-14 (SEQ ID NO:65), DOM4-122-15 (SEQ ID NO:66), DOM4-122-16 (SEQ ID NO:67), DOM4-122-17 (SEQ ID NO:68), DOM4-122-18 (SEQ ID NO:69), DOM4-122-19 (SEQ ID NO:70), DOM4-122-20 (SEQ ID NO:71), DOM4-122-21 (SEQ ID NO:72), DOM4-122-22 (SEQ ID NO:73), DOM4-122-25 (SEQ ID NO:74), DOM4-122-26 (SEQ ID NO:75), DOM4-122-27 (SEQ ID NO:76), DOM4-122-28 (SEQ ID NO:77), DOM4-122-29 (SEQ ID NO:78), DOM4-122-30 (SEQ ID NO:79), DOM4-122-31 (SEQ ID NO:80), DOM4-122-32 (SEQ ID NO:81), DOM4-122-33 (SEQ ID NO:82), DOM4-122-34 (SEQ ID NO:83), DOM4-122-35 (SEQ ID NO:84), DOM4-122-36 (SEQ ID NO:85), DOM4-122-37 (SEQ ID NO:86), DOM4-122-38 (SEQ ID NO:87), DOM4-122-39 (SEQ ID NO:88), DOM4-122-40 (SEQ ID NO:89), DOM4-122-41 (SEQ ID NO:90), DOM4-122-42 (SEQ ID NO:91), DOM4-122-43 (SEQ ID NO:92), DOM4-122-44 (SEQ ID NO:93), DOM4-122-45 (SEQ ID NO:94), DOM4-122-46 (SEQ ID NO:95), DOM4-122-47 (SEQ ID NO:96), DOM4-122-48 (SEQ ID NO:97), DOM4-122-49 (SEQ ID NO:98), DOM4-122-50 (SEQ ID NO:99), DOM4-122-51 (SEQ ID NO:100), DOM4-122-52 (SEQ ID NO:101), DOM4-122-54 (SEQ ID NO:102), DOM4-122-55 (SEQ ID NO:103), DOM4-122-56 (SEQ ID NO:104), DOM4-122-57 (SEQ ID NO:105), DOM4-122-58 (SEQ ID NO:106), DOM4-122-59 (SEQ ID NO:107), DOM4-122-60 (SEQ ID NO:108), DOM4-122-61 (SEQ ID NO:109), DOM4-122-62 (SEQ ID NO:110), DOM4-122-63 (SEQ ID NO:111), DOM4-122-64 (SEQ ID NO:112), DOM4-122-65 (SEQ ID NO:113), DOM4-122-66 (SEQ ID NO:114), DOM4-122-67 (SEQ ID NO:115), DOM4-122-68 (SEQ ID NO:116), DOM4-122-69 (SEQ ID NO:117), DOM4-122-70 (SEQ ID NO:118), DOM4-122-71 (SEQ ID NO:119), DOM4-122-72 (SEQ ID NO:120), and DOM4-122-73 (SEQ ID NO:121).

Preferably, the ligand comprises a dAb with an amino acid sequence that has at least about 90%, at lease about 91%, at least about 92%, at least about 93%, at least about 94%, at least about 95%, at least about 96%, at least about 97%, at least about 98% or at least about 99% amino acid sequence identity with an amino acid sequence selected from the group consisting of consisting of DOM4-122-23 (SEQ ID NO:49), DOM4-122-24 (SEQ ID NO:50), DOM4-122 (SEQ ID NO:51), DOM4-122-1 (SEQ ID NO:52), DOM4-122-2 (SEQ ID NO:53), DOM4-122-3 (SEQ ID NO:54), DOM4-122-4 (SEQ ID NO:55), DOM4-122-5 (SEQ ID NO:56), DOM4-122-6 (SEQ ID NO:57), DOM4-122-7 (SEQ ID NO:58), DOM4-122-8 (SEQ ID NO:59), DOM4-122-9 (SEQ ID NO:60), DOM4-122-10 (SEQ ID NO:61), DOM4-122-11 (SEQ ID NO:62), DOM4-122-12 (SEQ ID NO:63), DOM4-122-13 (SEQ ID NO:64), DOM4-122-14 (SEQ ID NO:65), DOM4-122-15 (SEQ ID NO:66), DOM4-122-16 (SEQ ID NO:67), DOM4-122-17 (SEQ ID NO:68), DOM4-122-18 (SEQ ID NO:69), DOM4-122-19 (SEQ ID NO:70), DOM4-122-20 (SEQ ID NO:71), DOM4-122-21 (SEQ ID NO:72), DOM4-122-22 (SEQ ID NO:73), DOM4-122-25 (SEQ ID NO:74), DOM4-122-26 (SEQ ID NO:75), DOM4-122-27 (SEQ ID NO:76), DOM4-122-28 (SEQ ID NO:77), DOM4-122-29 (SEQ ID NO:78), DOM4-122-30 (SEQ ID NO:79), DOM4-122-31 (SEQ ID NO:80), DOM4-122-32 (SEQ ID NO:81), DOM4-122-33 (SEQ ID NO:82), DOM4-122-34 (SEQ ID NO:83), DOM4-122-35 (SEQ ID NO:84), DOM4-122-36 (SEQ ID NO:85), DOM4-122-37 (SEQ ID NO:86), DOM4-122-38 (SEQ ID NO:87), DOM4-122-39 (SEQ ID NO:88), DOM4-122-40 (SEQ ID NO:89), DOM4-122-41 (SEQ ID NO:90), DOM4-122-42 (SEQ ID NO:91), DOM4-122-43 (SEQ ID NO:92), DOM4-122-44 (SEQ ID NO:93), DOM4-122-45 (SEQ ID NO:94), DOM4-122-46 (SEQ ID NO:95), DOM4-122-47 (SEQ ID NO:96), DOM4-122-48 (SEQ ID NO:97), DOM4-122-49 (SEQ ID NO:98), DOM4-122-50 (SEQ ID NO:99), DOM4-122-51 (SEQ ID NO:100), DOM4-122-52 (SEQ ID NO:101), DOM4-122-54 (SEQ ID NO:102), DOM4-122-55 (SEQ ID NO:103), DOM4-122-56 (SEQ ID NO:104), DOM4-122-57 (SEQ ID NO:105), DOM4-122-58 (SEQ ID NO:106), DOM4-122-59 (SEQ ID NO:107), DOM4-122-60 (SEQ ID NO:108), DOM4-122-61 (SEQ ID NO:109), DOM4-122-62 (SEQ ID NO:110), DOM4-122-63 (SEQ ID NO:111), DOM4-122-64 (SEQ ID NO:112), DOM4-122-65 (SEQ ID NO:113), DOM4-122-66 (SEQ ID NO:114), DOM4-122-67 (SEQ ID NO:115), DOM4-122-68 (SEQ ID NO:116), DOM4-122-69 (SEQ ID NO:117), DOM4-122-70 (SEQ ID NO:118), DOM4-122-71 (SEQ ID NO:119), DOM4-122-72 (SEQ ID NO:120), and DOM4-122-73 (SEQ ID NO:121).

Polypeptide Domains that Bind CD38

The invention also provides ligands (e.g., isolated dAbs) that have a binding domain (e.g., a domain comprising a binding site) with binding specificity for CD38. In a preferred embodiments, the ligand binds to CD38 with an affinity of 300 nM to 5 pM (ie, 3×10⁻⁷ to 5×10⁻¹²M) or 300 nM to 1 pM, preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10−5 s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance. In some embodiments, the polypeptide domain (e.g., dAb) binds to CD38 with low affinity, such as an affinity between about 1 0M to about 10 nM as determined by surface plasmon resonance. For example, the polypeptide domain can bind CD38 with an affinity of about 10 μM to about 300 nM, or about 10 μM to about 400 nM. In certain embodiments, the polypeptide domain binds CD38 with an affinity of about 300 nM to about 10 nM or 200 nM to about 10 nM.

The ligand can be a monospecific ligand, such as a dAb monomer, a dual specific ligand or a multispecific ligand, that is monovalent, bivalent or multivalent as described herein. Preferably, the ligand comprises an immunoglobulin single variable domain (dAb) that has binding specificity for CD38. The ligand can comprise any suitable immunoglobulin single variable domain that has binding specificity for CD38, such a an immunoglobulin heavy chain single variable domain (e.g., V_{H}, Camelid V_{HH}) or immunoglobulin light chain single variable domain (e.g., V_{λ}, V_{κ}). Preferably, the immunoglobulin single variable domain is a heavy chain single variable domain, such as a V_{H} (e.g., a human V_{H}) or a Camelid V_{HH}. Preferably, the immunoglobulin single variable domain binds CD38 (e.g., human CD38) with high affinity, and inhibits the activity of CD38 (e.g., is a CD38 antagonist) as described herein. Preferred ligands, generally comprises a human immunoglobulin single variable domain, or an immunoglobulin single variable domain that comprises human framework regions. In certain embodiments, the ligand comprises a human immunoglobulin single variable domain that comprises a universal framework, as described herein.

In some embodiments, the ligand comprises a humanized immunoglobulin single variable domain with binding specificity for CD38, preferably human CD38. In preferred embodiments, the ligand comprises a human immunoglobulin single variable domain with binding specificity for human CD38.

In some embodiment, the ligand is a dual specific ligand that comprises at least one first polypeptide domain that has a binding site with binding specificity for CD38, and at least one second polypeptide binding domain that has a binding site with binding specificity for another protein. For example, the second polypeptide domain can have a binding site with binding specificity for a receptor disclosed herein, for a receptor for a cytokine or growth factor as disclosed herein, or for a polypeptide that enhances serum half life in vivo (e.g., serum albumin). In particular embodiments, both the first polypeptide domain and the second polypeptide domain are immunoglobulin single variable domains, preferably single heavy chain variable domains, such as V_{H} (e.g., a human V_{H}) or a Camelid V_{HH}.

In one example of a dual specific ligand that has binding specificity for CD38, the ligand comprise at least one immunoglobulin single variable domain (dAb) that has binding specificity for CD38 (e.g., human CD38) and at least one immunoglobulin single variable domain that has binding specificity for serum albumin (e.g., human serum albumin). In particular embodiments, the immunoglobulin single variable domains are heavy chain variable domains (e.g., V_{H}, V_{HH}). For example, the ligand can contain two immunoglobulin single heavy chain variable domains (e.g., V_{H}, V_{HH}) that have binding specificity for CD38 and an immunoglobulin single heavy chain variable domain (e.g., V_{H}, V_{HH}) that has binding specificity for serum albumin. The immunoglobulin single variable domains that have binding specificity for CD38 can bind to the same or different epitopes on CD38 as desired. Additionally, the ligand can contain two or more copies of an immunoglobulin single variable domain that has binding specificity for CD38, or can contain two or more different immunoglobulin single variable domains that each have binding specificity for CD38.

In particular embodiments, the ligand that has a binding site with binding specificity for CD38 competes for binding to CD38 with a dAb that has binding specificity for CD38 such as any one of the anti-CD38 dAbs disclosed in International Application No. PCT/GB2006/004565, filed Dec. 5, 2006, which designates the United States, the entire contents of which are incorporated herein by reference. In some embodiments, the polypeptide domain that has a binding site with binding specificity for CD38 competes for binding to CD38 with a dAb selected from the group consisting of DOM11-14 (SEQ ID NO:122), DOM11-22 (SEQ ID NO:123), DOM11-23 (SEQ ID NO:124), DOM11-25 (SEQ ID NO:125), DOM11-26 (SEQ ID NO:126), DOM11-27 (SEQ ID NO:127), DOM11-29(SEQ ID NO:128), DOM11-3(SEQ ID NO:129), DOM11-30 (SEQ ID NO:130), DOM11-31(SEQ ID NO:131), DOM11-32(SEQ ID NO:132), DOM11-36 (SEQ ID NO:133), DOM11-4 (SEQ ID NO:134), DOM11-43 (SEQ ID NO:135), DOM11-44 (SEQ ID NO:136), DOM11-45 (SEQ ID NO:137), DOM11-5 (SEQ ID NO:138), DOM11-7 (SEQ ID NO:139), DOM11-1 (SEQ ID NO:140), DOM11-10 (SEQ ID NO:141), DOM11-16 (SEQ ID NO:142), DOM11-2 (SEQ ID NO:143), DOM11-20 (SEQ ID NO:144), DOM11-21 (SEQ ID NO:145), DOM11-24 (SEQ ID NO:146), DOM11-28 (SEQ ID NO:147), DOM11-33 (SEQ ID NO:148), DOM11-34 (SEQ ID NO:149), DOM11-35 (SEQ ID NO:150), DOM11-37 (SEQ ID NO:151), DOM11-38 (SEQ ID NO:152), DOM11-39 (SEQ ID NO:153), DOM11-41 (SEQ ID NO:154), DOM11-42 (SEQ ID NO:155), DOM11-6 (SEQ ID NO:156), DOM11-8 (SEQ ID NO:157), and DOM11-9 (SEQ ID NO:158).

In other embodiments, the polypeptide domain that has a binding site with binding specificity for CD38 competes for binding to CD38 with a dAb selected from the group consisting of DOM11-3-1 (SEQ ID NO:159), DOM11-3-2 (SEQ ID NO:160), DOM11-3-3 (SEQ ID NO:161), DOM11-3-4 (SEQ ID NO:162), DOM11-3-6 (SEQ ID NO:163), DOM11-3-9 (SEQ ID NO:164), DOM11-3-10 (SEQ ID NO:165), DOM11-3-11 (SEQ ID NO:166), DOM11-3-14 (SEQ ID NO:167), DOM11-3-15 (SEQ ID NO:168), DOM11-3-17 (SEQ ID NO:169), DOM11-3-19 (SEQ ID NO:170), DOM11-3-20 (SEQ ID NO:171), DOM11-3-21 (SEQ ID NO:172), DOM11-3-22 (SEQ ID NO:173), DOM1-3-23 (SEQ ID NO:174), DOM11-3-24 (SEQ ID NO:175), DOM1′-3-25 (SEQ ID NO:176), DOM11-3-26 (SEQ ID NO:177), DOM11-3-27 (SEQ ID NO:178), DOM11-3-28 (SEQ ID NO:179), DOM11-30-1 (SEQ ID NO:180), DOM11-30-2 (SEQ ID NO:181), DOM11-30-3 (SEQ ID NO:182), DOM11-30-5 (SEQ ID NO:183), DOM11-30-6 (SEQ ID NO:184), DOM1′-30-7 (SEQ ID NO:185), DOM11-30-8 (SEQ ID NO:186), DOM11-30-9 (SEQ ID NO:187), DOM11-30-10 (SEQ ID NO:188), DOM11-30-11 (SEQ ID NO:189), DOM11-30-12 (SEQ ID NO:190), DOM11-30-13 (SEQ ID NO:191), DOM11-30-14 (SEQ ID NO:192), DOM1-30-15 (SEQ ID NO:193), DOM11-30-16 (SEQ ID NO:194), and DOM11-30-17 (SEQ ID NO:195).

In some embodiments, the polypeptide domain that has a binding site with binding specificity for CD38 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of: consisting of DOM11-14 (SEQ ID NO:122), DOM11-22 (SEQ ID NO:123), DOM11-23 (SEQ ID NO:124), DOM11-25 (SEQ ID NO:125), DOM11-26 (SEQ ID NO:126), DOM11-27 (SEQ ID NO:127), DOM11-29(SEQ ID NO:128), DOM11-3(SEQ ID NO:129), DOM11-30 (SEQ ID NO:130), DOM11-31(SEQ ID NO:131), DOM11-32(SEQ ID NO:132), DOM1-36 (SEQ ID NO:133), DOM1-4 (SEQ ID NO:134), DOM1-43 (SEQ ID NO:135), DOM11-44 (SEQ ID NO:136), DOM11-45 (SEQ ID NO:137), DOM11-5 (SEQ ID NO:138), DOM11-7 (SEQ ID NO:139), DOM11-1 (SEQ ID NO:140), DOM11-10 (SEQ ID NO:141), DOM11-16 (SEQ ID NO:142), DOM11-2 (SEQ ID NO:143), DOM11-20 (SEQ ID NO:144), DOM11-21 (SEQ ID NO:145), DOM11-24 (SEQ ID NO:146), DOM11-28 (SEQ ID NO:147), DOM11-33 (SEQ ID NO:148), DOM11-34 (SEQ ID NO:149), DOM11-35 (SEQ ID NO:150), DOM11-37 (SEQ ID NO:151), DOM11-38 (SEQ ID NO:152), DOM11-39 (SEQ ID NO:153), DOM11-41 (SEQ ID NO:154), DOM11-42 (SEQ ID NO:155), DOM11-6 (SEQ ID NO:156), DOM11-8 (SEQ ID NO:157), and DOM11-9 (SEQ ID NO:158).

In other embodiments, the polypeptide domain that has a binding site with binding specificity for CD38 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM1-3-1 (SEQ ID NO:159), DOM11-3-2 (SEQ ID NO:160), DOM11-3-3 (SEQ ID NO:161), DOM11-3-4 (SEQ ID NO:162), DOM11-3-6 (SEQ ID NO:163), DOM11-3-9 (SEQ ID NO:164), DOM11-3-10 (SEQ ID NO:165), DOM11-3-11 (SEQ ID NO:166), DOM11-3-14 (SEQ ID NO:167), DOM11-3-15 (SEQ ID NO:168), DOM11-3-17 (SEQ ID NO:169), DOM11-3-19 (SEQ ID NO:170), DOM11-3-20 (SEQ ID NO:171), DOM11-3-21 (SEQ ID NO:172), DOM11-3-22 (SEQ ID NO:173), DOM11-3-23 (SEQ ID NO:174), DOM11-3-24 (SEQ ID NO:175), DOM11-3-25 (SEQ ID NO:176), DOM11-3-26 (SEQ ID NO:177), DOM11-3-27 (SEQ ID NO:178), DOM11-3-28 (SEQ ID NO:179), DOM11-30-1 (SEQ ID NO:180), DOM11-30-2 (SEQ ID NO:181), DOM11-30-3 (SEQ ID NO:182), DOM11-30-5 (SEQ ID NO:183), DOM11-30-6 (SEQ ID NO:184), DOM11-30-7 (SEQ ID NO:185), DOM11-30-8 (SEQ ID NO:186), DOM1-30-9 (SEQ ID NO:187), DOM11-30-10 (SEQ ID NO:188), DOM11-30-11 (SEQ ID NO:189), DOM11-30-12 (SEQ ID NO:190), DOM11-30-13 (SEQ ID NO:191), DOM11-30-14 (SEQ ID NO:192), DOM11-30-15 (SEQ ID NO:193), DOM11-30-16 (SEQ ID NO:194), and DOM11-30-17 (SEQ ID NO:195).

Polypeptide Domains that Bind CD 138

The invention also provides ligands (e.g., isolated dAbs) that have a binding domain (e.g., a domain comprising a binding site) with binding specificity for CD138. In a preferred embodiments, the ligand binds to CD138 with an affinity of 300 nM to 5 pM (ie, 3×10⁻⁷ to 5×10⁻¹²M) or 300 nM to 1 pM, preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance. In some embodiments, the polypeptide domain binds to CD138 with low affinity, such as an affinity between about 10 μM to about 10 nM as determined by surface plasmon resonance. For example, the polypeptide domain can bind CD138 with an affinity of about 10 μM to about 300 nM, or about 10 μM to about 400 nM. In certain embodiments, the polypeptide domain binds CD138 with an affinity of about 300 nM to about 10 nM or 200 nM to about 10 nM

The ligand can be a monospecific ligand, such as a dAb monomer, a dual specific ligand or a multispecific ligand, that is monovalent, bivalent or multivalent as described herein. Preferably the ligand comprises an immunoglobulin single variable domain (dAb) that has binding specificity for CD138. The ligand can comprise any suitable immunoglobulin single variable domain that has binding specificity for CD138, such a an immunoglobulin heavy chain single variable domain (e.g., V_{H}, Camelid V_{HH}) or immunoglobulin light chain single variable domain (e.g., V_{λ}, V_{κ}). Preferably, the immunoglobulin single variable domain is a heavy chain single variable domain, such as a V_{H} (e.g., a human V_{H}) or a Camelid V_{HH}. Preferably, the immunoglobulin single variable domain binds CD138 (e.g., human CD138) with high affinity, and inhibits the activity of CD138 (e.g., is a CD138 antagonist) as described herein. Preferred ligands, generally comprises a human immunoglobulin single variable domain, or an immunoglobulin single variable domain that comprises human framework regions. In certain embodiments, the ligand comprises a human immunoglobulin single variable domain that comprises a universal framework, as described herein.

In some embodiments, the ligand comprises a humanized immunoglobulin single variable domain with binding specificity for CD138, preferably human CD138. In preferred embodiments, the ligand comprises a human immunoglobulin single variable domain with binding specificity for human CD138.

In some embodiment, the ligand is a dual specific ligand that comprises at least one first polypeptide domain that has a binding site with binding specificity for CD138, and at least one second polypeptide binding domain that has a binding site with binding specificity for another protein. For example, the second polypeptide domain can have a binding site with binding specificity for a receptor disclosed herein, for a receptor for a cytokine or growth factor as disclosed herein, or for a polypeptide that enhances serum half life in vivo (e.g., serum albumin). In particular embodiments, both the first polypeptide domain and the second polypeptide domain are immunoglobulin single variable domains, preferably single heavy chain variable domains, such as V_{H} (e.g., a human V_{H}) or a Camelid V_{HH}.

In one example of a dual specific ligand that has binding specificity for CD138, the ligand comprise at least one immunoglobulin single variable domain (dAb) that has binding specificity for CD138 (e.g., human CD138) and at least one immunoglobulin single variable domain that has binding specificity for serum albumin (e.g., human serum albumin). In particular embodiments, the immunoglobulin single variable domains are heavy chain variable domains (e.g., V_{H}, V_{HH}). For example, the ligand can contain two immunoglobulin single heavy chain variable domains (e.g., V_{H}, V_{HH}) that have binding specificity for CD138 and an immunoglobulin single heavy chain variable domain (e.g., V_{H}, V_{HH}) that has binding specificity for serum albumin. The immunoglobulin single variable domains that have binding specificity for CD138 can bind to the same or different epitopes on CD138 as desired. Additionally, the ligand can contain two or more copies of an immunoglobulin single variable domain that has binding specificity for CD138, or can contain two or more different immunoglobulin single variable domains that each have binding specificity for CD138.

In particular embodiments, the ligand that has a binding site with binding specificity for CD138 competes for binding to CD138 with a dAb that has binding specificity for CD138 such as any one of the anti-CD138 dAbs disclosed in International Application No. PCT/GB2006/004565, filed Dec. 5, 2006, which designates the United States, the entire contents of which are incorporated herein by reference. In some embodiments, the a polypeptide domain that has a binding site with binding specificity for CD138 competes for binding to CD138 with a dAb selected from the group consisting of DOM12-1 (SEQ ID NO:196), DOM12-15 (SEQ ID NO:197), DOM12-17 (SEQ ID NO:198), DOM12-19 (SEQ ID NO:199), DOM12-2 (SEQ ID NO:200), DOM12-20 (SEQ ID NO:201), DOM12-21 (SEQ ID NO:202), DOM12-22 (SEQ ID NO:203), DOM12-3 (SEQ ID NO:204), DOM12-33 (SEQ ID NO:205), DOM12-39 (SEQ ID NO:206), DOM12-4 (SEQ ID NO:207), DOM12-40 (SEQ ID NO:208), DOM12-41 (SEQ ID NO:209), DOM12-42 (SEQ ID NO:210), DOM12-44 (SEQ ID NO:211), DOM12-46 (SEQ ID NO:212), DOM12-6 (SEQ ID NO:213), DOM12-7 (SEQ ID NO:214), DOM12-10 (SEQ ID NO:215), DOM12-11 (SEQ ID NO:216), DOM12-18 (SEQ ID NO:217), DOM12-23 (SEQ ID NO:218), DOM12-24 (SEQ ID NO:219), DOM12-25 (SEQ ID NO:220), DOM12-26 (SEQ ID NO:221), DOM12-27 (SEQ ID NO:222), DOM12-28 (SEQ ID NO:223), DOM12-29 (SEQ ID NO:224), DOM12-30 (SEQ ID NO:225), DOM12-31 (SEQ ID NO:226), DOM12-32 (SEQ ID NO:227), DOM12-34 (SEQ ID NO:228), DOM12-35 (SEQ ID NO:229), DOM12-36 (SEQ ID NO:230), DOM12-37 (SEQ ID NO:231), DOM12-38 (SEQ ID NO:232), DOM12-43 (SEQ ID NO:233), DOM12-45 (SEQ ID NO:234), DOM12-5 (SEQ ID NO:235), DOM12-8 (SEQ ID NO:236), and DOM12-9 (SEQ ID NO:237).

In some embodiments, the a polypeptide domain that has a binding site with binding specificity for CD138 competes for binding to CD138 with a dAb selected from the group consisting of DOM12-45-1 (SEQ ID NO:238), DOM12-45-2 (SEQ ID NO:239), DOM12-45-3 (SEQ ID NO:240), DOM12-45-4 (SEQ ID NO:241), DOM 12-45-5 (SEQ ID NO:242), DOM12-45-6 (SEQ ID NO:243), DOM12-45-8 (SEQ ID NO:244), DOM12-45-9 (SEQ ID NO:245), DOM 12-45-10 (SEQ ID NO:246), DOM 12-45-11 (SEQ ID NO:247), DOM 12-45-12 (SEQ ID NO:248), DOM 12-45-13 (SEQ ID NO:249), DOM 12-45-14 (SEQ ID NO:250), DOM 12-45-15 (SEQ ID NO:251), DOM 12-45-16 (SEQ ID NO:252), DOM 12-45-17 (SEQ ID NO:253), DOM 12-45-18 (SEQ ID NO:254), DOM 12-45-19 (SEQ ID NO:255), DOM 12-45-20 (SEQ ID NO:256), DOM 12-45-21 (SEQ ID NO:257), DOM 12-45-22 (SEQ ID NO:258), DOM 12-45-23 (SEQ ID NO:259), DOM 12-45-24 (SEQ ID NO:260), DOM 12-45-25 (SEQ ID NO:261), DOM 12-45-26 (SEQ ID NO:262), DOM 12-45-27 (SEQ ID NO:263), DOM 12-45-28 (SEQ ID NO:264), DOM 12-45-29 (SEQ ID NO:265), DOM 12-45-30 (SEQ ID NO:266), DOM 12-45-31 (SEQ ID NO:267), DOM 12-45-32 (SEQ ID NO:268), DOM 12-45-33 (SEQ ID NO:269), DOM 12-45-34 (SEQ ID NO:270), DOM 12-45-35 (SEQ ID NO:271), DOM 12-45-36 (SEQ ID NO:272), DOM 12-45-37 (SEQ ID NO:273), and DOM 12-45-38 (SEQ ID NO:274).

In some embodiments, the polypeptide domain that has a binding site with binding specificity for CD138 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM12-1 consisting of DOM12-1 (SEQ ID NO:196), DOM12-15 (SEQ ID NO:197), DOM12-17 (SEQ ID NO:198), DOM12-19 (SEQ ID NO:199), DOM12-2 (SEQ ID NO:200), DOM12-20 (SEQ ID NO:201), DOM12-21 (SEQ ID NO:202), DOM12-22 (SEQ ID NO:203), DOM12-3 (SEQ ID NO:204), DOM12-33 (SEQ ID NO:205), DOM12-39 (SEQ ID NO:206), DOM12-4 (SEQ ID NO:207), DOM12-40 (SEQ ID NO:208), DOM12-41 (SEQ ID NO:209), DOM12-42 (SEQ ID NO:210), DOM12-44 (SEQ ID NO:211), DOM12-46 (SEQ ID NO:212), DOM12-6 (SEQ ID NO:213), DOM12-7 (SEQ ID NO:214), DOM12-10 (SEQ ID NO:215), DOM12-11 (SEQ ID NO:216), DOM12-18 (SEQ ID NO:217), DOM12-23 (SEQ ID NO:218), DOM12-24 (SEQ ID NO:219), DOM12-25 (SEQ ID NO:220), DOM12-26 (SEQ ID NO:221), DOM12-27 (SEQ ID NO:222), DOM12-28 (SEQ ID NO:223), DOM12-29 (SEQ ID NO:224), DOM12-30 (SEQ ID NO:225), DOM12-31 (SEQ ID NO:226), DOM12-32 (SEQ ID NO:227), DOM12-34 (SEQ ID NO:228), DOM12-35 (SEQ ID NO:229), DOM12-36 (SEQ ID NO:230), DOM12-37 (SEQ ID NO:231), DOM12-38 (SEQ ID NO:232), DOM12-43 (SEQ ID NO:233), DOM12-45 (SEQ ID NO:234), DOM12-5 (SEQ ID NO:235), DOM12-8 (SEQ ID NO:236), and DOM12-9 (SEQ ID NO:237).

In some embodiments, the polypeptide domain that has a binding site with binding specificity for CD138 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM12-45-1 (SEQ ID NO:238), DOM12-45-2 (SEQ ID NO:239), DOM12-45-3 (SEQ ID NO:240), DOM12-45-4 (SEQ ID NO:241), DOM12-45-5 (SEQ ID NO:242), DOM12-45-6 (SEQ ID NO:243), DOM12-45-8 (SEQ ID NO:244), DOM12-45-9 (SEQ ID NO:245), DOM 12-45-10 (SEQ ID NO:246), DOM 12-45-11 (SEQ ID NO:247), DOM 12-45-12 (SEQ ID NO:248), DOM 12-45-13 (SEQ ID NO:249), DOM 12-45-14 (SEQ ID NO:250), DOM 12-45-15 (SEQ ID NO:251), DOM 12-45-16 (SEQ ID NO:252), DOM 12-45-17 (SEQ ID NO:253), DOM 12-45-18 (SEQ ID NO:254), DOM 12-45-19 (SEQ ID NO:255), DOM 12-45-20 (SEQ ID NO:256), DOM 12-45-21 (SEQ ID NO:257), DOM 12-45-22 (SEQ ID NO:258), DOM 12-45-23 (SEQ ID NO:259), DOM 12-45-24 (SEQ ID NO:260), DOM 12-45-25 (SEQ ID NO:261), DOM 12-45-26 (SEQ ID NO:262), DOM 12-45-27 (SEQ ID NO:263), DOM 12-45-28 (SEQ ID NO:264), DOM 12-45-29 (SEQ ID NO:265), DOM 12-45-30 (SEQ ID NO:266), DOM 12-45-31 (SEQ ID NO:267), DOM 12-45-32 (SEQ ID NO:268), DOM 12-45-33 (SEQ ID NO:269), DOM 12-45-34 (SEQ ID NO:270), DOM 12-45-35 (SEQ ID NO:271), DOM 12-45-36 (SEQ ID NO:272), DOM 12-45-37 (SEQ ID NO:273), and DOM 12-45-38 (SEQ ID NO:274).

Polypeptide Domains that Bind Carcinoembryonic Antigen (CEA).

The invention also provides ligands (e.g., isolated dAbs) that have a binding domain (e.g., a domain comprising a binding site) with binding specificity for CEA. In preferred embodiments, the ligand binds to CEA with an affinity of 300 nM to 5 pM (ie, 3×10⁻⁷ to 5×10⁻¹²M) or 300 nM to 1 pM, preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance. In some embodiments, the polypeptide domain binds to CEA with low affinity, such as an affinity between about 10 μM to about 10 nM as determined by surface plasmon resonance. For example, the polypeptide domain can bind CEA with an affinity of about 10 μM to about 300 nM, or about 10 μM to about 400 nM. In certain embodiments, the polypeptide domain binds CEA with an affinity of about 300 nM to about 10 nM or 200 nM to about 10 nM.

The ligand can be a monospecific ligand, such as a dAb monomer, a dual specific ligand or a multispecific ligand, that is monovalent, bivalent or multivalent as described herein. Preferably, the ligand comprises an immunoglobulin single variable domain (dAb) that has binding specificity for CEA. The ligand can comprise any suitable immunoglobulin single variable domain that has binding specificity for CEA, such a an immunoglobulin heavy chain single variable domain (e.g., V_{H}, Camelid V_{HH}) or immunoglobulin light chain single variable domain (e.g., V_{λ}, V_{κ}). Preferably, the immunoglobulin single variable domain is a heavy chain single variable domain, such as a V_{H} (e.g., a human V_{H}) or a Camelid V_{HH}. Preferably, the immunoglobulin single variable domain binds CEA (e.g., human CEA) with high affinity, and inhibits the activity of CEA (e.g., is a CEA antagonist) as described herein. Preferred ligands, generally comprises a human immunoglobulin single variable domain, or an immunoglobulin single variable domain that comprises human framework regions. In certain embodiments, the ligand comprises a human immunoglobulin single variable domain that comprises a universal framework, as described herein.

In some embodiments, the ligand comprises a humanized immunoglobulin single variable domain with binding specificity for CEA, preferably human CEA. In preferred embodiments, the ligand comprises a human immunoglobulin single variable domain with binding specificity for human CEA.

In some embodiment, the ligand is a dual specific ligand that comprises at least one first polypeptide domain that has a binding site with binding specificity for CEA, and at least one second polypeptide binding domain that has a binding site with binding specificity for another protein. For example, the second polypeptide domain can have a binding site with binding specificity for a receptor disclosed herein, for a receptor for a cytokine or growth factor as disclosed herein, or for a polypeptide that enhances serum half life in vivo (e.g., serum albumin). In particular embodiments, both the first polypeptide domain and the second polypeptide domain are immunoglobulin single variable domains, preferably single heavy chain variable domains, such as V_{H} (e.g., a human V_{H}) or a Camelid V_{HH}.

In one example of a dual specific ligand that has binding specificity for CEA, the ligand comprise at least one immunoglobulin single variable domain (dAb) that has binding specificity for CEA (e.g., human CEA) and at least one immunoglobulin single variable domain that has binding specificity for serum albumin (e.g., human serum albumin). In particular embodiments, the immunoglobulin single variable domains are heavy chain variable domains (e.g., V_{H}, V_{HH}). For example, the ligand can contain two immunoglobulin single heavy chain variable domains (e.g., V_{H}, V_{HH}) that have binding specificity for CEA and an immunoglobulin single heavy chain variable domain (e.g., V_{H}, V_{HH}) that has binding specificity for serum albumin. The immunoglobulin single variable domains that have binding specificity for CEA can bind to the same or different epitopes on CEA as desired. Additionally, the ligand can contain two or more copies of an immunoglobulin single variable domain that has binding specificity for CEA, or can contain two or more different immunoglobulin single variable domains that each have binding specificity for CEA.

In particular embodiments, the ligand that has a binding site with binding specificity for CEA competes for binding to CEA with a dAb that has binding specificity for CEA such as any one of the anti-CEA dAbs disclosed in International Application No. PCT/GB2006/004565, filed Dec. 5, 2006, which designates the United States, the entire contents of which are incorporated herein by reference. In some embodiments, the polypeptide domain that has a binding site with binding specificity for CEA competes for binding to CEA with a dAb selected from the group consisting of DOM13-1 (SEQ ID NO:275), DOM13-12 (SEQ ID NO:276), DOM13-13 (SEQ ID NO:277), DOM13-14 (SEQ ID NO:278), DOM13-15 (SEQ ID NO:279), DOM13-16 (SEQ ID NO:280), DOM13-17 (SEQ ID NO:281), DOM13-18 (SEQ ID NO:282), DOM13-19 (SEQ ID NO:283), DOM13-2 (SEQ ID NO:284), DOM13-20 (SEQ ID NO:285), DOM13-21 (SEQ ID NO:286), DOM13-22 (SEQ ID NO:287), DOM13-23 (SEQ ID NO:288), DOM13-24 (SEQ ID NO:289), DOM13-25 (SEQ ID NO:290), DOM13-26 (SEQ ID NO:291), DOM13-27 (SEQ ID NO:292), DOM13-28 (SEQ ID NO:293), DOM13-29 (SEQ ID NO:294), DOM13-3 (SEQ ID NO:295), DOM13-30 (SEQ ID NO:296), DOM13-31 (SEQ ID NO:297), DOM13-32 (SEQ ID NO:298), DOM13-33 (SEQ ID NO:299), DOM-13-34 (SEQ ID NO:300), DOM13-35 (SEQ ID NO:301), DOM13-36 (SEQ ID NO:302), DOM13-37 (SEQ ID NO:303), DOM13-4 (SEQ ID NO:304), DOM13-42 (SEQ ID NO:305), DOM13-43 (SEQ ID NO:306), DOM13-44 (SEQ ID NO:307), DOM13-45 (SEQ ID NO:308), DOM13-46 (SEQ ID NO:309), DOM13-47 (SEQ ID NO:310), DOM13-48 (SEQ ID NO:311), DOM13-49 (SEQ ID NO:312), DOM13-5 (SEQ ID NO:313), DOM13-50 (SEQ ID NO:314), DOM13-51 (SEQ ID NO:315), DOM13-52 (SEQ ID NO:316), DOM13-53 (SEQ ID NO:317), DOM13-54 (SEQ ID NO:318), DOM13-55 (SEQ ID NO:319), DOM13-56 (SEQ ID NO:320), DOM13-57 (SEQ ID NO:321), DOM13-58 (SEQ ID NO:322), DOM13-59 (SEQ ID NO:323), DOM13-6 (SEQ ID NO:324), DOM13-60 (SEQ ID NO:325), DOM13-61 (SEQ ID NO:326), DOM13-62 (SEQ ID NO:327), DOM13-63 (SEQ ID NO:328), DOM13-64 (SEQ ID NO:329), DOM13-65 (SEQ ID NO:330), DOM13-66 (SEQ ID NO:331), DOM13-67 (SEQ ID NO:332), DOM13-68 (SEQ ID NO:333), DOM13-69 (SEQ ID NO:334), DOM13-7 (SEQ ID NO:335), DOM13-70 (SEQ ID NO:336), DOM13-71 (SEQ ID NO:337), DOM13-72 (SEQ ID NO:338), DOM13-73 (SEQ ID NO:339), DOM13-74 (SEQ ID NO:340), DOM13-75 (SEQ ID NO:341), DOM13-76 (SEQ ID NO:342), DOM13-77 (SEQ ID NO:343), DOM13-78 (SEQ ID NO:344), DOM13-79 (SEQ ID NO:345), DOM13-8 (SEQ ID NO:346), DOM13-80 (SEQ ID NO:347), DOM13-81(SEQ ID NO:348), DOM13-82 (SEQ ID NO:349), DOM13-83 (SEQ ID NO:350), DOM13-84 (SEQ ID NO:351), DOM13-85 (SEQ ID NO:352), DOM13-86 (SEQ ID NO:353), DOM13-87 (SEQ ID NO:354), DOM13-88 (SEQ ID NO:355), DOM13-89 (SEQ ID NO:356), DOM13-90 (SEQ ID NO:357), DOM13-91 (SEQ ID NO:358), DOM13-92 (SEQ ID NO:359), DOM13-93 (SEQ ID NO:360), DOM13-94 (SEQ ID NO:361), and DOM13-95 (SEQ ID NO:362).

In certain embodiments, the polypeptide domain that has a binding site with binding specificity for CEA competes for binding to CEA with a dAb selected from the group consisting of DOM13-25-3 (SEQ ID NO:363), DOM 13-25-23 (SEQ ID NO:364), DOM 13-25-27 (SEQ ID NO:365), and DOM 13-25-80 (SEQ ID NO:366).

In some embodiments, the polypeptide domain that has a binding site with binding specificity for CEA comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM13-1 (SEQ ID NO:275), DOM13-12 (SEQ ID NO:276), DOM13-13 (SEQ ID NO:277), DOM13-14 (SEQ ID NO:278), DOM13-15 (SEQ ID NO:279), DOM13-16 (SEQ ID NO:280), DOM13-17 (SEQ ID NO:281), DOM13-18 (SEQ ID NO:282), DOM13-19 (SEQ ID NO:283), DOM13-2 (SEQ ID NO:284), DOM13-20 (SEQ ID NO:285), DOM13-21 (SEQ ID NO:286), DOM13-22 (SEQ ID NO:287), DOM13-23 (SEQ ID NO:288), DOM13-24 (SEQ ID NO:289), DOM13-25 (SEQ ID NO:290), DOM13-26 (SEQ ID NO:291), DOM13-27 (SEQ ID NO:292), DOM13-28 (SEQ ID NO:293), DOM13-29 (SEQ ID NO:294), DOM13-3 (SEQ ID NO:295), DOM13-30 (SEQ ID NO:296), DOM13-31 (SEQ ID NO:297), DOM13-32 (SEQ ID NO:298), DOM13-33 (SEQ ID NO:299), DOM-13-34 (SEQ ID NO:300), DOM13-35 (SEQ ID NO:301), DOM13-36 (SEQ ID NO:302), DOM13-37 (SEQ ID NO:303), DOM13-4 (SEQ ID NO:304), DOM13-42 (SEQ ID NO:305), DOM13-43 (SEQ ID NO:306), DOM13-44 (SEQ ID NO:307), DOM13-45 (SEQ ID NO:308), DOM13-46 (SEQ ID NO:309), DOM13-47 (SEQ ID NO:310), DOM13-48 (SEQ ID NO:311), DOM13-49 (SEQ ID NO:312), DOM13-5 (SEQ ID NO:313), DOM13-50 (SEQ ID NO:314), DOM13-51 (SEQ ID NO:315), DOM13-52 (SEQ ID NO:316), DOM13-53 (SEQ ID NO:317), DOM13-54 (SEQ ID NO:318), DOM13-55 (SEQ ID NO:319), DOM13-56 (SEQ ID NO:320), DOM13-57 (SEQ ID NO:321), DOM13-58 (SEQ ID NO:322), DOM13-59 (SEQ ID NO:323), DOM13-6 (SEQ ID NO:324), DOM13-60 (SEQ ID NO:325), DOM13-61 (SEQ ID NO:326), DOM13-62 (SEQ ID NO:327), DOM13-63 (SEQ ID NO:328), DOM13-64 (SEQ ID NO:329), DOM13-65 (SEQ ID NO:330), DOM13-66 (SEQ ID NO:331), DOM13-67 (SEQ ID NO:332), DOM13-68 (SEQ ID NO:333), DOM13-69 (SEQ ID NO:334), DOM13-7 (SEQ ID NO:335), DOM13-70 (SEQ ID NO:336), DOM13-71 (SEQ ID NO:337), DOM13-72 (SEQ ID NO:338), DOM13-73 (SEQ ID NO:339), DOM13-74 (SEQ ID NO:340), DOM13-75 (SEQ ID NO:341), DOM13-76 (SEQ ID NO:342), DOM13-77 (SEQ ID NO:343), DOM13-78 (SEQ ID NO:344), DOM13-79 (SEQ ID NO:345), DOM13-8 (SEQ ID NO:346), DOM13-80 (SEQ ID NO:347), DOM13-81(SEQ ID NO:348), DOM13-82 (SEQ ID NO:349), DOM13-83 (SEQ ID NO:350), DOM13-84 (SEQ ID NO:351), DOM13-85 (SEQ ID NO:352), DOM13-86 (SEQ ID NO:353), DOM13-87 (SEQ ID NO:354), DOM13-88 (SEQ ID NO:355), DOM13-89 (SEQ ID NO:356), DOM13-90 (SEQ ID NO:357), DOM13-91 (SEQ ID NO:358), DOM13-92 (SEQ ID NO:359), DOM13-93 (SEQ ID NO:360), DOM13-94 (SEQ ID NO:361), and DOM13-95 (SEQ ID NO:362).

In other embodiments, the polypeptide domain that has a binding site with binding specificity for CEA comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of: DOM13-25-3 (SEQ ID NO:363), DOM 13-25-23 (SEQ ID NO:364), DOM 13-25-27 (SEQ ID NO:365), and DOM 13-25-80 (SEQ ID NO:366).

Polypeptide Domains that Bind CD56

The invention also provides ligands (e.g., isolated dAbs) that have a binding domain (e.g., a domain comprising a binding site) with binding specificity for CD56. In preferred embodiments, the ligand binds to CD56 with an affinity of 300 nM to 5 pM (ie, 3×10⁻⁷ to 5×10⁻¹²M) or 300 nM to 1 pM, preferably 50 nM to 20 pM, more preferably 5 nM to 200 pM and most preferably 1 nM to 100 pM, for example 1×10⁻⁷ M or less, preferably 1×10⁻⁸ M or less, more preferably 1×10⁻⁹ M or less, advantageously 1×10⁻¹⁰ M or less and most preferably 1×10⁻¹¹ M or less; and/or a K_{off} rate constant of 5×10⁻¹ s⁻¹ to 1×10⁻⁷ s⁻¹, preferably 1×10⁻² s⁻¹ to 1×10⁻⁶ s⁻¹, more preferably 5×10⁻³ s⁻¹ to 1×10⁻⁵ s⁻¹, for example 5×10⁻¹ s⁻¹ or less, preferably 1×10⁻² s⁻¹ or less, advantageously 1×10⁻³ s⁻¹ or less, more preferably 1×10⁻⁴ s⁻¹ or less, still more preferably 1×10⁻⁵ s⁻¹ or less, and most preferably 1×10⁻⁶ s⁻¹ or less as determined by surface plasmon resonance. In some embodiments, the polypeptide domain binds to CD56 with low affinity, such as an affinity between about 10 μM to about 10 nM as determined by surface plasmon resonance. For example, the polypeptide domain can bind CD56 with an affinity of about 10 μM to about 300 nM, or about 10 μM to about 400 nM. In certain embodiments, the polypeptide domain binds CD56 with an affinity of about 300 nM to about 10 nM or 200 nM to about 10 nM.

The ligand can be a monospecific ligand, such as a dAb monomer, a dual specific ligand or a multispecific ligand, that is monovalent, bivalent or multivalent as described herein. Preferably, the ligand comprises an immunoglobulin single variable domain (dAb) that has binding specificity for CD56. The ligand can comprise any suitable immunoglobulin single variable domain that has binding specificity for CD56, such a an immunoglobulin heavy chain single variable domain (e.g., V_{H}, Camelid V_{HH}) or immunoglobulin light chain single variable domain (e.g., V_{λ}, V_{κ}). Preferably, the immunoglobulin single variable domain is a heavy chain single variable domain, such as a V_{H} (e.g., a human V_{H}) or a Camelid V_{HH}. Preferably, the immunoglobulin single variable domain binds CD56 (e.g., human CD56) with high affinity, and inhibits the activity of CD56 (e.g., is a CD56 antagonist) as described herein. Preferred ligands, generally comprises a human immunoglobulin single variable domain, or an immunoglobulin single variable domain that comprises human framework regions. In certain embodiments, the ligand comprises a human immunoglobulin single variable domain that comprises a universal framework, as described herein.

In some embodiments, the ligand comprises a humanized immunoglobulin single variable domain with binding specificity for CD56, preferably human CD56. In preferred embodiments, the ligand comprises a human immunoglobulin single variable domain with binding specificity for human CD56.

In some embodiment, the ligand is a dual specific ligand that comprises at least one first polypeptide domain that has a binding site with binding specificity for CD56, and at least one second polypeptide binding domain that has a binding site with binding specificity for another protein. For example, the second polypeptide domain can have a binding site with binding specificity for a receptor disclosed herein, for a receptor for a cytokine or growth factor as disclosed herein, or for a polypeptide that enhances serum half life in vivo (e.g., serum albumin). In particular embodiments, both the first polypeptide domain and the second polypeptide domain are immunoglobulin single variable domains, preferably single heavy chain variable domains, such as V_{H} (e.g, a human V_{H}) or a Camelid V_{HH}.

In one example of a dual specific ligand that has binding specificity for CD56, the ligand comprise at least one immunoglobulin single variable domain (dAb) that has binding specificity for CD56 (e.g., human CD56) and at least one immunoglobulin single variable domain that has binding specificity for serum albumin (e.g., human serum albumin). In particular embodiments, the immunoglobulin single variable domains are heavy chain variable domains (e.g., V_{H}, V_{HH}). For example, the ligand can contain two immunoglobulin single heavy chain variable domains (e.g., V_{H}, V_{HH}) that have binding specificity for CEA and an immunoglobulin single heavy chain variable domain (e.g., V_{H}, V_{HH}) that has binding specificity for serum albumin. The immunoglobulin single variable domains that have binding specificity for CD56 can bind to the same or different epitopes on CD56 as desired. Additionally, the ligand can contain two or more copies of an immunoglobulin single variable domain that has binding specificity for CD56, or can contain two or more different immunoglobulin single variable domains that each have binding specificity for CD56.

In particular embodiments, the ligand that has a binding site with binding specificity for CD56 competes for binding to CD56 with a dAb that has binding specificity for CD56 such as any one of the anti-CD56 dAbs disclosed in International Application No. PCT/GB2006/004565, filed Dec. 5, 2006, which designates the United States, the entire contents of which are incorporated herein by reference. In some embodiments, the polypeptide domain that has a binding site with binding specificity for CD56 competes for binding to CD56 with a dAb selected from the group consisting of DOM14-1 (SEQ ID NO:367), DOM14-10 (SEQ ID NO:368), DOM14-100 (SEQ ID NO:369), DOM14-11 (SEQ ID NO:370), DOM14-12 (SEQ ID NO:371), DOM14-13 (SEQ ID NO:372), DOM14-14 (SEQ ID NO:373), DOM14-15 (SEQ ID NO:374), DOM14-16 (SEQ ID NO:375), DOM14-17 (SEQ ID NO:376), DOM14-18 (SEQ ID NO:377), DOM14-19 (SEQ ID NO:378), DOM14-2 (SEQ ID NO:379), DOM14-20 (SEQ ID NO:380), DOM14-21 (SEQ ID NO:381), DOM14-22 (SEQ ID NO:382), DOM14-23 (SEQ ID NO:383), DOM14-24 (SEQ ID NO:384), DOM14-25 (SEQ ID NO:385), DOM14-26 (SEQ ID NO:386), DOM14-27 (SEQ ID NO:387), DOM14-28 (SEQ ID NO:388), DOM14-3 (SEQ ID NO:389), DOM14-31 (SEQ ID NO:390), DOM14-32 (SEQ ID NO:391), DOM14-33 (SEQ ID NO:392), DOM14-34 (SEQ ID NO:393), DOM14-35 (SEQ ID NO:394), DOM14-36 (SEQ ID NO:395), DOM14-37 (SEQ ID NO:396), DOM14-38 (SEQ ID NO:397), DOM14-39 (SEQ ID NO:398), DOM14-4 (SEQ ID NO:399), DOM14-40 (SEQ ID NO:400), DOM14-41 (SEQ ID NO:401), DOM14-42 (SEQ ID NO:402), DOM14-43 (SEQ ID NO:403), DOM14-44 (SEQ ID NO:404), DOM14-45 (SEQ ID NO:405), DOM14-46 (SEQ ID NO:406), DOM14-47 (SEQ ID NO:407), DOM14-48 (SEQ ID NO:408), DOM14-49 (SEQ ID NO:409), DOM14-50 (SEQ ID NO:410), DOM14-51 (SEQ ID NO:411), DOM14-52 (SEQ ID NO:412), DOM14-53 (SEQ ID NO:413), DOM14-54 (SEQ ID NO:414), DOM14-55 (SEQ ID NO:415), DOM14-56 (SEQ ID NO:416), DOM14-57 (SEQ ID NO:417), DOM14-58 (SEQ ID NO:418), DOM14-59 (SEQ ID NO:419), DOM14-60 (SEQ ID NO:420), DOM14-61 (SEQ ID NO:421), DOM14-62 (SEQ ID NO:422), DOM14-63 (SEQ ID NO:423), DOM14-64 (SEQ ID NO:424), DOM14-65 (SEQ ID NO:425), DOM14-66 (SEQ ID NO:426), DOM14-67 (SEQ ID NO:427), DOM14-70 (SEQ ID NO:428), DOM14-68 (SEQ ID NO:429), and DOM14-69 (SEQ ID NO:430).

In some embodiments, the polypeptide domain that has a binding site with binding specificity for CD56 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb selected from the group consisting of DOM14-1 (SEQ ID NO:367), DOM14-10 (SEQ ID NO:368), DOM14-100 (SEQ ID NO:369), DOM14-11 (SEQ ID NO:370), DOM14-12 (SEQ ID NO:371), DOM14-13 (SEQ ID NO:372), DOM14-14 (SEQ ID NO:373), DOM14-15 (SEQ ID NO:374), DOM14-16 (SEQ ID NO:375), DOM14-17 (SEQ ID NO:376), DOM14-18 (SEQ ID NO:377), DOM14-19 (SEQ ID NO:378), DOM14-2 (SEQ ID NO:379), DOM14-20 (SEQ ID NO:380), DOM14-21 (SEQ ID NO:381), DOM14-22 (SEQ ID NO:382), DOM14-23 (SEQ ID NO:383), DOM14-24 (SEQ ID NO:384), DOM14-25 (SEQ ID NO:385), DOM14-26 (SEQ ID NO:386), DOM14-27 (SEQ ID NO:387), DOM14-28 (SEQ ID NO:388), DOM14-3 (SEQ ID NO:389), DOM14-31 (SEQ ID NO:390), DOM14-32 (SEQ ID NO:391), DOM14-33 (SEQ ID NO:392), DOM14-34 (SEQ ID NO:393), DOM14-35 (SEQ ID NO:394), DOM14-36 (SEQ ID NO:395), DOM14-37 (SEQ ID NO:396), DOM14-38 (SEQ ID NO:397), DOM14-39 (SEQ ID NO:398), DOM14-4 (SEQ ID NO:399), DOM14-40 (SEQ ID NO:400), DOM14-41 (SEQ ID NO:401), DOM14-42 (SEQ ID NO:402), DOM14-43 (SEQ ID NO:403), DOM14-44 (SEQ ID NO:404), DOM14-45 (SEQ ID NO:405), DOM14-46 (SEQ ID NO:406), DOM14-47 (SEQ ID NO:407), DOM14-48 (SEQ ID NO:408), DOM14-49 (SEQ ID NO:409), DOM14-50 (SEQ ID NO:410), DOM14-51 (SEQ ID NO:411), DOM14-52 (SEQ ID NO:412), DOM14-53 (SEQ ID NO:413), DOM14-54 (SEQ ID NO:414), DOM14-55 (SEQ ID NO:415), DOM14-56 (SEQ ID NO:416), DOM14-57 (SEQ ID NO:417), DOM14-58 (SEQ ID NO:418), DOM14-59 (SEQ ID NO:419), DOM14-60 (SEQ ID NO:420), DOM14-61 (SEQ ID NO:421), DOM14-62 (SEQ ID NO:422), DOM14-63 (SEQ ID NO:423), DOM14-64 (SEQ ID NO:424), DOM14-65 (SEQ ID NO:425), DOM14-66 (SEQ ID NO:426), DOM14-67 (SEQ ID NO:427), DOM14-70 (SEQ ID NO:428), DOM14-68 (SEQ ID NO:429), and DOM14-69 (SEQ ID NO:430).

In some embodiments, the polypeptide domain that has a binding site with binding specificity for CD56 competes for binding to CD56 with the anti-CD56 dAb DOM14-70 (SEQ ID NO:431). In some embodiments, the polypeptide domain that has a binding site with binding specificity for CD56 comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of the anti-CD56 dAb DOM14-70 (SEQ ID NO:431).

Polypeptide Domains that Bind Serum Albumin

In addition to a receptor binding moiety, the ligands of the invention can further comprise a polypeptide domain (e.g., a dAb) that has binding specificity for serum albumin (SA). Preferably, the polypeptide domain binds serum albumin (SA) with an affinity of 1 nM to 500 μM (i.e., ×10⁻⁹ to 5×10⁻⁴), preferably 100 nM to 10 μM. Preferably, for a ligand comprising an anti-SA binding domain (e.g, anti-SA dAb), the binding (eg K_{d} and/or K_{off} as measured by surface plasmon resonance, e.g., using Biacore) of the ligand to its receptor target(s) is from 1 to 100000 times (preferably 100 to 100000, more preferably 1000 to 100000, or 10000 to 100000 times) stronger than for SA. Preferably, the serum albumin is human serum albumin (HSA). In one embodiment, the ligand comprises a dAb that binds SA (e.g., HSA) with a K_{d} of approximately 50 nM, preferably 70 nM, and more preferably 100 nM, 150 nM or 200 nM.

The polypeptide domain (e.g., a dAb) that has binding specificity for serum albumin can have binding specificity for serum albumin of a desired animal, for example serum albumin from dog, cat, horse, cow, chicken, sheep, pig, goat, deer, mink, monkey (e.g., cynomolgus monkey, Macaca fascicularis), mouse, rat and the like. Preferably, the polypeptide domain (e.g., a dAb) that has binding specificity for serum albumin binds human serum albumin. In some embodiments the polypeptide domain (e.g., a dAb) that has binding specificity for serum albumin binds serum albumin from more than one species. For example, human dAbs that have binding specificity for rat serum albumin and mouse serum albumin, and a dAb that has binding specificity for rat, mouse and human serum albumin have been produced. (See, WO 2005/118642 and U.S. application Ser. No. 11/628,149 at Table 1 and FIG. 7 which discloses the specificity of human dAbs that bind serum albumin from human, mouse and/or rat. The entire contents of WO 2005/118642 and U.S. application Ser. No. 11/628,149 are incorporated herein by reference.) Such dAbs provide the advantage of allowing preclinical and clinical studies using the same dAb and obviate the need to conduct preclinical studies with a suitable surrogate. In some embodiments, the ligand comprises a dAb or immunoglobulin single variable domain that binds human serum albumin and mouse serum albumin, human serum albumin and rat serum albumin, human serum albumin and pig serum albumin, or human serum albumin and cynomolgus monkey serum albumin.

The polypeptide binding domain can be any suitable immunoglobulin single variable domain that has binding specificity for SA, such a an immunoglobulin heavy chain single variable domain (e.g., V_{H}, Camelid V_{HH}) or immunoglobulin light chain single variable domain (e.g., V_{λ}, V_{κ}). Preferably, the immunoglobulin single variable domain is a heavy chain single variable domain, such as a V_{H} (e.g., a human V_{H}) or a Camelid V_{HH}. Preferred polypeptide domains that bind SA generally comprises a human immunoglobulin single variable domain, or an immunoglobulin single variable domain that comprises human framework regions. In certain embodiments, the ligand comprises a human immunoglobulin single variable domain that comprises a universal framework, as described herein.

In some embodiments, the ligand comprises a humanized immunoglobulin single variable domain with binding specificity for SA, preferably a human SA. In preferred embodiments, the ligand comprises a human immunoglobulin single variable domain with binding specificity for human SA.

In particular embodiments, the polypeptide domain with binding specificity for SA competes for binding to SA with a dAb that has binding specificity for SA such as any one of the anti-SA dAbs disclosed in International Application No. PCT/GB2005/004603, filed Dec. 1, 2005, which designates the United States, or in International Application No. PCT/GB2003/002804, filed Jun. 30, 2003, which designated the United States, the entire contents of each of the foregoing applications are incorporated herein by reference. For example, in some embodiments, the polypeptide domain with binding specificity for SA competes for binding to SA (e.g., human SA) with an anti-SA dAb selected from the group consisting of DOM7h-2 (SEQ ID NO:432), DOM7h-3 (SEQ ID NO:433), DOM7h-4 (SEQ ID NO:434), DOM7h-6 (SEQ ID NO:435), DOM7h-1 (SEQ ID NO:436), DOM7h-7 (SEQ ID NO:437), DOM7h-8 (SEQ ID NO:438), DOM7r-13 (SEQ ID NO:439), DOM7r-14 (SEQ ID NO:440), DOM7h-22 (SEQ ID NO:441), DOM7h-23 (SEQ ID NO:442), DOM7h-24 (SEQ ID NO:443), DOM7h-25 (SEQ ID NO:444), DOM7h-26 (SEQ ID NO:445), DOM7h-21 (SEQ ID NO:446), and DOM7h-27 (SEQ ID NO:447).

In certain embodiments, the polypeptide domain with binding specificity for SA is a dAb that binds SA (human SA) and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of DOM7h-2 (SEQ ID NO:432), DOM7h-3 (SEQ ID NO:433), DOM7h-4 (SEQ ID NO:434), DOM7h-6 (SEQ ID NO:435), DOM7h-1 (SEQ ID NO:436), DOM7h-7 (SEQ ID NO:437), DOM7h-8 (SEQ ID NO:438), DOM7r-13 (SEQ ID NO:439), DOM7r-14 (SEQ ID NO:440), DOM7h-22 (SEQ ID NO:441), DOM7h-23 (SEQ ID NO:442), DOM7h-24 (SEQ ID NO:443), DOM7h-25 (SEQ ID NO:444), DOM7h-26 (SEQ ID NO:445), DOM7h-21 (SEQ ID NO:446), and DOM7h-27 (SEQ ID NO:447).

Suitable Camelid V_{HH} that bind serum albumin include those disclosed in WO 2004/041862 (Ablynx N.V.) and herein, such as Sequence A (SEQ ID NO:448), Sequence B (SEQ ID NO:449), Sequence C (SEQ ID NO:450), Sequence D (SEQ ID NO:451), Sequence E (SEQ ID NO:452), Sequence F (SEQ ID NO:453), Sequence G (SEQ ID NO:454), Sequence H (SEQ ID NO:455), Sequence I (SEQ ID NO:456), Sequence J (SEQ ID NO:457), Sequence K (SEQ ID NO:458), Sequence L (SEQ ID NO:459), Sequence M (SEQ ID NO:460), Sequence N (SEQ ID NO:461), Sequence I (SEQ ID NO:462), Sequence P (SEQ ID NO:463), Sequence Q (SEQ ID NO:464). In certain embodiments, the Camelid V_{HH} binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with any one of SEQ ID NOS:448-464.

### Nucleic Acid Molecules, Vectors and Host Cells

The invention also provides isolated and/or recombinant nucleic acid molecules encoding ligands, (e.g., dAb monomers, dual-specific ligands, multispecific ligands) as described herein. Nucleic acids referred to herein as “isolated” are nucleic acids which have been separated away from the nucleic acids of the genomic DNA or cellular RNA of their source of origin (e.g., as it exists in cells or in a mixture of nucleic acids such as a library), and include nucleic acids obtained by methods described herein or other suitable methods, including essentially pure nucleic acids, nucleic acids produced by chemical synthesis, by combinations of biological and chemical methods, and recombinant nucleic acids which are isolated (see e.g., Daugherty, B. L. et al., Nucleic Acids Res., 19(9): 2471-2476 (1991); Lewis, A. P. and J. S. Crowe, Gene, 101: 297-302 (1991)).

Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes.

The invention also provides a vector comprising a recombinant nucleic acid molecule of the invention. In certain embodiments, the vector is an expression vector comprising one or more expression control elements or sequences that are operably linked to the recombinant nucleic acid of the invention. The invention also provides a recombinant host cell comprising a recombinant nucleic acid molecule or vector of the invention. Suitable vectors (e.g., plasmids, phagemids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., lac, tac, T3, T7 promoters for E. coli) and eucaryotic (e.g., simian virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin or replication. Genes encoding products which confer antibiotic or drug resistance are common selectable markers and may be used in procaryotic (e.g. lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g, COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acac. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NS0 cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In preferred embodiments, the host cell is a non-human host cell.

The invention also provides a method for producing a ligand (e.g., dual-specific ligand, multispecific ligand) of the invention, comprising maintaining a recombinant host cell comprising a recombinant nucleic acid of the invention under conditions suitable for expression of the recombinant nucleic acid, whereby the recombinant nucleic acid is expressed and a ligand is produced. In some embodiments, the method further comprises isolating the ligand.

### Preparation of Immunoglobulin Based Ligands

Ligands (e.g., dAb monomers, dual specific ligands, multispecific ligands) according to the invention can be prepared according to previously established techniques, used in the field of antibody engineering, for the preparation of scFv, “phage” antibodies and other engineered antibody molecules. Techniques for the preparation of antibodies are, for example, described in the following reviews and the references cited therein: Winter & Milstein, (1991) Nature 349:293-299; Pluckthun (1992) Immunological Reviews 13 0:151-188; Wright et al., (1992) Crti. Rev. Immunol. 12:125-168; Holliger, P. & Winter, G. (1993) Curr. Op. Biotechn. 4, 446-449; Carter, et al. (1995) J. Hematother. 4, 463-470; Chester, K. A. & Hawkins, R. E. (1995) Trends Biotechn. 13, 294-300; Hoogenboom, H. R. (1997) Nature Biotechnol. 15, 125-126; Fearon, D. (1997) Nature Biotechnol. 15, 618-619; Plückthun, A. & Pack, P. (1997) Immunotechnology 3, 83-105; Carter, P. & Merchant, A. M. (1997) Curr. Opin. Biotechnol. 8, 449-454; Holliger, P. & Winter, G. (1997) Cancer Immunol. Immunother. 45, 128-130.

Suitable techniques employed for selection of antibody variable domains with a desired specificity employ libraries and selection procedures which are known in the art. Natural libraries (Marks et al. (1991) J. Mol. Biol., 222: 581; Vaughan et al. (1996) Nature Biotech., 14: 309) which use rearranged V genes harvested from human B cells are well known to those skilled in the art. Synthetic libraries (Hoogenboom & Winter (1992) J. Mol. Biol., 227: 381; Barbas et al. (1992) Proc. Natl. Acad. Sci. USA, 89: 4457; Nissim et al. (1994) EMBO J, 13: 692; Griffiths et al. (1994) EMBO J, 13: 3245; De Kruif et al. (1995) J. Mol. Biol., 248: 97) are prepared by cloning immunoglobulin V genes, usually using PCR. Errors in the PCR process can lead to a high degree of randomisation. V_{H} and/or V_{L} libraries may be selected against target antigens or epitopes separately, in which case single domain binding is directly selected for, or together.

### Library Vector Systems

A variety of selection systems are known in the art which are suitable for use in the present invention. Examples of such systems are described below.

Bacteriophage lambda expression systems may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the invention. Whilst such expression systems can be used to screen up to 10⁶ different members of a library, they are not really suited to screening of larger numbers (greater than 10⁶ members). Of particular use in the construction of libraries are selection display systems, which enable a nucleic acid to be linked to the polypeptide it expresses. As used herein, a selection display system is a system that permits the selection, by suitable display means, of the individual members of the library by binding the generic and/or target.

Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encoding them) for the in vitro selection and amplification of specific antibody fragments that bind a target antigen (McCafferty et al., WO 92/01047). The nucleotide sequences encoding the variable regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) Nature, 348: 552; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad Sci U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci. U.S.A., 85: 5879-5883; Chaudhary et al. (1990) Proc. Natl. Acad. Sci. U.S.A., 87:1066-1070; McCafferty et al. (1990) supra; Clackson et al. (1991) Nature, 352: 624; Marks et al. (1991) J. Mol. Biol., 222: 581; Chiswell et al. (1992) Trends Biotech., 10: 80; Marks et al. (1992) J. Biol. Chem., 267). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference.

Other systems for generating libraries of polypeptides involve the use of cell-free enzymatic machinery for the in vitro synthesis of the library members. In one method, RNA molecules are selected by alternate rounds of selection against a target and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection.

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths (1998) Nature Biotechnol 16(7), 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

### Library Construction

Libraries intended for selection, may be constructed using techniques known in the art, for example as set forth above, or may be purchased from commercial sources. Libraries which are useful in the present invention are described, for example, in WO99/20749. Once a vector system is chosen and one or more nucleic acid sequences encoding polypeptides of interest are cloned into the library vector, one may generate diversity within the cloned molecules by undertaking mutagenesis prior to expression; alternatively, the encoded proteins may be expressed and selected, as described above, before mutagenesis and additional rounds of selection are performed. Mutagenesis of nucleic acid sequences encoding structurally optimized polypeptides is carried out by standard molecular methods. Of particular use is the polymerase chain reaction, or PCR, (Mullis and Faloona (1987) Methods Enzymol., 155: 335, herein incorporated by reference). PCR, which uses multiple cycles of DNA replication catalyzed by a thermostable, DNA-dependent DNA polymerase to amplify the target sequence of interest, is well known in the art. The construction of various antibody libraries has been discussed in Winter et al. (1994) Ann. Rev. Immunology 12, 433-55, and references cited therein.

PCR is performed using template DNA (at least 1 fg; more usefully, 1-1000 ng) and at least 25 pmol of oligonucleotide primers; it may be advantageous to use a larger amount of primer when the primer pool is heavily heterogeneous, as each sequence is represented by only a small fraction of the molecules of the pool, and amounts become limiting in the later amplification cycles. A typical reaction mixture includes: 2 μl of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler. The length and temperature of each step of a PCR cycle, as well as the number of cycles, is adjusted in accordance to the stringency requirements in effect. Annealing temperature and timing are determined both by the efficiency with which a primer is expected to anneal to a template and the degree of mismatch that is to be tolerated; obviously, when nucleic acid molecules are simultaneously amplified and mutagenised, mismatch is required, at least in the first round of synthesis. The ability to optimise the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1-5 minutes, depending on the length of the amplified product). Final extension is generally for 4 minutes at 72° C., and may be followed by an indefinite (0-24 hour) step at 4° C.

### Combining Single Variable Domains

Domains useful in the invention, once selected, may be combined by a variety of methods known in the art, including covalent and non-covalent methods. Preferred methods include the use of polypeptide linkers, as described, for example, in connection with scFv molecules (Bird et al., (1988) Science 242:423-426). Discussion of suitable linkers is provided in Bird et al. Science 242, 423-426; Hudson et al, Journal Immunol Methods 231 (1999) 177-189; Hudson et al, Proc Nat Acad Sci USA 85, 5879-5883. Linkers are preferably flexible, allowing the two single domains to interact. One linker example is a (Gly₄ Ser)ₙ linker, where n=1 to 8, e.g., 2, 3, 4, 5 or 7. The linkers used in diabodies, which are less flexible, may also be employed (Holliger et al., (1993) PNAS (USA) 90:6444-6448). In one embodiment, the linker employed is not an immunoglobulin hinge region.

Variable domains may be combined using methods other than linkers. For example, the use of disulphide bridges, provided through naturally-occurring or engineered cysteine residues, may be exploited to stabilize V_{H}-V_{H}, V_{L}-V_{L} or V_{H}-V_{L} dimers (Reiter et al., (1994) Protein Eng. 7:697-704) or by remodelling the interface between the variable domains to improve the “fit” and thus the stability of interaction (Ridgeway et al., (1996) Protein Eng. 7:617-621; Zhu et al., (1997) Protein Science 6:781-788). Other techniques for joining or stabilizing variable domains of immunoglobulins, and in particular antibody V_{H} domains, may be employed as appropriate.

### Characterisation of Ligands

The binding of a dual-specific ligand to the cell or the binding of each binding domain to each specific target can be tested by methods which will be familiar to those skilled in the art and include ELISA. In a preferred embodiment of the invention binding is tested using monoclonal phage ELISA. Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. (1994) Ann. Rev. Immunology 12, 433-55 and references cited therein.

The diversity of the selected phage monoclonal antibodies may also be assessed by gel electrophoresis of PCR products (Marks et al. 1991, supra; Nissim et al. 1994 supra), probing (Tomlinson et al., 1992, J. Mol. Biol. 227, 776) or by sequencing of the vector DNA.

### Structure of Ligands

In the case that each variable domains are selected from V-gene repertoires selected for instance using phage display technology as herein described, then these variable domains comprise a universal framework region, such that is they may be recognized by a specific generic dual-specific ligand as herein defined. The use of universal frameworks, generic ligands and the like is described in WO99/20749.

Where V-gene repertoires are used variation in polypeptide sequence is preferably located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

In general, nucleic acid molecules and vector constructs required for selection, preparation and formatting dual-specific ligands may be constructed and manipulated as set forth in standard laboratory manuals, such as Sambrook et al. (1989) Molecular Cloning: A Laboratory Manual, Cold Spring Harbor, USA.

The manipulation of nucleic acids useful in the present invention is typically carried out in recombinant vectors. As used herein, vector refers to a discrete element that is used to introduce heterologous DNA into cells for the expression and/or replication thereof. Methods by which to select or construct and, subsequently, use such vectors are well known to one of ordinary skill in the art. Numerous vectors are publicly available, including bacterial plasmids, bacteriophage, artificial chromosomes and episomal vectors. Such vectors may be used for simple cloning and mutagenesis; alternatively gene expression vector is employed. A vector of use according to the invention may be selected to accommodate a polypeptide coding sequence of a desired size, typically from 0.25 kilobase (kb) to 40 kb or more in length A suitable host cell is transformed with the vector after in vitro cloning manipulations. Each vector contains various functional components, which generally include a cloning (or “polylinker”) site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a dual-specific ligand according to the invention.

Both cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g., SV 40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Since the replication of vectors encoding a dual-specific ligand according to the present invention is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 or pUC19.

Expression vectors usually contain a promoter that is recognised by the host organism and is operably linked to the coding sequence of interest. Such a promoter may be inducible or constitutive. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Promoters for use in bacterial systems will also generally contain a Shine-Delgarno sequence operably linked to the coding sequence.

The preferred vectors are expression vectors that enable the expression of a nucleotide sequence corresponding to a polypeptide library member. Thus, selection with the first and/or second antigen or epitope can be performed by separate propagation and expression of a single clone expressing the polypeptide library member or by use of any selection display system. As described above, the preferred selection display system is bacteriophage display. Thus, phage or phagemid vectors may be used, e.g. pIT1 or pIT2. Leader sequences useful in the invention include pelB, stII, ompA, phoA, bla and pelA. One example are phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector contains a β-lactamase gene to confer selectivity on the phagemid and a lac promoter upstream of a expression cassette that consists (N to C terminal) of a pelB leader sequence (which directs the expressed polypeptide to the periplasmic space), a multiple cloning site (for cloning the nucleotide version of the library member), optionally, one or more peptide tag (for detection), optionally, one or more TAG stop codon and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or produce phage, some of which contain at least one copy of the polypeptide-pIII fusion on their surface.

Construction of vectors encoding dual-specific ligands according to the invention employs conventional ligation techniques. Isolated vectors or DNA fragments are cleaved, tailored, and religated in the form desired to generate the required vector. If desired, analysis to confirm that the correct sequences are present in the constructed vector can be performed in a known fashion. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridisation, immunocytochemistry or sequence analysis of nucleic acid or protein molecules. Those skilled in the art will readily envisage how these methods may be modified, if desired.

### Skeletons

Skeletons may be based on immunoglobulin molecules or may be non-immunoglobulin in origin as set forth above. Each domain of the dual-specific ligand may be a different skeleton. Preferred immunoglobulin skeletons as herein defined includes any one or more of those selected from the following: an immunoglobulin molecule comprising at least (i) the CL (kappa or lambda subclass) domain of an antibody; or (ii) the CH1 domain of an antibody heavy chain; an immunoglobulin molecule comprising the C_{H}1 and CH2 domains of an antibody heavy chain; an immunoglobulin molecule comprising the CH1, CH2 and CH3 domains of an antibody heavy chain; or any of the subset (ii) in conjunction with the CL (kappa or lambda subclass) domain of an antibody. A hinge region domain may also be included. Such combinations of domains may, for example, mimic natural antibodies, such as IgG or IgM, or fragments thereof, such as Fv, scFv, Fab or F(ab′)₂ molecules. Those skilled in the art will be aware that this list is not intended to be exhaustive.

### Protein Scaffolds

Each binding domain comprises a protein scaffold and one or more CDRs which are involved in the specific interaction of the domain with one or more epitopes. Advantageously, an epitope binding domain according to the present invention comprises three CDRs. Suitable protein scaffolds include any of those selected from the group consisting of the following: those based on immunoglobulin domains, those based on fibronectin, those based on affibodies, those based on CTLA4, those based on chaperones such as GroEL, those based on lipocallin and those based on the bacterial Fc receptors SpA and SpD, an SpA scaffold, an LDL receptor class A domain, an EGF domain, and avimer (see, e.g., U.S. Patent Application Publication Nos. 2005/0053973, 2005/0089932, 2005/0164301). Those skilled in the art will appreciate that this list is not intended to be exhaustive.

### Scaffolds for use in Constructing Ligands

### Selection of the Main-chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2 and L3 found in the majority of human antibodies (Chothia et al. (1992) J. Mol. Biol., 227: 799; Tomlinson et al. (1995) EMBO J, 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

Libraries of ligands and/or binding domains can be designed in which certain loop lengths and key residues have been chosen to ensure that the main-chain conformation of the members is known. Advantageously, these are real conformations of immunoglobulin superfamily molecules found in nature, to minimize the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on dual-specific ligand sequences and to choose residues for diversification which do not affect the canonical structure. It is known that, in the human V_{κ} domain, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} domain alone, different canonical structures can combine to create a range of different main-chain conformations. Given that the V_{λ} domain encodes a different range of canonical structures for the L1, L2 and L3 loops and that V_{κ} and V_{λ} domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a preferred aspect, the ligands of the invention possess a single known main-chain conformation.

The single main-chain conformation that is chosen is preferably commonplace among molecules of the immunoglobulin superfamily type in question. A conformation is commonplace when a significant number of naturally occurring molecules are observed to adopt it. Accordingly, in a preferred aspect of the invention, the natural occurrence of the different main-chain conformations for each binding loop of an immunoglobulin domain are considered separately and then a naturally occurring variable domain is chosen which possesses the desired combination of main-chain conformations for the different loops. If none is available, the nearest equivalent may be chosen. It is preferable that the desired combination of main-chain conformations for the different loops is created by selecting germline gene segments which encode the desired main-chain conformations. It is more preferable, that the selected germline gene segments are frequently expressed in nature, and most preferable that they are the most frequently expressed of all natural germline gene segments.

In designing ligands (e.g., ds-dAbs) or libraries thereof the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e. between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1-CS 1 (79% of the expressed repertoire), H2-CS 3 (46%), L1-CS 2 of V_{κ} (39%), L2-CS 1 (100%), L3-CS 1 of V_{κ} (36%) (calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}1. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five or for all six of the antigen binding loops can be determined. Here, it is preferable that the chosen conformation is commonplace in naturally occurring antibodies and most preferable that it observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

In particular embodiments, the ligands of the invention (e.g., dAb monomers) possess a heavy chain hypervariable loop having the canonical structure of the H3 loop of the human germline V_{H} segment 3-23 (DP47) and JH4b. In further embodiments, such ligands also comprise a heavy chain hypervariable loop having the canonical structure of the H2 loop of DP47. In yet another embodiment, a ligand that has a heavy chain hypervariable loop having the canonical structure of the H3 loop of DP47 and JH4b comprises a V_{H}3 domain. In a preferred embodiment, the ligand comprising a heavy chain hypervariable loop having the canonical structure of the H3 loop of DP47 and JH4b is a domain antibody (dAb) monomer that has binding specificity for EGFR.

### Diversification of the Canonical Sequence

Having selected several known main-chain conformations or, preferably a single known main-chain conformation, ligands (e.g., dAbs) or libraries for use in the invention can be constructed by varying each binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or are preferably selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomization has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

Preferably, only the residues that are directly involved in creating or modifying the desired function of each domain of the dual-specific ligand molecule are diversified. For many molecules, the function of each domain will be to bind a target and therefore diversity should be concentrated in the target binding site, while avoiding changing residues which are crucial to the overall packing of the molecule or to maintaining the chosen main-chain conformation.

### Diversification of the Canonical Sequence as it Applies to Antibody Domains

In the case of antibody based ligands (e.g., dAbs), the binding site for each target is most often the antigen binding site. Thus, preferably only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library for use according to the invention. This represents a significant improvement in terms of the functional diversity required to create a range of antigen binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) J. Mol. Biol., 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire can be created where some, but not all, of the residues in the antigen binding site are diversified. As used herein in this context, the term “naive” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

Naive repertoires of binding domains for the construction of dual-specific ligands in which some or all of the residues in the antigen binding site are varied are known in the art. (See, WO 2004/058821, WO 2004/003019, and WO 03/002609). The “primary” library mimics the natural primary repertoire, with diversity restricted to residues at the centre of the antigen binding site that are diverse in the germline V gene segments (germline diversity) or diversified during the recombination process (junctional diversity). Those residues which are diversified include, but are not limited to, H50, H52, H52a, H53, H55, H56, H58, H95, H96, H97, H98, L50, L53, L91, L92, L93, L94 and L96. In the “somatic” library, diversity is restricted to residues that are diversified during the recombination process (junctional diversity) or are highly somatically mutated). Those residues which are diversified include, but are not limited to: H31, H33, H35, H95, H96, H97, H98, L30, L31, L32, L34 and L96. All the residues listed above as suitable for diversification in these libraries are known to make contacts in one or more antibody-antigen complexes. Since in both libraries, not all of the residues in the antigen binding site are varied, additional diversity is incorporated during selection by varying the remaining residues, if it is desired to do so. It shall be apparent to one skilled in the art that any subset of any of these residues (or additional residues which comprise the antigen binding site) can be used for the initial and/or subsequent diversification of the antigen binding site.

In the construction of libraries for use in the invention, diversification of chosen positions is typically achieved at the nucleic acid level, by altering the coding sequence which specifies the sequence of the polypeptide such that a number of possible amino acids (all 20 or a subset thereof) can be incorporated at that position. Using the IUPAC nomenclature, the most versatile codon is NNK, which encodes all amino acids as well as the TAG stop codon. The NNK codon is preferably used in order to introduce the required diversity. Other codons which achieve the same ends are also of use, including the NNN codon, which leads to the production of the additional stop codons TGA and TAA.

A feature of side-chain diversity in the antigen binding site of human antibodies is a pronounced bias which favors certain amino acid residues. If the amino acid composition of the ten most diverse positions in each of the V_{H}, V_{κ} and V_{λ} regions are summed, more than 76% of the side-chain diversity comes from only seven different residues, these being, serine (24%), tyrosine (14%), asparagine (11%), glycine (9%), alanine (7%), aspartate (6%) and threonine (6%). This bias towards hydrophilic residues and small residues which can provide main-chain flexibility probably reflects the evolution of surfaces which are predisposed to binding a wide range of antigens or epitopes and may help to explain the required promiscuity of antibodies in the primary repertoire.

Since it is preferable to mimic this distribution of amino acids, the distribution of amino acids at the positions to be varied preferably mimics that seen in the antigen binding site of antibodies. Such bias in the substitution of amino acids that permits selection of certain polypeptides (not just antibody polypeptides) against a range of target antigens is easily applied to any polypeptide repertoire. There are various methods for biasing the amino acid distribution at the position to be varied (including the use of tri-nucleotide mutagenesis, see WO97/08320), of which the preferred method, due to ease of synthesis, is the use of conventional degenerate codons. By comparing the amino acid profile encoded by all combinations of degenerate codons (with single, double, triple and quadruple degeneracy in equal ratios at each position) with the natural amino acid use it is possible to calculate the most representative codon. The codons (AGT)(AGC)T, (AGT)(AGC)C and (AGT)(AGC)(CT)—that is, DVT, DVC and DVY, respectively using IUPAC nomenclature—are those closest to the desired amino acid profile: they encode 22% serine and 11% tyrosine, asparagine, glycine, alanine, aspartate, threonine and cysteine. Preferably, therefore, libraries are constructed using either the DVT, DVC or DVY codon at each of the diversified positions.

### Therapeutic and Diagnostic Compositions and Uses

The invention provides compositions comprising the ligands of the invention and a pharmaceutically acceptable carrier, diluent or excipient, and therapeutic and diagnostic methods that employ the ligands or compositions of the invention. The ligands according to the method of the present invention may be employed in in vivo therapeutic and prophylactic applications, in vivo diagnostic applications and the like.

Therapeutic and prophylactic uses of ligands of the invention involve the administration of ligands according to the invention to a recipient mammal, such as a human. The ligands bind to targets with high affinity and/or avidity. In some embodiments, such as IgG-like ligands, the ligands can allow recruitment of cytotoxic cells to mediate killing of cancer cells, for example by antibody dependent cellular cytoxicity.

Substantially pure ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the ligands may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

For example, the ligands, of the present invention will typically find use in preventing, suppressing or treating disease states. For example, ligands can be administered to treat, suppress or prevent a disease or disorder caused by receptor activity, or characterized by expression or overexpression of receptor, such as chronic inflammatory disease, allergic hypersensitivity, cancer, bacterial or viral infection, autoimmune disorders (which include, but are not limited to, Type I diabetes, asthma, multiple sclerosis, rheumatoid arthritis, juvenile rheumatoid arthritis, psoriatic arthritis, spondylarthropathy (e.g., ankylosing spondylitis), systemic lupus erythematosus, inflammatory bowel disease (e.g., Crohn's disease, ulcerative colitis), myasthenia gravis and Behcet's syndrome), psoriasis, endometriosis, and abdominal adhesions (e.g., post abdominal surgery).

The ligands are useful for treating infectious diseases in which cells infected with an infectious agent contain higher levels of receptor than uninfected cells or that express one or more receptors that are not present on ininfected cells, such as a protein that is encoded by the infectious agent (e.g., bacteria, virus).

Ligands according to the invention that are able to bind to receptor can be internalized by cells that express receptor (e.g., endocytosed), and can deliver therapeutic agents (e.g., a toxin) intracellularly. In addition, ligands, provide a means by which a binding domain (e.g., a dAb monomer) that is specificity able to bind to an intracellular target can be delivered to an intracellular environment. This strategy requires, for example, a binding domain with physical properties that enable it to remain functional inside the cell. Alternatively, if the final destination intracellular compartment is oxidising, a well folding ligand may not need to be disulphide free.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest. Treatment includes ameliorating symptoms associated with the disease, and also preventing or delaying the onset of the disease and also lessening the severity or frequency of symptoms of the disease.

The term “cancer” refers to the pathological condition in mammals that is typically characterized by dysregulated cellular proliferation or survival. Examples of cancer include, but are not limited to, carcinoma, lymphoma, blastoma, sarcoma, and leukemia and lymphoid malignancies. More particular examples of cancers include squamous cell cancer (e.g. epithelial squamous cell cancer), lung cancer (e.g., small-cell lung carcinoma, non-small cell lung carcinoma, adenocarcinoma of the lung, squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, gall bladder cancer, hepatoma, breast cancer, colon cancer, rectal cancer, colorectal cancer, multiple myeloma, chronic myelogenous leukemia, acute myelogenous leukemia, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, and the like. It is well-known that many cancers are characterized by expression or overexpression of certain receptors. For example, cancers characterized by expression of EGFR on the surface of cancerous cells (EGFR-expressing cancers) include, for example, bladder cancer, ovarian cancer, colorectal cancer (e.g., colorectal carcinoma), breast cancer, lung cancer (e.g., non-small cell lung carcinoma), gastric cancer, pancreatic cancer, prostate cancer, head and neck cancer, renal cancer and gall bladder cancer.

Animal model systems which can be used to assess efficacy of the ligands of the invention in preventing treating or suppressing disease (e.g., cancer) are available. Suitable models of cancer include, for example, xenograft and orthotopic models of human cancers in animal models, such as the SCID-hu myeloma model (Epstein J, and Yaccoby, S., Methods Mol Med 113:183-90 (2005), Tassone P, et al., Clin Cancer Res. 11 (11):4251-8 (2005)), mouse models of human lung cancer (e.g., Meuwissen R and Berns A, Genes Dev. 19(6):643-64 (2005)), and mouse models of metastatic cancers (e.g., Kubota T., J Cell Biochem. 56(1):4-8 (1994)).

Generally, the present ligands will be utilized in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition). A variety of suitable formulations can be used, including extended release formulations.

The ligand of the present invention may be used as separately administered compositions or in conjunction with other agents. The ligands can be administered and or formulated together with one or more additional therapeutic or active agents. When a ligand is administered with an additional therapeutic agent, the ligand can be administered before, simultaneously with or subsequent to administration of the additional agent. Generally, the ligand and additional agent are administered in a manner that provides an overlap of therapeutic effect. Additional agents that can be administered or formulated with the ligand of the invention include, for example, various immunotherapeutic drugs, such as cylcosporine, methotrexate, adriamycin or cisplatinum, antibiotics, antimycotics, anti-viral agents and immunotoxins. For example, when the antagonist is administered to prevent, suppress or treat lung inflammation or a respiratory disease, it can be administered in conjuction with phosphodiesterase inhibitors (e.g., inhibitors of phosphodiesterase 4), bronchodilators (e.g., beta2-agonists, anticholinergerics, theophylline), short-acting beta-agonists (e.g., albuterol, salbutamol, bambuterol, fenoterol, isoetherine, isoproterenol, levalbuterol, metaproterenol, pirbuterol, terbutaline and tornlate), long-acting beta-agonists (e.g., formoterol and salmeterol), short acting anticholinergics (e.g., ipratropium bromide and oxitropium bromide), long-acting anticholinergics (e.g., tiotropium), theophylline (e.g. short acting formulation, long acting formulation), inhaled steroids (e.g., beclomethasone, beclometasone, budesonide, flunisolide, fluticasone propionate and triamcinolone), oral steroids (e.g., methylprednisolone, prednisolone, prednisolon and prednisone), combined short-acting beta-agonists with anticholinergics (e.g., albuterol/salbutamol/ipratopium, and fenoterol/ipratopium), combined long-acting beta-agonists with inhaled steroids (e.g., salmeterol/fluticasone, and formoterol/budesonide) and mucolytic agents (e.g., erdosteine, acetylcysteine, bromheksin, carbocysteine, guiafenesin and iodinated glycerol).

The ligands of the invention can be coadministered (e.g., to treat cancer, an inflammatory disease or other disease) with a variety of suitable co-therapeutic agents, including cytokines, analgesics/antipyretics, antiemetics, and chemotherapeutics. Further suitable co-therapeutic agents include immunosuppressive agents selected from the group consisting of cyclosporine, azathioprine, mycophenolic acid, mycophenolate mofetil, corticosteroids, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, and anti-thymocyte globulin, anti-inflammatory agents selected from the group consisting of aspirin, other salicylates, steroidal drugs, NSAIDs (nonsteroidal anti-inflammatory drugs), Cox-2 inhibitors, and DMARDs (disease modifying antirheumatic drugs); anti-psoriasis agents selected from the group consisting of coal tar, A vitamin, anthralin, calcipotrien, tarazotene, corticosteroids, methotrexate, retinoids, cyclosporine, etanercept, alefacept, efaluzimab, 6-thioguanine, mycophenolate mofetil, tacrolimus (FK-506), and hydroxyurea.

Cytokines include, without limitation, a lymphokine, tumor necrosis factors, tumor necrosis factor-like cytokine, lymphotoxin, interferon, macrophage inflammatory protein, granulocyte monocyte colony stimulating factor, interleukin (including, without limitation, interleukin-1, interleukin-2, interleukin-6, interleukin-12, interleukin-15, interleukin-18), growth factors, which include, without limitation, (e.g., growth hormone, insulin-like growth factor 1 and 2 (IGF-1 and IGF-2), granulocyte colony stimulating factor (GCSF), platelet derived growth factor (PGDF), epidermal growth factor (EGF), and agents for erythropoiesis stimulation, e.g., recombinant human erythropoietin (Epoetin alfa), EPO, a hormonal agonist, hormonal antagonists (e.g., flutamide, tamoxifen, leuprolide acetate (LUPRON)), and steroids (e.g., dexamethasone, retinoid, betamethasone, cortisol, cortisone, prednisone, dehydrotestosterone, glucocorticoid, mineralocorticoid, estrogen, testosterone, progestin).

Analgesics/antipyretics can include, without limitation, (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like).

Antiemetics can also be coadministered to prevent or treat nausea and vomiting, e,g., suitable antiemetics include meclizine hydrochloride, nabilone, prochlorperazine, dimenhydrinate, promethazine hydrochloride, thiethylperazine, scopolamine, and the like).

Chemotherapeutic agents, as that term is used herein, include, but are not limited to, for example antimicrotubule agents, e.g., taxol (paclitaxel), taxotere (docetaxel); alkylating agents, e.g., cyclophosphamide, carmustine, lomustine, and chlorambucil; cytotoxic antibiotics, e.g., dactinomycin, doxorubicin, mitomycin-C, and bleomycin; antimetabolites, e.g., cytarabine, gemcitatin, methotrexate, and 5-fluorouracil; antimiotics, e.g., vincristine vinca alkaloids, e.g., etoposide, vinblastine, and vincristine; and others such as cisplatin, dacarbazine, procarbazine, and hydroxyurea; and combinations thereof.

The ligands of the invention can be used to treat cancer in combination with another therapeutic agent. Fore example, a ligand of the invention can be administered in combination with a chemotherapeutic agent or an antineoplastic composition comprising a (at least one) chemotherapeutic agent. Advantageously, in such a therapeutic approach, the amount of chemotherapeutic agent that must be administered to be effective can be reduced. Thus the invention provides a method of treating cancer comprising administering to a patient in need thereof a therapeutically effective amount to a ligand of the invention and a chemotherapeutic agent, wherein the chemotherapeutic agent is administered at a low dose. Generally the amount of chemotherapeutic agent that is coadministered with a ligand of the invention is about 80%, or about 70%, or about 60%, or about 50%, or about 40%, or about 30%, or about 20%, or about 10% or less, of the dose of chemotherapeutic agent alone that is normally administered to a patient. Thus, cotherapy is particularly advantageous when the chemotherapeutic agent causes deleterious or undesirable side effects that may be reduced or eliminated at a lower doses.

Pharmaceutical compositions can include “cocktails” of various cytotoxic or other agents in conjunction with ligands of the present invention, or even combinations of ligands according to the present invention having different specificities, such as ligands selected using different target antigens or epitopes, whether or not they are pooled prior to administration.

The route of administration of pharmaceutical compositions according to the invention may be any suitable route, such as any of those commonly known to those of ordinary skill in the art. For therapy, including without limitation immunotherapy, the ligands of the invention can be administered to any patient in accordance with standard techniques. The administration can be by any appropriate mode, including parenterally, intravenously, intramuscularly, intraperitoneally, transdermally, intrathecally, intraarticularly, via the pulmonary route, or also, appropriately, by direct infusion (e.g., with a catheter). The dosage and frequency of administration will depend on the age, sex and condition of the patient, concurrent administration of other drugs, counterindications and other parameters to be taken into account by the clinician. Administration can be local (e.g., local delivery to the lung by pulmonary administration, (e.g., intranasal administration) or local injection directly into a tumor) or systemic as indicated.

In some embodiments, the pharmaceutical composition comprises a vehicle for intraarterial, intravenous, intraarticular, subcutaneous, intranasal, vaginal, or rectal administration.

The ligands of this invention can be lyophilised for storage and reconstituted in a suitable carrier prior to use. This technique has been shown to be effective with conventional immunoglobulins and art-known lyophilisation and reconstitution techniques can be employed. It will be appreciated by those skilled in the art that lyophilisation and reconstitution can lead to varying degrees of antibody activity loss (e.g with conventional immunoglobulins, IgM antibodies tend to have greater activity loss than IgG antibodies) and that use levels may have to be adjusted upward to compensate.

The invention also relates to a method of antagonizing a receptor without substantially agonizing the receptor. The method comprises combining a cell that expresses the receptor with a ligand that has binding specificity for said receptor under conditions suitable for the binding of said ligand to said receptor. In some embodiments, the method is performed on a patient in need thereof, and comprises comprising administering to a patient in need thereof a therapeutically effective dose of a ligand under conditions suitable for the binding of said ligand to said receptor. In particular embodiments, the ligand comprises a dAb that has binding specificity for the receptor. In some embodiment, the ligand inhibits the binding of cognate ligand to said receptor, inhibits receptor clustering and/or inhibits receptor signalling. In a particular embodiment, the ligand comprises a dAb monomer (e.g., a PEGylated dAb monomer, a dual specific ligand comprising a dAb that binds receptor and a dAb that binds serum albumin.

The compositions containing the ligands can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's health, but generally range from 0.005 to 5.0 mg of ligand per kilogram of body weight, with doses of 0.05 to 2.0 mg/kg/dose being more commonly used. For prophylactic applications, compositions containing the present ligands or cocktails thereof may also be administered in similar or slightly lower dosages, to prevent, inhibit or delay onset of disease (e.g., to sustain remission or quiescence, or to prevent acute phase). The skilled clinician will be able to determine the appropriate dosing interval to treat, suppress or prevent disease. When a ligand is administered to treat, suppress or prevent a disease, it can be administered up to four times per day, twice weekly, once weekly, once every two weeks, once a month, or once every two months, at a dose off, for example, about 10 μg/kg to about 80 mg/kg, about 100 μg/kg to about 80 mg/kg, about 1 mg/kg to about 80 mg/kg, about 1 mg/kg to about 70 mg/kg, about 1 mg/kg to about 60 mg/kg, about 1 mg/kg to about 50 mg/kg, about 1 mg/kg to about 40 mg/kg, about 1 mg/kg to about 30 mg/kg, about 1 mg/kg to about 20 mg/kg, about 1 mg/kg to about 10 mg/kg, about 110 μg/kg to about 10 mg/kg, about 10 μg/kg to about 5 mg/kg, about 10 μg/kg to about 2.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg. In particular embodiments, the dual-specific ligand is administered to treat, suppress or prevent a chronic inflammatory disease once every two weeks or once a month at a dose of about 10 μg/kg to about 10 mg/kg (e.g., about 10 μg/kg, about 100 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.)

In particular embodiments, the ligand of the invention is administered at a dose that provides saturation of receptor (e.g., EGFR, TNFR1, IL-1R1) or a desired serum concentration in vivo. The skilled physician can determine appropriate dosing to achieve saturation, for example by titrating ligand and monitoring the amount of free binding sites on receptor expressing cells or the serum concentration of ligand. Therapeutic regiments that involve administering a therapeutic agent to achieve target saturation or a desired serum concentration of agent are common in the art, particularly in the field of oncology.

Treatment or therapy performed using the compositions described herein is considered “effective” if one or more symptoms are reduced (e.g., by at least 10% or at least one point on a clinical assessment scale), relative to such symptoms present before treatment, or relative to such symptoms in an individual (human or model animal) not treated with such composition or other suitable control. Symptoms will obviously vary depending upon the disease or disorder targeted, but can be measured by an ordinarily skilled clinician or technician. Such symptoms can be measured, for example, by monitoring the level of one or more biochemical indicators of the disease or disorder (e.g., levels of an enzyme or metabolite correlated with the disease, affected cell numbers, etc.), by monitoring physical manifestations (e.g., inflammation, tumor size, etc.), or by an accepted clinical assessment scale, for example, the Expanded Disability Status Scale (for multiple sclerosis), the Irvine Inflammatory Bowel Disease Questionnaire (32 point assessment evaluates quality of life with respect to bowel function, systemic symptoms, social function and emotional status—score ranges from 32 to 224, with higher scores indicating a better quality of life), the Quality of Life Rheumatoid Arthritis Scale, or other accepted clinical assessment scale as known in the field. A sustained (e.g., one day or more, preferably longer) reduction in disease or disorder symptoms by at least 10% or by one or more points on a given clinical scale is indicative of “effective” treatment. Similarly, prophylaxis performed using a composition as described herein is “effective” if the onset or severity of one or more symptoms is delayed, reduced or abolished relative to such symptoms in a similar individual (human or animal model) not treated with the composition.

A composition containing ligands according to the present invention may be utilized in prophylactic and therapeutic settings to aid in the alteration, inactivation, killing or removal of a select target cell population in a mammal. In addition, the ligands and selected repertoires of polypeptides described herein may be used extracorporeally or in vitro selectively to kill, deplete or otherwise effectively remove a target cell population from a heterogeneous collection of cells. Blood from a mammal may be combined extracorporeally with the ligands, e.g. antibodies, cell-surface receptors or binding proteins thereof whereby the undesired cells are killed or otherwise removed from the blood for return to the mammal in accordance with standard techniques.

In particular embodiments, the invention relates to a method of treating cancer comprising administering to a subject in need thereof a therapeutically effective amount of a ligand, as described herein, that has binding specificity for receptor (e.g., EGFR). In particular embodiments, the patient has an EGFR-expressing cancer, such as, bladder cancer, ovarian cancer, colorectal cancer, breast cancer, lung cancer (e.g., non-small cell lung carcinoma), gastric cancer, pancreatic cancer, prostate cancer, head and neck cancer, renal cancer and gall bladder cancer. In certain embodiments, the patient has an EGFR-expressing cancer selected from the group consisting of breast cancer, ovarian cancer, lung cancer, colon cancer, and head and neck cancer.

In other embodiments, the invention relates to a method for treating cancer, comprising administering to a subject in need thereof a therapeutically effective amount of ligand, as described herein, (e.g., a ligand that has binding specificity for a receptor, a ligand that has binding specificity for EGFR, a ligand that has binding specificity for EGFR and for a target that is different than EGFR) and an anti-neoplastic composition, wherein said anti-neoplastic composition comprises at least one chemotherapeutic agent selected from the group consisting of alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. In some embodiments, the chemotherapeutic agent is selected from the group consisting of cisplatin, dicarbazine, dactinomycin, mechlorethamine, streptozocin, cyclophosphamide, capecitabine, carmustine, lomustine, doxorubicin, daunorubicin, procarbazine, mitomycin, cytarabine, etoposide, methotrexate, 5-fluorouracil, vinbiastine, vincristine, bleomycin, paclitaxel, docetaxel, doxetaxe, aldesleukin, asparaginase, busulfan, carboplatin, cladribine, dacarbazine, floxuridine, fludarabine, hydroxyurea, ifosfamide, interferon alpha, irinotecan, leuprolide, leucovorin, megestrol, melphalan, mercaptopurine, oxaliplatin, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, taxol, an additional growth factor receptor antagonist, and a combination of any of the foregoing.

The invention also relates to a drug delivery device comprising the composition (e.g., pharmaceutical composition) of the invention or a ligand (e.g., dAb monomer) of the invention. In some embodiments, the drug device comprises a plurality of therapeutically effective doses of ligand.

In other embodiments, the drug delivery device is selected from the group consisting of a parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, rectal delivery device, a syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, and a catheter.

### Assays for Evaluating Ligands

The ligands of the invention can be assayed using any suitable in vitro or in vivo assay, for example, using the receptor binding assays or bioassays described herein.

### EXAMPLES

The invention is further described, for the purposes of illustration only, in the following examples.

### Example 1

### Selection of a Dual Specific scFv Antibody (K8) Directed Against Human Serum Albumin (HSA) and β-Galactosidase (β-Gal)

This example explains a method for making a dual specific antibody directed against β-gal and HSA in which a repertoire of V_{κ} variable domains linked to a germline (dummy) V_{H} domain is selected for binding to β-gal and a repertoire of V_{H} variable domains linked to a germline (dummy) V_{κ} domain is selected for binding to HSA. The selected variable V_{H} HSA and V_{κ}β-gal domains are then combined and the antibodies selected for binding to β-gal and HSA. HSA is a half-life increasing protein found in human blood.

Four human phage antibody libraries were used in this experiment.

| | | |
|---|---|---|
| Library 1 | Germline V_{κ}/DVT V_{H} | 8.46 × 10⁷ |
| Library 2 | Germline V_{κ}/NNK V_{H} | 9.64 × 10⁷ |
| Library 3 | Germline V_{H}/DVT V_{κ} | 1.47 × 10⁸ |
| Library 4 | Germline V_{H}/NNK V_{κ} | 1.45 × 10⁸ |

All libraries are based on a single human framework for V_{H} (V3-23/DP47 and JH4b) and V_{κ} (O12/O02/DPK9 and J_{κ}1) with side chain diversity incorporated in complementarity determining regions (CDR2 and CDR3).

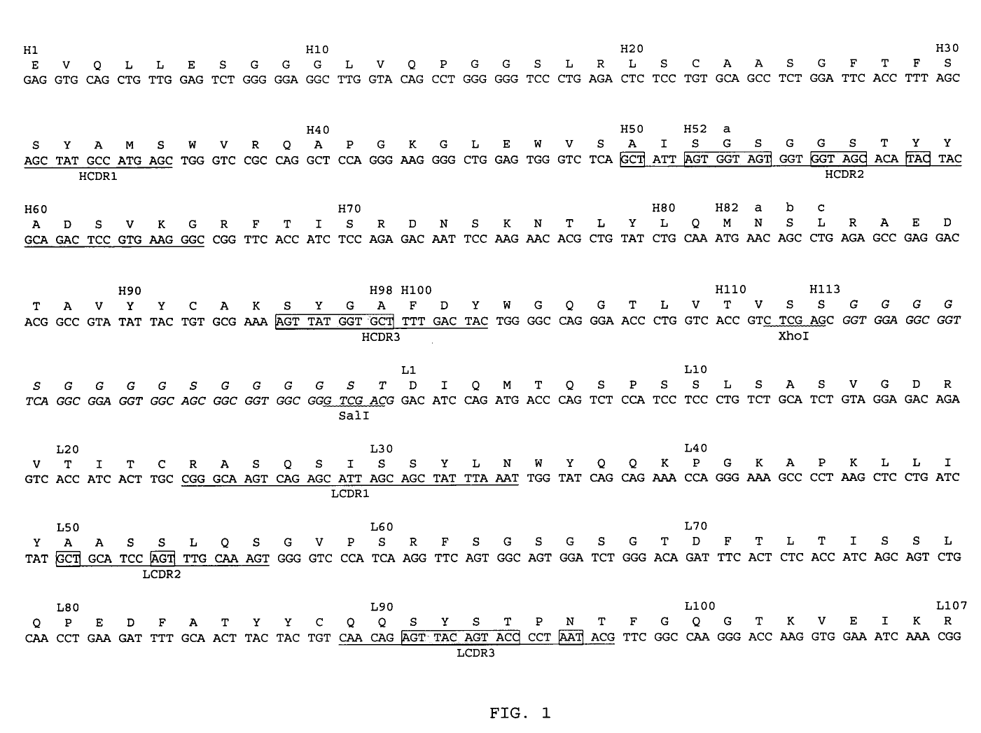

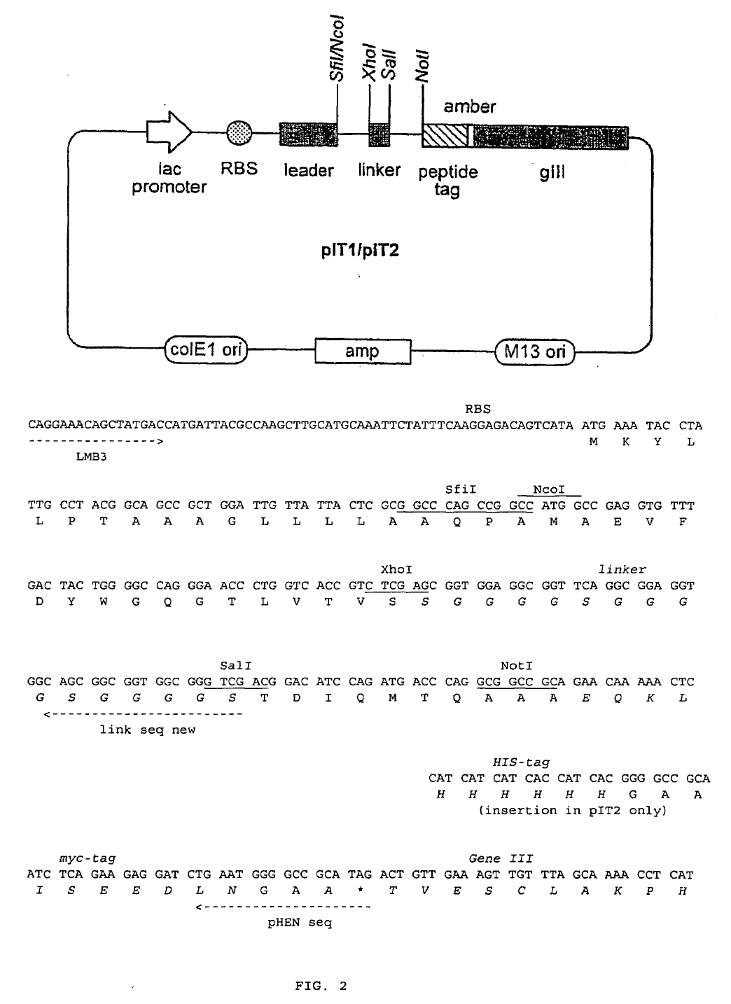

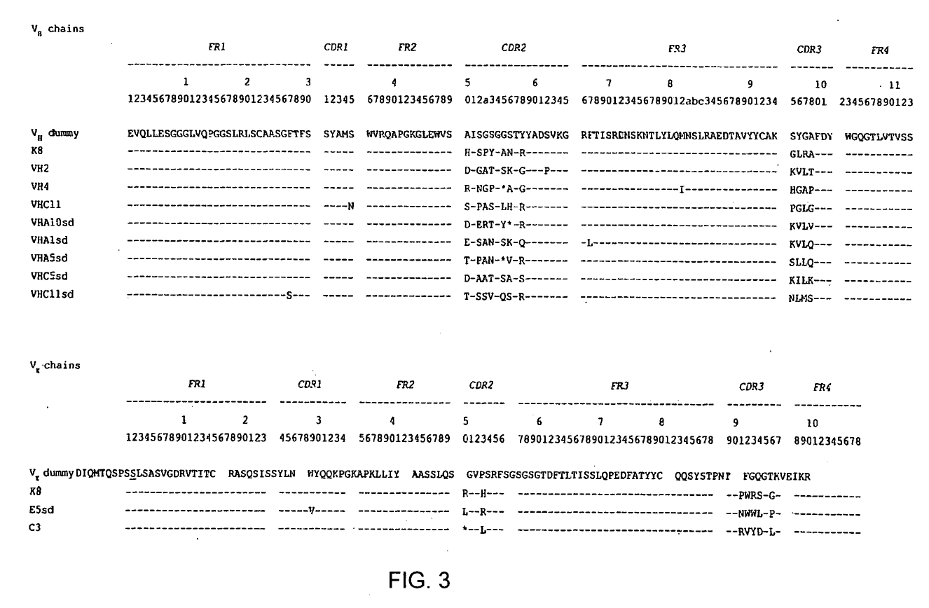

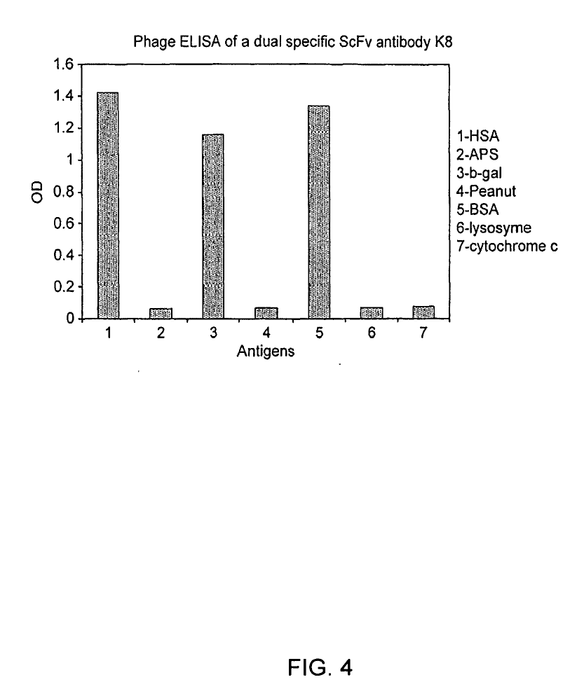

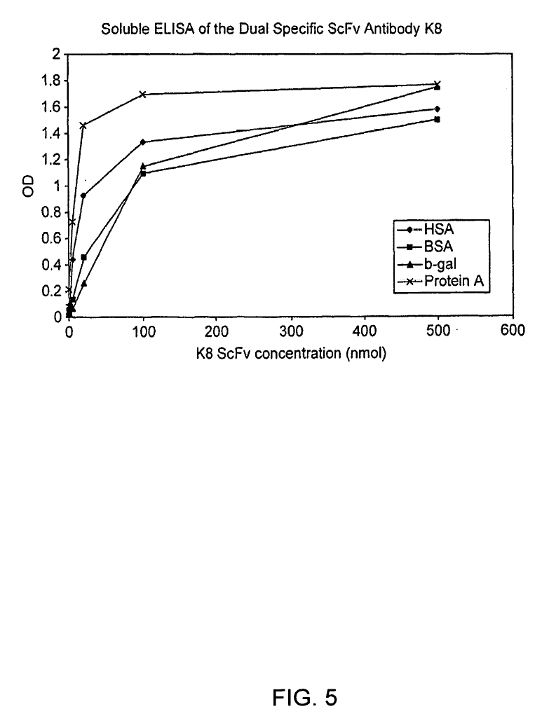

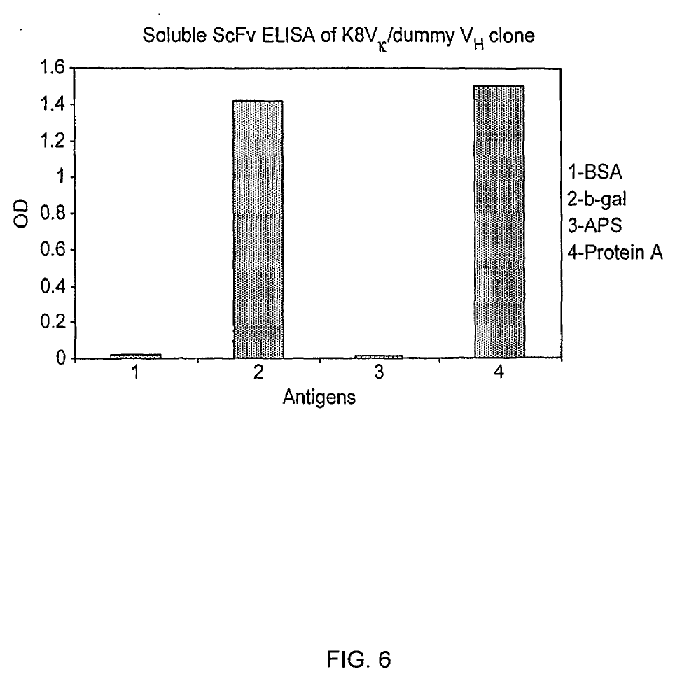

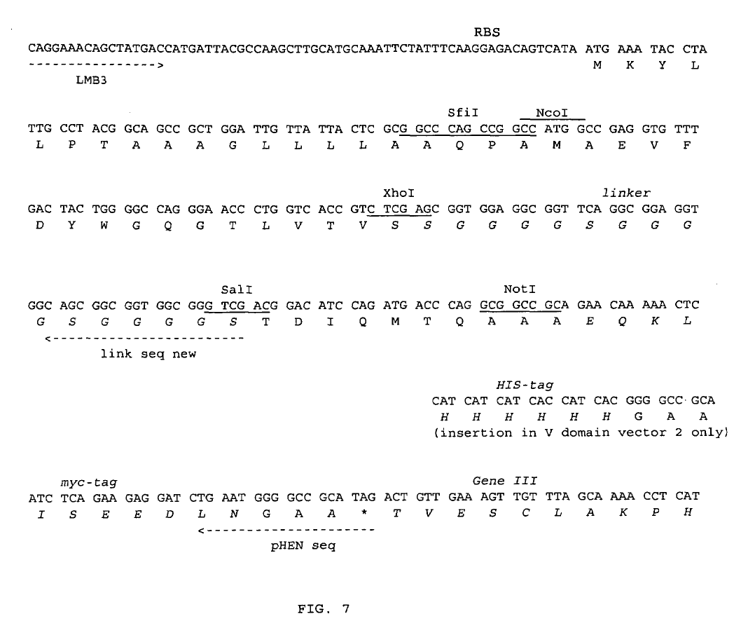

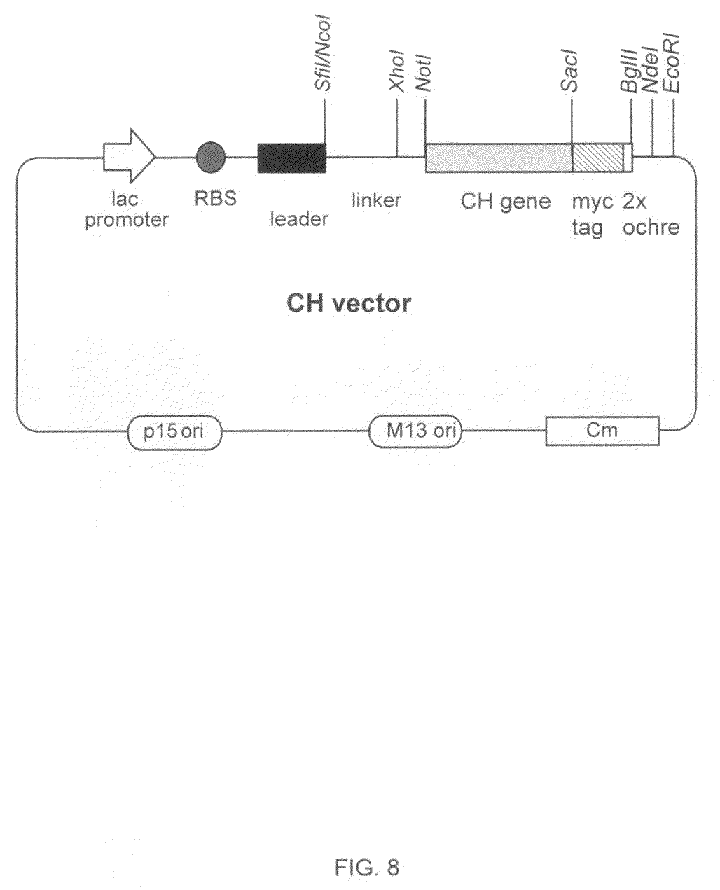

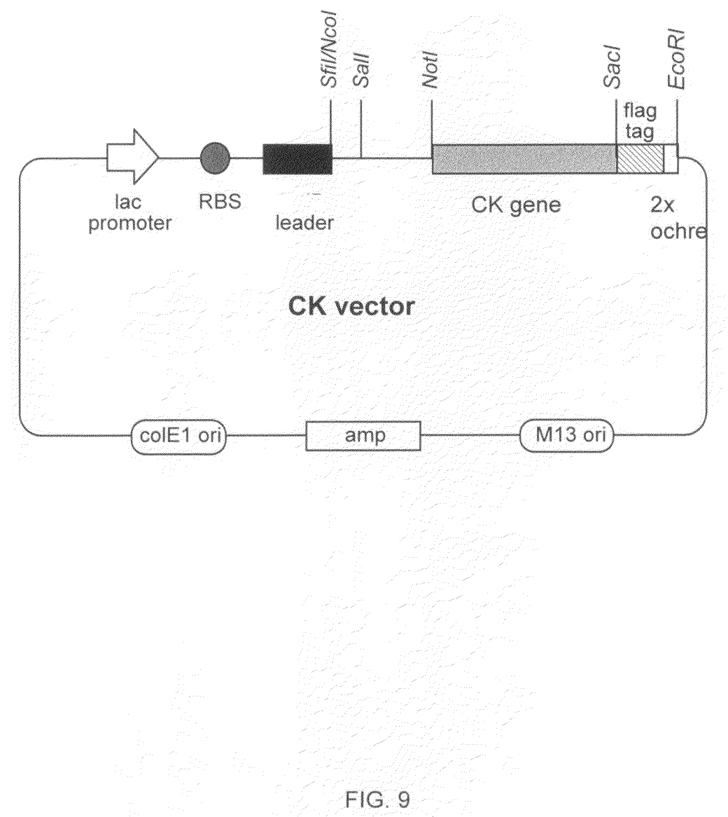

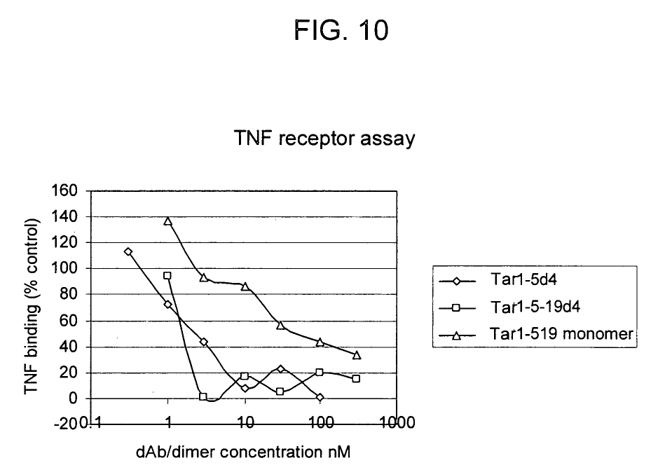

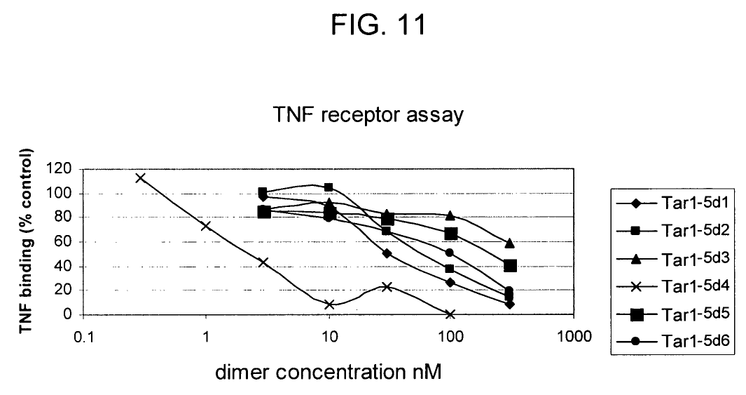

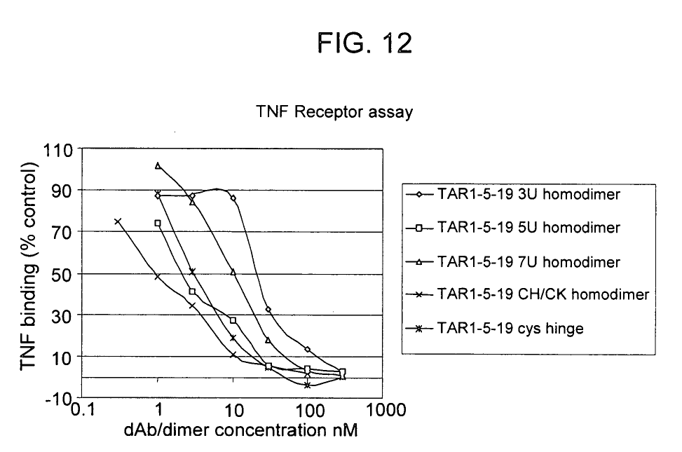

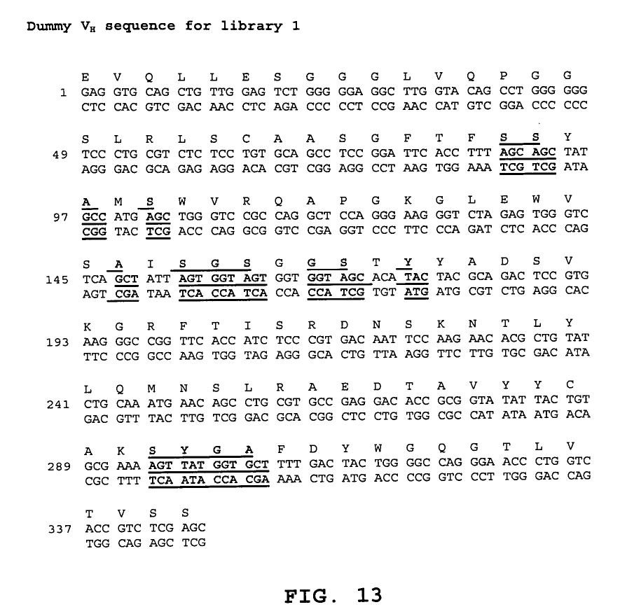

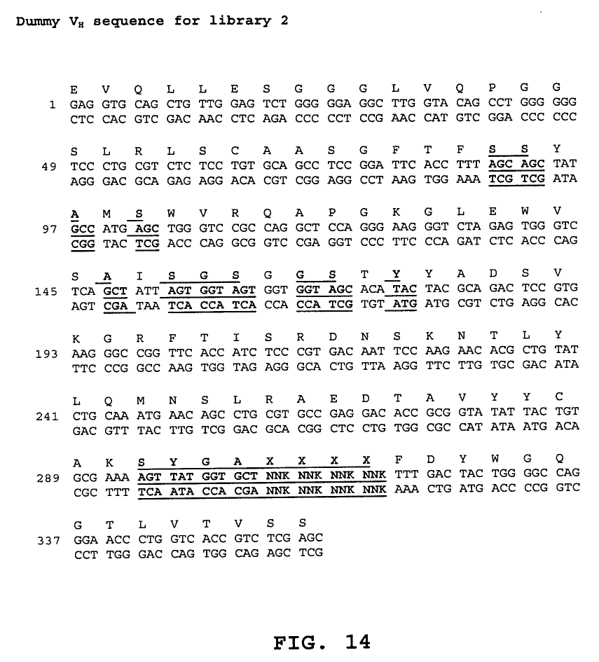

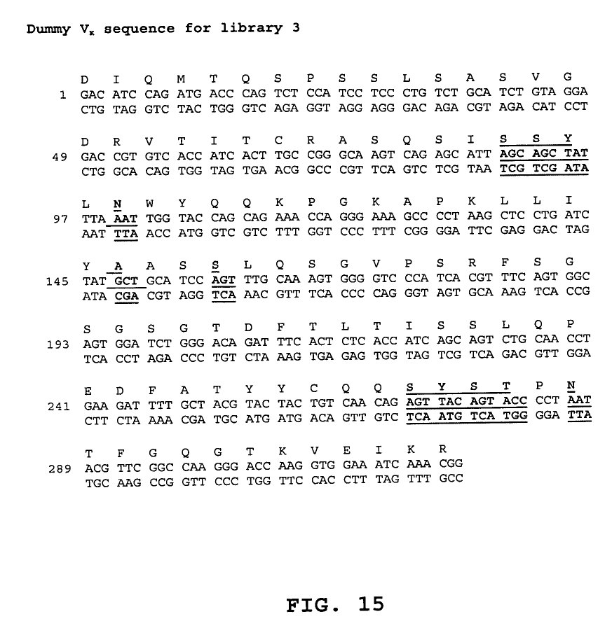

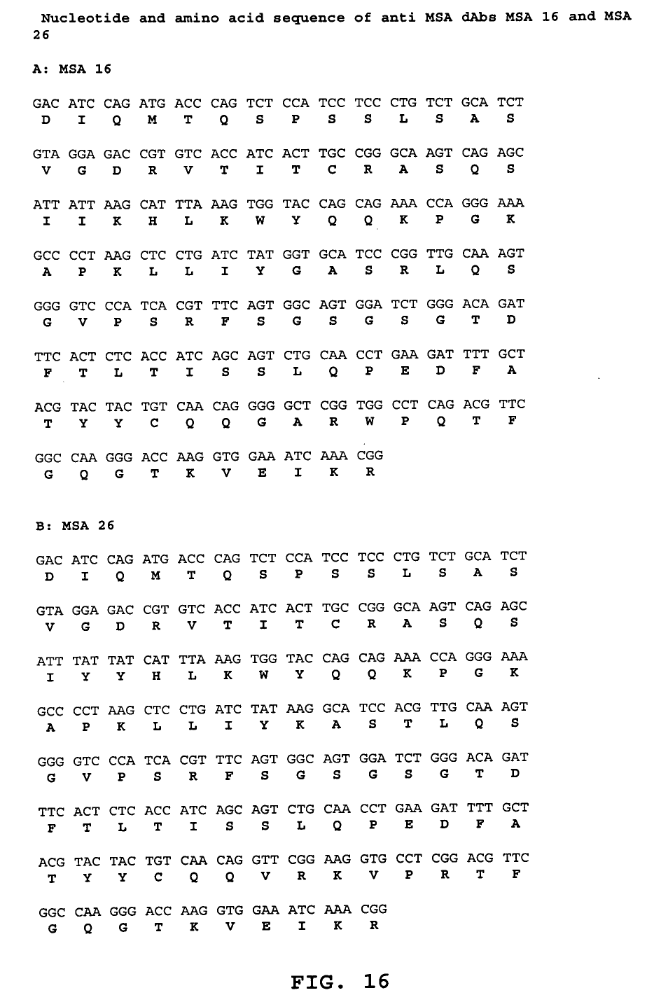

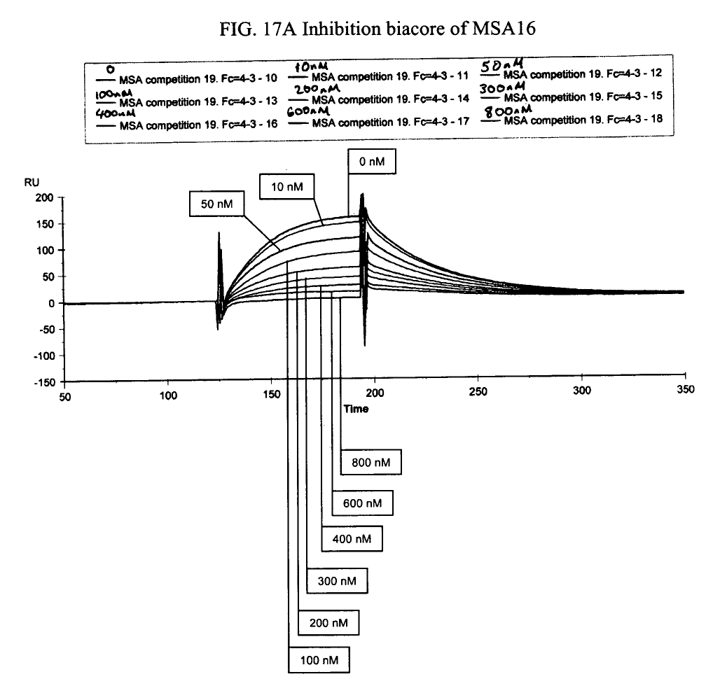

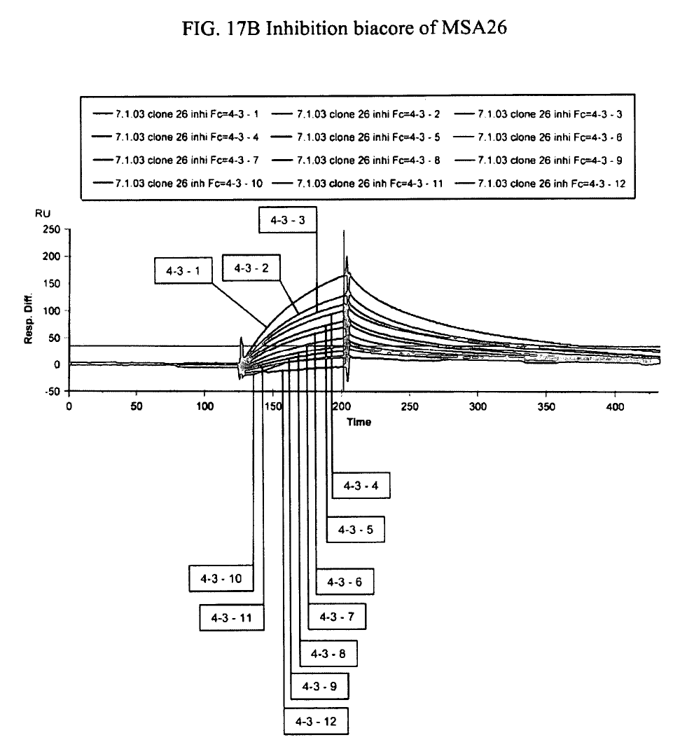

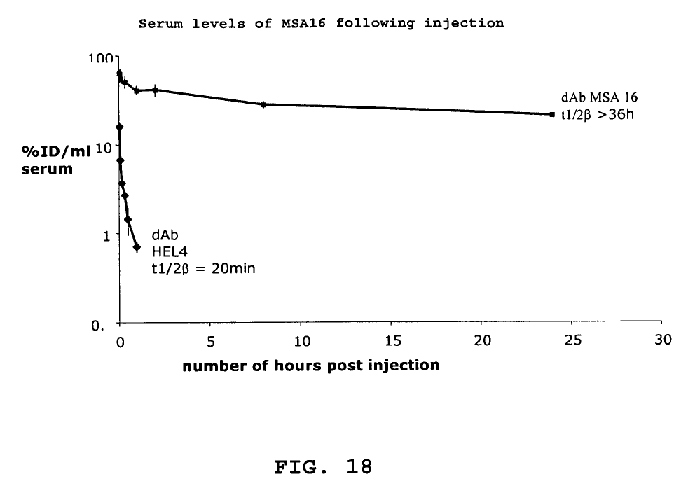

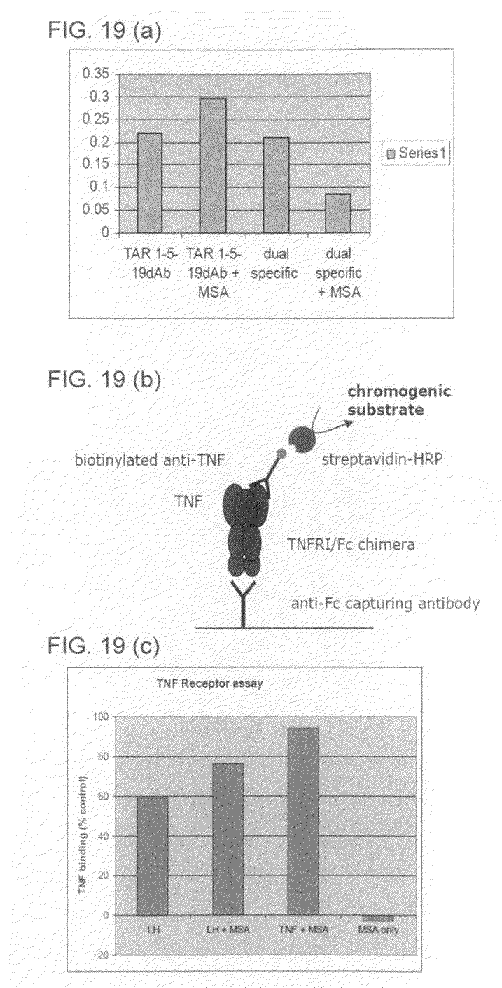

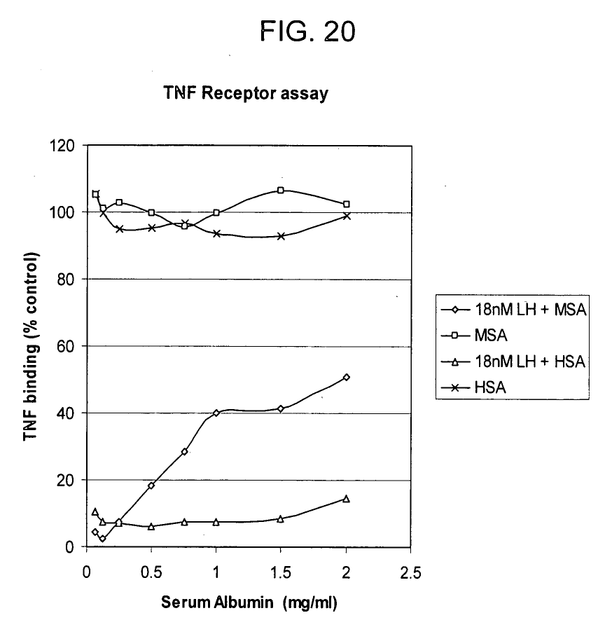

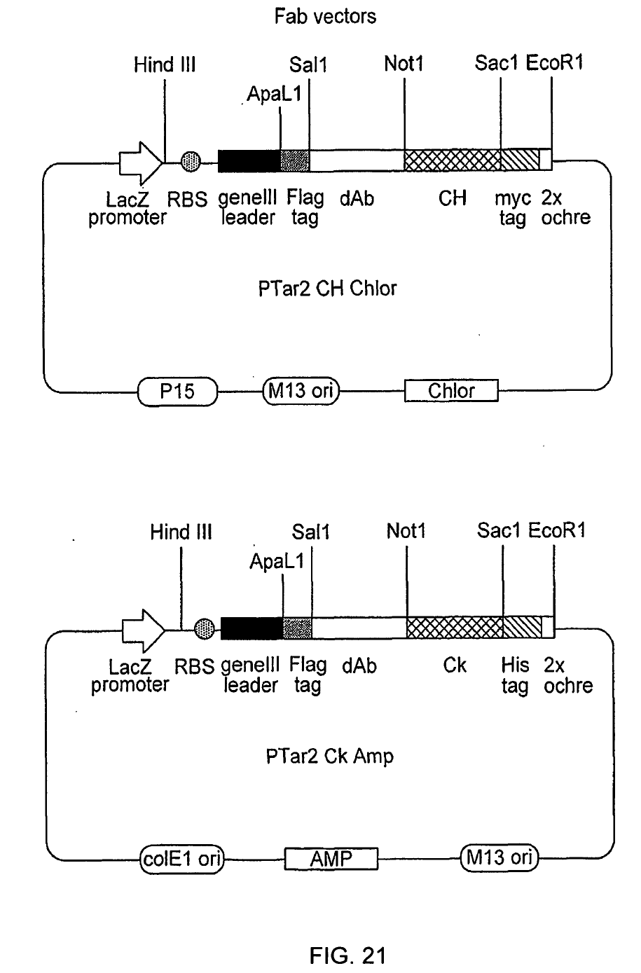

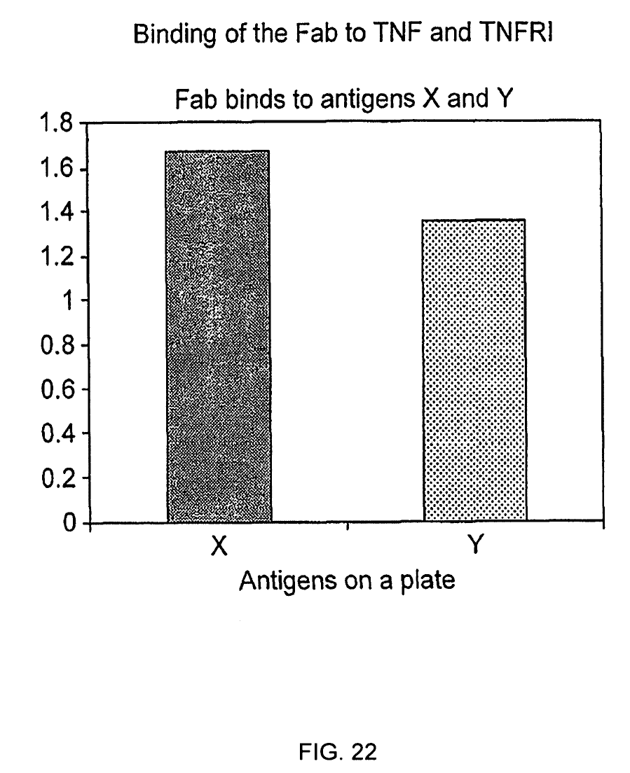

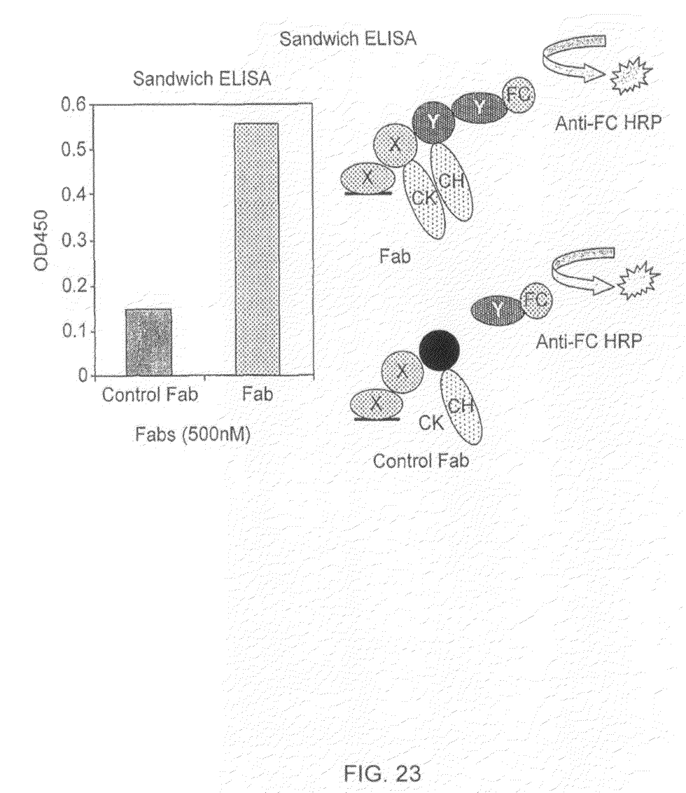

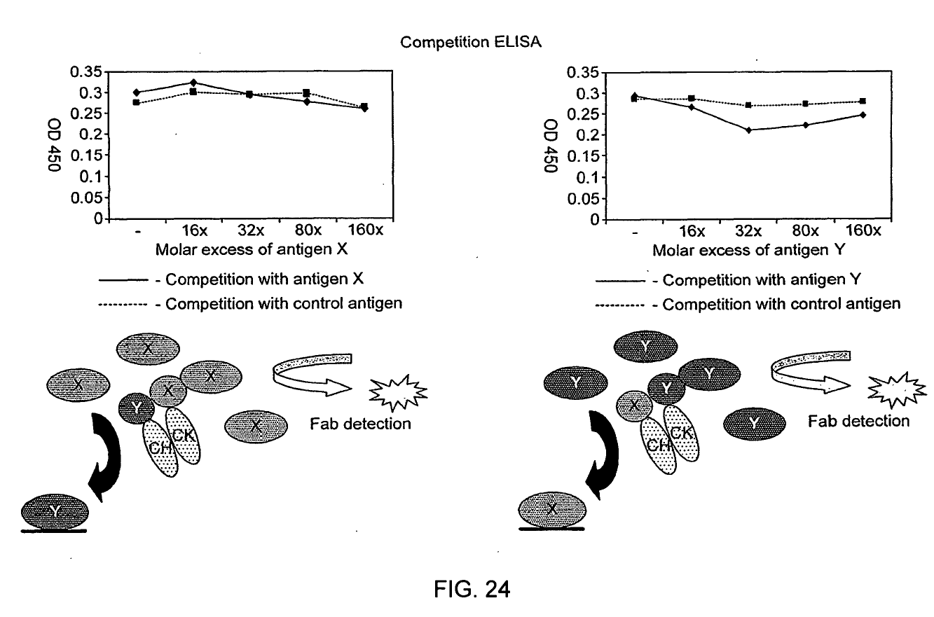

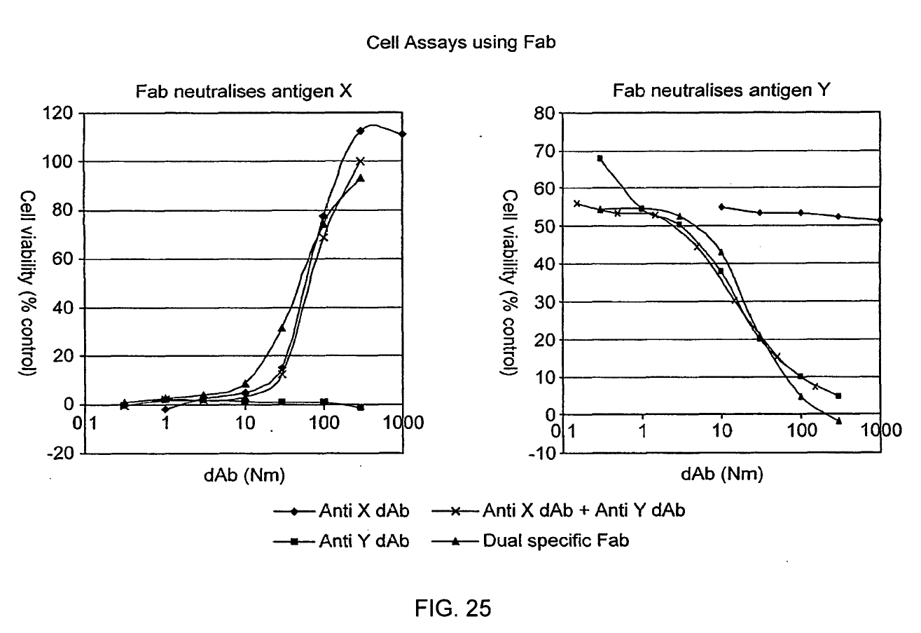

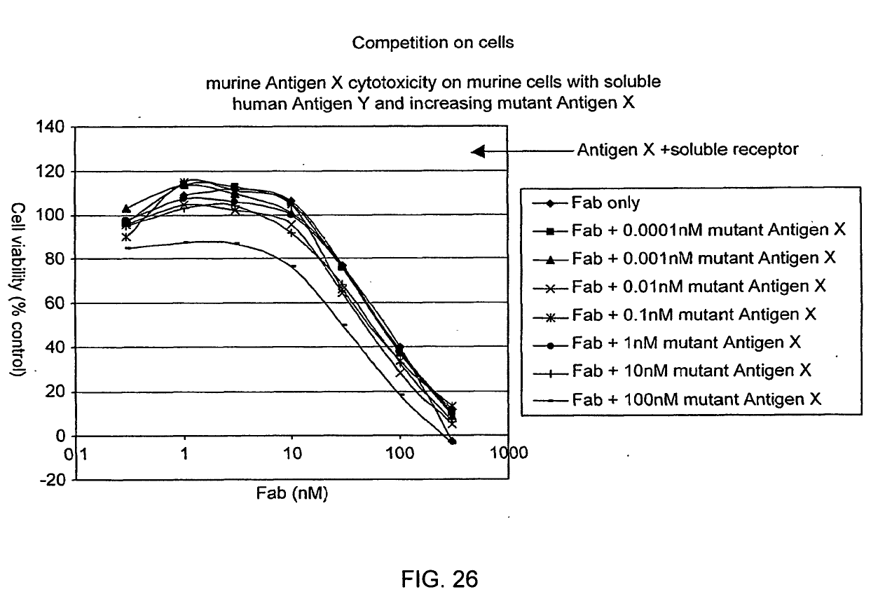

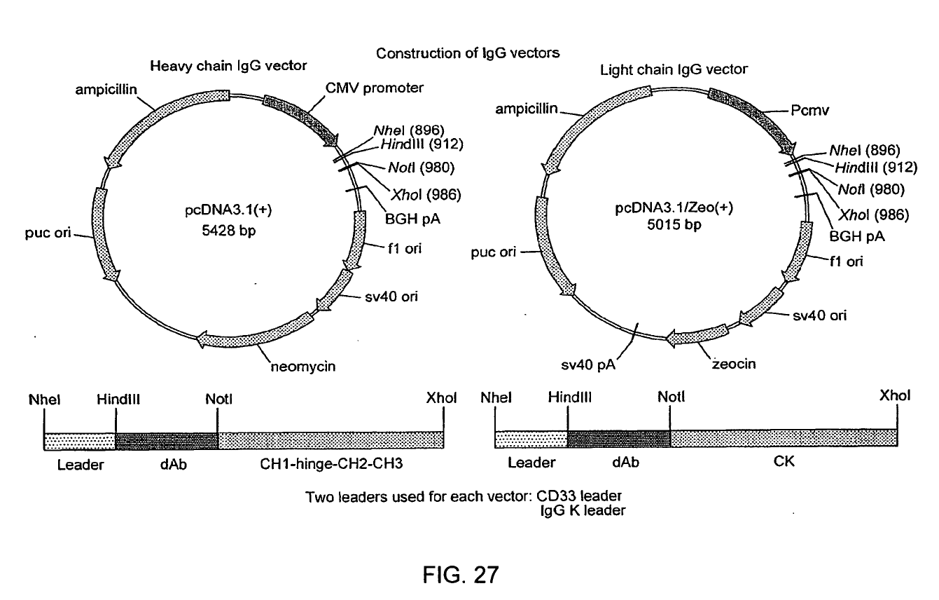

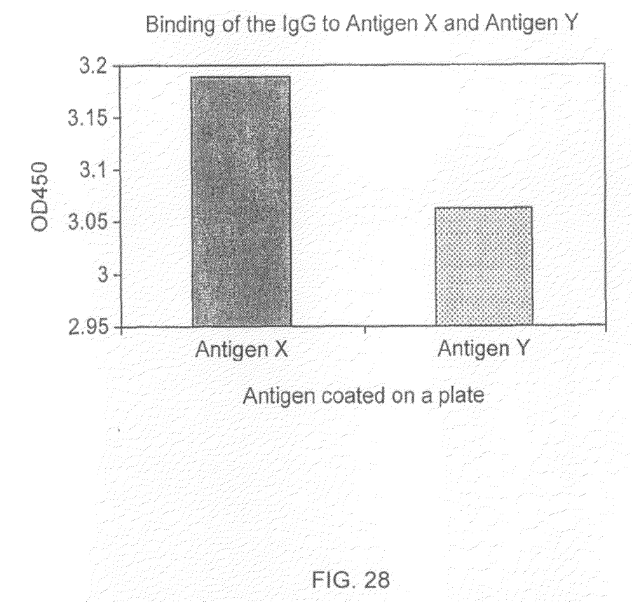

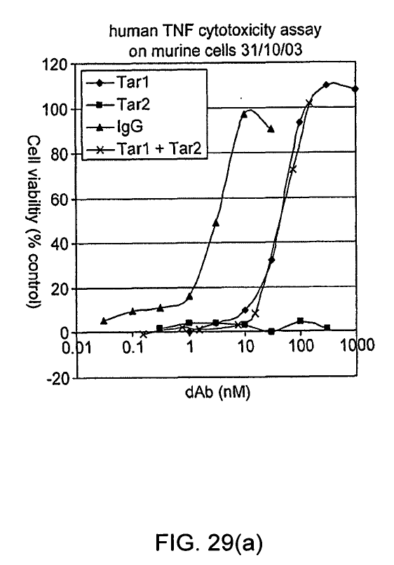

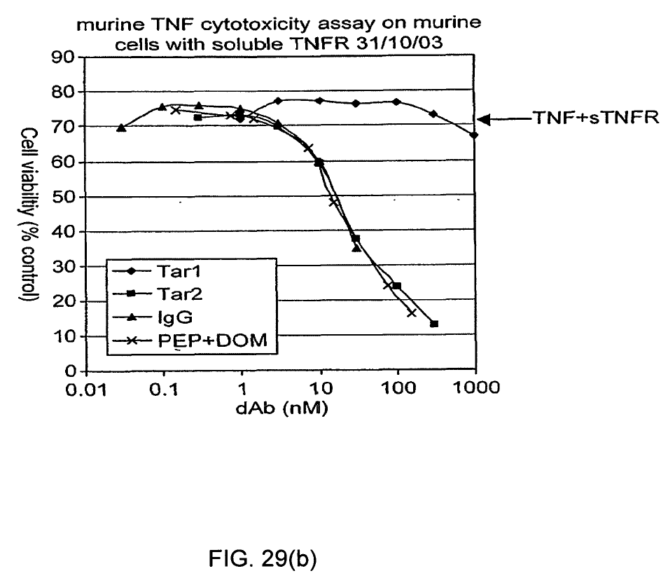

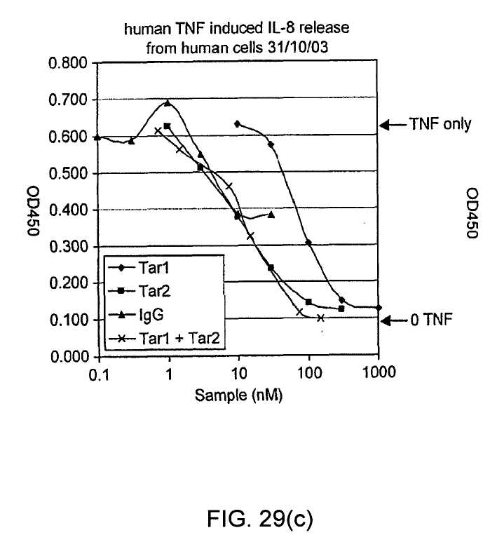

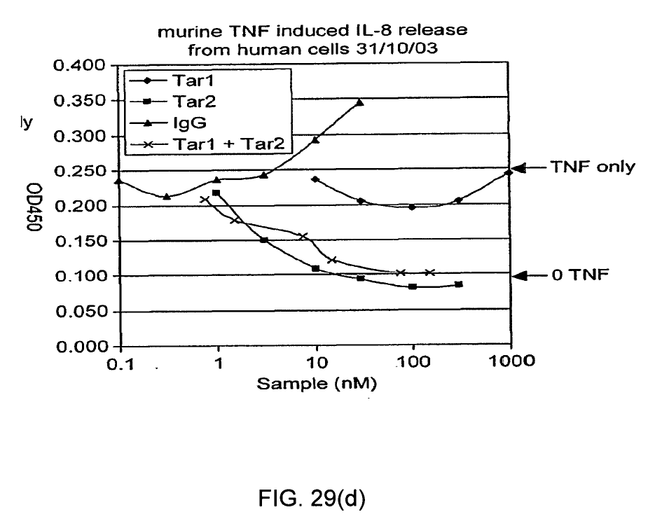

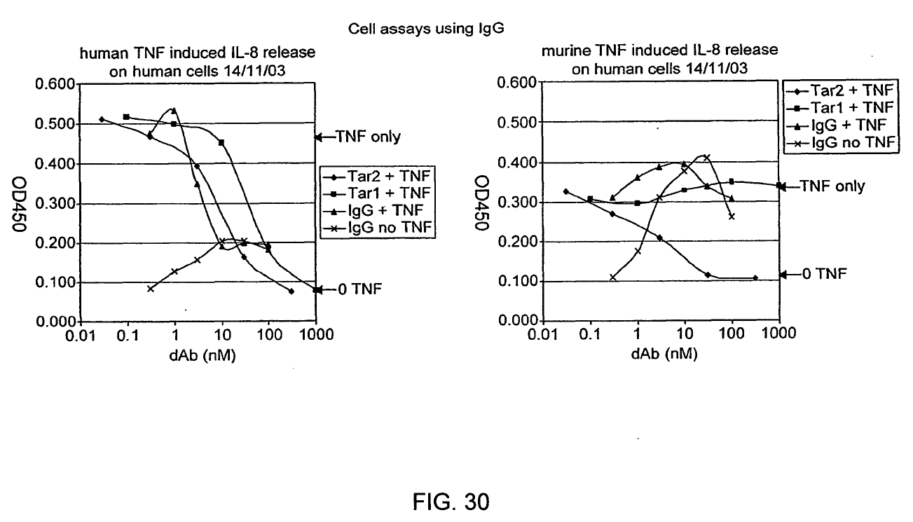

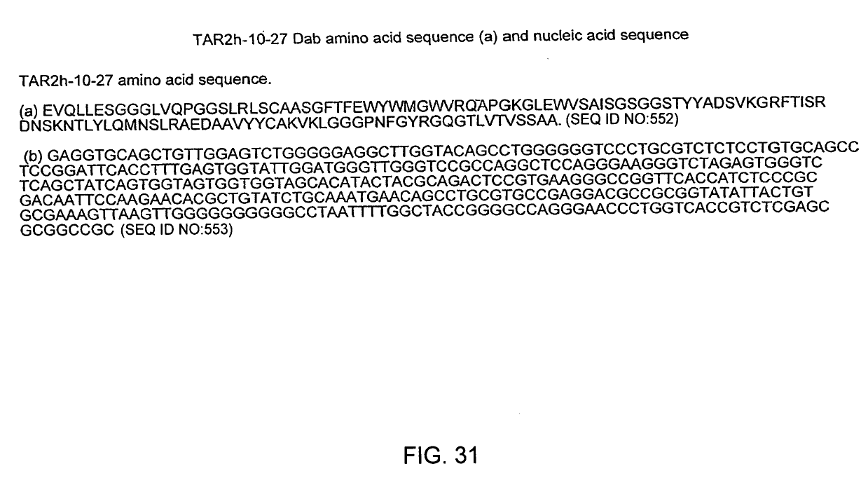

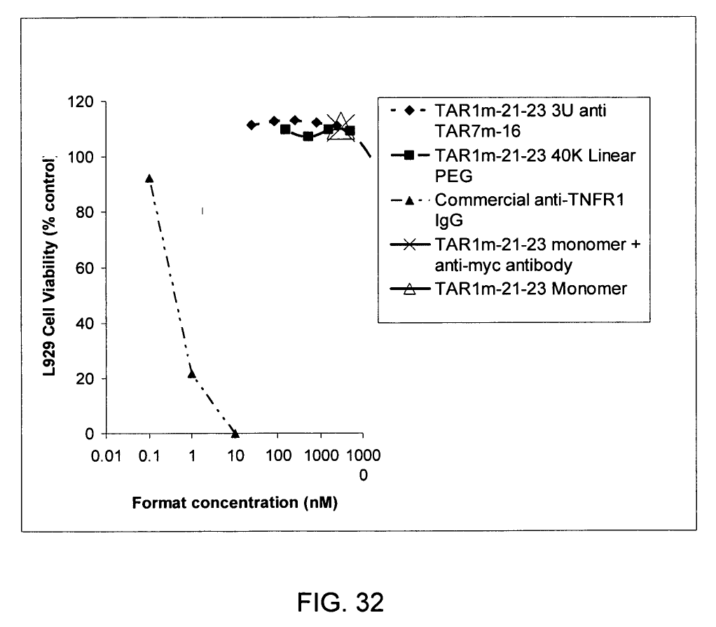

Library 1 and Library 2 contain a dummy V_{κ} sequence, whereas the sequence of V_{H} is diversified at positions H50, H52, H52a, H53, H55, H56, H58, H95, H96, H97 and H98 (DVT or NNK encoded, respectively) (FIG. 1). Library 3 and Library 4 contain a dummy V_{H} sequence, whereas the sequence of V_{κ} is diversified at positions L50, L53, L91, L92, L93, L94 and L96 (DVT or NNK encoded, respectively) (FIG. 1). The libraries are in phagemid pIT2/ScFv format (FIG. 2) and have been preselected for binding to generic ligands, Protein A and Protein L, so that the majority of clones in the unselected libraries are functional. The sizes of the libraries shown above correspond to the sizes after preselection. Library 1 and Library 2 were mixed prior to selections on antigen to yield a single V_{H}/dummy V_{κ} library and Library 3 and Library 4 were mixed to form a single V_{κ}/dummy V_{H} library.

Three rounds of selections were performed on β-gal using V_{κ}/dummy V_{H} library and three rounds of selections were performed on HSA using V_{H}/dummy V_{κ} library. In the case of β-gal the phage titres went up from 1.1×10⁶ in the first round to 2.0×10⁸ in the third round. In the case of HSA the phage titres went up from 2×10⁴ in the first round to 1.4×10⁹ in the third round. The selections were performed as described by Griffith et al., (1993), except that KM13 helper phage (which contains a pIII protein with a protease cleavage site between the D2 and D3 domains) was used and phage were eluted with 1 mg/ml trypsin in PBS. The addition of trypsin cleaves the pIII proteins derived from the helper phage (but not those from the phagemid) and elutes bound scFv-phage fusions by cleavage in the c-myc tag (FIG. 2), thereby providing a further enrichment for phages expressing functional scFvs and a corresponding reduction in background (Kristensen & Winter, Folding & Design 3: 321-328, Jul. 9, 1998). Selections were performed using immunotubes coated with either HSA or β-gal at 100 μg/ml concentration.

To check for binding, 24 colonies from the third round of each selection were screened by monoclonal phage ELISA. Phage particles were produced as described by Harrison et al., Methods Enzymol. 1996; 267:83-109. 96-well ELISA plates were coated with 100 μl of HSA or β-gal at 10 μg/ml concentration in PBS overnight at 4° C. A standard ELISA protocol was followed (Hoogenboom et al., 1991) using detection of bound phage with anti-M13-HRP conjugate. A selection of clones gave ELISA signals of greater than 1.0 with 50 μl supernatant.

Next, DNA preps were made from V_{H}/dummy V_{κ} library selected on HSA and from V_{κ}/dummy V_{H} library selected on β-gal using the QIAprep Spin Miniprep kit (Qiagen). To access most of the diversity, DNA preps were made from each of the three rounds of selections and then pulled together for each of the antigens. DNA preps were then digested with SalI/NotI overnight at 37° C. Following gel purification of the fragments, V_{κ} chains from the V_{κ}/dummy V_{H} library selected on β-gal were ligated in place of a dummy V_{κ} chain of the V_{H}/dummy V_{κ} library selected on HSA creating a library of 3.3×10⁹ clones.

This library was then either selected on HSA (first round) and β-gal (second round), HSA/β-gal selection, or on β-gal (first round) and HSA (second round), β-gal/HSA selection. Selections were performed as described above. In each case after the second round 48 clones were tested for binding to HSA and β-gal by the monoclonal phage ELISA (as described above) and by ELISA of the soluble scFv fragments. Soluble antibody fragments were produced as described by Harrison et al., (1996), and standard ELISA protocol was followed Hoogenboom et al. (1991) Nucleic Acids Res., 19: 4133, except that 2% Tween/PBS was used as a blocking buffer and bound scFvs were detected with Protein L-HRP. Three clones (E4, E5 and E8) from the HSA/β-gal selection and two clones (K8 and K10) from the β-gal/HSA selection were able to bind both antigens. scFvs from these clones were PCR amplified and sequenced as described by Ignatovich et al., (1999) J Mol Biol 1999 Nov. 26; 294(2):457-65, using the primers LMB3 and pHENseq. Sequence analysis revealed that all clones were identical. Therefore, only one clone encoding a dual specific antibody (K8) was chosen for further work (FIG. 3).

### Example 2

### Characterisation of the Binding Properties of the K8 Antibody

Firstly, the binding properties of the K8 antibody were characterised by the monoclonal phage ELISA. A 96-well plate was coated with 100 μl of HSA and β-gal alongside with alkaline phosphatase (APS), bovine serum albumin (BSA), peanut agglutinin, lysozyme and cytochrome c (to check for cross-reactivity) at 10 μg/ml concentration in PBS overnight at 4° C. The phagemid from K8 clone was rescued with KM13 as described by Harrison et al., (1996) and the supernatant (50 μl) containing phage assayed directly. A standard ELISA protocol was followed (Hoogenboom et al., 1991) using detection of bound phage with anti-M13-HRP conjugate. The dual specific K8 antibody was found to bind to HSA and β-gal when displayed on the surface of the phage with absorbance signals greater than 1.0 (FIG. 4). Strong binding to BSA was also observed (FIG. 4). Since HSA and BSA are 76% homologous on the amino acid level, it is not surprising that K8 antibody recognised both of these structurally related proteins. No cross-reactivity with other proteins was detected (FIG. 4).

Secondly, the binding properties of the K8 antibody were tested in a soluble scFv ELISA. Production of the soluble scFv fragment was induced by IPTG as described by Harrison et al., (1996). To determine the expression levels of K8 scFv, the soluble antibody fragments were purified from the supernatant of 50 ml inductions using Protein A-Sepharose columns as described by Harlow and Lane, Antibodies: a Laboratory Manual, (1988) Cold Spring Harbor. OD₂₈₀ was then measured and the protein concentration calculated as described by Sambrook et al., (1989). K8 scFv was produced in supernatant at 19 mg/l.

A soluble scFv ELISA was then performed using known concentrations of the K8 antibody fragment. A 96-well plate was coated with 100 μl of HSA, BSA and β-gal at 10 μg/ml and 100 μl of Protein A at 1 μg/ml concentration. 50 μl of the serial dilutions of the K8 scFv was applied and the bound antibody fragments were detected with Protein L-HRP. ELISA results confirmed the dual specific nature of the K8 antibody (FIG. 5).

To confirm that binding to β-gal is determined by the V_{κ} domain and binding to HSA/BSA by the V_{H} domain of the K8 scFv antibody, the V_{κ} domain was cut out from K8 scFv DNA by SalI/NotI digestion and ligated into a SalI/NotI digested pIT2 vector containing dummy V_{H} chain (FIGS. 1 and 2). Binding characteristics of the resulting clone K8V_{κ}/dummy V_{H} were analysed by soluble scFv ELISA. Production of the soluble scFv fragments was induced by IPTG as described by Harrison et al., (1996) and the supernatant (50%) containing scFvs assayed directly. Soluble scFv ELISA was performed as described in Example 1 and the bound scFvs were detected with Protein L-HRP. The ELISA results revealed that this clone was still able to bind β-gal, whereas binding to BSA was abolished (FIG. 6).

### Example 3

### Selection of Single V_{H} Domain Antibodies Antigens A and B and Single V_{κ} Domain Antibodies Directed Against Antigens C and D

This example describes a method for making single V_{H} domain antibodies directed against antigens A and B and single V_{κ} domain antibodies directed against antigens C and D by selecting repertoires of virgin single antibody variable domains for binding to these antigens in the absence of the complementary variable domains.

Selections and characterisation of the binding clones is performed as described previously (see Example 5, PCT/GB 02/003014). Four clones are chosen for further work:

VH1—Anti A V_{H}

VH2—Anti B V_{H}

VK1—AntiC V_{κ}

VK2—Anti D V_{κ}

The procedures described above in Examples 1-3 may be used, in a similar manner as that described, to produce dimer molecules comprising combinations of V_{H} domains (i.e., V_{H}-V_{H} ligands) and combinations of V_{L} domains (V_{L}-V_{L} ligands).

### Example 4

### Creation and Characterisation of the Dual Specific ScFv Antibodies (V_{H} I V_{H}2 Directed Against Antigens A and B and VK1/VK2 Directed Against Antigens C and D)

This example demonstrates that dual specific ScFv antibodies (VH1/V_{H}2 directed against antigens A and B and VK1/VK2 directed against antigens C and D) could be created by combining V_{κ} and V_{H} single domains selected against respective antigens in a ScFv vector.

To create dual specific antibody VH1/V_{H}2, V_{H}1 single domain is excised from variable domain vector 1 (FIG. 7) by NcoI/XhoI digestion and ligated into NcoI/XhoI digested variable domain vector 2 (FIG. 7) to create V_{H}1/variable domain vector 2. V_{H}2 single domain is PCR amplified from variable domain vector 1 using primers to introduce SalI restriction site to the 5′ end and NotI restriction site to the 3′ end. The PCR product is then digested with SalI/NotI and ligated into SalI/NotI digested V_{H}1/variable domain vector 2 to create VH1/V_{H}2/variable domain vector 2.

VK1/VK2/variable domain vector 2 is created in a similar way. The dual specific nature of the produced V_{H}1/V_{H}2 ScFv and VK1/VK2 ScFv is tested in a soluble ScFv ELISA as described previously (see Example 6, PCT/GB 02/003014). Competition ELISA is performed as described previously (see Example 8, PCT/GB 02/003014).

Possible outcomes:

VH1/V_{H}2 ScFv is able to bind antigens A and B simultaneously

VK1/VK2 ScFv is able to bind antigens C and D simultaneously

VH1/VH2 ScFv binding is competitive (when bound to antigen A, VH1/V1H2 ScFv cannot bind to antigen B)

VK1/VK2 ScFv binding is competitive (when bound to antigen C, VK1/VK2 ScFv cannot bind to antigen D)

### Example 5

### Construction of Dual Specific V_{H}11V_{H}2 Fab and VK/VK2 Fab and Analysis of their Binding Properties

To create V_{H}11V_{H}2 Fab, VH1 single domain is ligated into NcoI/XhoI digested CH vector (FIG. 8) to create V_{H}1/CH and V_{H}2 single domain is ligated into SalI/NotI digested CK vector (FIG. 9) to create V_{H}2/CK. Plasmid DNA from V_{H}1/CH and V_{H}2/CK is used to co-transform competent E. coli cells as described previously (see Example 8, PCT/GB02/003014).

The clone containing V_{H}1/CH and V_{H}2/CK plasmids is then induced by IPTG to produce soluble VH1/V_{H}2 Fab as described previously (see Example 8, PCT/GB 02/003014).

VK1/VK2 Fab is produced in a similar way. Binding properties of the produced Fabs are tested by competition ELISA as described previously (see Example 8, PCT/GB 02/003014).

Possible outcomes:

VH1/VH2 Fab is able to bind antigens A and B simultaneously

VK1/VK2 Fab is able to bind antigens C and D simultaneously

VH1/VH2 Fab binding is competitive (when bound to antigen A, VH1/VH2 Fab cannot bind to antigen B)

VK1/VK2 Fab binding is competitive (when bound to antigen C, VK1/VK2 Fab cannot bind to antigen D)

### Example 6

### Chelating dAb Dimers

Summary

VH and VK homo-dimers are created in a dAb-linker-dAb format using flexible polypeptide linkers. Vectors were created in the dAb linker-dAb format containing glycine-serine linkers of different lengths 3U:(Gly₄Ser)₃, 5U:(Gly₄Ser)₅, 7U:(Gly₄Ser)₇. Dimer libraries were created using guiding dAbs upstream of the linker: TAR1-5 (VK), TAR1-27(VK), TAR2-5(VH) or TAR2-6(VK) and a library of corresponding second dAbs after the linker. Using this method, novel dimeric dAbs were selected. The effect of dimerisation on antigen binding was determined by ELISA and BIAcore studies and in cell neutralisation and receptor binding assays. Dimerisation of both TAR1-5 and TAR1-27 resulted in significant improvement in binding affinity and neutralisation levels.

1.0 Methods

1.1 Library Generation

1.1.1 Vectors

pEDA3U, pEDA5U and pEDA7U vectors were designed to introduce different linker lengths compatible with the dAb-linker-dAb format. For pEDA3U, sense and anti-sense 73-base pair oligo linkers were annealed using a slow annealing program (95° C.-5 mins, 80° C.-10 mins, 70° C.-15 mins, 56° C.-15 mins, 42° C. until use) in buffer containing 0.1 MNaCl, 10 mM Tris-HCl pH7.4 and cloned using the Xho1 and Not1 restriction sites. The linkers encompassed 3 (Gly₄Ser) units and a stuffer region housed between Sal1 and Not1 cloning sites (scheme 1). In order to reduce the possibility of monomeric dAbs being selected for by phage display, the stuffer region was designed to include 3 stop codons, a Sac1 restriction site and a frame shift mutation to put the region out of frame when no second dAb was present. For pEDA5U and 7U due to the length of the linkers required, overlapping oligo-linkers were designed for each vector, annealed and elongated using Klenow. The fragment was then purified and digested using the appropriate enzymes before cloning using the Xho1 and Not1 restriction sites.

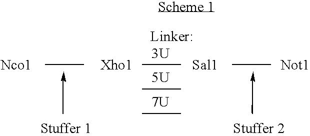

1.1.2 Library Preparation

The N-terminal V gene corresponding to the guiding dAb was cloned upstream of the linker using Nco1 and Xho1 restriction sites. V_{H} genes have existing compatible sites, however cloning VK genes required the introduction of suitable restriction sites. This was achieved by using modifying PCR primers (VK-DLIBF: 5′ cggccatggcgtcaacggacat (SEQ ID NO:465); VKXho1R: 5′ atgtgcgctcgagcgtttgat 3′ (SEQ ID NO:466)) in 30 cycles of PCR amplification using a 2:1 mixture of SuperTaq (HTBiotechnology Ltd) and pfu turbo (Stratagene). This maintained the Nco1 site at the 5′ end while destroying the adjacent SalI site and introduced the Xho1 site at the 3′ end. 5 guiding dAbs were cloned into each of the 3 dimer vectors: TAR1-5 (VK), TAR1-27(VK), TAR2-5(VH), TAR2-6(VK) and TAR2-7(VK). All constructs were verified by sequence analysis.

Having cloned the guiding dAbs upstream of the linker in each of the vectors (pEDA3U, 5U and 7U): TAR1-5 (VK), TAR1-27(VK), TAR2-5(VH) or TAR2-6(VK) a library of corresponding second dAbs were cloned after the linker. To achieve this, the complimentary dAb libraries were PCR amplified from phage recovered from round 1 selections of either a V_{κ} library against Human TNFα (at approximately 1×10⁶ diversity after round 1) when TAR1-5 or TAR1-27 are the guiding dAbs, or a V_{H} or VK library against human p55 TNF receptor (both at approximately 1×10⁵ diversity after round 1) when TAR2-5 or TAR2-6 respectively are the guiding dAbs. For VK libraries PCR amplification was conducted using primers in 30 cycles of PCR amplification using a 2:1 mixture of SuperTaq and pfu turbo. V_{H} libraries were PCR amplified using primers in order to introduce a SalI restriction site at the 5′ end of the gene. The dAb library PCRs were digested with the appropriate restriction enzymes, ligated into the corresponding vectors down stream of the linker, using Sal1/Not1 restriction sites and electroporated into freshly prepared competent TG1 cells.

The titres achieved for each library are as follows:

TAR1-5: pEDA3U=4×10⁸, pEDA5U=8×10⁷, pEDA7U=1×10⁸

TAR1-27: pEDA3U=6.2×10⁸, pEDA5U=1×10⁸, pEDA7U=1×10⁹

TAR2h-5: pEDA3U=4×10⁷, pEDA5U=2×10⁸, pEDA7U=8×10⁷

TAR2h-6: pEDA3U=7.4×10⁸, pEDA5U=1.2×10⁸, pEDA7U=2.2×10⁸

1.2 Selections

1.2.1 TNFα

Selections were conducted using human TNFα passively coated on immunotubes. Briefly, Immunotubes are coated overnight with 1-4 mls of the required antigen. The immunotubes were then washed 3 times with PBS and blocked with 2% milk powder in PBS for 1-2 hrs and washed a further 3 times with PBS. The phage solution is diluted in 2% milk powder in PBS and incubated at room temperature for 2 hrs. The tubes are then washed with PBS and the phage eluted with 1 mg/ml trypsin-PBS. Three selection strategies were investigated for the TAR1-5 dimer libraries. The first round selections were carried out in immunotubes using human TNFα coated at 1 μg/ml or 20 μg/ml with 20 washes in PBS 0.1% Tween. TG1 cells are infected with the eluted phage and the titres are determined (eg, Marks et al J Mol Biol. 1991 Dec. 5; 222(3):581-97, Richmann et al Biochemistry. 1993 Aug. 31; 32(34):8848-55).

The titres recovered were:

pEDA3U=2.8×10⁷ (1 μg/ml TNF) 1.5×10⁸ (20 μg/mlTNF),

pEDA5U=1.8×10⁷ (1 μg/ml TNF), 1.6×10⁸ (20 μg/ml TNF)

pEDA7U=8×10⁶ (1 μg/ml TNF), 7×10⁷ (20 μg/ml TNF).

The second round selections were carried out using 3 different methods.

In immunotubes, 20 washes with overnight incubation followed by a further 10 washes.

In immunotubes, 20 washes followed by 1 hr incubation at RT in wash buffer with (1 μg/ml TNFα) and 10 further washes.

Selection on streptavidin beads using 33 pmoles biotinylated human TNFα (Henderikx et al., 2002, Selection of antibodies against biotinylated antigens. Antibody Phage Display Methods and protocols, Ed. O'Brien and Atkin, Humana Press). Single clones from round 2 selections were picked into 96 well plates and crude supernatant preps were made in 2 ml 96 well plate format.

TABLE 1

| | Round 1 | | | |
|---|---|---|---|---|
| | Human | | | |
| | TNFαimmunotube | Round 2 | Round 2 | Round 2 |
| | coating | selection | selection | selection |
| | concentration | method 1 | method 2 | method 3 |
| pEDA3U | 1 μg/ml | 1 × 10⁹ | 1.8 × 10⁹ | 2.4 × 10¹⁰ |
| pEDA3U | 20 μg/ml | 6 × 10⁹ | 1.8 × 10¹⁰ | 8.5 × 10¹⁰ |
| pEDA5U | 1 μg/ml | 9 × 10⁸ | 1.4 × 10⁹ | 2.8 × 10¹⁰ |
| pEDA5U | 20 μg/ml | 9.5 × 10⁹ | 8.5 × 10⁹ | 2.8 × 10¹⁰ |
| pEDA7U | 1 μg/ml | 7.8 × 10⁸ | 1.6 × 10⁸ | 4 × 10¹⁰ |
| pEDA7U | 20 μg/ml | 1 × 10¹⁰ | 8 × 10⁹ | 1.5 × 10¹⁰ |

For TAR1-27, selections were carried out as described previously with the following modifications. The first round selections were carried out in immunotubes using human TNFα coated at 1 μg/ml or 20 μg/ml with 20 washes in PBS 0.1% Tween. The second round selections were carried out in immunotubes using 20 washes with overnight incubation followed by a further 20 washes. Single clones from round 2 selections were picked into 96 well plates and crude supernatant preps were made in 2 ml 96 well plate format.

TAR1-27 titres are as follows:

TABLE 2

| | Human | | |
|---|---|---|---|
| | TNFαimmunotube | | |
| | coating conc | Round 1 | Round 2 |
| pEDA3U | 1 μg/ml | 4 × 10⁹ | 6 × 10⁹ |
| pEDA3U | 20 μg/ml | 5 × 10⁹ | 4.4 × 10¹⁰ |
| pEDA5U | 1 μg/ml | 1.5 × 10⁹ | 1.9 × 10¹⁰ |
| pEDA5U | 20 μg/ml | 3.4 × 10⁹ | 3.5 × 10¹⁰ |
| pEDA7U | 1 μg/ml | 2.6 × 10⁹ | 5 × 10⁹ |
| pEDA7U | 20 μg/ml | 7 × 10⁹ | 1.4 × 10¹⁰ |

1.2.2 TNF Receptor 1 (p55 Receptor; TAR2)

Selections were conducted as described previously for the TAR2h-5 libraries only. 3 rounds of selections were carried out in immunotubes using either 1 μg/ml human p55 TNF receptor or 10 μg/ml human p55 TNF receptor with 20 washes in PBS 0.1% Tween with overnight incubation followed by a further 20 washes. Single clones from round 2 and 3 selections were picked into 96 well plates and crude supernatant preps were made in 2 ml 96 well plate format.

TAR2h-5 titres are as follows:

TABLE 3

| | Round 1 human | | | |
|---|---|---|---|---|
| | p55 TNF | | | |
| | receptor | | | |
| | immunotube | | | |
| | coating | | | |
| | concentration | Round 1 | Round 2 | Round 3 |
| pEDA3U | 1 μg/ml | 2.4 × 10⁶ | 1.2 × 10⁷ | 1.9 × 10⁹ |
| pEDA3U | 10 μg/ml | 3.1 × 10⁷ | 7 × 10⁷ | 1 × 10⁹ |
| pEDA5U | 1 μg/ml | 2.5 × 10⁶ | 1.1 × 10⁷ | 5.7 × 10⁸ |
| pEDA5U | 10 μg/ml | 3.7 × 10⁷ | 2.3 × 10⁸ | 2.9 × 10⁹ |
| pEDA7U | 1 μg/ml | 1.3 × 10⁶ | 1.3 × 10⁷ | 1.4 × 10⁹ |
| pEDA7U | 10 μg/ml | 1.6 × 10⁷ | 1.9 × 10⁷ | 3 × 10¹⁰ |

1.3 Screening

Single clones from round 2 or 3 selections were picked from each of the 3U, 5U and 7U libraries from the different selections methods, where appropriate. Clones were grown in 2×TY with 100 μg/ml ampicillin and 1% glucose overnight at 37° C. A 1/100 dilution of this culture was inoculated into 2 mls of 2×TY with 100 μg/ml ampicillin and 0.1% glucose in 2 ml, 96 well plate format and grown at 37° C. shaking until OD600 was approximately 0.9. The culture was then induced with 1 mM IPTG overnight at 30° C. The supernatants were clarified by centrifugation at 400 rpm for 15 mins in a sorval plate centrifuge. The supernatant preps the used for initial screening.

1.3.1 ELISA

Binding activity of dimeric recombinant proteins was compared to monomer by Protein A/L ELISA or by antigen ELISA. Briefly, a 96 well plate is coated with antigen or Protein A/L overnight at 4° C. The plate washed with 0.05% Tween-PBS, blocked for 2 hrs with 2% Tween-PBS. The sample is added to the plate incubated for 1 hr at room temperature. The plate is washed and incubated with the secondary reagent for 1 hr at room temperature. The plate is washed and developed with TMB substrate. Protein A/L-HRP or India-HRP was used as a secondary reagent. For antigen ELISAs, the antigen concentrations used were 1 μg/ml in PBS for Human TNFα and human THF receptor 1. Due to the presence of the guiding dAb in most cases dimers gave a positive ELISA signal therefore off rate determination was examined by BIAcore.

1.3.2 BIAcore

BIAcore analysis was conducted for TAR1-5 and TAR2h-5 clones. For screening, Human TNFα was coupled to a CM5 chip at high density (approximately 10000 RUs). 50 μl of Human TNFα (50 μg/ml) was coupled to the chip at 5 μl/min in acetate buffer—pH5.5. Regeneration of the chip following analysis using the standard methods is not possible due to the instability of Human TNFα, therefore after each sample was analysed, the chip was washed for 10 mins with buffer. For TAR1-5, clones supernatants from the round 2 selection were screened by BIAcore. 48 clones were screened from each of the 3U, 5U and 7U libraries obtained using the following selection methods:

R1: 1 μg/ml human TNFα immunotube, R2 1 μg/ml human TNFα immunotube, overnight wash.

R1: 20 μg/ml human TNFα immunotube, R2 20 μg/ml human TNFα immunotube, overnight wash.

R1: 1 μg/ml human TNFα immunotube, R2 33 pmoles biotinylated human TNFα on beads.

R1: 20 μg/ml human TNFα immunotube, R2 33 pmoles biotinylated human TNFα beads.

For screening, human p55 TNF receptor was coupled to a CM5 chip at high density (approximately 4000 RUs). 100 μl of human p55 TNF receptor (10 μg/ml) was coupled to the chip at 5 μl/min in acetate buffer—pH5.5. Standard regeneration conditions were examined (glycine pH2 or pH3) but in each case antigen was removed from the surface of the chip therefore as with TNFα, therefore after each sample was analysed, the chip was washed for 10 mins with buffer.

For TAR2-5, clones supernatants from the round 2 selection were screened. 48 clones were screened from each of the 3U, 5U and 7U libraries, using the following selection methods:

R1: 1 μg/ml human p55 TNF receptor immunotube, R2 1 μg/ml human p55 TNF receptor immunotube, overnight wash.

R1: 10 μg/ml human p55 TNF receptor immunotube, R2 10 μg/ml human p55 TNF receptor immunotube, overnight wash.

1.3.3 Receptor and Cell Assays

The ability of the dimers to neutralise in the receptor assay was conducted as follows:

Receptor Binding

Anti-TNF dAbs were tested for the ability to inhibit the binding of TNF to recombinant TNF receptor 1 (p55). Briefly, Maxisorp plates were incubated overnight with 30 mg/ml anti-human Fc mouse monoclonal antibody (Zymed, San Francisco, USA). The wells were washed with phosphate buffered saline (PBS) containing 0.05% Tween-20 and then blocked with 1% BSA in PBS before being incubated with 100 ng/ml TNF receptor 1 Fc fusion protein (R&D Systems, Minneapolis, USA). Anti-TNF dAb was mixed with TNF which was added to the washed wells at a final concentration of 10 ng/ml. TNF binding was detected with 0.2 mg/ml biotinylated anti-TNF antibody (HyCult biotechnology, Uben, Netherlands) followed by 1 in 500 dilution of horse radish peroxidase labelled streptavidin (Amersham Biosciences, UK) and then incubation with TMB substrate (KPL, Gaithersburg, USA). The reaction was stopped by the addition of HCl and the absorbance was read at 450 nm. Anti-TNF dAb activity lead to a decrease in TNF binding and therefore a decrease in absorbance compared with the TNF only control.

L929 Cytotoxicity Assay

Anti-TNF dAbs were also tested for the ability to neutralise the cytotoxic activity of TNF on mouse L929 fibroblasts (Evans, T. (2000) Molecular Biotechnology 15, 243-248). Briefly, L929 cells plated in microtitre plates were incubated overnight with anti-TNF dAb, 100 pg/ml TNF and 1 mg/ml actinomycin D (Sigma, Poole, UK). Cell viability was measured by reading absorbance at 490 nm following an incubation with [3-(4,5-dimethylthiazol-2-yl)-5-(3-carbboxymethoxyphenyl)-2-(4-sulfophenyl)-2H-tetrazolium (Promega, Madison, USA). Anti-TNF dAb activity lead to a decrease in TNF cytotoxicity and therefore an increase in absorbance compared with the TNF only control.

In the initial screen, supernatants prepared for BIAcore analysis, described above, were also used in the receptor assay. Further analysis of selected dimers was also conducted in the receptor and cell assays using purified proteins.

HeLa IL-8 Assay

Anti-TNFR1 or anti-TNF alpha dAbs were tested for the ability to neutralise the induction of IL-8 secretion by TNF in HeLa cells (method adapted from that of Akeson, L. et al (1996) Journal of Biological Chemistry 271, 30517-30523, describing the induction of IL-8 by IL-1 in HUVEC; here we look at induction by human TNF alpha and we use HeLa cells instead of the HUVEC cell line). Briefly, HeLa cells plated in microtitre plates were incubated overnight with dAb and 300 pg/ml TNF. Post incubation the supernatant was aspirated off the cells and IL-8 concentration measured via a sandwich ELISA (R&D Systems). Anti-TNFR1 dAb activity lead to a decrease in IL-8 secretion into the supernatant compared with the TNF only control.

The L929 assay is used throughout the following experiments; however, the use of the HeLa IL-8 assay is preferred to measure anti-TNF receptor 1 (p55) ligands; the presence of mouse p55 in the L929 assay poses certain limitations in its use.

1.4 Sequence Analysis

Dimers that proved to have interesting properties in the BIAcore and the receptor assay screens were sequenced. Sequences are detailed in the sequence listing.

1.5 Formatting

1.5.1 TAR1-5-19 Dimers

The TAR1-5 dimers that were shown to have good neutralisation properties were re-formatted and analysed in the cell and receptor assays. The TAR1-5 guiding dab was substituted with the affinity matured clone TAR1-5-19. To achieve this TAR1-5 was cloned out of the individual dimer pair and substituted with TAR1-5-19 that had been amplified by PCR. In addition, TAR1-5-19 homodimers were also constructed in the 3U, 5U and 7U vectors. The N terminal copy of the gene was amplified by PCR and cloned as described above and the C-terminal gene fragment was cloned using existing Sal1 and Not1 restriction sites.

1.5.2 Mutagenesis

The amber stop codon present in dAb2, one of the C-terminal dAbs in the TAR1-5 dimer pairs was mutated to a glutamine by site-directed mutagenesis.

1.5.3 Fabs

The dimers containing TAR1-5 or TAR1-5-19 were re-formatted into Fab expression vectors. dAbs were cloned into expression vectors containing either the CK or CH genes using Sfi1 and Not1 restriction sites and verified by sequence analysis. The CK vector is derived from a pUC based ampicillin resistant vector and the CH vector is derived from a pACYC chloramphenicol resistant vector. For Fab expression the dAb-CH and dAb-CK constructs were co-transformed into HB2151 cells and grown in 2×TY containing 0.1% glucose, 100 μg/ml ampicillin and 10 μg/ml chloramphenicol.

1.5.3 Hinge Dimerisation

Dimerisation of dAbs via cystine bond formation was examined. A short sequence of amino acids EPKSGDKTHTCPPCP (SEQ ID NO:467) a modified form of the human IgGC1 hinge was engineered at the C terminal region on the dAb. An oligo linker encoding for this sequence was synthesised and annealed, as described previously. The linker was cloned into the pEDA vector containing TAR1-5-19 using Xho1 and Not1 restriction sites. Dimerisation occurs in situ in the periplasm.

1.6 Expression and Purification

1.6.1 Expression

Supernatants were prepared in the 2 ml, 96-well plate format for the initial screening as described previously. Following the initial screening process selected dimers were analysed further. Dimer constructs were expressed in TOP10F∝ or HB2151 cells as supernatants. Briefly, an individual colony from a freshly streaked plate was grown overnight at 37° C. in 2×TY with 100 μg/ml ampicillin and 1% glucose. A 1/100 dilution of this culture was inoculated into 2×TY with 100 μg/ml ampicillin and 0.1% glucose and grown at 37° C. shaking until OD600 was approximately 0.9. The culture was then induced with 1 mM IPTG overnight at 30° C. The cells were removed by centrifugation and the supernatant purified with protein A or L agarose.

Fab and cysteine hinge dimers were expressed as periplasmic proteins in HB2152 cells. A 1/100 dilution of an overnight culture was inoculated into 2×TY with 0.1% glucose and the appropriate antibiotics and grown at 30° C. shaking until OD600 was approximately 0.9. The culture was then induced with 1 mM IPTG for 3-4 hours at 25° C. The cells were harvested by centrifugation and the pellet resuspended in periplasmic preparation buffer (30 mM Tris-HCl pH8.0, 1 mM EDTA, 20% sucrose). Following centrifugation the supernatant was retained and the pellet resuspended in 5 mM MgSO₄. The supernatant was harvested again by centrifugation, pooled and purified.

1.6.2 Protein A/L purification

Optimisation of the purification of dimer proteins from Protein L agarose (Affitech, Norway) or Protein A agarose (Sigma, UK) was examined. Protein was eluted by batch or by column elution using a peristaltic pump. Three buffers were examined 0.1M Phosphate-citrate buffer pH2.6, 0.2M Glycine pH2.5 and 0.1M Glycine pH2.5. The optimal condition was determined to be under peristaltic pump conditions using 0.1M Glycine pH2.5 over 10 column volumes. Purification from protein A was conducted peristaltic pump conditions using 0.1M Glycine pH2.5.

1.6.3 FPLC Purification

Further purification was carried out by FPLC analysis on the AKTA Explorer 100 system (Amersham Biosciences Ltd). TAR1-5 and TAR1-5-19 dimers were fractionated by cation exchange chromatography (1 ml Resource S—Amersham Biosciences Ltd) eluted with a 0-1M NaCl gradient in 50 mM acetate buffer pH4. Hinge dimers were purified by ion exchange (1 ml Resource Q Amersham Biosciences Ltd) eluted with a 0-1M NaCl gradient in 25 mMTris HCl pH 8.0. Fabs were purified by size exclusion chromatography using a superose 12 (Amersham Biosciences Ltd) column run at a flow rate of 0.5 ml/min in PBS with 0.05% tween. Following purification samples were concentrated using vivaspin 5K cut off concentrators (Vivascience Ltd).

2.0 Results

2.1 TAR1-5 Dimers

6×96 clones were picked from the round 2 selection encompassing all the libraries and selection conditions. Supernatant preps were made and assayed by antigen and Protein L ELISA, BIAcore and in the receptor assays. In ELISAs, positive binding clones were identified from each selection method and were distributed between 3U, 5U and 7U libraries. However, as the guiding dAb is always present it was not possible to discriminate between high and low affinity binders by this method therefore BIAcore analysis was conducted.

BIAcore analysis was conducted using the 2 ml supernatants. BIAcore analysis revealed that the dimer K_{off} rates were vastly improved compared to monomeric TAR1-5. Monomer K_{off} rate was in the range of 10⁻M compared with dimer Koff rates which were in the range of 10⁻³-10⁻⁴M. 16 clones that appeared to have very slow off rates were selected, these came from the 3U, 5U and 7U libraries and were sequenced. In addition the supernatants were analysed for the ability to neutralise human TNFα in the receptor assay.

6 lead clones (d1-d6 below) that neutralised in these assays and have been sequenced. The results shows that out of the 6 clones obtained there are only 3 different second dAbs (dAb1, dAb2 and dAb3) however where the second dAb is found more than once they are linked with different length linkers.

TAR1-5d1: 3U linker 2^{nd} dAb=dAb1-1 μg/ml Ag immunotube overnight wash

TAR1-5d2: 3U linker 2^{nd} dAb=dAb2-1 μg/ml Ag immunotube overnight wash

TAR1-5d3: 5U linker 2^{nd} dAb=dAb2-1 μg/ml Ag immunotube overnight wash

TAR1-5d4: 5U linker 2^{nd} dAb=dAb3-20 μg/ml Ag immunotube overnight wash

TAR1-5d5: 5U linker 2^{nd} dAb=dAb1-20 μg/ml Ag immunotube overnight wash

TAR1-5d6: 7U linker 2^{nd} dAb=dAb1-R1:1 μg/ml Ag immunotube overnight wash, R2:beads

The 6 lead clones were examined further. Protein was produced from the periplasm and supernatant, purified with protein L agarose and examined in the cell and receptor assays. The levels of neutralisation were variable (Table 4). The optimal conditions for protein preparation were determined. Protein produced from HB2151 cells as supernatants gave the highest yield (approximately 10 mgs/L of culture). The supernatants were incubated with protein L agarose for 2 hrs at room temperature or overnight at 4° C. The beads were washed with PBS/NaCl and packed onto an FPLC column using a peristaltic pump. The beads were washed with 10 column volumes of PBS/NaCl and eluted with 0.1M glycine pH2.5. In general, dimeric protein is eluted after the monomer.

TAR1-5d1-6 dimers were purified by FPLC. Three species were obtained, by FPLC purification and were identified by SDS PAGE. One species corresponds to monomer and the other two species corresponds to dimers of different sizes. The larger of the two species is possibly due to the presence of C terminal tags. These proteins were examined in the receptor assay. The data presented in Table 4 represents the optimum results obtained from the two dimeric species (FIG. 11).

The three second dAbs from the dimer pairs (i.e., dAb1, dAb2 and dAb3) were cloned as monomers and examined by ELISA and in the cell and receptor assay. All three dAbs bind specifically to TNF by antigen ELISA and do not cross react with plastic or BSA. As monomers, none of the dAbs neutralise in the cell or receptor assays.

2.1.2 TAR1-5-19 Dimers

TAR1-5-19 was substituted for TAR1-5 in the 6 lead clones. Analysis of all TAR1-5-19 dimers in the cell and receptor assays was conducted using total protein (protein L purified only) unless otherwise stated (Table 5). TAR1-5-19d4 and TAR1-5-19d3 have the best ND₅₀ (˜5 nM) in the cell assay, this is consistent with the receptor assay results and is an improvement over TAR1-5-19 monomer (ND₅₀˜30 nM). Although purified TAR1-5 dimers give variable results in the receptor and cell assays TAR1-5-19 dimers were more consistent. Variability was shown when using different elution buffers during the protein purification. Elution using 0.1 M Phosphate-citrate buffer pH2.6 or 0.2M Glycine pH2.5 although removing all protein from the protein L agarose in most cases rendered it less functional.

TAR1-5-19d4 was expressed in the fermenter and purified on cation exchange FPLC to yield a completely pure dimer. As with TAR1-5d4 three species were obtained, by FPLC purification corresponding to monomer and two dimer species. This dimer was amino acid sequenced. TAR1-5-19 monomer and TAR1-5-19d4 were then examined in the receptor assay and the resulting IC50 for monomer was 30 nM and for dimer was 8 nM. the results of the receptor assay comparing TAR1-5-19 monomer, TAR1-519d4 and TAR1-5d4 is shown in FIG. 10.

TAR1-5-19 homodimers were made in the 3U, 5U and 7U vectors, expressed and purified on Protein L. The proteins were examined in the cell and receptor assays and the resulting IC₅₀s (for receptor assay) and ND₅₀s (for cell assay) were determined. (Table 6, FIG. 12)

2.2 Fabs

TAR1-5 and TAR1-5-19 dimers were also cloned into Fab format, expressed and purified on protein L agarose. Fabs were assessed in the receptor assays (Table 7). The results showed that for both TAR1-5-19 and TAR1-5 dimers the neutralisation levels were similar to the original Gly₄Ser linker dimers from which they were derived. A TAR1-5-19 Fab where TAR1-5-19 was displayed on both CH and CK was expressed, protein L purified and assessed in the receptor assay. The resulting IC50 was approximately 1 nM.

2.3 TAR1-27 Dimers

3×96 clones were picked from the round 2 selection encompassing all the libraries and selection conditions. 2 ml supernatant preps were made for analysis in ELISA and bioassays. Antigen ELISA gave 71 positive clones. The receptor assay of crude supernatants yielded 42 clones with inhibitory properties (TNF binding 0-60%). In the majority of cases inhibitory properties correlated with a strong ELISA signal. 42 clones were sequenced, 39 of these have unique second dAb sequences. The 12 dimers that gave the best inhibitory properties were analysed further.

The 12 neutralising clones were expressed as 200 ml supernatant preps and purified on protein L. These were assessed by protein L and antigen ELISA, BIAcore and in the receptor assay. Strong positive ELISA signals were obtained in all cases. BIAcore analysis revealed all clones to have fast on and off rates. The off rates were improved compared to monomeric TAR1-27, however the off rate of TAR1-27 dimers was faster (Koff is approximately in the range of 10⁻¹ and 10⁻²M) than the TAR1-5 dimers examined previously (Koff is approximately in the range of 10⁻³-10⁻⁴M). The stability of the purified dimers was questioned and therefore in order to improve stability, the addition on 5% glycerol, 0.5% Triton X100 or 0.5% NP40 (Sigma) was included in the purification of 2 TAR1-27 dimers (d2 and d16). Addition of NP40 or Triton X100™ improved the yield of purified product approximately 2 fold. Both dimers were assessed in the receptor assay. TAR1-27d2 gave IC50 of ˜30 nM under all purification conditions. TAR 1-27d16 showed no neutralisation effect when purified without the use of stabilising agents but gave an IC50 of ˜50 nM when purified under stabilising conditions. No further analysis was conducted.

2.4 TAR2-5 Dimers

3×96 clones were picked from the second round selections encompassing all the libraries and selection conditions. 2 ml supernatant preps were made for analysis. Protein A and antigen ELISAs were conducted for each plate. 30 interesting clones were identified as having good off-rates by BIAcore (Koff ranges between 10⁻²-10⁻³M). The clones were sequenced and 13 unique dimers were identified by sequence analysis.

TABLE 4
TAR1-5 dimers.

| | | | Protein | | Receptor/ |
|---|---|---|---|---|---|
| Dimer | Cell type | Purification | Fraction | Elution conditions | Cell assay |
| TAR1-5d1 | HB2151 | Protein L + | small dimeric | 0.1M glycine | RA ~30 nM |
| | | FPLC | species | pH 2.5 | |
| TAR1-5d2 | HB2151 | Protein L + | small dimeric | 0.1M glycine | RA ~50 nM |
| | | FPLC | species | pH 2.5 | |
| TAR1-5d3 | HB2151 | Protein L + | large dimeric | 0.1M glycine | RA ~300 nM |
| | | FPLC | species | pH 2.5 | |
| TAR1-5d4 | HB2151 | Protein L + | small dimeric | 0.1M glycine | RA ~3 nM |
| | | FPLC | species | pH 2.5 | |
| TAR1-5d5 | HB2151 | Protein L + | large dimeric | 0.1M glycine | RA ~200 nM |
| | | FPLC | species | pH 2.5 | |
| TAR1-5d6 | HB2151 | Protein L + | Large dimeric | 0.1M glycine | RA ~100 nM |
| | | FPLC | species | pH 2.5 | |

*note dimer 2 and dimer 3 have the same second dAb (called dAb2), however have different linker lengths (d2 = (Gly₄Ser)₃, d3 = (Gly₄Ser)₃). dAb1 is the partner dAb to dimers 1, 5 and 6. dAb3 is the partner dAb to dimer4. None of the partner dAbs neutralise alone. FPLC purification is by cation exchange unless otherwise stated. The optimal dimeric species for each dimer obtained by FPLC was determined in these assays.

TABLE 5
TAR1-5-19 dimers

| | | | | Elution | Receptor/ |
|---|---|---|---|---|---|
| Dimer | Cell type | Purification | Protein Fraction | conditions | Cell assay |
| TAR1-5-19 d1 | TOP10F′ | Protein L | Total | 0.1M glycine | RA ~15 nM |
| | | | protein | pH 2.0 | |
| TAR1-5-19 d2 | TOP10F′ | Protein L | Total | 0.1M glycine | RA ~2 nM |
| (no stop codon) | | | protein | pH 2.0 + | |
| | | | | 0.05% NP40 | |
| TAR1-5-19d3 | TOP10F′ | Protein L | Total | 0.1M glycine | RA ~8 nM |
| (no stop codon) | | | protein | pH 2.5 + | |
| | | | | 0.05% NP40 | |
| TAR1-5-19d4 | TOP10F′ | Protein L + | FPLC | 0.1M glycine | RA ~2-5 nM |
| | | FPLC | purified | pH 2.0 | CA ~12 nM |
| | | | fraction | | |
| TAR1-5-19d5 | TOP10F′ | Protein L | Total | 0.1M glycine | RA ~8 nM |
| | | | protein | pH 2.0 + NP40 | CA ~10 nM |
| TAR1-5-19 d6 | TOP10F′ | Protein L | Total | 0.1M glycine | RA ~10 nM |
| | | | protein | pH 2.0 | |

TABLE 6
TAR1-5-19 homodimers

| | | | Protein | Elution | Receptor/Cell |
|---|---|---|---|---|---|
| Dimer | Cell type | Purification | Fraction | conditions | assay |
| TAR1-5-19 3U | HB2151 | Protein L | Total | 0.1M glycine | RA ~20 nM |
| homodimer | | | protein | pH 2.5 | CA ~30 nM |
| TAR1-5-19 5U | HB2151 | Protein L | Total | 0.1M glycine | RA ~2 nM |
| homodimer | | | protein | pH 2.5 | CA ~3 nM |
| TAR1-5-19 7U | HB2151 | Protein L | Total | 0.1M glycine | RA ~10 nM |
| homodimer | | | protein | pH 2.5 | CA ~15 nM |
| TAR1-5-19 cys | HB2151 | Protein L + FPLC | FPLC | 0.1M glycine | RA ~2 nM |
| hinge | | | purified | pH 2.5 | |
| | | | dimer | | |
| | | | fraction | | |
| TAR1-5-19CH/ | HB2151 | Protein | Total | 0.1M glycine | RA ~1 nM |
| TAR1-5-19 CK | | | protein | pH 2.5 | |

TABLE 7
TAR1-5/TAR1-5-19 Fabs

| | | | Protein | Elution | |
|---|---|---|---|---|---|
| Dimer | Cell type | Purification | Fraction | conditions | Receptor/Cell assay |
| TAR1-5CH/ | HB2151 | Protein L | Total | 0.1M citrate | RA ~90 nM |
| dAb1 CK | | | protein | pH 2.6 | |
| TAR1-5CH/ | HB2151 | Protein L | Total | 0.1M glycine | RA ~30 nM |
| dAb2 CK | | | protein | pH 2.5 | CA ~60 nM |
| dAb3CH/ | HB2151 | Protein L | Total | 0.1M citrate | RA ~100 nM |
| TAR1-5CK | | | protein | pH 2.6 | |
| TAR1-5-19CH/ | HB2151 | Protein L | Total | 0.1M glycine | RA ~6 nM |
| dAb1 CK | | | protein | pH 2.0 | |
| dAb1 CH/ | HB2151 | Protein L | 0.1M | Myc/flag | RA ~6 nM |
| TAR1-5-19CK | | | glycine | | |
| | | | pH 2.0 | | |
| TAR1-5-19CH/ | HB2151 | Protein L | Total | 0.1M glycine | RA ~8 nM |
| dAb2 CK | | | protein | pH 2.0 | CA ~12 nM |
| TAR1-5-19CH/ | HB2151 | Protein L | Total | 0.1M glycine | RA ~3 nM |
| dAb3CK | | | protein | pH 2.0 | |

### Example 7

### dAb Dimerisation by Terminal Cysteine Linkage

Summary

For dAb dimerisation, a free cysteine has been engineered at the C-terminus of the protein. When expressed the protein forms a dimer which can be purified by a two step purification method.

PCR construction of TAR1-5-19CYS Dimer

See example 8 describing the dAb trimer. The trimer protocol gives rise to a mixture of monomer, dimer and trimer.

Expression and Purification of TAR1-5-19CYS Dimer

The dimer was purified from the supernatant of the culture by capture on Protein L agarose as outlined in the example 8.

Separation of TAR1-5-19CYS Monomer from the TAR1-5-19CYS Dimer

Prior to cation exchange separation, the mixed monomer/dimer sample was buffer exchanged into 50 mM sodium acetate buffer pH 4.0 using a PD-10 column (Amersham Pharmacia), following the manufacturer's guidelines. The sample was then applied to a 1 mL Resource S cation exchange column (Amersham Pharmacia), which had been pre-equilibrated with 50 mM sodium acetate pH 4.0. The monomer and dimer were separated using the following salt gradient in 50 mM sodium acetate pH 4.0:

150 to 200 mM sodium chloride over 15 column volumes

200 to 450 mM sodium chloride over 10 column volumes

450 to 1000 mM sodium chloride over 15 column volumes

Fractions containing dimer only were identified using SDS-PAGE and then pooled and the pH increased to 8 by the addition of ⅕ volume of 1M Tris pH 8.0.

In vitro functional binding assay: TNF receptor assay and cell assay

The affinity of the dimer for human TNFα was determined using the TNF receptor and cell assay. IC50 in the receptor assay was approximately 0.3-0.8 nM; ND50 in the cell assay was approximately 3-8 nM.

Other possible TAR1-5-19CYS dimer formats

PEG Dimers and Custom Synthetic Maleimide Dimers

Nektar (Shearwater) offer a range of bi-maleimide PEGs [mPEG2-(MAL)2 or mPEG-(MAL)2] which would allow the monomer to be formatted as a dimer, with a small linker separating the dAbs and both being linked to a PEG ranging in size from 5 to 40 kDa. It has been shown that the 5 kDa mPEG-(MAL)₂ (i.e., [TAR1-5-19]-Cys-maleimide-PEG×2, wherein the maleimides are linked together in the dimer) has an affinity in the TNF receptor assay of ˜1-3 nM. Also the dimer can also be produced using TMEA (Tris[2-maleimidoethyl]amine) (Pierce Biotechnology) or other bi-functional linkers.

It is also possible to produce the disulphide dimer using a chemical coupling procedure using 2,2′-dithiodipyridine (Sigma Aldrich) and the reduced monomer.

Addition of a polypeptide linker or hinge to the C-terminus of the dAb.

A small linker, either (Gly₄Ser)ₙ where n=1 to 10, eg, 1, 2, 3, 4, 5, 6 or 7, an immunoglobulin (eg, IgG hinge region or random peptide sequence (eg, selected from a library of random peptide sequences) can be engineered between the dAb and the terminal cysteine residue. This can then be used to make dimers as outlined above.

### Example 8

### dAb Trimerisation

Summary

For dAb trimerisation, a free cysteine is required at the C-terminus of the protein. The cysteine residue, once reduced to give the free thiol, can then be used to specifically couple the protein to a trimeric maleimide molecule, for example TMEA (Tris[2-maleimidoethyl]amine).

PCR Construction of TAR1-5-19CYS

The following oligonucleotides were used to specifically PCR TAR1-5-19 with a SalI and BamHI sites for cloning and also to introduce a C-terminal cysteine residue:

| | |
|---|---|
| | Sal I |
| | ~~~~~~~~ |
| | Trp Ser Ala Ser Thr Asp* Ile Gln Met Thr Gln Ser Pro Ser Ser Leu Ser Ala Ser Val |
| 1 | TGG AGC GCG TCG ACG GAC ATC CAG ATG ACC CAG TCT CCA TCC TCT CTG TCT GCA TCT GTA |
| | ACC TCG CGC AGC TGC CTG TAG GTC TAC TGG GTC AGA GGT AGG AGA GAC AGA CGT AGA CAT |
| | Gly Asp Arg Val Thr Ile Thr Cys Arg Ala Ser Gln Ser Ile Asp Ser Tyr Leu His Trp |
| 61 | GGA GAC CGT GTC ACC ATC ACT TGC CGG GCA AGT CAG AGC ATT GAT AGT TAT TTA CAT TGG |
| | CCT CTG GCA CAG TGG TAG TGA ACG GCC CGT TCA GTC TCG TAA CTA TCA ATA AAT GTA ACC |
| | Tyr Gln Gln Lys Pro Gly Lys Ala Pro Lys Leu Leu Ile Tyr Ser Ala Ser Glu Leu Gln |
| 121 | TAC CAG CAG AAA CCA GGG AAA GCC CCT AAG CTC CTG ATC TAT AGT GCA TCC GAG TTG CAA |
| | ATG GTC GTC TTT GGT CCC TTT CGG GGA TTC GAG GAC TAG ATA TCA CGT AGG CTC AAC GTT |
| | Ser Gly Val Pro Ser Arg Phe Ser Gly Ser Gly Ser Gly Thr Asp Phe Thr Leu Thr Ile |
| 181 | AGT GGG GTC CCA TCA CGT TTC AGT GGC AGT GGA TCT GGG ACA GAT TTC ACT CTC ACC ATC |
| | TCA CCC CAG GGT AGT GCA AAG TCA CCG TCA CCT AGA CCC TGT CTA AAG TGA GAG TGG TAG |
| | Ser Ser Leu Gln Pro Glu Asp Phe Ala Thr Tyr Tyr Cys Gln Gln Val Val Trp Arg Pro |
| 241 | AGC AGT CTG CAA CCT GAA GAT TTT GCT ACG TAC TAC TGT CAA CAG GTT GTG TGG CGT CCT |
| | TCG TCA GAC GTT GGA CTT CTA AAA CGA TGC ATG ATG ACA GTT GTC CAA CAC ACC GCA GGA |
| | BamHI |
| | ~~~~~~~~ |
| | Phe Thr Phe Gly Gln Gly Thr Lys Val Glu Ile Lys Arg Cys *** *** Gly Ser Gly |
| 301 | TTT ACG TTC GGC CAA GGG ACC AAG GTG GAA ATC AAA CGG TGC TAA TAA GGA TCC GGC |
| | AAA TGC AAG CCG GTT CCC TGG TTC CAC CTT TAG TTT GCC ACG ATT ATT CCT AGG CCG |

(*start of TAR1-5-19CYS sequence; TAR1-5-19CYS amino acid sequence (SEQ ID NO:468);
TAR1-5-19CYS nucleotide sequences (SEQ ID NO:469, coding strand; SEQ ID NO:470,
noncoding strand))

Forward primer

(SEQ ID NO:471)

5′-TGGAGCGCGTCGACGGACATCCAGATGACCCAGTCTCCA-3′
Reverse primer

(SEQ ID NO:472)

5′-TTAGCAGCCGGATCCTTATTAGCACCGTTTGATTTCCAC-3′

The PCR reaction (50 μL volume) was set up as follows: 200 μM dNTPs, 0.4 μM of each primer, 5 μL of 10× PfuTurbo buffer (Stratagene), 100 ng of template plasmid (encoding TAR1-5-19), 1 μL of PfuTurbo enzyme (Stratagene) and the volume adjusted to 50 μL using sterile water. The following PCR conditions were used: initial denaturing step 94° C. for 2 mins, then 25 cycles of 94° C. for 30 secs, 64° C. for 30 sec and 72° C. for 30 sec. A final extension step was also included of 72° C. for 5 mins. The PCR product was purified and digested with SalI and BamHI and ligated into the vector which had also been cut with the same restriction enzymes. Correct clones were verified by DNA sequencing.

Expression and Purification of TAR 1-5-19CYS

TAR1-5-19CYS vector was transformed into BL21 (DE3) pLysS chemically competent cells (Novagen) following the manufacturer's protocol. Cells carrying the dAb plasmid were selected for using 100 μg/mL carbenicillin and 37 μg/mL chloramphenicol. Cultures were set up in 2L baffled flasks containing 500 mL of terrific broth (Sigma-Aldrich), 100 μg/mL carbenicillin and 37 μg/mL chloramphenicol. The cultures were grown at 30° C. at 200 rpm to an O.D.600 of 1-1.5 and then induced with 1 mM IPTG (isopropyl-beta-D-thiogalactopyranoside, from Melford Laboratories). The expression of the dAb was allowed to continue for 12-16 hrs at 30° C. It was found that most of the dAb was present in the culture media. Therefore, the cells were separated from the media by centrifugation (8,000×g for 30 mins), and the supernatant used to purify the dAb. Per litre of supernatant, 30 mL of Protein L agarose (Affitech) was added and the dAb allowed to batch bind with stirring for 2 hours. The resin was then allowed to settle under gravity for a further hour before the supernatant was siphoned off. The agarose was then packed into a XK 50 column (Amersham Phamacia) and was washed with 10 column volumes of PBS. The bound dAb was eluted with 100 mM glycine pH 2.0 and protein containing fractions were then neutralized by the addition of ⅕ volume of 1 M Tris pH 8.0. Per litre of culture supernatant 20 mg of pure protein was isolated, which contained a 50:50 ratio of monomer to dimer.

Trimerisation of TAR1-5-19CYS

2.5 ml of 100 pM TAR1-5-19CYS was reduce with 5 mM dithiothreitol and left at room temperature for 20 minutes. The sample was then buffer exchanged using a PD-10 column (Amersham Pharmacia). The column had been pre-equilibrated with 5 mM EDTA, 50 mM sodium phosphate pH 6.5, and the sample applied and eluted following the manufactures guidelines. The sample was placed on ice until required. TMEA (Tris[2-maleimidoethyl]amine) was purchased from Pierce Biotechnology. A 20 mM stock solution of TMEA was made in 100% DMSO (dimethyl sulphoxide). It was found that a concentration of TMEA greater than 3:1 (molar ratio of dAb:TMEA) caused the rapid precipitation and cross-linking of the protein. Also the rate of precipitation and cross-linking was greater as the pH increased. Therefore using 100 μM reduced TAR1-5-19CYS, 25 μM TMEA was added to trimerise the protein and the reaction allowed to proceed at room temperature for two hours. It was found that the addition of additives such as glycerol or ethylene glycol to 20% (v/v), significantly reduced the precipitation of the trimer as the coupling reaction proceeded. After coupling, SDS-PAGE analysis showed the presence of monomer, dimer and trimer in solution.

Purification of the Trimeric TAR1-5-19CYS

40 μL of 40% glacial acetic acid was added per mL of the TMEA-TAR1-5-19cys reaction to reduce the pH to ˜4. The sample was then applied to a 1 mL Resource S cation exchange column (Amersham Pharmacia), which had been pre-equilibrated with 50 mM sodium acetate pH 4.0. The dimer and trimer were partially separated using a salt gradient of 340 to 450 mM Sodium chloride, 50 mM sodium acetate pH 4.0 over 30 column volumes. Fractions containing trimer only were identified using SDS-PAGE and then pooled and the pH increased to 8 by the addition of ⅕ volume of 1M Tris pH 8.0. To prevent precipitation of the trimer during concentration steps (using 5K cut off Viva spin concentrators; Vivascience), 10% glycerol was added to the sample.

In vitro functional binding assay: TNF receptor assay and cell assay

The affinity of the trimer for human TNFα was determined using the TNF receptor and cell assay. IC50 in the receptor assay was 0.3 nM; ND50 in the cell assay was in the range of 3 to 10 nM (eg, 3 nM).

Other possible TAR1-5-19CYS trimer formats

TAR1-5-19CYS may also be formatted into a trimer using the following reagents:

PEG Trimers and Custom Synthetic Maleimide Trimers

Nektar (Shearwater) offer a range of multi arm PEGs, which can be chemically modified at the terminal end of the PEG. Therefore using a PEG trimer with a maleimide functional group at the end of each arm would allow the trimerisation of the dAb in a manner similar to that outlined above using TMEA. The PEG may also have the advantage in increasing the solubility of the trimer thus preventing the problem of aggregation. Thus, one could produce a dAb trimer in which each dAb has a C-terminal cysteine that is linked to a maleimide functional group, the maleimide functional groups being linked to a PEG trimer.

Addition of a polypeptide linker or hinge to the C-terminus of the dAb

A small linker, either (Gly₄Ser)ₙ where n=1 to 10, eg, 1, 2, 3, 4, 5, 6 or 7, an immunoglobulin (eg, IgG hinge region or random peptide sequence (eg, selected from a library of random peptide sequences) could be engineered between the dAb and the terminal cysteine residue. When used to make multimers (eg, dimers or trimers), this again would introduce a greater degree of flexibility and distance between the individual monomers, which may improve the binding characteristics to the target, eg a multisubunit target such as human TNFα.

### Example 9

### Selection of a Collection of Single Domain Antibodies (dAbs) Directed Against Human Serum Albumin (HSA) and Mouse Serum Albumin (MSA)

This example explains a method for making a single domain antibody (dAb) directed against serum albumin. Selection of dAbs against both mouse serum albumin (MSA) and human serum albumin (HSA) is described. Three human phage display antibody libraries were used in this experiment, each based on a single human framework for V_{H} (see FIG. 13: sequence of dummy V_{H} based on V3-23/DP47 and JH4b) or V_{κ} (see FIG. 15: sequence of dummy V_{κ} based on o12%2/DPK9 and Jk1) with side chain diversity encoded by NNK codons incorporated in complementarity determining regions (CDR1, CDR2 and CDR3).

Library 1 (V_{H}): Diversity at positions: H30, H31, H33, H35, H50, H52, H52a, H53, H55, H56, H58, H95, H97, H98. Library size:6.2×10⁹

Library 2 (V_{H}): Diversity at positions: H30, H31, H33, H35, H50, H52, H52a, H53, H55, H56, H58, H95, H97, H98, H99, H100, H100a, H100b. Library size: 4.3×10⁹

Library 3 (V_{κ}): Diversity at positions: L30, L31, L32, L34, L50, L53, L91, L92, L93, L94, L96 Library size:2×10⁹

The V_{H} and V_{κ} libraries have been preselected for binding to generic ligands protein A and protein L respectively so that the majority of clones in the unselected libraries are functional. The sizes of the libraries shown above correspond to the sizes after preselection.

Two rounds of selection were performed on serum albumin using each of the libraries separately. For each selection, antigen was coated on immunotube (nunc) in 4 ml of PBS at a concentration of 100 μg/ml. In the first round of selection, each of the three libraries was panned separately against HSA (Sigma) and MSA (Sigma). In the second round of selection, phage from each of the six first round selections was panned against (i) the same antigen again (eg 1^{st} round MSA, 2^{nd} round MSA) and (ii) against the reciprocal antigen (eg 1^{st} round MSA, 2^{nd} round HSA) resulting in a total of twelve 2^{nd} round selections. In each case, after the second round of selection 48 clones were tested for binding to HSA and MSA. Soluble dAb fragments were produced as described for scFv fragments by Harrison et al, Methods Enzymol. 1996; 267:83-109 and standard ELISA protocol was followed (Hoogenboom et al. (1991) Nucleic Acids Res., 19: 4133) except that 2% tween PBS was used as a blocking buffer and bound dAbs were detected with either protein L-HRP (Sigma) (for the V_{κS}) and protein A—HRP (Amersham Pharmacia Biotech) (for the V_{H}_{^{S}}).

dAbs that gave a signal above background indicating binding to MSA, HSA or both were tested in ELISA insoluble form for binding to plastic alone but all were specific for serum albumin. Clones were then sequenced (Table 8) revealing that 21 unique dAb sequences had been identified. The minimum similarity (at the amino acid level) between the V_{κ} dAb clones selected was 86.25% ((69/80)×100; the result when all the diversified residues are different, eg clones 24 and 34). The minimum similarity between the V_{H} dAb clones selected was 94% ((127/136)×100).

Next, the serum albumin binding dAbs were tested for their ability to capture biotinylated antigen from solution. ELISA protocol (as above) was followed except that ELISA plate was coated with 1 μg/ml protein L (for the V_{κ} clones) and 1 μg/ml protein A (for the V_{H} clones). Soluble dAb was captured from solution as in the protocol and detection was with biotinylated MSA or HSA and streptavidin HRP. The biotinylated MSA and HSA had been prepared according to the manufacturer's instructions, with the aim of achieving an average of 2 biotins per serum albumin molecule. Twenty four clones were identified that captured biotinylated MSA from solution in the ELISA. Two of these (clones 2 and 38 below) also captured biotinylated HSA. Next, the dAbs were tested for their ability to bind MSA coated on a CM5 biacore chip. Eight clones were found that bound MSA on the biacore.

TABLE 8

| dAb (all | | | | | Binds | |
|---|---|---|---|---|---|---|
| capture | | | | | MSA | Captures |
| biotinylated | H | | | | in | biotinylated |
| MSA) | or κ | CDR1 | CDR2 | CDR3 | biacore? | HSA? |
| Vκ library 3 | κ | XXXLX | XASXLQS | QQXXXXPXT | | |
| template | | (SEQ ID NO: 473) | (SEQ ID | (SEQ ID NO: 475) | | |
| (dummy) | | | NO: 474) | | | |
| 2, 4, 7, 41, | κ | SSYLN | RASPLQS | QQTYSVPPT | | ✓aLL 4 bind |
| | | (SEQ ID NO: 476) | (SEQ ID | (SEQ ID NO: 478) | | |
| | | | NO: 477) | | | |
| 38,54 | κ | SSYLN | RASPLQS | QQTYRIPPT | | ✓ both bind |
| | | (SEQ ID NO: 476) | (SEQ ID | (SEQ ID NO: 479) | | |
| | | | NO: 477) | | | |
| 46, 47, 52, 56 | κ | FKSLK | NASYLQS | QQVVYWPVT | | |
| | | (SEQ ID NO: 480) | (SEQ ID | (SEQ ID NO: 482) | | |
| | | | NO: 481) | | | |
| 13,15 | κ | YYHLK | KASTLQS | QQVRKVPRT | | |
| | | (SEQ ID NO: 483) | (SEQ ID | (SEQ ID NO: 485) | | |
| | | | NO: 484) | | | |
| 30, 35 | κ | RRYLK | QASVLQS | QQGLYPPIT | | |
| | | (SEQ ID NO: 486) | (SEQ ID | (SEQ ID NO: 488) | | |
| | | | NO: 487) | | | |
| 19, | κ | YNWLK | RASSLQS | QQNVVIPRT | | |
| | | (SEQ ID NO: 489) | (SEQ ID | (SEQ ID NO: 491) | | |
| | | | NO: 490) | | | |
| 22, | κ | LWHLR | HASLLQS | QQSAVYPKT | | |
| | | (SEQ ID NO: 492) | (SEQ ID | (SEQ ID NO: 494) | | |
| | | | NO: 493) | | | |
| 23, | κ | FRYLA | HASHLQS | QQRLLYPKT | | |
| | | (SEQ ID NO: 495) | (SEQ ID | (SEQ ID NO: 497) | | |
| | | | NO: 496) | | | |
| 24, | κ | FYHLA | PASKLQS | QQRARWPRT | | |
| | | (SEQ ID NO: 498) | (SEQ ID | (SEQ ID NO: 500) | | |
| | | | NO: 499) | | | |
| 31, | κ | IWHLN | RASRLQS | QQVARVPRT | | |
| | | (SEQ ID NO: 501) | (SEQ ID | (SEQ ID NO: 503) | | |
| | | | NO: 502) | | | |
| 33, | κ | YRYLR | KASSLQS | QQYVGYPRT | | |
| | | (SEQ ID NO: 504) | (SEQ ID | (SEQ ID NO: 506) | | |
| | | | NO: 505) | | | |
| 34, | κ | LKYLK | NASHLQS | QQTTYYPIT | | |
| | | (SEQ ID NO: 507) | (SEQ ID | (SEQ ID NO: 509) | | |
| | | | NO: 508) | | | |
| 53, | κ | LRYLR | KASWLQS | QQVLYYPQT | | |
| | | (SEQ ID NO: 510) | (SEQ ID | (SEQ ID NO: 512) | | |
| | | | NO: 511) | | | |
| 11, | κ | LRSLK | AASRLQS | QQVVYWPAT | ✓ | |
| | | (SEQ ID NO: 513) | (SEQ ID | (SEQ ID NO: 515) | | |
| | | | NO: 514) | | | |
| 12, | κ | FRHLK | AASRLQS | QQVALYPKT | ✓ | |
| | | (SEQ ID NO: 516) | (SEQ ID | (SEQ ID NO: 517) | | |
| | | | NO: 514) | | | |
| 17, | κ | RKYLR | TASSLQS | QQNLFWPRT | ✓ | |
| | | (SEQ ID NO: 518) | (SEQ ID | (SEQ ID NO: 520) | | |
| | | | NO: 519) | | | |
| 18, | κ | RRYLN | AASSLQS | QQMLFYPKT | ✓ | |
| | | (SEQ ID NO: 521) | (SEQ ID | (SEQ ID NO: 523) | | |
| | | | NO: 522) | | | |
| 16, 21 | κ | IKHLK | GASRLQS | QQGARWPQT | ✓ | |
| | | (SEQ ID NO: 524) | (SEQ ID | (SEQ ID NO: 526) | | |
| | | | NO: 525) | | | |
| 25, 26 | κ | YYHLK | KASTLQS | QQVRKVPRT | ✓ | |
| | | (SEQ ID NO: 483) | (SEQ ID | (SEQ ID NO: 485) | | |
| | | | NO: 484) | | | |
| 27, | κ | YKHLK | NASHLQS | QQVGRYPKT | ✓ | |
| | | (SEQ ID NO: 527) | (SEQ ID | (SEQ ID NO: 528) | | |
| | | | NO: 508) | | | |
| 55, | κ | FKSLK | NASYLQS | QQVVYWPVT | ✓ | |
| | | (SEQ ID NO: 480) | (SEQ ID | (SEQ ID NO: 482) | | |
| | | | NO: 481) | | | |
| V_{H} library 1 | H | XXYXXX | XIXXXGXXTXYADS | XXXX(XXXX)FDY | | |
| (and 2) | | (SEQ ID | VKG (SEQ ID | (SEQ ID NO: 531) | | |
| template | | NO: 529) | NO: 530) | | | |
| (dummy) | | | | | | |
| 8,10 | H | WVYQMD | SISAFGAKTLYADS | LSGKFDY | | |
| | | (SEQ ID | VKG (SEQ ID | (SEQ ID NO: 534) | | |
| | | NO: 532) | NO: 533) | | | |
| 36, | H | WSYQMT | SISSFGSSTLYADS | GRDHNYSLFDY | | |
| | | (SEQ ID | VKG (SEQ ID | (SEQ ID NO: 537) | | |
| | | NO: 535) | NO: 536) | | | |

In all cases the frameworks were identical to the frameworks in the corresponding dummy sequence, with diversity in the CDRs as indicated in Table 8 above.

Of the eight clones that bound MSA on the biacore, two clones that are highly expressed in E. coli (clones MSA16 and MSA26) were chosen for further study (see example 10). Full nucleotide and amino acid sequences for MSA16 and 26 are given in FIG. 16.

### Example 10

### Determination of Affinity and Serum Half-Life in Mouse of MSA Binding dAbs MSA16 and MSA26

dAbs MSA16 and MSA26 were expressed in the periplasm of E. coli and purified using batch absorption to protein L-agarose affinity resin (Affitech, Norway) followed by elution with glycine at pH 2.2. The purified dAbs were then analysed by inhibition biacore to determine K_{d}. Briefly, purified MSA16 and MSA26 were tested to determine the concentration of dAb required to achieve 200RUs of response on a biacore CM5 chip coated with a high density of MSA. Once the required concentrations of dAb had been determined, MSA antigen at a range of concentrations around the expected K_{d} was premixed with the dAb and incubated overnight. Binding to the MSA coated biacore chip of dAb in each of the premixes was then measured at a high flow-rate of 30 μl/minute. The resulting curves were used to create Klotz plots, which gave an estimated K_{d} of 200 nM for MSA16 and 70 nM for MSA 26 (FIGS. 17 A & B).

Next, clones MSA 16 and MSA26 were cloned into an expression vector with the HA tag (nucleic acid sequence: TATCCTTATGATGTTCCTGATTATGCA (SEQ ID NO:538) and amino acid sequence: YPYDVPDYA (SEQ ID NO:539)) and 2-10 mg quantities were expressed in E. coli and purified from the supernatant with protein L-agarose affinity resin (Affitech, Norway) and eluted with glycine at pH2.2. Serum half life of the dAbs was determined in mouse. MSA26 and MSA16 were dosed as single i.v. injections at approx 1.5 mg/kg into CD1 mice. Analysis of serum levels was by goat anti-HA (Abcam, UK) capture and protein L-HRP (invitrogen) detection ELISA which was blocked with 4% Marvel. Washing was with 0.05% tween PBS. Standard curves of known concentrations of dAb were set up in the presence of 1× mouse serum to ensure comparability with the test samples. Modelling with a 2 compartment model showed MSA-26 had a t1/2α of 0.16 hr, a t1/2β of 14.5 hr and an area under the curve (AUC) of 465 hr.mg/ml (data not shown) and MSA-16 had a t1/2α of 0.98 hr, a t1/2β of 36.5 hr and an AUC of 913 hr.mg/ml (FIG. 18). Both anti-MSA clones had considerably lengthened half life compared with HEL4 (an anti-hen egg white lysozyme dAb) which had a t1/2α of 0.06 hr, and a t1/2β of 0.34 hr.

### Example 11

### Creation of V_{H}-V_{H} and Vκ-Vκ Dual Specific Fab Like Fragments

This example describes a method for making V_{H}-V_{H} and Vκ-Vκ dual specifics as Fab like fragments. Before constructing each of the Fab like fragments described, dAbs that bind to targets of choice were first selected from dAb libraries similar to those described in example 9. A V_{H} dAb, HEL4, that binds to hen egg lysozyme (Sigma) was isolated and a second V_{H} dAb (TAR2h-5) that binds to TNFα receptor (R and D systems) was also isolated. The sequences of these are given in the sequence listing. A V_{κ} dAb that binds TNFα (TAR1-5-19) was isolated by selection and affinity maturation and the sequence is also set forth in the sequence listing. A second V_{κ} dAb (MSA 26) described in example 9 whose sequence is in FIG. 16B was also used in these experiments.

DNA from expression vectors containing the four dAbs described above was digested with enzymes SalI and NotI to excise the DNA coding for the dAb. A band of the expected size (300-400 bp) was purified by running the digest on an agarose gel and excising the band, followed by gel purification using the Qiagen gel purification kit (Qiagen, UK). The DNA coding for the dAbs was then inserted into either the C_{H} or Cκ vectors (FIGS. 8 and 9) as indicated in Table 9.

TABLE 9

| | | dAb V_{H} | | | |
|---|---|---|---|---|---|
| | | or dAb | Inserted | tag (C | Antibiotic |
| dAb | Target antigen | Vκ | into vector | terminal) | resistance |
| HEL4 | Hen egg lysozyme | V_{H} | C_{H} | myc | Chloramphenicol |
| TAR2-5 | TNF receptor | V_{H} | Cκ | flag | Ampicillin |
| TAR1-5-19 | TNF α | Vκ | C_{H} | myc | Chloramphenicol |
| MSA 26 | Mouse serum | Vκ | Cκ | flag | Ampicillin |
| | albumin | | | | |

The V_{H}C_{H} and V_{H}Cκ constructs were cotransformed into HB2151 cells. Separately, the Vκ C_{H} and Vκ Cκ constructs were cotransformed into HB2151 cells. Cultures of each of the cotransformed cell lines were grown overnight (in 2xTy containing 5% glucose, 10 μg/ml chloramphenicol and 100 μg/ml ampicillin to maintain antibiotic selection for both C_{H} and Cκ plasmids). The overnight cultures were used to inoculate fresh media (2×Ty, 10 μg/ml chloramphenicol and 100 μg/ml ampicillin) and grown to OD 0.7-0.9 before induction by the addition of IPTG to express their CH and Cκ constructs. Expressed Fab like fragment was then purified from the periplasm by protein A purification (for the contransformed V_{H}C_{H} and V_{H}Cκ) and MSA affinity resin purification (for the contransformed VκC_{H} and VκCκ).

V_{H}-V_{H} Dual Specific

Expression of the V_{H}C_{H} and V_{H}Cκ dual specific was tested by running the protein on a gel. The gel was blotted and a band the expected size for the Fab fragment could be detected on the Western blot via both the myc tag and the flag tag, indicating that both the V_{H}C_{H} and V_{H}Cκ parts of the Fab like fragment were present. Next, in order to determine whether the two halves of the dual specific were present in the same Fab-like fragment, an ELISA plate was coated overnight at 4° C. with 100 μl per well of hen egg lysozyme (HEL) at 3 mg/ml in sodium bicarbonate buffer. The plate was then blocked (as described in example 1) with 2% tween PBS followed by incubation with the V_{H}C_{H}V_{H}Cκ dual specific Fab like fragment. Detection of binding of the dual specific to the HEL was via the non cognate chain using 9e10 (a monoclonal antibody that binds the myc tag, Roche) and anti mouse IgG-HRP (Amersham Pharmacia Biotech). The signal for the V_{H}C_{H} V_{H}Cκ dual specific Fab like fragment was 0.154 compared to a background signal of 0.069 for the V_{H}Cκ chain expressed alone. This demonstrates that the Fab like fragment has binding specificity for target antigen.

V_{κ}-V_{κ} Dual Specific

After purifying the contransformed VκC_{H} and VκCκ dual specific Fab like fragment on an MSA affinity resin, the resulting protein was used to probe an ELISA plate coated with 1 μg/ml TNFα and an ELISA plate coated with 10 μg/ml MSA. As predicted, there was signal above background when detected with protein L-HRP on both ELISA plates (data not shown). This indicated that the fraction of protein able to bind to MSA (and therefore purified on the MSA affinity column) was also able to bind TNFα in a subsequent ELISA, confirming the dual specificity of the antibody fragment. This fraction of protein was then used for two subsequent experiments. Firstly, an ELISA plate coated with 1 μg/ml TNFα was probed with dual specific VκC_{H} and VκCκ Fab like fragment and also with a control TNFα binding dAb at a concentration calculated to give a similar signal on the ELISA. Both the dual specific and control dAb were used to probe the ELISA plate in the presence and in the absence of 2 mg/ml MSA. The signal in the dual specific well was reduced by more than 50% but the signal in the dAb well was not reduced at all. The same protein was also put into the receptor assay with and without MSA and competition by MSA was also shown. This demonstrates that binding of MSA to the dual specific is competitive with binding to TNFα.

### Example 12

### Creation of a Vκ-Vκ Dual Specific cys Bonded Dual Specific with Specificity for Mouse Serum Albumin and TNFα

This example describes a method for making a dual specific antibody fragment specific for both mouse serum albumin and TNFα by chemical coupling via a disulphide bond. Both MSA16 (from example 1) and TAR1-5-19 dAbs were recloned into a pET based vector with a C terminal cysteine and no tags. The two dAbs were expressed at 4-10 mg levels and purified from the supernatant using protein L-agarose affinity resin (Affitiech, Norway). The cysteine tagged dAbs were then reduced with dithiothreitol. The TAR1-5-19 dAb was then coupled with dithiodipyridine to block reformation of disulphide bonds resulting in the formation of PEP 1-5-19 homodimers. The two different dAbs were then mixed at pH 6.5 to promote disulphide bond formation and the generation of TAR1-5-19, MSA16 cys bonded heterodimers. This method for producing conjugates of two unlike proteins was originally described by King et al. (King T P, Li Y Kochoumian L Biochemistry. 1978 vol 17:1499-506 Preparation of Protein Conjugates Via Intermolecular Disulfide Bond Formation.) Heterodimers were separated from monomeric species by cation exchange. Separation was confirmed by the presence of a band of the expected size on a SDS gel. The resulting heterodimeric species was tested in the TNF receptor assay and found to have an IC50 for neutralising TNF of approximately 18 nM. Next, the receptor assay was repeated with a constant concentration of heterodimer (18 nM) and a dilution series of MSA and HSA. The presence of HSA at a range of concentrations (up to 2 mg/ml) did not cause a reduction in the ability of the dimer to inhibit TNFα. However, the addition of MSA caused a dose dependant reduction in the ability of the dimer to inhibit TNFα. This demonstrates that MSA and TNFα compete for binding to the cys bonded TAR1-5-19, MSA16 dimer.

### Example 13

### Cloning and Expression of the TAR1/TAR2 Dual Specific Fab

TAR1-5-19 dAb (specific to human TNF alpha) was cloned into pDOM3 CK Amp vector (FIG. 21) as a SalI/NotI fragment. TAR2h-10-27 V_{H} dAb (specific to human TNFR1) was cloned into pDOM3 CH Chlor vector (FIG. 21) as a SallNotl fragment.

The two vectors with cloned in dAbs were used to co-transform competent HB2151 cells. Amp/Chlor resistant clones (containing both plasmids) were used to make a large scale (101) fermentor prep of the Fab.

The produced Fab was isolated from the culture supernatant (after 3 hours induction at 25C) using sequential Protein A/Protein L purification. The yield of the Fab was 1. 5 mg.

### Example 14

### Analysis of Fab properties in ELISA

a) Binding of the Fab to TAR1 and TAR2

Binding of the TAR1/TAR2 Fab to TNF and TNFR1 was tested in ELISA. A 96 well plate was coated with 100 ul of TNF and TNFR1 at 1 ug/ml concentration in PBS overnight at 4C. 50 ul (3 uM) of Fab was then added to the wells and bound Fab was detected via non-cognate chain, ie using Protein A-HRP on TNF coated wells and Protein L-HRP on TNFR1 coated wells. ELISA demonstrated the ability of the Fab to bind both antigens (FIG. 22).

b) Sandwich ELISA

To test the ability of the TAR1/TAR2 Fab to bind both antigens simultaneously a sandwich ELISA was performed. Here a 96 well plate was coated with mutant TNF (that does not bind to TNFR1, but does bind to PEP1-5-19, data no shown; mutant TNF contains a single point mutation (N141Y) which renders it incapable of binding to TNFR1 (Yamadishi et al., 1990, Protein Eng., 3, 713-9)) at 1 ug/ml concentration in PBS overnight at 4C. 50 ul of Fab (0.5 uM) was then added. This was followed by addition of TNFR 1-Fc fusion protein (R&D Systems) and detection with Anti-Fc-HRP. The same sandwich ELISA was performed using a control Fab containing TAR1/Ck chain and an irrelevant V_{H} fused to the CH chain. ELISA results demonstrated the ability of the Fab to engage both antigens (TNF and TNFR1) simultaneously, suggesting an open conformation of the molecule (FIG. 23).

c) Competition ELISA

To test the ability of the TAR1/TAR2 Fab to bind both antigens simultaneously two competition ELISAs were performed. A 96 well plate was coated with 100 μl of TNFR1 at 1 ug/ml concentration in PBS overnight at 4C. A dilution of Fab was chosen such that OD450 of 0.3 was achieved upon detection with Protein L-HRP. This concentration was 6 nM. The Fab was pre-incubated for an hour at room temperature with increasing concentrations of mutant TNF (up to 160× molar excess). As a negative control Fab was subjected to the same incubation with BSA. Following these incubations the mixtures were then put onto TNFR1 coated ELISA plate and incubated for another hour. Bound TAR1/TAR2 Fab was detected using ProteinL-HRP. ELISA demonstrated that TAR1/TAR2 Fab binding to TNFR1 was not affected by competing antigen (FIG. 24).

A 96 well plate was coated with 100 μl of mutant TNF at 1 ug/ml concentration in PBS overnight at 4C. A dilution of Fab was chosen such that OD450 of 0.3 was achieved upon detection with 9E10 (Sigma) followed by anti mo-HRP (Sigma). This concentration was 25 nM. The Fab was pre-incubated for an hour at room temperature with increasing concentrations of soluble TNFR1 (up to 10× molar excess). As a negative control Fab was subjected to the same incubation with BSA. Following these incubations the mixtures were then put onto mutant TNF coated ELISA plate and incubated for another hour.

Bound TAR1/TAR2 Fab was detected using 9E10 followed by anti mo-HRP. ELISA demonstrated that TAR1/TAR2 Fab binding to mutant TNF was not affected by competing antigen (FIG. 24).

### Example 15

### Analysis of Fab Properties in Cell Assays (FIG. 25)

To check the degree of functionality of each dAb in a TAR1/TAR2 Fab, the performance of the dual specific molecule was tested in the following cell assays:

Human TNF Cytotoxicity on Murine Cells.

This assay tests the activity of TAR1 Dab, as TAR2 Dab cannot bind to murine TNF receptor expressed on the surface of the cells. TAR1-5-19 Dab and TAR2h-10-27 dAbs as well as TAR1-5-19+TAR2h-10-27 dAb mixture were used as controls in this assay. The results demonstrate that TAR1-5-19 Dab in a Fab behaves as well as a monomeric TAR1-5-19 dAb.

Murine TNF Cytotoxicity Assay on Murine Cells with Human Soluble TNF Receptor.

This assay tests the activity of TAR2h-10-27 (in this assay binding to soluble human TNFR1). TAR1-5-19 and TAR2h-10-27 dAbs as well as TAR1-5-19+TAR2h-10-27 dAb mixture were used as controls in this assay. The results demonstrate that TAR2h-10-27 in a Fab behaves as well as a monomeric TAR2h-10-27 dAb.

Murine TNF Induced IL-8 Secretion on Human Cells.

This assay tests the activity of TAR2h-10-27 Dab (in this assay binding to membrane bound human TNFR1). TAR1-5-19 and TAR2h-10-27 dAbs as well as TAR1+TAR2 dAb mixture were used as controls in this assay. The results demonstrate that TAR2h-10-27 in a Fab behaves as well as a monomeric TAR2h-10-27 dAb.

Human TNF Induced IL-8 Secretion on Human Cells.

This assay tests the activity of both TAR1-5-19 and TAR2h-10-27 Dabs. TAR1-5-19 Dab and TAR2h-10-27 dAbs as well as TAR1-5-19+TAR2h-10-27 dAb mixture were used as controls in this assay. The results demonstrate that Fab has a similar effect to the TAR2h-10-27 dAb and TAR1-5-19+TAR2h-10-27 dAb mixture.

Murine TNF cytotoxicity on murine cells with soluble human TNFR1 and increasing concentrations of mutant TNF (competition on cells).

This assay was performed to test whether increasing concentration of mutant TNF (binding to TAR 1-5-19 Dab) will compromise binding of TAR2h-10-27 Dab to TNFR1 in solution. The results of the assay indicate that that is not the case, thus the Fab is able to engage two antigens simultaneously (FIG. 26).

The assays described above demonstrate that each dAb in a Fab molecule functions as well as a monomeric dAb.

### Example 16

### Construction of IgG Vectors

pcDNA3.1 (+) and pcDNA3.1/Zeo (+) backbones (Invitrogen) were used for cloning IgG1 heavy chain constant region and light chain kappa constant region, respectively. The overview of the vectors is shown in FIG. 27.

Leaders: Two alternative types of leaders were used to facilitate secretion of the expressed protein: CD33 leader IgG K-chain leader The leaders were assembled by the annealing of the two complementary oligos and were cloned into pcDNA3.1 (+) and pcDNA3.1/Zeo (+) as NheI/HindIII fragments (FIG. 27).

IgG1 heavy chain cloning: CH1 domain was PCR amplified from the CH vector (as described in WO 03/002609) using primers shown below.

Hinge region, C_{H}2 and CH3 domains were PCR amplified from pIgplus vector (Novagen) using primers shown below.

The two products were then PCR assembled to create an IgG1 heavy chain constant region which was cloned into pcDNA3.1 (+) as a NotI/XhoI fragment (FIG. 27).

Kappa Light Chain Cloning:

CK domain was PCR amplified from the CK vector (see WO 03/002609) using primers shown below. It was then cloned into pcDNA3.1/Zeo (+) as a NotI/XhoI fragment (FIG. 27).

SEQ
ID
NO:

| | |
|---|---|
| CkbckNot | |
| 5′ AAGGAAAAAAGCGGCCGCAACTGTGGCTGCACCATC 3′ | 540 |
| CkforXho | |
| 5′ CCGCTCGAGTCAACACTCTCCCCTGTTGAAGCTCTTTGTG 3′ | 541 |
| Ch1bckNot | |
| 5′ AAGGAAAAAAGCGGCCGCCTCCACCAAGGGCCCATCGGTC 3′ | 542 |
| Ch1for | |
| 5′ GTGAGGTTTGTCACAAGATTTGGGCTCAACTTTCTTGTCCAC | 543 |
| C 3′ | |
| Fcbck | |
| 5′ CCCAAATCTTGTGACAAACCTCAC 3′ | 544 |
| FcforXho | |
| 5′ CCGCTCGAGTCATTTACCCGGAGACAGGGAG 3′ | 545 |
| LEADER CD33: | |
| Leacd1 | |
| 5′PCTAGCCACCATGCCGCTGCTACTGGCTGCCACTGCTGTGGGCA | 546 |
| GGAGCACTGGCTATGGATA 3′ | |
| Leacd2 | |
| 5′PAGCTTATCCATAGCCAGTGCTCCTGCCCACAGCAGTGGCAGCA | 547 |
| GTAGCAGCAGCGGCATGGTGG 3′ | |
| LEADER IGGK: | |
| Leak1 | |
| 5′PCTAGCCACCATGGAGACAGACACACTCCTGCTATGGGTACTGC | 548 |
| TGCTCTGGGTTCCAGGTTCCACTGGTGACA3′ | |
| Leak2 | |
| 5′PAGCTTGTCACCAGTGGAACCTGGAACCCAGAGCAGCAGTACCC | 549 |
| ATAGCAGGAGTGTGTGCTGTCTCCATGGTGG 3′ | |
| SEQBACK | |
| 5′ TAATACGACTCACTATAGGG 3′ | 550 |
| SEQFOR | |
| 5′ TAGAAGGCACAGTCGAGG 3′ | 551 |

### Example 17

### Cloning of TAR1-5-19 and TAR2h-10-27 dAbs into IgG Vectors and Production of IgG

TAR1-5-19 V_{κ} dAb (specific to human TNF alpha) was cloned into IgG kappa vectors (with CD33 and IgK leaders) as a HindIII/NotI fragment (FIG. 27). TAR2h-10-27 V_{H} dAb (specific to human TNFR1) was cloned into IgG heavy chain vectors (with CD33 and IgK leaders) as a HindIII/NotI fragment (FIG. 27). Heavy and light chain plasmids were then co-transfected into COS7 cells and IgG was expressed transiently for five days. The produced IgG was purified using streamline Protein A. Expression level-250 ng/ml. CD33 and IgG K leaders gave the same level of expression. Purified IgG was checked on a reducing and non-reducing SDS gel (produced bands of expected size) (data not shown).

### Example 18

### Analysis of IgG Properties in ELISA

a) Binding of the IgG to TNF and TNFR1

Binding of the TAR1/TAR2 IgG to TNF and TNFR1 was tested in ELISA. A 96 well plate was coated with 100 ul of TNF and TNFR1 at 1 ug/ml concentration in PBS overnight at 4C. 50 ul (200 nM) of IgG was then added to the wells and bound IgG was detected via anti-Fc-HRP. ELISA demonstrated the ability of the IgG to bind both antigens (FIG. 28).

### Example 19

### Analysis of IgG Properties in Cell Assays

To check the degree of functionality of each dAb in a TAR1/TAR2 IgG, the performance of the dual specific molecule was tested in the following cell assays:

Human TNF Cytotoxicity on Murine Cells.

This assay tests the activity of TAR1-5-19 Dab, as TAR2h-10-27 Dab cannot bind to murine TNF receptor expressed on the surface of the cells. TAR1-5-19 and TAR2h-10-27 dabs as well as TAR1-5-19+TAR2h-10-27 dAb mixture were used as controls in this assay. The results demonstrate that TAR1-5-19 in the IgG behaves better than monomeric TAR1-5-19 dAb, which indicates that IgG is able to simultaneously engage two molecules of TNF (ND50 of the dimeric molecule) (FIG. 29).

Murine TNF cytotoxicity assay on murine cells with human soluble TNF receptor.

This assay tests the activity of TAR2h-10-27 (in this assay binding to soluble human TNFR1). TAR1-5-19 and TAR2h-10-27 dAbs as well as TAR1-5-19+TAR2h-10-27 dAb mixture were used as controls in this assay. The results demonstrate that TAR2h-10-27 in IgG behaves as well as a monomeric TAR2h-10-27 dAb (FIG. 29).

Murine TNF Induced IL-8 Secretion on Human Cells.

This assay tests the activity of TAR2h-10-27 (in this assay binding to membrane bound human TNFR1). TAR1-5-19 and TAR2h-10-27 dAbs as well as TAR1-5-19+TAR2h-10-27 dAb mixture were used as controls in this assay. The results demonstrate that IgG is able to engage two molecules of TNFR1 on the surface of the cell (agonistic activity) (FIG. 29). This assay was also repeated with no human TNF present. The results demonstrate that the IgG induces IL-8 release on human cells up to a concentration of 30 nM after which the agonistic activity goes down (FIG. 30).

Human TNF Induced IL-8 Secretion on Human Cells.

This assay tests the activity of both TAR1-5-19 and TAR2h-10-27. TAR1-5-19 and TAR2h-10-27 dAbs as well as TAR1-5-19+TAR2h-10-27 dAb mixture were used as controls in this assay. The results demonstrate that IgG has a similar effect to the TARh-10-27 dAb and TAR1-5-19+TAR2h-10-27 dAb mixture (FIG. 29). This assay was also repeated with no murine TNF present. The results demonstrate that the IgG induces IL-8 release on human cells up to a concentration of 30 nM after which the agonistic activity goes down (FIG. 30).

Data Summary

A summary of data obtained in the experiments set forth in the foregoing examples is set forth in the Annex.

### Example 20

### EGFR Binding

EGFR Binding Assay

25 ul of ligand (e.g., dAb) were plated into a 96 well plate and then 25 ul streptavidin-Alexa Fluor (1 ug/ml) (Molecular Probes) and 25 ul A431 cells (ATCC No, CRL-1555) (8×10⁵/ml) were added. All reagents were prepared in PBS/1% BSA. The plate was incubated for 30 minutes at room temperature.

Without disturbing the cells, 25 ul biotinylated EGF (Invitrogen) at 40 ng/ml was added to each well, and the plate was incubated for three hours at room temperature. Fluoresecence was measured using the AB8200 Cellular Detection System (Applied Biosystems).

Ligands (e.g., dAbs) that inhibited the binding of biotinylated EGF to EGFR expressed on A431 cells resulted in lower fluorescence counts. Wells without ligand provided a reference of the maximum fluorescence (i.e., biotinyulated EGF binding) and wells without ligand or biotinylated EGF provide a reference or the background level of fluorescence. These controls were included in all assays.

Results obtained in this assay using certain anti-EGFR dAbs are presented in Table 10.

EGFR Kinase Assay

In a 96 well plate, 5×10⁴ A431 cells (ATCC No, CRL-1555) were plated per well in RPMI-1640 supplemented with 10% foetal calf serum. The plate was incubated overnight at 37° C./5% CO₂ to allow the cells to adhere, then the medium was replaced with RPMI-1640. The plate was incubated for 4 hours at 37° C./5% CO₂. The ligand (prepared in RPMI-1640) was added to the wells and the plate was incubated for 45 minutes at 37° C./5% CO₂. EGF (Invitrogen) was added to the wells to give a final concentration of 100 ng/ml and the plate was incubated for 10 minutes at room temperature. The wells were washed twice with ice cold PBS. Cold lysis buffer (1% NP-40, 20 mM Tris, 137 mM NaCl, 10% glycerol, 2 mM EDTA, 1 mM sodium orthovanadate, 10 ug/ml aprotinin, 10 ug/ml leupeptin) was added and the plate was incubated on ice for 10 minutes.

The supernatants were transferred to an ELISA plate which had been coated overnight with anti-EGFR antibody (R&D Systems) at 1 ug/ml in carbonate buffer. The ELISA plate was incubated for 2 hours at room temperature. The plate was washed three times with PBS/0.05% tween 20. Anti-phosphotyrosine antibody conjugated to horse-radish peroxidase (Upstate Biotechnology) at 1 ug/ml was added and the plate was incubated for 1 hour at room temperature. The plate was washed three times with PBS/Tween and three times with PBS. The reaction was developed with SureBlue TMB 1-component microwell peroxidase substrate (KPL) and the reaction was stopped with 1M HCl after 25 minutes. The absorbance was read using a Wallac plate reader.

Results obtained in this assay using certain anti-EGFR dAbs are presented in the Table 10.

TABLE 10

| | | RECEPTOR | |
|---|---|---|---|
| | | BINDING ASSAY | KINASE ASSAY |
| DAB | KD (NM) | IC50 (NM)* | IC50 (NM)* |
| DOM16-39 | 27.3 | 28.68 TO 112.6 | 31.16 TO 100.9 |
| | | (56.84) | (56.07) |
| DOM16-200 | 15.3 | 12.47 TO 37.88 | 30.29 TO 111.9 |
| | | (21.74) | (58.21) |
| DOM16-39-87 | 6.81 | 4.471 TO 10.39 (6.8) | 11.95 TO 252.4 |
| | | | (54.92) |
| DOM16-39-100 | 1.24 | 1.007 TO 2.757 | 9.142 T0 17.56 |
| | | (1.67) | (12.67) |
| DOM16-39-107 | 7.09 | 1.472 TO 4.208 (2.49) | 12.00 TO 34.99 |
| | | | (20.49) |
| DOM16-39-109 | 1.01 | 0.746 TO 1.472 | 6.817 TO 11.08 |
| | | (1.05) | (8.69) |
| DOM16-39-115 | 6.90 | 1.085 TO 6.886 | 21.52 TO 83.34 |
| | | (2.73) | (42.35) |
| ERBITUX | — | 1.422 TO 5.388 | 3.875 TO 7.689 |
| (CETUXIMAB, | | (2.77) | (5.46) |
| IMCLONE | | | |
| SYSTEMS, INC.) | | | |

*the data presented are the lowest to highest values obtained and the (average)

### Example 21

Further studies confirmed that anti-TNFR1 dAbs do not agonize TNFR1 (act as TNFR1 agonists) in the absence of TNFα. L929 cells were cultured in media that contained a range of concentrations of either TAR2m-21-23 monomer, TAR2m-21-23 monomer cross-linked to a commercially available anti-myc antibody (9E10), TAR2m-21-23 3U TAR7m-16 or TAR2m-21-23 40K PEG. In the case of TAR2m-21-23 monomer cross-linked with the anti-myc antibody, the dAb and antibody were mixed in a 2:1 ratio and pre-incubated for one hour at room-temperature to simulate the effects of in vivo immune cross-linking prior to culture. TAR2m-21-23 monomer was incubated with the L929 cells at a concentration of 3000 nM. TAR2m-21-23 monomer and anti-Myc antibody were incubated at a dAb concentration of 3000 nM. TAR2m-21-23 3U TAR7m-16 was incubated with the cells at 25 nM, 83.3 nM, 250 nM, 833 nM and 2500 nM concentrations. TAR2m-21-23 40K PEG was incubated with the cells at 158.25 nM, 527.5 nM, 1582.5 nM, 5275 nM and 15825 nM concentrations. After incubation overnight, cell viability was assessed as described for the L929 cell cytotoxicity assay. The results revealed that incubation with various amounts of dAbs did not result in an increase in the number of non-viable cells in the cultures. The incubation of L929 cells with 10 nM, 1 nM and 0.1 nM of a commercially-available anti-TNFR1 IgG antibody resulted in a dose-dependent increase in non-viable cells thereby demonstrating the sensitivity of these cells to TNFR1-mediated agonism. (FIG. 32).

### Example 22

### IL-1R1 Cell Assay

Isolated dAbs were tested for their ability to inhibit IL-1-induced IL-8 release from cultured MRC-5 cells (ATCC catalogue no. CCL-171). Briefly, 5000 trypsinised MRC-5 cells in RPMI media were placed in the well of a tissue-culture microtitre plate and mixed with IL-1α or β (R&D Systems, 200 pg/ml final concentration) and a dilution of the dAb to be tested. The mixture was incubated overnight at 37° C. and IL-8 released by the cells into to culture media was quantified in an ELISA (DuoSet®, R&D Systems). Anti-IL-1R1 dAb activity caused a decrease in IL-1 binding and a corresponding reduction in IL-8 release.

### IL-1R1 Human Whole Blood Assay

Whole human blood was incubated with a dilution series of the dAb to be tested, and the mixture was incubated for 30 min at 37° C./5% CO₂. Next, 270 or 900 pM (final concentration) IL-1α or IL-1β was added and the mixture, and then the mixtures was incubated at 37° C./5% CO₂ for an additional 20 hours. The blood was then centrifuged (500×g, 5 min) and the IL-6 released into the supernatant was quantified in an ELISA (DuoSet®, R&D Systems). Anti-IL-1R1 dAb activity caused a decrease in IL-1 binding and a corresponding reduction in IL-6 release.

### Annex

Data Summary

| | | Equilibrium | | IC50 for | ND50 for cell |
|---|---|---|---|---|---|
| | | dissocation constant | | ligand | based neutralisn |
| TARGET | dAb | (Kd = Koff/Kon) | Koff | assay | assay |
| TAR1 | TAR1 | 300 nM to 5 pM | 5 × 10⁻¹ to | 500 nM | 500 nM to 50 pM |
| | monomers | (ie, 3 × 10⁻⁷ to | 1 × 10⁻⁷ | to | |
| | | 5 × 10⁻¹²), preferably | | 100 pM | |
| | | 50 nM to 20 pM | | | |
| | TAR1 | As TAR1 monomer | As TAR1 | As | As TAR1 |
| | dimers | | monomer | TAR1 | monomer |
| | | | | monomer | |
| | TAR1 | As TAR1 monomer | As TAR1 | As | As TAR1 |
| | trimers | | monomer | TAR1 | monomer |
| | | | | monomer | |
| | TAR1-5 | | | | |
| | TAR1-27 | | | | |
| | TAR1-5- | 30 nM | | | |
| | 19 | | | | |
| | monomer | | | | |
| | TAR1-5- | | | With | =30 nM |
| | 19 | | | (Gly₄Ser)₃ | =3 nM |
| | homodimer | | | linker = 20 nm | =15 nM |
| | | | | With | |
| | | | | (Gly₄Ser)₅ | |
| | | | | linker = 2 nm | |
| | | | | With | |
| | | | | (Gly₄Ser)₇ | |
| | | | | linker = 10 nm | |
| | | | | In Fab | |
| | | | | format = 1 nM | |
| | TAR1-5- | | | With | =12 nM |
| | 19 | | | (Gly₄Ser)ₙ | =10 nM |
| | heterodimers | | | linker | =12 nM |
| | | | | TAR1-5- | |
| | | | | 19 d2 = 2 nM | |
| | | | | TAR1-5- | |
| | | | | 19 d3 = 8 nM | |
| | | | | TAR1-5- | |
| | | | | 19 d4 = 2-5 nM | |
| | | | | TAR1-5- | |
| | | | | 19 d5 = 8 nM | |
| | | | | In Fab | |
| | | | | format | |
| | | | | TAR1-5- | |
| | | | | 19CH | |
| | | | | d1CK = 6 nM | |
| | | | | TAR1-5- | |
| | | | | 19CK | |
| | | | | d1CH = 6 nM | |
| | | | | TAR1-5- | |
| | | | | 19CH | |
| | | | | d2CK = 8 nM | |
| | | | | TAR1-5- | |
| | | | | 19CH | |
| | | | | d3CK = 3 nM | |
| | TAR1-5 | | | With | =60 nM |
| | heterodimers | | | (Gly₄Ser)ₙ | |
| | | | | linker | |
| | | | | TAR1- | |
| | | | | 5d1 = 30 nM | |
| | | | | TAR1- | |
| | | | | 5d2 = 50 nM | |
| | | | | TAR1- | |
| | | | | 5d3 = 300 nM | |
| | | | | TAR1- | |
| | | | | 5d4 = 3 nM | |
| | | | | TAR1- | |
| | | | | 5d5 = 200 nM | |
| | | | | TAR1- | |
| | | | | 5d6 = 100 nM | |
| | | | | In Fab | |
| | | | | format | |
| | | | | TAR1- | |
| | | | | 5CH | |
| | | | | d2CK = 30 nM | |
| | | | | TAR1- | |
| | | | | 5CK | |
| | | | | d3CH = 100 nM | |
| | TAR1-5- | | | 0.3 nM | 3-10 nM (eg, |
| | 19 | | | | 3 nM) |
| | homotrimer | | | | |
| TAR2 | TAR2 | As TAR1 monomer | As TAR1 | 500 nM | 500 nM to 50 pM |
| | monomers | | monomer | to | |
| | | | | 100 pM | |
| | TAR2-10 | | | | |
| | TAR2-5 | | | | |
| Serum | Anti-SA | 1 nM to 500 μM, | | 1 nM to | |
| Albumin | monomers | preferably 100 nM to | | 500 μM, | |
| | | 10 μM | | preferably | |
| | | In Dual Specific | | 100 nM | |
| | | format, target affinity | | to 10 μM | |
| | | is 1 to 100,000 × affinity | | In Dual | |
| | | of SA dAb | | Specific | |
| | | eg 100 pM | | format, | |
| | | (target) and 10 μM | | target | |
| | | SA affinity. | | affinity | |
| | | | | is 1 to | |
| | | | | 100,000 × affinity | |
| | | | | of SA | |
| | | | | dAb | |
| | | | | affinity, | |
| | | | | eg | |
| | | | | 100 pM | |
| | | | | (target) | |
| | | | | and 10 μM | |
| | | | | SA | |
| | | | | affinity. | |
| | MSA-16 | 200 nM | | | |
| | MSA-26 | 70 nM | | | |

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A domain antibody (dAb) monomer that has binding specificity for a receptor.

2. The dAb monomer of claim 1, wherein said dAb is a human dAb.

3. The dAb monomer of claim 1, wherein said dAb inhibits said receptor.

4. The dAb monomer of claim 3, wherein said dAb inhibits ligand binding.

5. The dAb monomer of claim 3, wherein said dAb inhibits receptor clustering.

6. The dAb monomer of claim 3, wherein said dAb inhibits receptor signalling.

7. The dAb monomer of claim 3, wherein said receptor is a human receptor.

8. The dAb monomer of claim 3, wherein said receptor is a dimeric receptor or multimeric receptor.

9. The dAb monomer of claim 3, wherein said receptor is a receptor for a cytokine or growth factor.

10. The dAb monomer of claim 1, wherein said dAb comprises a V_{H}3-family variable domain.

11. The dAb monomer of claim 10, wherein said dAb comprises a DP47, DP45 or DP38 V_{H} segment.

12. The dAb monomer of claim 1, wherein said dAb comprises a heavy chain hypervariable loop having the canonical structure of the H3 loop of DP47 and JH4b.

13. The dAb monomer of claim 12, further comprising a heavy chain hypervariable loop having the canonical structure of the H2 loop of DP47.

14. A ligand comprising the dAb monomer of claim 1, wherein said ligand is monovalent for receptor.

15. The ligand of claim 14, wherein said ligand further comprises a second dAb.

16. The ligand of claim 14, wherein said ligand further comprises an antibody constant domain.

17. The ligand of claim 16, wherein said ligand is an IgG format.

18. The ligand of claim 14, wherein said ligand is a dual specific ligand.

19. The ligand of claim 18, wherein said dual specific ligand further comprises a second dAb that binds an antigen or epitope which increases the half-life of said ligand.

20. The ligand of claim 19, wherein said second dAb binds serum albumin.

21. The ligand of claim 14, wherein said ligand further comprises a toxin.

22. A composition comprising the ligand of claim 14 and a pharmacologically acceptable carrier.

23. The composition of claim 24 further comprising a cytotoxic agent.

24. An isolated or recombinant nucleic acid encoding a ligand of claim 14 or a dAb of claim 1.

25. A recombinant vector comprising the recombinant nucleic acid of claim 24.

26. A host cell comprising the vector of claim 25.

27. The dAb monomer of claim 1, wherein said dAb antagonizes the activity of receptor in a standard cell assay with an ND50 of ≦100 nM, and at a concentration of ≦10 μM the dAb agonizes the activity of said receptor by ≦5% in the assay.

28. The dAb monomer of claim 1, wherein said dAb monomer binds said receptor with a Kd of 300 nM to 5 pM.

29. The ligand of claim 19, wherein said second dAb binds mouse serum albumin and human serum albumin.

30. The ligand of claim 19, wherein said second dAb binds human serum albumin, and further binds rat serum albumin, pig serum albumin or cynomolgus monkey serum albumin.

31. The ligand of claim 14, wherein said ligand further comprises an antibody Fc region.

32. The dAb monomer of claim 1, wherein said isolated dAb has binding specificity for membrane bound receptor.

33. The dual specific ligand of claim 18, wherein said dual specific ligand further comprises a second dAb that has binding specificity for another target, wherein said ligand can bind said receptor and said target simultaneously.

34. The dual specific ligand of claim 33, wherein said target is another receptor.

35. A method for antagonizing a receptor in a patient without substantially agonizing the receptor, comprising administering to a patient in need thereof a therapeutically effective dose of a ligand that is monovalent for said receptor, said ligand comprising a dAb that binds said receptor, under conditions suitable for the binding of said dAb to said receptor.

36. The method of claim 35, wherein said ligand inhibits the binding of cognate ligand to said receptor.

37. The method of claim 35, wherein said dAb inhibits receptor clustering.

38. The method of claim 35, wherein said dAb inhibits receptor signalling.

39. The method of claim 35, wherein said ligand is a dAb monomer.

40. A method for antagonizing a receptor without substantially agonizing the receptor, comprising combining a cell that expresses the receptor with a ligand that is monovalent for said receptor, wherein said ligand comprises a dAb that binds said receptor, under conditions suitable for the binding of said dAb to said receptor.

41. A ligand comprising at least one immunoglobulin single variable domain that has binding specificity for a receptor, and at least one immunoglobulin single variable domain that has binding specificity for an antigen or epitope which increases the half-life of said ligand.

42. The ligand of claim 41, wherein said ligand comprises two immunoglobulin single variable domains that have binding specificity for a receptor, and one immunoglobulin single variable domain that has binding specificity for an antigen or epitope which increases the half-life of said ligand.

43. The ligand of claim 42, wherein said ligand antagonizes the activity of receptor in a standard cell assay with an ND50 of ≦100 nM, and at a concentration of ≦10 μM the dAb agonizes the activity of said receptor by ≦5% in the assay.

44. The ligand of claim 42, wherein said ligand does not cause receptor clustering.

45. The ligand of claim 42, wherein the immunoglobulin single variable domain that has binding specificity for an antigen or epitope which increases the half-life of said ligand is an immumoglobulin single variable domain that has binding specificity for serum albumin.

46. The ligand of claim 41, wherein said receptor is a dimeric receptor or multimeric receptor.

47. The ligand of claim 46, wherein said receptor binds a cognate ligand and said cognate ligand is a cytokine or growth factor.

48. The ligand of claim 47, wherein said receptor is a receptor for a cytokine or growth factor.

49. A composition comprising the ligand of claim 42 and a pharmacologically acceptable carrier.

50. The composition of claim 49 further comprising a cytotoxic agent.

51. An isolated or recombinant nucleic acid encoding a ligand of claim 41.

52. A recombinant vector comprising the recombinant nucleic acid of claim 51.

53. A host cell comprising the vector of claim 52.


SEQUENCE LISTING (553 sequences)

SEQ ID NO: 1 - DNA, 720 bp, Homo sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagttatggtgcttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctaatacgttcggccaagggaccaaggtggaaatcaaacgg

SEQ ID NO: 2 - protein, 240 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 3 - DNA, 359 bp, Artificial Sequence
  Phagemide pIP1/pIT2
caggaaacagctatgaccatgattacgccaagcttgcatgcaaattctatttcaaggagacagtcataatgaaatacctattgcctacggcagccgctggattgttattactcgcggcccagccggccatggccgaggtgtttgactactggggccagggaaccctggtcaccgtctcgagcggtggaggcggttcaggcggaggtggcagcggcggtggcgggtcgacggacatccagatgacccaggcggccgcagaacaaaaactccatcatcatcaccatcacggggccgcaatctcagaagaggatctgaatggggccgcatagactgttgaaagttgtttagcaaaacctcat

SEQ ID NO: 4 - protein, 96 aa, Artificial Sequence
  Phagemide pIP1/pIT2
MKYLLPTAAAGLLLLAAQPAMAEVFDYWGQGTLVTVSSGGGGSGGGGSGGGGSTDIQMTQAAAEQKLHHHHHHGAAISEEDLNGAATVESCLAKPH

SEQ ID NO: 5 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 6 - protein, 116 aa, Homo sapiens
EVQLLESGGGLVQPGGSLRLSCAASGFTFSSYAMSWVRQAPGKGLEWVSHISPYGANTRYADSVKGRFTISRDNSKNTLYLQMNSLRAEDTAVYYCAKGLRAFDYWGQGTLVTVSS

SEQ ID NO: 7 - protein, 116 aa, Homo sapiens
EVQLLESGGGLVQPGGSLRLSCAASGFTFSSYAMSWVRQAPGKGLEWVSDIGATGSKTGYADPVKGRFTISRDNSKNTLYLQMNSLRAEDTAVYYCAKKVLTFDYWGQGTLVTVSS

SEQ ID NO: 8 - protein, 115 aa, Homo sapiens
EVQLLESGGGLVQPGGSLRLSCAASGFTFSSYAMSWVRQAPGKGLEWVSRINGPGATGYADSVKGRFTISRDNSKNTLYLQINSLRAEDTAVYYCAKHGAPFDYWGQGTLVTVSS

SEQ ID NO: 9 - protein, 116 aa, Homo sapiens
EVQLLESGGGLVQPGGSLRLSCAASGFTFSSYAMNWVRQAPGKGLEWVSSIPASGLHTRYADSVKGRFTISRDNSKNTLYLQMNSLRAEDTAVYYCAKPGLGFDYWGQGTLVTVSS

SEQ ID NO: 10 - protein, 115 aa, Homo sapiens
EVQLLESGGGLVQPGGSLRLSCAASGFTFSSYAMSWVRQAPGKGLEWVSDIERTGYTRYADSVKGRFTISRDNSKNTLYLQMNSLRAEDTAVYYCAKKVLVFDYWGQGTLVTVSS

SEQ ID NO: 11 - protein, 116 aa, Homo sapiens
EVQLLESGGGLVQPGGSLRLSCAASGFTFSSYAMSWVRQAPGKGLEWVSEISANGSKTQYADSVKGRLTISRDNSKNTLYLQMNSLRAEDTAVYYCAKKVLQFDYWGQGTLVTVSS

SEQ ID NO: 12 - protein, 115 aa, Homo sapiens
EVQLLESGGGLVQPGGSLRLSCAASGFTFSSYAMSWVRQAPGKGLEWVSTIPANGVTRYADSVKGRFTISRDNSKNTLYLQMNSLRAEDTAVYYCAKSLLQFDYWGQGTLVTVSS

SEQ ID NO: 13 - protein, 116 aa, Homo sapiens
EVQLLESGGGLVQPGGSLRLSCAASGFTFSSYAMSWVRQAPGKGLEWVSDIAATGSATSYADSVKGRFTISRDNSKNTLYLQMNSLRAEDTAVYYCAKKILKFDYWGQGTLVTVSS

SEQ ID NO: 14 - protein, 116 aa, Homo sapiens
EVQLLESGGGLVQPGGSLRLSCAASGSTFSSYAMSWVRQAPGKGLEWVSTISSVGQSTRYADSVKGRFTISRDNSKNTLYLQMNSLRAEDTAVYYCAKNLMSFDYWGQGTLVTVSS

SEQ ID NO: 15 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 16 - protein, 108 aa, Homo sapiens
DIQMTQSPSSLSASVGDRVTITCRASQSISSYLNWYQQKPGKAPKLLIYRASHLQSGVPSRFSGSGSGTDFTLTISSLQPEDFATYYCQQPWRSPGTFGQGTKVEIKR

SEQ ID NO: 17 - protein, 108 aa, Homo sapiens
DIQMTQSPSSLSASVGDRVTITCRASQSVSSYLNWYQQKPGKAPKLLIYLASRLQSGVPSRFSGSGSGTDFTLTISSLQPEDFATYYCQQNWWLPPTFGQGTKVEIKR

SEQ ID NO: 18 - protein, 107 aa, Homo sapiens
DIQMTQSPSSLSASVGDRVTITCRASQSISSYLNWYQQKPGKAPKLLIYASLLQSGVPSRFSGSGSGTDFTLTISSLQPEDFATYYCQQRVYDPLTFGQGTKVEIKR

SEQ ID NO: 19 - DNA, 348 bp, Homo sapiens
gaggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgtgacaattccaagaacacgctgtatctgcaaatgaacagcctgcgtgccgaggacaccgcggtatattactgtgcgaaaagttatggtgcttttgactactggggccagggaaccctggtcaccgtctcgagc

SEQ ID NO: 20 - DNA, 348 bp, Homo sapiens
gctcgagacggtgaccagggttccctggccccagtagtcaaaagcaccataacttttcgcacagtaatataccgcggtgtcctcggcacgcaggctgttcatttgcagatacagcgtgttcttggaattgtcacgggagatggtgaaccggcccttcacggagtctgcgtagtatgtgctaccaccactaccactaatagctgagacccactctagacccttccctggagcctggcggacccagctcatggcatagctgctaaaggtgaatccggaggctgcacaggagagacgcagggaccccccaggctgtaccaagcctcccccagactccaacagctgcacctc

SEQ ID NO: 21 - protein, 120 aa, Homo sapiens
  VARIANT 103, 104, 105, 106: Xaa = Any Amino Acid
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAXXXXFDYWGQGTLVTVSS

SEQ ID NO: 22 - DNA, 360 bp, Homo sapiens
  misc_feature 309, 312, 315, 318: K = G or T
gaggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgtgacaattccaagaacacgctgtatctgcaaatgaacagcctgcgtgccgaggacaccgcggtatattactgtgcgaaaagttatggtgctnnknnknnknnktttgactactggggccagggaaccctggtcaccgtctcgagc

SEQ ID NO: 23 - DNA, 360 bp, Homo sapiens
  misc_feature 43, 46, 49, 52: K = G or T
gctcgagacggtgaccagggttccctggccccagtagtcaaaknnknnknnknnagcaccataacttttcgcacagtaatataccgcggtgtcctcggcacgcaggctgttcatttgcagatacagcgtgttcttggaattgtcacgggagatggtgaaccggcccttcacggagtctgcgtagtatgtgctaccaccactaccactaatagctgagacccactctagacccttccctggagcctggcggacccagctcatggcatagctgctaaaggtgaatccggaggctgcacaggagagacgcagggaccccccaggctgtaccaagcctcccccagactccaacagctgcacctc

SEQ ID NO: 24 - DNA, 324 bp, Homo sapiens
gacatccagatgacccagtctccatcctccctgtctgcatctgtaggagaccgtgtcaccatcacttgccgggcaagtcagagcattagcagctatttaaattggtaccagcagaaaccagggaaagcccctaagctcctgatctatgctgcatccagtttgcaaagtggggtcccatcacgtttcagtggcagtggatctgggacagatttcactctcaccatcagcagtctgcaacctgaagattttgctacgtactactgtcaacagagttacagtacccctaatacgttcggccaagggaccaaggtggaaatcaaacgg

SEQ ID NO: 25 - DNA, 324 bp, Homo sapiens
ccgtttgatttccaccttggtcccttggccgaacgtattaggggtactgtaactctgttgacagtagtacgtagcaaaatcttcaggttgcagactgctgatggtgagagtgaaatctgtcccagatccactgccactgaaacgtgatgggaccccactttgcaaactggatgcagcatagatcaggagcttaggggctttccctggtttctgctggtaccaatttaaatagctgctaatgctctgacttgcccggcaagtgatggtgacacggtctcctacagatgcagacagggaggatggagactgggtcatctggatgtc

SEQ ID NO: 26 - DNA, 324 bp, Homo sapiens
gacatccagatgacccagtctccatcctccctgtctgcatctgtaggagaccgtgtcaccatcacttgccgggcaagtcagagcattattaagcatttaaagtggtaccagcagaaaccagggaaagcccctaagctcctgatctatggtgcatcccggttgcaaagtggggtcccatcacgtttcagtggcagtggatctgggacagatttcactctcaccatcagcagtctgcaacctgaagattttgctacgtactactgtcaacagggggctcggtggcctcagacgttcggccaagggaccaaggtggaaatcaaacgg

SEQ ID NO: 27 - protein, 108 aa, Homo sapiens
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 28 - DNA, 324 bp, Homo sapiens
gacatccagatgacccagtctccatcctccctgtctgcatctgtaggagaccgtgtcaccatcacttgccgggcaagtcagagcatttattatcatttaaagtggtaccagcagaaaccagggaaagcccctaagctcctgatctataaggcatccacgttgcaaagtggggtcccatcacgtttcagtggcagtggatctgggacagatttcactctcaccatcagcagtctgcaacctgaagattttgctacgtactactgtcaacaggttcggaaggtgcctcggacgttcggccaagggaccaaggtggaaatcaaacgg

SEQ ID NO: 29 - protein, 108 aa, Homo sapiens
DIQMTQSPSSLSASVGDRVTITCRASQSIYYHLKWYQQKPGKAPKLLIYKASTLQSGVPSRFSGSGSGTDFTLTISSLQPEDFATYYCQQVRKVPRTFGQGTKVEIKR

SEQ ID NO: 30 - protein, 182 aa, Homo sapiens
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 31 - protein, 183 aa, Mus musculus
LVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDSGTA

SEQ ID NO: 32 - DNA, 323 bp, Homo sapiens
gacatccagatgacccagtctccatcctccctgtctgcatctgtaggagaccgtgtcaccatcacttgccgggcaagtcagaatattgattctcgtttaagttggtaccagcagaaaccagggaaagcccctaagctcctgatctataggacgtccgttttgcaaagtggggtcccatcacgtttcagtggcagtggatctgggacagatttcactctcaccatcagcagtctgcaacctgaagattttgctacgtactactgtcaacagtgggatatgtttcctttgacgttcggccaagggaccaaggtggaagtcaaacg

SEQ ID NO: 33 - protein, 108 aa, Homo sapiens
DIQMTQSPSSLSASVGDRVTITCRASQWIGILLDWYQQKPGEAPKLLIYYASFLQSGVPSRFSGSGSGTDFTLTISSLQPEDFATYYCQQANPAPLTFGQGTKVEIKR

SEQ ID NO: 34 - protein, 108 aa, Homo sapiens
DIQMTQSPSSLSASVGDRVTITCRASQWIGILVDWYQQKPGEAPKLLIYYASFLQSGVPSRFSGSGSGTDFTLTISSLQPEDFATYYCQQANPAPLRFGQGTKVEIKR

SEQ ID NO: 35 - protein, 108 aa, Homo sapiens
DIQMTQSPSSLSASVGDRVTITCRASQWIGILVDWYQQKPGEAPKLLIYYASFLQSGVPSRFSGSGFGTDFTLTISSLHPEDFATYYCQQANPAPLTFGQGTKVEIKR

SEQ ID NO: 36 - protein, 108 aa, Homo sapiens
DIQMTQSPSSLSASVGDRVTITCRASQWIGILVDWYQQKPGEAPKLLIYYASFLQSGVPSRFSGSGSGTDFTLTISSLQPEDFATYYCQQANPAPLTFGQGTKVEIKR

SEQ ID NO: 37 - protein, 108 aa, Homo sapiens
DIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKR

SEQ ID NO: 38 - protein, 108 aa, Homo sapiens
DIQMTQSPSSLSASVGDRVTITCRASQWIGILLDWYQQKPGEAPKLLIYYASFLQSGVPSRFSGSGYGTDFTLTISSLQPEDFATYYCQQANPAPLTFGQGTKVEIKR

SEQ ID NO: 39 - protein, 108 aa, Homo sapiens
DIQMTQSPSSLSASVGDRVTITCRASQWIGILLDWYQQKPGKAPKLLIYYASFLQSGVPSRFSGSGSGTDFTLTISSLQPEDFATYYCQQANPAPLTFGQGTKVEIKR

SEQ ID NO: 40 - protein, 116 aa, Homo sapiens
EVQLLESGGGLVQPGGSLRLSCAASGFTFDLYNMFWVRQAPGKGLEWVSFISQTGRLTWYADSVKGRFTISRDNSKNTLYLQMNSLRAEDTAVYYCAKTLEDFDYWGQGTLVTVSS

SEQ ID NO: 41 - protein, 120 aa, Mus musculus
EVQLLESGGGLVQPGGSLRLSCAASGFTFEWYWMGWVRQAPGKGLEWVSAISGSGGSTYYADSVKGRFTISRDNSKNTLYLQMNSLRAEDTAVYYCAKVKLGGGPNFDYWGQGTLVTVSS

SEQ ID NO: 42 - protein, 120 aa, Homo sapiens
EVQLLESGGGLVQPGGSLRLSCAASGFTFEWYWMGWVRQAPGKGLEWVSAISGSGGSTYYADSVKGRFTISRDNSKNTLYLQMNSLRAEDAAVYYCAKVKLGGGPNFGYRGQGTLVTVSS

SEQ ID NO: 43 - protein, 119 aa, Homo sapiens
EVQLLESGGGLIQPGGSLRLSCAASGFTFAHETMVWVRQAPGKGLEWVSHIDRVGQDTYYADSVKGRFTISRDNSKNTLYLQMNSLRAEDTAVYYCAKLPKRGPWFDYRGQGTLVTVSS

SEQ ID NO: 44 - protein, 121 aa, Homo sapiens
EVRLLESGGGLVQPGGSLRLSCVASGFTFGKSTMTWVRQAPGKGLEWVSHISDDGNSTYYADSVKGRFTISRDNSKNTLYLQMNSLRAEDTAVYYCAKVPILAPRNLFDYWGQGTLVTVSS

SEQ ID NO: 45 - protein, 116 aa, Homo sapiens
EVQLLESGGGLVQPGGSLRLSCAASGFTFDAYNMFWFRQAPGKGPEWVSAIGPSGRETYYADSVKGRFTITRDNSKNTLYLQMNSLRAEDTAVYYCAKRYPDFDYWGQGTLVTVSS

SEQ ID NO: 46 - protein, 119 aa, Homo sapiens
EVQLLESGGGLVQPGGSLRLSCAASGFTFEHEGMVWVRQAPGKGLEWVSHIGEDGQSTYYADSVKGRFTISRDNSRNTLYLQMNSLRAEDTAVYYCANIPKAGPSFDYWGQGTLVTVSS

SEQ ID NO: 47 - protein, 119 aa, Homo sapiens
EVQLLESGGGMVQPGGSLRLSCAASGFTFEHEGMVWVRQAPGKGLEWVSHIGEDGQSTYYADSVKGRFTISRDNSKNTLYLQMNSLRAEDTAVYYCANIPKAGPSFDYWGQGTLVTVSS

SEQ ID NO: 48 - protein, 122 aa, Homo sapiens
EVQLLESGGGLVQPGGSLRLSCAASGFTFARYNMGWVRQAPGKGLEWVSLIDPSGGHTYYADSVKGRFTISRNNSKNTLYLQMNSLRAEDTAVYYCGKPVFSDWPAVEFDYWGQGTVVTVSS

SEQ ID NO: 49 - protein, 108 aa, Homo sapiens
DIQMTQSPSSLSASVGDRVTITCRASQWIGRELRWYQQKPGKAPMFLIYHASRLQRGVPSRFSGSGSGTDFTLTISSLQPEDFATYYCQQYHGWPLTFGQGTKVEIKR

SEQ ID NO: 50 - protein, 108 aa, Homo sapiens
DIQMTQSPSSLSASVGDRVTITCRASQWIGRELRWYQQKPGKAPKFLIYHASRLQRGVPSRFSGSGSGTDFTLTISSLQPEDFATYYCQQYHGWPLTFGQGTKVEIKR

SEQ ID NO: 51 - protein, 108 aa, Homo sapiens
DIQMTQSPSSLSASVGDRVTITCRASQNIDRELRWYQQKPGKAPMLLIYHASRLQSGVPSRFSGSGSGTDFTLTISSLQPEDFATYYCQQYANWPLTFGQGTKVEIKR

SEQ ID NO: 52 - protein, 108 aa, Homo sapiens
DIQMTQSPSSLSASVGDRVTITCRASQSIGRELRWYQQKPGKAPMFLIYHASRLQSGVPSRFSGSGSGTDFTLTISSLQPEDFATYYCQQYHSWPLTFGQGTKVEIKR

SEQ ID NO: 53 - protein, 108 aa, Homo sapiens
DIQMTQSPSSLSASVGDRVTITCRASQSIGRELRWYQQKPGKAPMFLIYHASRLQSGVPSRFSGSGSGTDFTLTISSLQPEDFATYYCQQYFHWPLTFGQGTKVEIKR

SEQ ID NO: 54 - protein, 108 aa, Homo sapiens
DIQMTQSPSSLSASVGDRVTITCRASQSIGRELRWYQQKPGKAPMFLIYHASRLQRGVPSRFSGSGSGTDFTLTISSLQPEDFATYYCQQYHSWPLTFGQGTKVEIKR

SEQ ID NO: 55 - protein, 108 aa, Homo sapiens
DIQMTQSPSSLSASVGDRVTITCRASQNIGRELRWYQQKPGKAPMFLIYHASRLQSGVPSRFSGSGSGTDFTLTISSLQPEDFATYYCQQYNSWPLTFGQGTKVEIKR

SEQ ID NO: 56 - protein, 108 aa, Homo sapiens
DIQMTQSPSSLSASVGDRVTITCRASQHIGVELRWYQQKPGEAPMFLIYHASRLQSGVPSRFSGSGSGTDFTLTISSLQPEDFATYYCQQYYGWPLTFGQGTKVEIKR

SEQ ID NO: 57 - protein, 108 aa, Homo sapiens
DIQMTQSPSSLSASVGDRVTITCRASQWIGRELRWYQQKPGKAPMFLIYHASRLQSGVPSRFSGSGSGTDFTLTISSLQPEDFATYYCQQYHGWPLTFGQGTKVEIKR

SEQ ID NO: 58 - protein, 108 aa, Homo sapiens
DIQMTQSPSSLSASVGDRVTITCRASQSITKELRWYQQKPGKAPMLLIYHASRLQRGVPSRFSGSGSGTDFTLTISSLQPEDFATYYCQQYHSWPLTFGQGTKVEIKR

SEQ ID NO: 59 - protein, 108 aa, Homo sapiens
DIQMTQSPSSLSASVGDRVAITCRASQGIGRELRWYQQKPGKAPMLLIHHASRLQSGVPSRFSGSGSGTDFTLTISSLQPEDFATYYCQQYNRWPLTFGQGTKVEIKR

SEQ ID NO: 60 - protein, 108 aa, Homo sapiens
DIQMTQSPSSLSASVGDRVTITCRASQNIGRELRWYQQKPGEAPMLLIYHASRLQSGVPSRFSGSGSGTDFTLTISSLQPEDFATYYCQQYFYWPLTFGQGTKVEIKR

SEQ ID NO: 61 - protein, 108 aa, Homo sapiens
DIQMTQSPSSLSASVGDRVTITCRASQNITRELRWYQQKPGKAPMLLIYHASRLQSGVPSRFSGSGSGTDFTLTISSLQPEDFATYYCQQYHGWPLTFGQGTKVEIKR

SEQ ID NO: 62 - protein, 108 aa, Homo sapiens
DIQMTQSPSSLSASVGDRVTITCRASQAIGRELRWYQQKPGKAPMLLIHHASRLQSGVPSRFSGSGSGTDFTLTISSLQPEDFATYYCQQYNGWPLTFGQGTKVEIKR

SEQ ID NO: 63 - protein, 108 aa, Homo sapiens
DIQMTQSPSSLSASVGDRVTITCRASQDIGRELRWYQQKPGKAPMFLIYHASRLQSGVPSRFSGSGSGTDFTLTISSLQPEDFATYYCQQYGFWPLTFGQGTKVEIKR

SEQ ID NO: 64 - protein, 108 aa, Homo sapiens
DIQMTQSPSSLSASVGDRVTITCRASQDITKELRWYQQKPGKAPMLLIHHASRLQSGVPSRFSGSGSGTDFTLTISSLQPEDFATYYCQQYHSWPLTFGQGTKVEIKR

SEQ ID NO: 65 - protein, 108 aa, Homo sapiens
DIQMTQSPSSLSASVGDRVTITCRASQSIGRELRWYQQKPGKAPMFLIYHASRLQSGVPSRFSGSGSGTDFTLTISSLQPEDFATYYCQQYYLWPLTFGQGTKVEIKR

SEQ ID NO: 66 - protein, 108 aa, Homo sapiens
DIQMTQSPSSLSASVGDRVTITCRASQRIGVELRWYQQKPGKAPMPLIYHASRLQSGVPSRFSGSGSGTDFTLTISSLQPEDFATYYCQQYFGWPLTFGQGTKVEIKR

SEQ ID NO: 67 - protein, 108 aa, Homo sapiens
DIQMTQSPSSLSASVGDRVTITCRASQWIDRELRWYQQKPGKAPMLLIYHASRLQSGVPSRFSGSGSGTDFTLTISSLQPEDFATYYCQQYHGWPLTFGQGTKVEIKR

SEQ ID NO: 68 - protein, 108 aa, Homo sapiens
DIQMTQSPSSLSASVGDRVTITCRASQSIFKELRWYQQKPEEAPMLLIHHASRLQSGVPSRFSGSGSGTDFTLTISSLQPEDFATYYCQQYHGWPLTFGQGTKVEIKR

SEQ ID NO: 69 - protein, 108 aa, Homo sapiens
DIQMTQSPSSLSASVGDRVTITCRASQEIGRELRWYQQKPGEAPMPLIYHASRLQSGVPSRFSGSGSGTDFTLTISSLQPEDFATYYCQQYFTWPLTFGQGTKVEIKR

SEQ ID NO: 70 - protein, 108 aa, Homo sapiens
DIQMTQSPSSLSASVGDRVTITCRASQPIGIELRWYQQKPGKAPMPLIYHASRLQSGVPSRFSGSGSGTDFTLTISSLQPEDFATYYCQQYHGWPLTFGQGTKVEIKQ

SEQ ID NO: 71 - protein, 108 aa, Homo sapiens
DIQMTQSPSSLSASVGDRVTITCRASQSIGRELRWYQQKPGKAPMLLIYHASRLRSGVPSRFSGSGSGTDFTLTISSLQPEDFATYYCQQYNGWPLTFGQGTKVEIKR

SEQ ID NO: 72 - protein, 108 aa, Homo sapiens
DIQMTQSPSSLSASVGDRVTITCRASQSIGRELRWYQQKPGKAPMFLIYHASRLQSGVPSRFSGSGSGTDFTLTISSLQPEDFATYYCQQYYDWPLTFGQGTKVEIKR

SEQ ID NO: 73 - protein, 108 aa, Homo sapiens
DIQMTQSPSSLSASVGDRVTITCRASQPIGRELRWYQQKPGKAPMFLIYHASRLQSGVPSRFSGSGSGTDFTLTISSLQPEDFATYYCQQYSGWPLTFGQGTKVEIKR

SEQ ID NO: 74 - protein, 108 aa, Homo sapiens
DIQMTQSPSSLSASVGDRVTITCRASQWIGRELRWYQQKPGKAPMLLIYHASRLQRGVPSRFSGSGSGTDFTLTISSLQPEDFATYYCQQYHGWPLTFGQGTKVEIKR

SEQ ID NO: 75 - protein, 108 aa, Homo sapiens
DIQMTQSPSSLSASVGDRVTITCRASQWIGRELRWYQQKPGKAPKLLIYHASRLQRGVPSRFSGSGSGTDFTLTISSLQPEDFATYYCQQYHGWPLTFGQGTKVEIKR

SEQ ID NO: 76 - protein, 108 aa, Homo sapiens
DIQMTQSPSSLSASVGDRVTITCRASQWIGRELRWYQQKPGEAPMFLIYHASRLQRGVPSRFSGSGSGTDFTLTISSLQPEDFATYYCQQYHGWPLTFGQGTKVEIKR

SEQ ID NO: 77 - protein, 108 aa, Homo sapiens
DIQMTQSPSSLSASVGDRVTITCRASQWIGRELRWYQQKPGKAPMFLIHHASRLQRGVPSRFSGSGSGTDFTLTISSLQPEDFATYYCQQYHGWPLTFGQGTKVEIKR

SEQ ID NO: 78 - protein, 108 aa, Homo sapiens
DIQMTQSPSSLSASVGDRVTITCRASQWIGRELRWYQQKPGEAPMFLIHHASRLQRGVPSRFSGSGSGTDFTLTISSLQPEDFATYYCQQYHGWPLTFGQGTKVEIKR

SEQ ID NO: 79 - protein, 108 aa, Homo sapiens
DIQMTQSPSSLSASVGDRVTITCRASQWIGRELRWYQQKPGKAPKFLIYHASRLIKGVPSRFSGSGSGTDFTLTISSLQPEDFATYYCQQYHGWPLTFGQGTKVEIKR

SEQ ID NO: 80 - protein, 108 aa, Homo sapiens
DIQMTQSPSSLSASVGDRVTITCRASQWIGRELRWYQQKPGKAPKFLIYHASRLQRGVPSRFSGSGSGTDFTLTISSLQPEDFATYYCQQYHGFPLTFGQGTKVEIKR

SEQ ID NO: 81 - protein, 108 aa, Homo sapiens
DIQMTQSPSSLSASVGDRVTITCRASQWIGRELRWYQQKPGKAPKFLIYHASRLYKGVPSRFSGSGSGTDFTLTISSLQPEDFATYYCQQYHGWPLTFGQGTKVEIKR

SEQ ID NO: 82 - protein, 108 aa, Homo sapiens
DIQMTQSPSSLSASVGDRVTITCRASQWIGRELRWYQQKPGKAPKFLIYHASRLQSGVPSRFSGSGSGTDFTLTISSLQPEDFATYYCQQYHGWPLTFGQGTKVEIKR

SEQ ID NO: 83 - protein, 108 aa, Homo sapiens
DIQMTQSPSSLSASVGDRVTITCRASQWIGRELRWYQQKPGKAPKLLIYHASRLQSGVPSRFSGSGSGTDFTLTISSLQPEDFATYYCQQYHGWPLTFGQGTKVEIKR

SEQ ID NO: 84 - protein, 108 aa, Homo sapiens
DIQMTQSPSSLSASVGDRVTITCRASAWIGRELRWYQQKPGKAPKFLIYHASRLIKGVPSRFSGSGSGTDFTLTISSLQPEDFATYYCQQYHGWPLTFGQGTKVEIKR

SEQ ID NO: 85 - protein, 108 aa, Homo sapiens
DIQMTQSPSSLSASVGDRVTITCRASGWIGRELRWYQQKPGKAPKFLIYHASRLYKGVPSRFSGSGSGTDFTLTISSLQPEDFATYYCQQYHGWPLTFGQGTKVEIKR

SEQ ID NO: 86 - protein, 108 aa, Homo sapiens
DIQMIQSPSSLSASVGDRVTITCRASQWIGRELRWYQQKPGKAPKLLIHHASRLQRGVPSRFSGSGSGTDFTLTISSLQPEDFTTYYCQQYHGWPLTFGQGTKVEIKR

SEQ ID NO: 87 - protein, 108 aa, Homo sapiens
DIQMTQSPSSLSASVGDRVTITCRASQWIGRELRWYQQKPGKAPKPLIYHASRLQRGVPSRFSGSGSGTDFTLTISSLQPEDFATYYCQQYHGWPLTFGQGTKVEIER

SEQ ID NO: 88 - protein, 108 aa, Homo sapiens
DIQMTQSPSSLSASVGDRVTITCRASQWIGRELRWYQQKPGKDPKPLIYHASRLQRGVPSRFSGSGSGTGFTLTISSLQPEDFATYYCQQYHGWPLTFGQGTKVEIKR

SEQ ID NO: 89 - protein, 108 aa, Homo sapiens
DIQMTQSPSSLSASVGDRVTITCRASQWIGRELRWYQQKPGKDPKVLIFHASRLQRGVPSRFSGSGSGTDFTLTISSLQPEDFATYYCQQYHGWPLTFGQGTKVEIKR

SEQ ID NO: 90 - protein, 108 aa, Homo sapiens
DIQMTQSPSSLSASVGDRVTITCRASQWIGRELRWYQQKPGKAPKQLIYHASRLQRGVPSRFSGSGSGTDFTLTISSLQPEDFTTYYCQQYHGWPLTFGQGTKVEIKR

SEQ ID NO: 91 - protein, 108 aa, Homo sapiens
GIQMTQSPSSLSASVGDRVTITCRASQWIGRELRWYQQKPGKASKPLIYHASRLQRGVPSRFSGSGSGTDFTLTISSLQPEDFATYYCQQYHGWPLTFGQGTKVEIKR

SEQ ID NO: 92 - protein, 108 aa, Homo sapiens
DIQMTQSPSSLSASVGDRVTITCRASQWIGRELRWYQQKPGKDPKLLIHHASRLQRGVPSRFSGSGSGTDFTLTISSLQPEDFATYYCQQYHGWPLTFGQGTKVEIKR

SEQ ID NO: 93 - protein, 108 aa, Homo sapiens
DIQMTQFPSSLSASVGDRVSITCRASQWIGRELRWYQQKPGKDPKPLIYHASRLQRGVPSRFSGSGSGTDFTLTISSLQPEDLATYYCQQYHGWPLTFGQGTKVEIKR

SEQ ID NO: 94 - protein, 108 aa, Homo sapiens
DIQMTQSPSSLSASVGDRVTITCRASQWIGRELRWYQQKPGKAPKPLIYHASRLQRGVPSRFSGSGSGTDFTLTISSLQPEDFATYYCQQYHGWPLTFGQGTKVEIKR

SEQ ID NO: 95 - protein, 108 aa, Homo sapiens
DIQMTQSPSSLSASVGDRVIITCRASQWIGRELRWYQQKPGKAPKLLIFHASRLQRGVPSRFSGSGSGTDFTLTISSLQPEDFATYYCQQYHGWPLTFGQGTKVEIKR

SEQ ID NO: 96 - protein, 108 aa, Homo sapiens
DIQMTQSPSSLSASVGDRVTITCRASQWIGRELRWYQQKPGKSPKPLIYHASRLQRGVPSRFSGSGSGTDFTLIISSLQPEDFATYYCQQYHGWPLTLGQGTKVEIKR

SEQ ID NO: 97 - protein, 108 aa, Homo sapiens
DIQMTQSPPSLSASVGDRVTITCRASQWIGRELRWCQQKPGKAPKLLIFHASRLQRGVPSRFSGSGSGTDFTLTISSLQPEDFATYYCQQYHGWPLTFGQGTKVEIKR

SEQ ID NO: 98 - protein, 108 aa, Homo sapiens
DIQMTQSPSSLSASVGDRVTITCRASQWIGRELRWYQQKPGKAPKLLIHHASRLQRGVPSRFSGSGSGTDFTLTISSLQPEDFATYYCQQYHGWPLTFGQGTKVEIKR

SEQ ID NO: 99 - protein, 108 aa, Homo sapiens
DIQMTQSPSSLSASVGDRVTITCRASQWIGRELRWYQQKPGKAPKLLIHHASRLQRGVPSRFSGSGSGADFTLTISSLQPEDFATYYCQQYHGWPLTFGQWTKVEIKR

SEQ ID NO: 100 - protein, 108 aa, Homo sapiens
DIQMTQSPSSLSASVGDRVTITCRASQWIGRELRWYQQKPGKAPKLLFHHASRLQRGVPSRFSGSGSGTDFTLTISSLQPEDFATYYCQQYHGWPLTFGQGTKVEIKR

SEQ ID NO: 101 - protein, 108 aa, Homo sapiens
DIQMTQSPSSLSASVGDRVTITCRASQWIGRELRWYQQKPGKAPKLLIFHASRLQRGVPSRFSGSGSGTDFTLTISSLQPEDFATYYCQQYHGWPLTFGQGTKVEIKR

SEQ ID NO: 102 - protein, 108 aa, Homo sapiens
DIQMTQSPSSLSASVGDRVTITCRASQWIGRELRWYQQKPGKAPMPLIYHASRLQRGVPSRFSGSGSGTDFTLTISSLQPEDFATYYCQQYHGWPLTFGQGTKVEIKR

SEQ ID NO: 103 - protein, 108 aa, Homo sapiens
DIQMTQSPSSLSASVGDRVTITCRASQWIGRELRWYQQKPGKAPKLLIFHASRLQRGVPSRFSGSGSGTDFTLTISSLQPEDFAAYYCQQYHGWPLTFGQGTKVEIKR

SEQ ID NO: 104 - protein, 108 aa, Homo sapiens
DIQMTQSPSSLSASVGDRVTITCRTSQWIGRELRWYQQKPGKAPKLLIHHASRLQRGVPSRFSGSGSGTDFTLTISSLQPEDFATYYCQQYHGWPLTFGQGTKVEIKR

SEQ ID NO: 105 - protein, 108 aa, Homo sapiens
DIQMTQSPSSLSASVGDHVTITCRASQWIGRELRWDQQKPGKAPKLLIYHASRLQRGVPSRFSGSGSGTDFTLTISSLQPEDFATYYCQQYHGWPLTFGQGTKVEIKR

SEQ ID NO: 106 - protein, 108 aa, Homo sapiens
DIQMTQSPSSLSASVGDRVTITCRASQWIGRELRWYQQKPGKAPTPLIYHASRLQRGVPSRFSGSGSGTDFTLTISSLQPEDFATYYCQQYHGWPLTFGQGTKVEIKR

SEQ ID NO: 107 - protein, 108 aa, Homo sapiens
DIQMTQSPSSLSASVGDRVTITCRASQWIGRELRWYQQKPGKAPKQLIYHASRLQRGVPSRFSGSGSGSDFTLTISSLQPEDFATYYCQQYHGWPLTFGQGTKAEIKR

SEQ ID NO: 108 - protein, 108 aa, Homo sapiens
DIQMTQSPSSLSASVGDRVAITCRASQWIGRELRWYQQKPGKAPKLLIFHASRLQRGVPSRFSGSGSGTDFTLTISSLQPEDFATYYCQQYHGWPLTFGQGTKVEIKR

SEQ ID NO: 109 - protein, 108 aa, Homo sapiens
GIQMTQSPSSLSASVGDRVTITCRASQWIGRELRWYQQKPGKDPKPLIYHASRLQRGVPSRFSGSGSGTDFTLTISSLQPEDFATYYCQQYHGWPLTFGQGTKVEIKR

SEQ ID NO: 110 - protein, 108 aa, Homo sapiens
DIQMTQSPSSLSASVGDRVTITCRASQWIGRELRWYQQKPGKAPKVLIFHASRLQRGVPSRFSGSGSGTDFTLTISSLQPEDFATYYCQQYHGWPLTFGQGTKVEIKR

SEQ ID NO: 111 - protein, 108 aa, Homo sapiens
DIQMTQSPSSLSASVGDRVTITCRASQWIGRELRWYQQKPGKDPKPLIYHASRLQRGVPSRFSGSGSGTDFTLTISSLQPEDFATYYCQQYHGWPLTSGQGTKVEIKR

SEQ ID NO: 112 - protein, 108 aa, Homo sapiens
DIQMTQSPSSLSASVGDRVTITCRASQWIGRELRWYQQEPGEAPKPLIYHASRLQRGVPSRFSGSGSGTDFTLTISSLQPDDSATYYCQQYHGWPLTFGQGTRVEIKR

SEQ ID NO: 113 - protein, 108 aa, Homo sapiens
DIQMTQSPSSLSASVGDRVTITCRASQWIGRELRWYQQEPGKAPKLLIHHASRLQRGVPSRFSGSGSGTDFTLTISSLQPEDFATYYCQQYHGWPLTFGQGTKVEIKR

SEQ ID NO: 114 - protein, 108 aa, Homo sapiens
DIQMTQSPSSLSASVGDRVTITCRASQWIGRELRWYQQKPGKAPKLLIFHASRLQRGVPSRFSGSGSGTDFTLTISSLQPEDYATYYCQQYHGWPLTFGQGTKVEIKR

SEQ ID NO: 115 - protein, 108 aa, Homo sapiens
DIQMTQSPSSLSASVGDRVTITCRASQWIGRELRWYQQKPGKAPNPLIYHASRLQRGVPSRFSGSGSGTDFTLTISSLQPEDFATYYCQQYHGWPLTFGQGTKVEIKR

SEQ ID NO: 116 - protein, 108 aa, Homo sapiens
DIQMTQSPSSLSASVGDRVTITCRASQWIGRELRWYQQKPGKAPKQLIYHASRLQRGVPSRFSGSGSGTDFTLTISSLQPEDFATYYCQQYHGWPLTSGQGTKVEIKR

SEQ ID NO: 117 - protein, 108 aa, Homo sapiens
DIQMTQSPSSLSASVGDRVTITCRASQWIDRELRWYQQKPGKAPKQLIYHASRLQRGVPSRFSGSGSGTDFTLTISSLQPEDFATYYCQQYHGWPLTFGQGTKVEIKR

SEQ ID NO: 118 - protein, 108 aa, Homo sapiens
DIQMTQSPSSLSASVGDRVTITCRASQWIGRELRWYQQKPGKAPKPLIYHASRLQRGVPSRFSGSRSGTDFTLTISSLQPEDFATYYCQQYHGWPLTFGQGTKVEIKR

SEQ ID NO: 119 - protein, 108 aa, Homo sapiens
DIQMTQSPSSLSASVGDRVTITCRASQWVGRELRWYQQIPGKAPKQLIYHASRLQRGVPSRFSGSGSGTDFTLTIGSLQPEDFATYYCQQYHGWPLTFGQGTKVEIKR

SEQ ID NO: 120 - protein, 108 aa, Homo sapiens
DIQMTQSPSSLSASVGDHVTITCRASQWIGRELRWDQQKPGKAPKFLIYHASRLQRGVPSRFSGSGSGTDFTLTISSLQPEDFATYYCQQYHGWPLTFGQGTKVEIKR

SEQ ID NO: 121 - protein, 108 aa, Homo sapiens
DIQMTQSPSSLSASVGDRVTITCRASQWIGRELRWYQQKPGKAPKGLIYHASRLQRGVPSRFSGSGSGTDFTLTISSLQPEDFATYYCQQYHGWPLTFGQGTKVEIKR

SEQ ID NO: 122 - DNA, 324 bp, Homo sapiens
gacatccagatgacccagtctccatcctccctgtctgcatctgtaggagaccgtgtcaccatcacttgccgggcaagtcagtcgattgggaataatttactttggtaccagcagaaaccagggaaagcccctaagctcctgatctattatacgtccaggttgcaaagtggggtcccatcacgtttcagtggcagtggatctgggacagatttcactctcaccatcagcagtctgcaacctgaagattttgctacgtactactgtcaacagcgtcggactcatcctcatacgttcggccaagggaccaaggtggaaatcaaacgg

SEQ ID NO: 123 - DNA, 324 bp, Homo sapiens
gacatccagatgacccagtctccatcctccctgtctgcatctgtaggagaccgtgtcaccatcacttgccgggcaagtcaggatatttttactaagttaaggtggtaccagcagaaaccagggaaagcccctaggctcctgatctatgcgggttcccgtttgcaaagtggggtcccatcacgtttcagtggcagtggatctgggacagatttcactctcaccatcagcagtctgcaacctgaagattttgctacgtactactgtcaacaggttaagcagaagccttggacgttcggccaagggaccaaggtggaaatcaaacgg

SEQ ID NO: 124 - DNA, 324 bp, Homo sapiens
gacatccagatgacccagtctccatcctccctgtctgcatctgtaggagaccgtgtcaccattacttgccgggcaagtcagcatattgggagtatgttagagtggtaccagcagaaaccagggaaagcccctaagctcctgatctatcgtgcgtcctttttgcaaagtggggtcccatcacgtttcagtggcagtggatctgggacagatttcactctcaccatcagcagtctgcaacctgaagattttgctacgtactactgtcaacaggggcgggcgcttccttttacgtttggccaagggaccaaggtggaaatcaaacgg

SEQ ID NO: 125 - DNA, 324 bp, Homo sapiens
gacatccagatgacccagtctccatcctccctgtctgcatctgtaggagaccgtgtcaccatcacttgccgggcaagtcagaatattgagtcttggttaaggtggtaccagcagaaaccagggaaagcccctaagctcctgatctatcattcgtccaggttgcaaagtggggtcccatcacgtttcagtggcagtggatctgggacagatttcactctcaccatcagcagtctgcaacctgaagattttgctacgtactactgtcaacagtctagggttcgtccttttacgttcggccaagggaccaaggtggaaatcaaacgg

SEQ ID NO: 126 - DNA, 324 bp, Homo sapiens
gacatccagatgacccagtctccatcctccctgtctgcatctgtaggagaccgtgtcaccatcacttgccgggcaagtcagagcattagcagctatttaaattggtaccagcagaaaccagggaaagcccctaagctcctgatcaggcgtggttcccttttgcaaagtggggtcccatcacgtttcagtggcagtggatctgggacagatttcactctcaccatcagcagtctgcaacctgaagattttgctacgtactactgtcgtcagggtatggctcgtccttggacgttcggccaagggaccaaggtggaaatcaaacgg

SEQ ID NO: 127 - DNA, 324 bp, Homo sapiens
gacatccagatgacccagtctccatcctccctgtctgcatctgtaggagaccgtgtcaccatcacttgccgggcaagtcagaatattgataggaggttactttggtaccagcagaaaccagggaaagcccctaagctcctgatctatggttcttccaagttgcaaagtggggtcccatcacgtttcagtggcagtggatctgggacagatttcactctcaccatcagcagtctgcaacctgaagattttgctacgtactactgtcaacagaggatttatgatcctcatacgttcggccaagggaccaaggtggaaatcaaacgg

SEQ ID NO: 128 - DNA, 324 bp, Homo sapiens
gacatccagatgacccagtctccatcctccctgtctgcatctgtaggagaccgtgtcaccatcacttgccgggcaagtcagtctatttcgaagaatttactttggtatcagcagaaaccagggaaagcccctaagctcctgatctatcattcttcctttttgcaaagtggggtcccatcacgttttagtggcagtggatctgggacagatttcactctcaccatcagcagtctgcaacctgaagattttgctacgtactactgtcaacagcgttttcggtatcctcatacgttcggccaagggaccaaggtggaaatcaaacgg

SEQ ID NO: 129 - DNA, 324 bp, Homo sapiens
gacatccagatgacccagtctccatcctccctgtctgcatctgtaggagaccgtgtcaccatcacttgccgagcaagtcagaggatttctacgtatttaaattggtaccagcagaaaccagggaaagcccctaagctcctgatctatcgtagttccatgttgcaaagtggggtcccatcacgtttcagtggcagtggatctgggacagatttcactctcaccatcagcagtctgcaacctgaagatttcgctacgtactactgtcaacagtattctttttctcctcttacgttcggccaagggaccaaggtggaaatcaaacgg

SEQ ID NO: 130 - DNA, 324 bp, Homo sapiens
gacatccagatgacccagtctccatcctccctgtctgcatctgtaggagaccgtgtcaccatcacttgccgggcaagtcagaatattaagaggtatttatattggtaccagcagaaaccagggaaagcccctaagctcctgatctatcatatttccactttgcaaagtggggtcccatcacgtttcagtggcagtggatctgggacagatttcactctcaccatcagcagtctgcaacctgaagattttgctacgtactactgtcaacagtcttttcgggctcctattacgttcggccaagggaccaaggtggaaatcaaacgg

SEQ ID NO: 131 - DNA, 324 bp, Homo sapiens
gacatccagatgacccagtctccatcctccctgtctgcatctgtaggagaccgtgtcaccatcacttgccgggcaagtcagactattcgtaagaggttacattggtaccagcagaaaccagggaaagcccctaagctcctgatctatcatgcgtccaagttgcaaagtggggtcccatcacgtttcagtggcagtggatctgggacagatttcactctcaccatcagcagtctgcaacctgaagattttgctacgtactactgtcaacagcgttctgatcctccttatacgttcggccaagggaccaaggtggaaatcaaacgg

SEQ ID NO: 132 - DNA, 322 bp, Homo sapiens
gacatccagatgacccagtctccatcctccctgtctgcatctgtaggagaccgtgtcaccatcacttgccgggcaagtcagaatattcgtaggtatttagtttggtatcagcagaaaccagggaaagcccctaagctcctgatctataatgcgtcccatttgcaaagtggggtcccatcacgtttcagtggcagtggatctgggacagatttcactctcaccatcagcagtctgcaacctgaagattttgctacgtactactgtcaacagatttatctttctccttttacgttcggccaagggaccaaggtggaaatcaaac

SEQ ID NO: 133 - DNA, 324 bp, Homo sapiens
gacatccagatgacccagtccccatcctccctgtctgcatctgtaggagaccgtgtcaccatcacttgccgggcaagtcaggagattaggaagcggttaaggtggtaccagcagaaaccagggaaagcccctaagctcctgatctatcgggcttccactttgcaaagtggggtcccatcacgtttcagtggcagtggatctgggacagatttcactctcaccattagcagtctgcaacctgaagattttgctacgtactactgtcaacagctttttcagtcgccttggacgttcggccaagggaccaaggtagaaatcaaacgg

SEQ ID NO: 134 - DNA, 324 bp, Homo sapiens
gacatccagatgacccagtctccatcctccctgtctgcatctgtaggagaccgtgtcaccatcacttgccgggcaagtcaggagattcataagcgtttactttggtaccagcagaaaccagggaaagcccctaagctcctgatctatagtggttccactttgcaaagtggggtcccatcacgtttcagtggcagtggatctgggacagatttcactctcaccatcagcagtctgcaacctgaagattttgctacgtactactgtcaacagcgttatctgcagcctcatacgttcggccaagggaccaaggtggaaatcaaacgg

SEQ ID NO: 135 - DNA, 324 bp, Homo sapiens
gacatccagatgacccagtctccatcctccctgtctgcatctgtaggagaccgtgtcaccatcacttgccgggcaagtcagcatattggtcgtaggttactgtggtaccagcagaaaccagggaaagcccctaagctcctgatctattatagttccaagttgcaaagtggggtcccatcacgtttcagtggcagtggatctgggacagatttcactctcaccatcagcagcctgcaacctgaagattttgctacgtactactgtcaacagcggactattcagcctcatacgttcggccaagggaccaaggtggaaatcaaacgg

SEQ ID NO: 136 - DNA, 324 bp, Homo sapiens
gacatccagatgacccagtctccatcctccctgtctgcatctgtaggagaccgtgtcaccatcacttgccgggcaagtcagtctatttttaagcggttacggtggtaccagcagaaaccagggaaagcccctaagctcctgatctatgcttcttccgtgttgcaaagtggggtcccatcacgtttcagtggcagtggatctgggacagatttcactctcaccatcagcagtctgcaacctgaagattttgctacgtactactgtcaacagaatgttgctattccttttacgttcggccaagggaccaaggtggaaatcaaacgg

SEQ ID NO: 137 - DNA, 324 bp, Homo sapiens
gacatccagatgacccagtctccatcctccctgtctgcatctgtaggagaccgtgtcaccatcacttgccgggcaagtcaaccgattggtcatcggttacgttggtatcagcagaaaccagggaaagcccctaagctcctgatctatcgggcgtccaagttgcaaagtggggtcccatcacgtttcagtggcagtggatctgggacagatttcactctcaccatcagcagtctgcaacctgaagattttgctacgtactactgtcaacagctttataagcagcctttgacgttcggccaagggaccaaggtggaaatcaaacgg

SEQ ID NO: 138 - DNA, 324 bp, Homo sapiens
gacatccagatgacccagtctccatcctccctgtctgcatctgtaggagaccgtgtcaccatcacttgccgggcaagtcagtggattaatgataggttatcttggtatcagcagaaaccagggaaagcccctaagctcctgatctatcgtaagtccggtttgcaaagtggggtcccatcacgtttcagtggcagtggatctgggacagatttcactctcaccatcagcagtctgcaacctgaagattttgctacgtactactgtcaacagtttcggaatattccttttacgttcggccaagggaccaaggtggaaatcaaacgg

SEQ ID NO: 139 - DNA, 324 bp, Homo sapiens
gacatccagatgacccagtctccatcctccctgtctgcatctgtaggagaccgtgtcaccatcacttgccgggcaagtcaggatattggtacggcgttattgtggtaccagcagaaaccagggaaagaccctaggctcctgatctataggggttcccatttgcaaagtggggtcccatcacgtttcagtggcagtggatctgggacagatttcactctcaccatcagcagtctgcaacctgaagattttgctacgtactcctgtcaacagtatcggtatgagcctatgacgttcggccaagggaccaaggtggaaatcaaacgg

SEQ ID NO: 140 - DNA, 324 bp, Homo sapiens
gacatccagatgacccagtctccatcctccctgtctgcatccgtaggagaccgtgtcaccatcacttgccgggcaagtcagcctattagtaggaggttattgtggtatcagcagaaaccagggaaagcccctaagctcctgatctatggtgcttccaggttgcaaagtggggttccatcacgtttcagtggcagtggatctgggacagatttcactctcaccatcagcagtctgcaacctgaagatttagctacgtactactgtcaacagagggagacgaatcctcatacgttcggccaagggaccaaggtggaaatcaaacgg

SEQ ID NO: 141 - DNA, 324 bp, Homo sapiens
gacatccagatggcccagtctccatcctccctgtctgcatctgtaggagaccgtgtcaccatcacttgccgggcaagtcaggttattggtaaggagttagcttggtaccagcagaaaccagggaaagcccctaagctcctgatctatcatgtgtcccggttgcgaagtggggtcccatcacgtttcagtggcagtggatctgggacagatttcactctcaccatcagcagtctacaacctgaagattctgctacgtactactgtcaacagaaggttgcttatccttttacgttcggccaagggaccaaggtggaaatcaaacgg

SEQ ID NO: 142 - DNA, 324 bp, Homo sapiens
gacatccagatgacccagtctccatcctccctgtctgcatctgtaggagaccgtgtcaccatcacttgccgggcaagtcaggatattgttgataggttatcttggtatcagcagaaaccggggaaagcccctaagctcctgatctatcggtcgtcccggttgcgaagtggggtcccatcacgtttcagtggcagtggatctgggacagatttcactctcaccatcagcagtctgcaacctgaagattttgctacgtactactgtcaacagcgtcttcgttttcctattacgttcggccaagggaccaaggtggaaatcaaacgg

SEQ ID NO: 143 - DNA, 324 bp, Homo sapiens
gacatccagatgacccagtctccatcctccctgtctgcatctgtaggagaccgtgtcaccatcacttgccgggcaagtcaggcgatttggcgttctttaaattggtaccagcagaagccagggaaagcccctaagctcctgatctatcggtcgtcccgtttgcaaagtggggtcccatcacgtttcagtggcagtggatctgggacagatttcactctcaccatcagcagtctgcaacctgaagatttcgctacgtactactgtcaacagtattctaatcggccttatacgttcggccaagggaccaaggtggaaatcaaacgg

SEQ ID NO: 144 - DNA, 324 bp, Homo sapiens
gacatccagatgactcagtctccatcctccctgtctgcatctgtaggagaccgtgtcaccatcacttgccgggcaagtcagaagattgggcagcatttacattggtaccagcagaaaccagggaaagcccctaagctcctgatctatcgtacttccattttgcaaagtggggtcccatcacgtttcagtggcagtggatctgggacagatttcactctcaccatcagcagtctgcaacctgaagattttgctacgtactactgtcaacagaatcataggcgtccttatacgttcggccaagggaccaaggtggaaatcaaacgg

SEQ ID NO: 145 - DNA, 324 bp, Homo sapiens
gacatccagatgacccagtctccatcctccctgtctgcatctgtaggagaccgtgtcaccatcacttgccgggcaagtcagaatattgataggaggttactttggtaccagcagaaaccagggaaagcccctaagctcctgatctatggttcttccaagttgcaaagtggggtcccatcacgtttcagtggcagtcgatctgggacagatttcactctcaccatcagcagtctgcaacctgaagattttgctacgtactactgtcaacagaggatttatgatcctcatacgttcggccaagggaccaaggtggaaatcaaacgg

SEQ ID NO: 146 - DNA, 324 bp, Homo sapiens
gacatccagatgacccagtctccatcctccctgtctgcatctgtaggagaccgtgtcaccatcacttgccgggcaagtcaggatattggtgataggttacggtggtatcagcagaaaccagggaaagcccctaagctcctgatctatcatgggtccaggttggaaagtggggtcccatcacgtttcagtggcagtagatctgggacagatttcactcttaccatcagcagtctgcaacctgaagattttgctacgtactactgtcaacagcagtggtttcgtccttatacgttcggccaagggaccaaggtggaaatcaaacgg

SEQ ID NO: 147 - DNA, 324 bp, Homo sapiens
gacatccagatgacccagtctccatcctccctgtctgcatctgtaggagaccgtgtcaccatcacttgccgggcaagtcaggatattgggaggaatttattgtggtaccagcagaaaccagggaaagcccctaggctcctgatctattatagttcccggttgcaaagtggggtcccatcacgttttagtggcagtggatctgggacagatttcactctcaccatcagcagtctgcaacctgaagatttagctacgtactactgtcaacagcgttcgcgtaatccttttacgttcggccaagggaccaaggtggaaatcaaacgg

SEQ ID NO: 148 - DNA, 324 bp, Homo sapiens
gacatccaaatgacccagtctccatcctccctgtctgcatctgtaggagaccgtgtcaccatcacttgccgggcaagtcaggatattggtgggaggttacattggtaccagcagaaaccagggaaagcccctaagctcctgatctatcaggcttccaagttgcaaagtggggtcccatcacgtttcagtggcagtggatctgggacagatttcactctcaccatcagcagtctgcaacctgaagactttgctacgtactactgtcaacagaagcggcggcagcctcatacgttcggccaagggaccaaggtggaaatcaaacgg

SEQ ID NO: 149 - DNA, 324 bp, Homo sapiens
gacatccagatgacccagtctccatcctccctgtctgcatctgtaggagaccgtgtcaccatcacttgccgggcaagtcagtcgattgataggcgtttagggtggtaccagcagaaaccagggaaagcccctaagctcctgatctcgggttcttccaggttgcaaagtggggtcccatcacgtttcagtggcagtggatctgggacagatttcactctcaccatcagcagtctgcaacctgaagattttgctacgtactactgtgtgcagcggcagcgtctgccttatacgttcggccaagggaccaaggtggaaatcaaacgg

SEQ ID NO: 150 - DNA, 324 bp, Homo sapiens
gacatccagatgacccagtctccatcctccctgtctgcatctgtaggagaccgtgtcaccatcacttgccgggcaagtcagttgattaataggcgtttatcgtggtaccagcagaaaccagggaaaccccctaagctcctgatctatcatcattccaggttgcaaagtggggtcccatcacgtttcagtggcagtggatctgggacagatttcactctcaccatcagcagtctgcaacctgaagattttgctacgtactactgtcaacagacgcgtattaggcctcatacgttcggccaagggaccaaggtggaaatcaaacgg

SEQ ID NO: 151 - DNA, 322 bp, Homo sapiens
gacatccagatgacccagtctccatcctccctgtctgcatctgtaggagaccgtgtcaccatcacttgccgggcaagtcagagtattgggcgttatatatattggtaccagcagaaaccagggaaagcccctaagctcctgatctataatgtttcctatttgcaaagtggggtcccatcacgtttcagtggcagtggatctgggacagatttcactctcaccatcagcagtctgcaacctgaagattttgctacgtactactgtcaacagtgttttcgggggccttgtacgttcggccaagggaccaaggtggaaatcaaac

SEQ ID NO: 152 - DNA, 324 bp, Homo sapiens
gacatccagatgacccagtctccatcctccctgtctgcatctgtaggagaccgtgtcaccatcacttgccgggcaagtcagcctattcagggttggttaaattggtatcagcagaaaccagggaaagcccctaagctcctgatctattattcttccctgttgcaaagtggggtcccatcacgtttcagaggcagtggatctgggacagatttcactctcaccatcagcagtctgcaacctgaagatttcgctacgtactactgtcaacagagggaggtgaagccttttacgttcggccaagggaccaaggtggaaatcaaacgg

SEQ ID NO: 153 - DNA, 323 bp, Homo sapiens
gacatccagatgacccagtctccatcctccctgtctgcatctgtaggagaccgtgtcaccatcacttgccgggcaagtcagcggattagtcatgcgttacggtggtatcagcagaaaccagggaaagcccctaagctcctgatctatcgtgcttccgctttgcaaagtggggtcccatcacgtttcagtggcagtggatctgggacagatttcactctcaccatcagcagtctgcaacctgaagattttgctacgtactactgtcaacagaatcgttcggtgccttttacgttcggccaagggaccaaggtggaaatcagacg

SEQ ID NO: 154 - DNA, 324 bp, Homo sapiens; identical to SEQ ID NO: 148

SEQ ID NO: 155 - DNA, 324 bp, Homo sapiens; identical to SEQ ID NO: 148

SEQ ID NO: 156 - DNA, 324 bp, Homo sapiens
gacatccagatgacccagtctccatcctccctgtctgcatctgtaggagaccgtgtcaccatcacttgccgggcaagtcaggagattgataggaggttactgtggtatcagcagaaaccagggaaagcccctaagctcctgatctattctgcttccaggttgcaaagtggggtcccatcacgtttcagtggcagtggatctgggacagatttcactctcaccatcagcagtctgcaacctgaagattttgctacgtactactgtcaacagcggtatcatatgcctcatacgttcggccaagggaccaaggtgaaaatcaaacgg

SEQ ID NO: 157 - DNA, 324 bp, Homo sapiens
gacatccagatgacccagtctccatcctccctgtctgcatctgtaggagaccgtgtcaccatcacttgccgggcaagtcagaagattgggaagcggttacgttggtaccagcagaaaccagggaaagcccctaagctcctgatctatggggcttccaggttgcaaagtggggtcccatcacgtttcagtggcagtggatctgggacagatttcactctcaccatcagcagtctgcaacctgaagattttgctacatactactgtcaacagaatttggagcggcctaatacgttcggccaagggaccaaggtggaaatcaaacgg

SEQ ID NO: 158 - DNA, 324 bp, Homo sapiens
gacatccagatgacccagtctccatcctccctgtctgcatctgtaggagaccgtgtcaccatcacttgccgggcaagtcagccgattgggagtaggatactgtggtaccagcagaaaccagggagagcccctaagctcctgatctatcatgcttccaagttgcaaagtggggtcccatcacgtttcagtggcagtggatctgggacagatttcactctcaccatcagcagtctgcaacctgaagattttgctacgtactactgtcaacagcgtaagtatcagcctcatacgttcggccaaggaaccaaggtggaaatcaaacgg

SEQ ID NO: 159 - protein, 108 aa, Homo sapiens
DIQMTQSPSSLSASVGDRVTITCRASHNIDSRLSWYQQKPGKAPKLLIYRASVLQSGVPSRFRGSGSGTDFTLTISSLQPEDFATYYCQQWDMFPLSFGQGTKVEIKR

SEQ ID NO: 160 - protein, 108 aa, Homo sapiens
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLMFGQGTKVEIKR

SEQ ID NO: 161 - protein, 108 aa, Homo sapiens
DIQMTQSPSSLSASVGDRVTITCRASQNIDSRLSWYQQKPGKAPKLLIYRTSVLQSGVPSRFSGSGSGTVFTLTISSLQPEDFATYYCQQWDMFPLAFGKGTKVEIKR

SEQ ID NO: 162 - protein, 108 aa, Homo sapiens
DIQVTQSPSSLSASVGDRVTITCRASQNIDSRLSWYQQKPGKAPKLLIYRATVLQSGVPSRFSGSGSGTDFTLTISSLQPEDFATYYCQQWDMFPLTFGQGTKVEIKR

SEQ ID NO: 163 - protein, 108 aa, Homo sapiens
DIQMTQSPSSLSASVGDRVTITCRASQNIDSRLSWYQQKPGKAPKLLIYRASVLQSGVPSRFSGSGSGTDFTLTISSLQPEDFATYYCQQWDMFPLSFGHGTKVEIKR

SEQ ID NO: 164 - protein, 108 aa, Homo sapiens
DIQMTQSPSSLSASVGDRVTITCRASQNIDSRLSWYQQKPGKAPKLLIYRTSVLQSGVPTRFSGSGSGTDFTLTISSLQPEDFATYYCQQWDMFPLTFGQGTKVEIKR

SEQ ID NO: 165 - protein, 108 aa, Homo sapiens; identical to SEQ ID NO: 160

SEQ ID NO: 166 - protein, 108 aa, Homo sapiens
DIQMTQSPSSLSASVGDRVTITCRASQNIDSRLSWYQQKPGKAPKLLVYRASVLQSGVPSRFSGSGSGTDFTLTISSLQPEDFATYHCQQWDMFPLTLGQGTKVEIKR

SEQ ID NO: 167 - protein, 108 aa, Homo sapiens
DIQMTQSPSSLSASVGDRVTITCRASQNIDSRLSWYQQKPGKAPKLLIYRASVLQSGVPSRFSGSGSGTDFTLTISSLQPEDFATYYCQQWDMFPLAFGQGTKVEIKR

SEQ ID NO: 168 - protein, 108 aa, Homo sapiens
DIQMTQSPSSLSASVGDRVTITCRASQNIDSRLSWYQQKPGKAPKLLIYRASVLQSGVPSRFSGTGSGTDFTLTISSLQPEDFATYYCQQWDMFPLSFGQGTKVEIKR

SEQ ID NO: 169 - protein, 108 aa, Homo sapiens
DIQMTQSPSSLSASVGDRVTITCRASQNIDSRLSWYQEKPGKAPKLLIYRASVLQSGVSSRFSGSGSGTDFTLTISSLQPEDFATYYCQQWDMFPLTFGRGTKVEIKR

SEQ ID NO: 170 - protein, 108 aa, Homo sapiens
DIQMTQSPSSLSASVGDRVTITCRASQNIDSRLSWYQQKPGKDPKLLIYRSSVLQSGVPSRFSGSGSGTDFTLTISSLQPEDFATYYCQQWDMFPLTFGQGTKVEIKR

SEQ ID NO: 171 - protein, 108 aa, Homo sapiens
DIQTTQSPSSLSASVGDRVTITCRASQNIDSRLSWYQQKPGKAPKLLIYRSSVLQSGVPSRFSGSGSGTDFTLTISSLQPEDFATYYCQQWDMFPLMFGQGTKVEIKR

SEQ ID NO: 172 - protein, 108 aa, Homo sapiens
DIQMTQSPSSLSASVGDRVTITCRASQNIDSRLSWYQQKPGKAPKLLIYRSSILQSGVPSRFSGSGSGTDFTLTISSLQPEDFATYHCQQWDMFPLTFGQGTKVEIKR

SEQ ID NO: 173 - protein, 108 aa, Homo sapiens
DIQMTQSPSSLSASVGDRVTITCRASQNIDSRLSWYQQKPGKDPKLLIYRASVLQSGVPSRFSGSGSGTDFTLTISSLQPEDFATYYCQQWDMFPLTFSQGTKVEIKR

SEQ ID NO: 174 - protein, 108 aa, Homo sapiens
DIQMTQSPSSLSASVGDRVTITCRASQNIDSRLSWYQQKPGKAPKLLIYRASVLQSGVPSRFSGSGSGTDFTLTISSLQPEDFATYYCQQWDMFPLAFGQGTRVEIKR

SEQ ID NO: 175 - protein, 108 aa, Homo sapiens
DIQMTQSPSSLSASVGDRVTITCRASRNIDSRLSWYQQKPGKAPKLLIYRTSVLQSGVPSRFSGSGSGTDFTLTISSLQPEDFATYYCQQWDMFPLMFGQGTKVEIKR

SEQ ID NO: 176 - protein, 108 aa, Homo sapiens
DIQMTQSPSSLSASVGDRVTITCRASQNIDSRLSWYQEKPGKAPKLLIYRTSVLQSGVPSRFSGSGSGTDFTLTISSLQPEDFATYYCQQWDMFPLTFGQGTRVEIKR

SEQ ID NO: 177 - protein, 108 aa, Homo sapiens
DIQMTQSPSSLSASVGDRVTITCRASQNIDSRLSWYQQKPGKAPKLLIYRASVLQSGVPSRFSGSGSGTDFTLTISSLQPEDFATYHCQQWDMFPLTFGQGTKVEIKR

SEQ ID NO: 178 - protein, 108 aa, Homo sapiens
DIQMTQSPSSLSASVGDRVTITCRASQNIDSRLSWYQQKPGKAPKLLIYRTSVLQSGVPSRFSGSGSGTEFTLTISSLQPEDFATYYCQRWDMFPLTFGQGTKVEIKR

SEQ ID NO: 179 - protein, 108 aa, Homo sapiens
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGHGTKVEIKR

SEQ ID NO: 180 - protein, 108 aa, Homo sapiens
DIQMTQSPSSLSASVGDRVTITCRASLKIENDLAWYQQKPGKAPKLLIYYTSILQSGVPSRFSGSGSGTDFTLTISSLQPEDFATYYCQQRRYAPATFGQGTKVEIKR

SEQ ID NO: 181 - protein, 108 aa, Homo sapiens
DIQMTQSPSSLSASVGDRVTITCRTSQKIENDLAWYQQKPGKAPKLLIYYTSILQSGVPSRFSGSGSGTDFTLTISSLQPEDFATYYCQQRRYVPATFGQGTKVEIKR

SEQ ID NO: 182 - protein, 108 aa, Homo sapiens
DIQMTQSPSSLSASVGDRVTITCRASQKIENDLAWYQQKPGNAPKLLIYYTSILHSGVPSRFSGSGSGTDFTLTISSLQPEDFATYYCQQRRYVPATFGQGTKVEIKR

SEQ ID NO: 183 - protein, 108 aa, Homo sapiens
DIQMTQSPSSLSASVGDRVTITCRTSQKIENDLAWYQQRPGKAPKLLIYYTSILQSGVPSRFSGSGSGTDFTLTISSLQPEDFATYYCQQRRYVPATFGQGTKVEIKR

SEQ ID NO: 184 - protein, 108 aa, Homo sapiens
DIQMTQSPSSLSASVGDRVTITCRASQKIENDLAWYQQKPGKAPKLLIYYTSILQSGIPSRFSGSGSGTDFTLTISSLQPEDFATYYCQQRRYAPATFGQGTKVEIKR

SEQ ID NO: 185 - protein, 108 aa, Homo sapiens
DIQMTQSPSSLSASVGDRVTITCRASQKIENDLAWYQQKPGKAPKLLIYYTSILQSGVPSRFSGSGSGTDFTLTISSLQPEDFATYYCQQRRYAPATFGQGTKVEIKR

SEQ ID NO: 186 - protein, 108 aa, Homo sapiens
DIQMTQSPSSLSASVGDRVTITCRASQKIENDLAWYQQKPGKAPKLLIYYTSILQSGVPSRFSGSGSGTDFTLTISSLQPEDFATYYCQQRRYVPASFGQGTKVEIKR

SEQ ID NO: 187 - protein, 108 aa, Homo sapiens; identical to SEQ ID NO: 185

SEQ ID NO: 188 - protein, 108 aa, Homo sapiens
DIQMTQSPSSLSASVGDRVTITCRASQKIENDLAWYQQKPGKAPKLLIYYTSILQRGVPSRFSGSGSGTDFTLTISSLQPEDFATYYCQQRRYAPATFGQGTKVEIKR

SEQ ID NO: 189 - protein, 108 aa, Homo sapiens
DIQMTQSPSSLSASVGDRVTITCRTSQKIENDLAWYQQKPGKAPKLLIYYTSILQSGVPSRFSGSGSGTDFTLTISSLQPEDFATYYCQQRRYAPATFGQGTKVEIKR

SEQ ID NO: 190 - protein, 108 aa, Homo sapiens
DIQMTQSPSSLSASVGDRVTITCRANQKIENDLAWYQQKPGKAPKLLIYYTSILQSGVPSRFRGSGSGTDFTLTISSLQPEDFATYYCQQRRYVPATFGQGTKVEIKR

SEQ ID NO: 191 - protein, 108 aa, Homo sapiens
DIQMTQSPSSLSASVGDRVTITCRASQKIENDLAWYQQKPGKAPKLLIYYTSILHSGVPSRFSGSGSGTDFTLTISSLQPEDFATYYCQQRRYVPATFGQGTKVEIKR

SEQ ID NO: 192 - protein, 108 aa, Homo sapiens
DIQMTQSPSSLSASVGDRVTITCQASKKIENDLAWYQQKPGKAPKLLIYYTSILQSGVPSRFSGSGSGTDFTLTISSLQPEDFATYYCQQRRYVPATFGQGTKVEIKR

SEQ ID NO: 193 - protein, 108 aa, Homo sapiens
DIQLTQSPSSLSASVGDRVTITCRASQKIENDLAWYQQKPGKAPKLLIYYTSILQRGVPSRFSGSGSGTDFTLTISSLQPEDIATYYCQQRRYVPATFGQGTKVEIKR

SEQ ID NO: 194 - protein, 108 aa, Homo sapiens
DIQMTQSPSSLSASVGDRVTITCRASQKIENDLAWYQQKPGKAPKLLIYYTSILQSGVPSRFIGSGSGTDFTLTISSLQPEDFATYYCQQRRYVPATFGPGTKVEIKR

SEQ ID NO: 195 - protein, 108 aa, Homo sapiens
DIQMTQAPSSLSASVGDRVTITCRSSQKIENDLAWYQQKPGKAPKLLIYYTSILQSGVPSRFSGSGSGTDFTLTISSLQPEDFATYYCQQRRYVPATFGQGTKVEIKR

SEQ ID NO: 196 - DNA, 324 bp, Homo sapiens
gacatccagatgacccagtccccatcctccctgtctgcatctgtaggagaccgtgtcaccatcacttgccgggcaagtcagcggattcataataggttatcttggtaccagcagaaaccagggaaagcccctaagctcctgatctatgcggcgtccaaattgcaaagtggggtcccatcacgtttcagtggcagtggatctgggacagatttcactctcaccatcagcagtctgcaacctgaagattttgctacgtactactgtcaacagactagttataggcctcatacgttcggccaagggaccaaggtggaaatcaaacgg

SEQ ID NO: 197 - DNA, 324 bp, Homo sapiens
gacatccagatgacccagtctccatcctccctgtctgcatctgtaggagaccgtgtcaccatcacttgccgggcaagtcagaatattaatgagcgtttattgtggtaccagcagaaaccagggaaagcccctacgctcctgatctatcattcgtcccggttgcaaagtggggtcccatcacgtttcagtggcagtggatctgggacagatttcactctcaccatcagcagtctgcaacctgaagattttgctacgtactactgtcaacagaagtataagcgtccttatacgttcggccaagggaccaaggtggaaatcaaacgg

SEQ ID NO: 198 - DNA, 324 bp, Homo sapiens
gacatccagatgacccagtctccatcctccctgtctgcatctgtaggagaccgtgtcaccatcacttgccgggcaagtcagatgattgggaagcggttaaggtggtaccagcagaaaccagggaaagcccctaagctcctgatctattttgcttcccggttgcaaagtggggtcccatcacgtttcagtggcagtggatctgggacagatttcactctcaccatcagcagtctgcaacctgaagattttgctacgtactactgtcaacagtctaggcagcatcctcatacgttcggccaagggaccaaggtggaaatcaaacgg

SEQ ID NO: 199 - DNA, 324 bp, Homo sapiens
gacatccagatgacccagtctccatcctccctgtctgcatctgtaggagaccgtgtcaccatcacttgccgggcaagtcagaatattgggcggaagttaaggtggtatcagcagaaaccagggaaagcccctaagctcctgatctatgggacgtcccgtttgcaaagtggggtcccatcacgtttcagtggcagtggatctgggacagatttcactctcaccatcagcagtctgcaacctgaagattttgctacgtactactgtcaacagaatttgcatctgccttctacgttcggccaagggaccaaggtggaaatcaaacgg

SEQ ID NO: 200 - DNA, 324 bp, Homo sapiens
gacatccagatgacccagtctccatcctccctgtctgcatctgtaggagaccgtgtcaccatcacttgccgggcaagtcaggatattgagcggcgtttactgtggtatcagcagaaaccagggaaagcccctaagctcctgatctattcgacgtcccgtttgcaaagtggggtcccatcacgtttcagtggcagtggatctgggacagatttcactctcaccataagcagtctgcaacctgaagattttgctacgtactactgtcaacagaggcatacgtcgcctcatacgttcggccaagggaccaaggtggaaatcaaacgg

SEQ ID NO: 201 - DNA, 324 bp, Homo sapiens
gacatccagatgacccagtctccatcctccctgtctgcatctgtaggagaccgtgtcaccatcacctgccgggcaagtcagaatattactaatcggttacggtggtaccagcagaaaccagggaaagcccctaagctcctgatctatcgtagttccgttttgcaaagtggggtcccatcacgtttcagtggcggtggatctgggacagatttcactctcaccatcagcagtctgcaacctgaagattttgctacgtactactgtcaacagcataattatcagcctcatacgttcggccaagggaccaaggtggaaatcaaacgg

SEQ ID NO: 202 - DNA, 324 bp, Homo sapiens
gacatccagatgacccagtctccatcctccctgtctgcatctgtaggagaccgtgtcaccatcacttgccgggcaagtcagtcgattgggaggggtttagcgtggtatcagcagaaaccagggaaagcccctaagctcctgatctatatggggtcccgtttgcaaagtggggtcccatcacgttttagtggcagtggatctgggacagatttcactctcaccatcagcagtctgcaacctgaagatttcgctacgtactactgtcaacagcagaggcatcttcctcggacgttcggccaagggaccaaggtggaaatcaaacgg

SEQ ID NO: 203 - DNA, 324 bp, Homo sapiens
  misc_feature 322: n = A, T, C or G
gacatccagatgacccagtctccatcctccctgtctgcatctgtaggagaccgtgtcaccatcacttgccgggcaagtcggccgatttctactagtttagtttggtaccagcagaaaccagggaaagcccctaagctcctgatctataatgcgtccaatttgcaaagtggggtcccatcacgtttcagtggcagtggatctgggacagatttcactctcaccatcagcagtctgcaacctgaagattttgctacgtactactgtcaacagtcgcagactcttcctgttacgttcggccaagggaccaaggtggaaatcaaangg

SEQ ID NO: 204 - DNA, 324 bp, Homo sapiens
gacatccagatgacccagtctccatcctccctgtctgcatctgtaggagaccgtgtcaccatcacttgccgggcaagtcagagtattgggcggcggttaaattggtatcagcagaaaccagggaaagcccctaagctcctgatctatcggacgtccacgttgcaaagtggggtcccgtcacgtttcagtggcagtggatctgggacagatttcactctcaccatcagcagtctgcaacctgaagattttgctacgtactactgtcaacagacttctcgtgtgccttatacgttcggccaagggaccaaggtggaaatcaaacgg

SEQ ID NO: 205 - DNA, 324 bp, Homo sapiens
gacatccagatgacccagtctccatcctccctgtctgcatctgtaggagaccgtgttaccatcacttgccgggcaagtcaggatattaagaagcatttattgtggtaccagcagagaccagggaaagcccctaagctcctgatctattatagttcccgtttgcaaagtggggtcccatcacgtttcagtggcagtggatctgggacagatttcactctcaccatcagcagtctgcaacctgaagattttgctacgtactactgtcaacagcggcatcatgatccttttacgttcggccaagggaccaaggtggaaatcaaacgg

SEQ ID NO: 206 - DNA, 324 bp, Homo sapiens
gacatccagatgacccagtctccatcctccctgtctgcatctgtaggagaccgtgtcaccatcacttgccgggcaagtcagtctattgatcggaggttactttggtatcagcagaaaccagggaaagcccctaagctcctgatctatagggcttccaggttgcaaagtggggtcccatcacgtttcagtggcagtggatctgggacagatttcactctcaccatcagcagtctgcaacctgaagattttgctacgtactactgtcaacagacttatgcgcggcctaacacgttcggccaagggaccaaggtggaaatcaaacgg

SEQ ID NO: 207 - DNA, 324 bp, Homo sapiens
gacatccagatgacccagtctccatcctccctgtctgcatctgtaggagaccgtgtcaccatcacttgccgggcaagtcagagtattggtccgtggttaagttggtatcagcagaaaccagggaaagcccctaagctcctgatctatcaggtttcccgtctgcaaagtggggtcccatcacgtttcagtggcagtggatctgggacagatttcactctcaccatcagcagtctgcaacctgaagattttgctacgtactactgtcaacagaatcttgcgcctccttatacgttcggccaagggaccaaggtggaaatcaaacgg

SEQ ID NO: 208 - DNA, 324 bp, Homo sapiens
gacatccagatgacccagtctccatcctccctgtctgcatctgtaggagaccgtgtcaccatcacttgccgggcaagtcagggtattatgtatcatttaaggtggtaccagcagaaaccagggaaagcccctaggctcctgatctatcatgggtccactttgcaaagtggggtcccagcacgtttcagtggcagtggatctgggacagattttactctcaccatcagcagtctgcaacctgaagattttgctacgtactactgtcaacagacttggaatgcgcctttgacgttcggccaagggaccaaggtggaaatcaaacgg

SEQ ID NO: 209 - DNA, 324 bp, Homo sapiens
gacatccagatgacccagtctccatcctccctgtctgcatctgtaggagaccgggtcaccatcacttgccgggcaagtcagggtattggtaatagtttacggtggtatcagcagaaaccagggaaagcccctaagctcctgatctattattcttcccatttgcaaagtggggtcccatcacgtttcagtggcagtggatctgggacagatttcactctcaccatcagcagtctgcaacctgaagattttgctacgtactactgtcaacagattaggacgaagccttttacgttcggccaagggaccaaggtggaaatcaaacgg

SEQ ID NO: 210 - DNA, 324 bp, Homo sapiens
gacatccagatgacccagtctccatcctccctgtctgcatctgtaggagaccgtgtcaccatcacttgccgggcaagtcagaagattatgacgcatttacgttggtatcagcagaaaccagggaaagcccctaagctcctgatctatggtgggtcccatttgcaaagtggggtcccatcacgtttcagtggcagtggatctgggacagatttcactctcaccattagcagtctgcaacctgaagattttgctacgtactactgtcaacagacgtgggtgtcgcctatgacgttcggccaagggaccaaggtggaaatcagacgg

SEQ ID NO: 211 - DNA, 324 bp, Homo sapiens
gacatccagatgacccagtctccatcctccctgtctgcatctgtaggagaccgtgtcaccatcacttgccgggcaagtcagtctattgggacgctgttaaattggtatcagcagaaaccagggaaagcccctaagctcctgatctatgcttcttcccgtttgcaaagtggggtcccatcacgtttcagtggcagtggatctgggacagatttcactctcaccattagcagtctgcaacctgaagattttgctacgtactactgtcaacagatgaatagggttcctattacgttcggccaagggaccaaggtggaaatcaaacgg

SEQ ID NO: 212 - DNA, 324 bp, Homo sapiens
gacatccagatgacccagtctccatcctccctgtctgcatctgtaggagaccgtgtcaccatcacttgccgggcaagtcagtctattgggatgctgttatcgtggtaccagcagaaaccagggaaagcccctaagctcctgatctatgctgtgtcccgtttgcaaagtggggtcccatcacgtttcagtggcagtggatctgggacagatttcactctcaccatcagcagtctgcaacctgaagattatgctacgtactactgtcaacagatgcagcgtcctcctattacgttcggccaagggaccaaggtagaaatcaaacgg

SEQ ID NO: 213 - DNA, 324 bp, Homo sapiens
gacatccagatgacccagtctccatcctccctgtctgcatctgtaggagaccgtgtcaccatcacttgccgggcaagtcagccgattaagatgatgttatcgtggtatcagcagaaaccagggaaagcccctaagctcctgatctataataattccactttgcaaagtggggtcccatcacgtttcagtggcagtggatctgggacagatttcactctcaccatcagcagtctgcaacctgaagattttgctacgtactactgtcaacagtataggaggtggccttatacgttcagccaagggactaaggtggaaatcaaacgg

SEQ ID NO: 214 - DNA, 324 bp, Homo sapiens
gacatccagatgacccagtctccatcctccctgtctgcatctgtaggagaccgtgtcaccatcacttgccgggcaagtcaagatattggtaatatgttagcgtggtatcagcagaaaccagggaaagcccctaagcccctgatctattatgcgtcctatttgcaaagtggggtcccatcacgtttcagtggcagtggatctgggacagatttcactctcaccatcagcagtctgcaacctgaagatttcgctacgtactactgtcaacagatgcgtgattatcctgtgacgttcggccaagggaccaaggtggaaatcaaacgg

SEQ ID NO: 215 - DNA, 324 bp, Homo sapiens
gacatccagatgtcccagtctccatcctccctgtctgcatctgtaggagaccgtgtcaccatcacttgccgggcaagtcaagatattggtaatatgttagcgtggtatcagcagaaaccagggaaagcccctaagcccctgatctattatgcgtcctatttgcaaagtggggtcccatcacgtttcagtggcagtggatctgggacagatttcactctcaccatcagcagtctgcaacctgaagattttgctacgtactactgtcaacagctgggtgcgaagcctcatacgttcggccaagggaccaaggtggaaatcaaacgg

SEQ ID NO: 216 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgtcatacgtcgccttttacgttcggccaagggaccaaggtggaaatcaaacgg

SEQ ID NO: 217 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgctacctgaagattttgctacgtactactgtcaacagcgtcatagtgcgccttatacgttcggccaagggaccaaggtggaaatcaaacgg

SEQ ID NO: 218 - DNA, 324 bp, Homo sapiens
gacatccagatgacccagtctccatcctccctgtctgcatctgtaggagaccgtgtcaccatcacttgccgggcaagtcagaatattgggggtcgtttggtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagaggcagcagcagccttatacgttcggccaagggaccaaggtggaaatcaaacgg

SEQ ID NO: 219 - DNA, 324 bp, Homo sapiens
gacatccagatgacccagtctccatcctccctgtctgcatctgtaggagaccgcgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagagggcttctcggccttatacgttcggccaagggaccaaggtggaaatcaaacgg

SEQ ID NO: 220 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgttatgtgcagccttatacgttcggccaagggaccaaggtggaaatcaaacgg

SEQ ID NO: 221 - DNA, 324 bp, Homo sapiens
gacgtccagatgacccagtctccatcctccctgtctgcatctgtaggagaccgtgtcaccatcacttgccgggcaagtcagcttattcgtaagaggttacgttggtaccagcagaaaccagggaaagcccctaagctcctgatctatcattcgtccaagttgcaaagtggggtcccatcacgtttcagtggcagtggatctgggacagatttcactctcaccatcagcagtctgcaacctgaagattttgctacgtactactgtcaacaggggcatagtcggccttttacgttcggccaagggaccaaggtggaaatcaaacgg

SEQ ID NO: 222 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgttataagccgccttatacgttcggccaagggaccaaggtgaaaatcaaacgg

SEQ ID NO: 223 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcgggttagggcgccttatacgttcggccaagggaccaaggtggaaatcaaacgg

SEQ ID NO: 224 - DNA, 324 bp, Homo sapiens
gacatccagatgacccagtctccatcctccctgtctgcatctgtaggagaccgtgtcaccatcacttgccgggcaagtcagaatattggttctaagttagtgtggtatcagcagaaaccagggaaagcctctaagctcctgatctatactccttccaggttgcaaagtggggtcccatcacgtttcagtggcagtggatctgggacagatttcactctcaccatcagcagtctgcaacctgaagatttcgctacgtactactgtcaacagcggtttatgactccttatacgttcggccaagggaccaaggtggaaatcaaacgg

SEQ ID NO: 225 - DNA, 324 bp, Homo sapiens
gacatccagatgacccagactccatcctccctgtctgcatctgtaggagaccgtgtcaccatcacttgccgggcaagtcagaatattgggaagcagttattgtggtaccagcagaaaccagggaaagcccctaggctcctgatctattgtcctcccccgttgcaaagtggggtcccatcacgtttcagttgcagtggatctgggacagatttcactctcaccatcagcagtctgcaacctgaagattttgctacgtactactgtcaacagcatgcttctaggccttttacgttcggccaagggaccaaggtggaaatcaaacgg

SEQ ID NO: 226 - DNA, 324 bp, Homo sapiens
gacatccagatgacccagtctccatcctccctgtctgcatctgtaggagaccgtgtcaccatcacttgccgggcaagtcagaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattctgctacgtactactgtcaacagcgttattcgctgccttttacgttcggccaagggaccaaggtggaaatcaaacgg

SEQ ID NO: 227 - DNA, 324 bp, Homo sapiens
gacatccagatgacccagtctccatcctccctgtctgcatctgtaggagaccgtgtcaccatcagttgccgggcaagtcagaatattggtacgcagttacattggtatcagcagaaaccagggaaagcccctaggctcctgatctatggtagttcctttttgcaaagtggggtcccatcacgtttcagtggcagtggatctgggacagatttcactctcaccatcagcagtctgcaacctgaagattttgctacgtactactgtcaacaggttatgttggggcctacgacgttcggccaagggaccaaggtggaaatcaaacgg

SEQ ID NO: 228 - DNA, 324 bp, Homo sapiens
gacatccagatgacccagtctccatcctccctgtctgcatctgtaggagaccgtgtcaccatcacttgccgggcaagtcagaatattcatgggatgttaaggtggtaccagcaaaaaccagggaaagcccctaagctcctgatctatacgccgtccccttcccaaagtggggtcccatcacgtttcagtggcagtggatctggcacagatttcactctcaccatcagcagtctgcaacctgaagattttgctacgtactactgtcaacagactgctacttggccttttacgttcggccaagggaccaaggtggaaatcaaacgg

SEQ ID NO: 229 - DNA, 324 bp, Homo sapiens
gacatccagatgacccagtcaccatcctccctgtctgcatctgtaggagaccgtgtcaccatcacttgccgagcaagtcagcctattgggaataagttacgttggtaccagcagaaaccagggaaagcccctaagctcctgatctatagtccgtccccgttgcaaagtggggtcccatcacgtttcagtggcagtggatctgggacagattacactctcaccatcagcagtctgcaacctgaagattttgctacgtactactgtcaacagacttggtcttttcctggtacgttcggccaagggaccaaggtggaaatcaaacgg

SEQ ID NO: 230 - DNA, 324 bp, Homo sapiens
  misc_feature 275: n = A, T, C or G
gacatccagatgacccagtctccatcctccctgtctgcatctgtaggagaccgtgtcaccatcacttgccaggcaagtcagcctattgatgggaggttagtttggtaccagcagaaaccagggaaagcctctaagctcctgatctatgttccgtccgggttgcaaagtggggtcccatcacgtttcagtggcagtggatctgggacagatttcactctcaccatcagcagtctgcaacctgaagattttgctacgtactactgtcaacagcggcntactcctccttatacgttcggccaagggaccaaggtggaaatcaaacgg

SEQ ID NO: 231 - DNA, 324 bp, Homo sapiens
gacatccagatgacccagtctccatcctccctgtctgcatctgtaggagaccgtgtcaccatcacttgccgggcaagtcagtctattgggggtcgtttagtgtggtaccagcagaaaccagggaaagcccctaagctcctgatctatactccgccccctttgcaaagtggggtcccatcacgtttcagtggcagtggatctgggacagatttcactctcaccatcagcagtctgcaacctgaagattttgctacgtactactgtcaacagcggtatcttaggccttttacgttcggccaagggaccaaggtggaaatcaaacgg

SEQ ID NO: 232 - DNA, 324 bp, Homo sapiens
gacatccagatgacccagtccccatcctccctgtctgcatctgtaggagaccgtgtcaccatcacttgccgggcaagtcggaatattgggggtcgtttagtgtggtaccagcagaaaccagggaaagcccctaagctcctgatctatactccgtcccctttgcaaagtggggtcccatcacgtttcagtggcagtggatctgggacagatttcactctcaccatcagcagtctgcaacctgaagattttgctacgtactactgtcaacagcggcataatgagccttttacgttcggccaagggaccaaggtggaaatcaaacgg

SEQ ID NO: 233 - DNA, 324 bp, Homo sapiens
  misc_feature 90, 102, 108, 151, 153, 183, 273, 287, 288: n = A, T, C, or G
gacatccagatgacccagtctccatcctccctgtctgcatctgtaggagaccgtgtcaccatcacttgccgggcaagtcagaatattttnactttgttaaantggtancagcagaaaccagggaaagcccctaagctcctgatctatgctncntcccgtttgcaaagtggggtcccatcacgnttcagtggcagtggatctgggacagatttcactctcaccatcagcagtctgcaacctgaagattttgctacgtactactgtcaacaggcntataggcatcctannacgctcggccaagggaccaaggtggaaatcaaacgg

SEQ ID NO: 234 - DNA, 324 bp, Homo sapiens; identical to SEQ ID NO: 156

SEQ ID NO: 235 - DNA, 324 bp, Homo sapiens
gacatccagatgacccagtctccatcctccctgtctgcatctgtgggagaccgtgtcaccatcacttgccgggcaagtcaggatattaagtcgcatttacgttggtatcagcagaaaccagggaaagcccctaagctcctgatctatactccttcctctttgcaaagtggggtcccatcacgtttcagtggcagtggatctgggacagatttcactctcaccatcagcagtctgcaacctgaagattctgctacgtactactgtcaacaggtgttgacggttccttatacgttcggccaagggaccaaggtggaaatcaaacgg

SEQ ID NO: 236 - DNA, 324 bp, Homo sapiens
gacatccagatgacccagtctccatcctccctgtctgcatctgtaggagaccgtgtcaccatcacttgccgggcaagtcaggatattgggcgttggttatcgtggtaccagcagaaaccagggaaagcccctaagctcctgatctatgcgggttcccagttgcaaagtggggtcccatcacgtttcagtggcagtggatctgggacagatttcactctcaccatcagcagtctgcaacctgaagattttgctacgtactactgtcaacggtcgtgggatcctcctacgacgttcggccaagggaccaaggtggaaatcaaacgg

SEQ ID NO: 237 - DNA, 324 bp, Homo sapiens
gacatccagatgacccagtctccatcctccctgtctgcatctgtaggagaccgtgtcaccatcacttgccgggcaagtcagccgattgggagtatgttagtgtggtatcagcagaaaccagggaaagcccctaagctcctgatctatacgccgtcctctttgcaaagtggggtcccatcacgtttcagtggcagtggatctgggacagatttcactctcaccatcagcagtctgcaacctgaagattttgctacgtactactgtcaacagaagtatatggagcctcatacgttcggccaagggaccaaggtggaaatcaaacag

SEQ ID NO: 238 - protein, 108 aa, Homo sapiens
DIQMTQSPSSLSASVGDRVTITCRASQDIGDRLRWYQQNPGKAPKLLIYHGSRLESGVPSRFSGNRSGTDFTLTISSLQPEDFATYYCQQQWFRPYTFGQGTKVEIKR

SEQ ID NO: 239 - protein, 108 aa, Homo sapiens
DIQMTQSPSSLSASVGDRVTITCRASQDIGGRLRWYQQKPGKAPKLLIYHGSRLDSGVPSRFSGSRSGADFTLTISSLQPEDFATYYCQQQWYRPYTFGQGTKVEIKR

SEQ ID NO: 240 - protein, 108 aa, Homo sapiens
DIQMTQSPSSLSASVGDRVTITCRASQDIGDRLRWYQQKPGKAPKLLIYHGSRLESGVPSRFSGSRSGTDFTLTISSLQPEDFATYYCQQQWLRPYTFGQGTRVEIKR

SEQ ID NO: 241 - protein, 108 aa, Homo sapiens
DIQMTQSPSSLSASVGDRVTITCGASQDIGDRLRWYQQKPGKAPKLLIYHGSRLESGVPSRFSGSRSGTDFTLTISSLQPEDFATYYCQQQWFRPYTFGQGTKVEIKR

SEQ ID NO: 242 - protein, 108 aa, Homo sapiens
DIQMTQSPSSLSASVGDRVTITCRASQDIGDRLRWYQQKPGKAPKLLIYHGSRLDSGVPSRFSGSRSGTDYTLTISSLQPEDFATYYCQQQWFRPYTFGQGTKVEIKR

SEQ ID NO: 243 - protein, 108 aa, Homo sapiens
DIQMTQSPSSLSASVGDRVTITCRASQDIGDRLRWYQQKPGRAPKLLIYHGSRLESGVPSRFSGSRSVTDFTLTISSLQPEDFATYYCQQQWFRPYTFGQGTKVEIKR

SEQ ID NO: 244 - protein, 108 aa, Homo sapiens
DIQMTQSPSSLSASVGDRVTITCRASQVIGDRLRWYQQKPGKAPKLLIYHGSRLESGVPSRFSGSRSGTDFTLTISSLQPEDFATYYCQQQWFRPYTFGQGTKVEIKR

SEQ ID NO: 245 - protein, 108 aa, Homo sapiens
DIQMTQSPSSLSASVGDRVTITCRASQDIGDRLRWYQQKPGKAPKLLIYHGSRLESGVPSRFSGNRSGTVFTLTISSLQPEDFATYYCQQQWFRPYTFGQGTKVEIKR

SEQ ID NO: 246 - protein, 108 aa, Homo sapiens
DIQMTQSPSSLSASVGDRVTITCRASQDIGDRLRWYQQKPGKAPKLLIYHGSRLESGVPSRFRGSRSGTDFTLTISSLQPEDFATYYCQQQWFRPYTFGQGTKVEIKR

SEQ ID NO: 247 - protein, 108 aa, Homo sapiens
DIQMTQSPSSLSASVGDRVTITCRASQDIGDRLRWYQQKPGKAPKLLIYHGSRLESGVPSRFSGSRYGTNFTLTISSLQPEDFATYYCQQQWFRPYTFGQGTKVEIKR

SEQ ID NO: 248 - protein, 108 aa, Homo sapiens
DIQMTQSPSSLSASVGDRVTITCRASQDIGDRLRWYQQKPGKAPKLLIYHGSRLESGVPSRFSGSRFGTDFTLTISSLQPDDFATYYCQQQRFRPYTFGQGTKVEIKR

SEQ ID NO: 249 - protein, 108 aa, Homo sapiens
DIQMTQSPSSLSASVGDRVTITCRASQDIGDRLRWYQQKPGKAPKLMIYHGSRLESGVPSRFSGSRSGTDFTLTISSLQPEDFATYYCQQQWFRPYTFGQGTKVEIKR

SEQ ID NO: 250 - protein, 108 aa, Homo sapiens
DIQMTQSPSSLSASVGDRVTITCRASQDIGDRLRWYQQKPGKAPKLLIYHGSRLESGVPSRFSGGRSGTDFTLTISSLQLEDFATYYCQQQWFRPYTFGQGTKVEIKR

SEQ ID NO: 251 - protein, 108 aa, Homo sapiens
DIQMTQSPSSLSASVGDRVTITCRASRDIGDRLRWYQQKPGKAPKLLIYHGSRLESGVPSRFSGSRSGTDFTLTISSLQPEDFATYYCQQQWFRPYTFGQGTKVEIKR

SEQ ID NO: 252 - protein, 108 aa, Homo sapiens
DIQMTQSPSSLSASVGDRVTITCRASQDIGYRLRWYQQKPGKAPMLLIYHGSRLESGVPSRFSGRRSGTDFTLTISSLQPEDFATYYCQQQWFRPYTFGQGTKVEIKR

SEQ ID NO: 253 - protein, 108 aa, Homo sapiens
DIQMTQSPSSLSASVGDRVTITCRASQDIGDRLRWYQQKPGKAPKLLIYHGSRLESGVPSRFSGSRSGTDFTLTISSLLPEDFATYYCQQQRFRPYTFGQGTKVEIKR

SEQ ID NO: 254 - protein, 108 aa, Homo sapiens
DIQMTQSPSSLSASVGDRVSITCRASQDIGDRLRWYQQKPGKAPKLLIYHGSRLESGVPSRFRGSRSGTDFNLTISSLQPGDFATYYCQQQWFRPYTFGQGTKVEIKR

SEQ ID NO: 255 - protein, 108 aa, Homo sapiens
DIQMTQSPSSLSASVGDRVTITCRASQDIGDRLRWYQQKPGKAPKLLIYHGSRLESRVPSRFSGNRSGTDFTLSISSLQPEDFATYYCQQQRFRPYTFGQGTKVEIKR

SEQ ID NO: 256 - protein, 108 aa, Homo sapiens
DIQMTQSPSSLSASVGDRVTITCRASQDIGDRLRWYQQKPGKAPKLMIYHGSRLESGVPSRFSGSRSGTDFTLTISSLQPEDFATYYCQQQWSRPYTFGQGTKVEIKR

SEQ ID NO: 257 - protein, 108 aa, Homo sapiens
DIQMTQSPSSLSASVGDRVAITCRASQDIGDRLRWYHQKPGKAPKLLIYHGSRLESGVPSRFSGSRSGTDFTLTISSLQPEDFATYYCQQQWFRPYTFGQGTKVEIKR

SEQ ID NO: 258 - protein, 108 aa, Homo sapiens
DIQMTQSPSSLSASVGDRITITCRASQDIGDRLRWYQQRPGKAPKLLVYHGSRLESGVPSRFSGSRSGTDFTLTISSLQPEDYATYYCQQQWFRPYTFGQGTKVEIKR

SEQ ID NO: 259 - protein, 108 aa, Homo sapiens
DIQMTQSPSSLSASVGDRVTITCRASQDIGDRLRWYQQKPGKAPKLLIYHGSRLESGVPSRFSGGRSGTDFTLTISSLQPEDSATYYCQQQWLRPYTFGQGTKVEIKR

SEQ ID NO: 260 - protein, 108 aa, Homo sapiens; identical to SEQ ID NO: 249

SEQ ID NO: 261 - protein, 108 aa, Homo sapiens
DIQMTQSPSSLSASVGDRVTITCRASQDIGDRLRWYQQKPGKAPKLMIYHGSRLESGVPPRFSGSRSGTDFTLNISSLQPDDFATYYCQQQWFRPYTFGQGTKVEIKR

SEQ ID NO: 262 - protein, 108 aa, Homo sapiens
DIQMTQSPSSLSASVGDRVTITCRASQDIGDRLRWYQQKPGKAPRLLIYHGSRLESGVPSRFSGSRSGTDFTLTFSSLQPEDFATYYCQQQWIRPYTFGQGTKVEIKR

SEQ ID NO: 263 - protein, 108 aa, Homo sapiens; identical to SEQ ID NO: 251

SEQ ID NO: 264 - protein, 108 aa, Homo sapiens
DIQMTQSPTSLSASVGDRVTITCRASQDIGDRLRWYQQRPGKAPKLLIYHGTRLESGVPSRFSGSRSGTDFTLTISSLQPEDFATYYCQQQWFRPYTFGQGTKVEIKR

SEQ ID NO: 265 - protein, 108 aa, Homo sapiens
DIQMTQSPSSLSASVGDRVTITCRASQDIGDRLRWYQQKPGKAPKLLIYHGSRLESGIPSRFSGSRSGTDFTLTISSLQPEDFATYYCQQQWFRPYTFGQGTKVEIKR

SEQ ID NO: 266 - protein, 108 aa, Homo sapiens
DIQMTQSPSSLSASVGDRVTITCRASQDIDDRLRWYQQKPGKAPKLLIYHGSRLDSGVPSRFSGSRSGTDFTLTISSLQPEDFATYYCQQQWFRPYTFGQGTKVEIKR

SEQ ID NO: 267 - protein, 108 aa, Homo sapiens
DIQMTQSPSSLSASVGDRVTITCRASQDIGDRLRWYQQKPGKAPKLLIYHGSRLDSGVPSRLSGSRSGTDFTLTISSLQPEDFATYYCQQQWFRPYTFGQGTKVEIKR

SEQ ID NO: 268 - protein, 108 aa, Homo sapiens
DIQMTQSPSSLSASVGDRVTITCRASQDIGDRLRWYQQKPGKAPKLLIYHGSRLESGVPSRFSGGRSGTDFTLTIRSLQPEDFATYYCQQQWFRPYTFGQGTKVEIKR

SEQ ID NO: 269 - protein, 108 aa, Homo sapiens
DIQMTQSPSSLSASVGDRVTITCRASRDIGDRLRWYQQKPGKAPKLLIYHGSRLESGVPSRFSGSRSGTDFTLTISRLQPEDFATYYCQQQWFRPYTFGQGTKVEIKR

SEQ ID NO: 270 - protein, 108 aa, Homo sapiens
DIQMTQSPSSLSASVGDRVTITCRASQDIGDRLRWYQQKPGKAPKLLIYHGSRLESGVPSRFSGGRSGTDFTLTISSLQPEDFATYYCQQQRFRPYTFGQGTKVEIKR

SEQ ID NO: 271 - protein, 108 aa, Homo sapiens
DIQMTQSPSSLSATVGDRVTITCRASQDIGDRLRWYQQKTGKAPKLLIYHGSRLESGVPSRFSGSRSGTDFTLTISSLQPEDFATYYCQQQWFRPYTFGQGTKVEIKR

SEQ ID NO: 272 - protein, 108 aa, Homo sapiens
DIQMTQSPSRLSATVGDRVTITCRASQDISDRLRWYQQKPGKAPKLLIYHGSRLESGVPSRFSGSRSGTDFALTISSLQPEDFATYYCQQQWFRPYTFGQGTKVEIKR

SEQ ID NO: 273 - protein, 108 aa, Homo sapiens
DIQMTQSPSSLSASVGDRVTITCRASQDIGDRLRWYQQKPGKAPKLLIYHGSRLESGVPSRFSGSRSGTDFALTISSLQPEDFATYYCQQQWFRPYTFGPGTKVEIKR

SEQ ID NO: 274 - protein, 108 aa, Homo sapiens; identical to SEQ ID NO: 251

SEQ ID NO: 275 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRHTSPFTFGQGTKVEIKR

SEQ ID NO: 276 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLLPEDFATYYCQQRHSAPYTFGQGTKVEIKR

SEQ ID NO: 277 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRQQQPYTFGQGTKVEIKR

SEQ ID NO: 278 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRASRPYTFGQGTKVEIKR

SEQ ID NO: 279 - protein, 106 aa, Homo sapiens
DIQMTQSPSSLSASVGDRVTITCRASQNIGGRLVWYQQKPGKAPKLLIYTPSPLQVPSRFSGSGSGTDFTLTISSLQPEDFATYYCQQRYVQPYTFGQGTKVEIKR

SEQ ID NO: 280 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRYKPPYTFGQGTKVKIKR

SEQ ID NO: 281 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRVRAPYTFGQGTKVEIKR

SEQ ID NO: 282 - protein, 108 aa, Homo sapiens
DIQMTQSPSSLSASVGDRVTITCRASQNIGSKLVWYQQKPGKASKLLIYTPSRLQSGVPSRFSGSGSGTDFTLTISSLQPEDFATYYCQQRFMTPYTFGQGTKVEIKR

SEQ ID NO: 283 - protein, 108 aa, Homo sapiens
DIQMTQTPSSLSASVGDRVTITCRASQNIGKQLLWYQQKPGKAPRLLIYCPPPLQSGVPSRFSCSGSGTDFTLTISSLQPEDFATYYCQQHASRPFTFGQGTKVEIKR

SEQ ID NO: 284 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDSATYYCQQRYSLPFTFGQGTKVEIKR

SEQ ID NO: 285 - protein, 108 aa, Homo sapiens
DIQMTQSPSSLSASVGDRVTISCRASQNIGTQLHWYQQKPGKAPRLLIYGSSFLQSGVPSRFSGSGSGTDFTLTISSLQPEDFATYYCQQVMLGPTTFGQGTKVEIKR

SEQ ID NO: 286 - protein, 108 aa, Homo sapiens
DIQMTQSPSSLSASVGDRVTITCRASQNIHGMLRWYQQKPGKAPKLLIYTPSPSQSGVPSRFSGSGSGTDFTLTISSLQPEDFATYYCQQTATWPFTFGQGTKVEIKR

SEQ ID NO: 287 - protein, 108 aa, Homo sapiens
DIQMTQSPSSLSASVGDRVTITCRASQPIGNKLRWYQQKPGKAPKLLIYSPSPLQSGVPSRFSGSGSGTDYTLTISSLQPEDFATYYCQQTWSFPGTFGQGTKVEIKR

SEQ ID NO: 288 - protein, 108 aa, Homo sapiens
  VARIANT 92: Xaa = Any Amino Acid
DIQMTQSPSSLSASVGDRVTITCQASQPIDGRLVWYQQKPGKASKLLIYVPSGLQSGVPSRFSGSGSGTDFTLTISSLQPEDFATYYCQQRXTPPYTFGQGTKVEIKR

SEQ ID NO: 289 - protein, 108 aa, Homo sapiens
DIQMTQSPSSLSASVGDRVTITCRASQSIGGRLVWYQQKPGKAPKLLIYTPPPLQSGVPSRFSGSGSGTDFTLTISSLQPEDFATYYCQQRYLRPFTFGQGTKVEIKR

SEQ ID NO: 290 - protein, 108 aa, Homo sapiens
DIQMTQSPSSLSASVGDRVTITCRASQSIGPWLSWYQQKPGKAPKLLIYQVSRLQSGVPSRFSGSGSGTDFTLTISSLQPEDFATYYCQQNLAPPYTFGQGTKVEIKR

SEQ ID NO: 291 - protein, 108 aa, Homo sapiens
DIQMTQSPSSLSASVGDRVTITCRASRNIGGRLVWYQQKPGKAPKLLIYTPSPLQSGVPSRFSGSGSGTDFTLTISSLQPEDFATYYCQQRHNEPFTFGQGTKVEIKR

SEQ ID NO: 292 - protein, 108 aa, Homo sapiens
  VARIANT 30, 34, 36, 51, 61, 91, 96: Xaa = Any Amino Acid
DIQMTQSPSSLSASVGDRVTITCRASQNIXTLLXWXQQKPGKAPKLLIYAXSRLQSGVPSXFSGSGSGTDFTLTISSLQPEDFATYYCQQXYRHPXTLGQGTKVEIKR

SEQ ID NO: 293 - protein, 108 aa, Homo sapiens
DIQMTQSPSSLSASVGDRVTITCRASQDIKSHLRWYQQKPGKAPKLLIYTPSSLQSGVPSRFSGSGSGTDFTLTISSLQPEDSATYYCQQVLTVPYTFGQGTKVEIKR

SEQ ID NO: 294 - protein, 108 aa, Homo sapiens
DIQMTQSPSSLSASVGDRVTITCRASQDIGRWLSWYQQKPGKAPKLLIYAGSQLQSGVPSRFSGSGSGTDFTLTISSLQPEDFATYYCQRSWDPPTTFGQGTKVEIKR

SEQ ID NO: 295 - protein, 108 aa, Homo sapiens
DIQMTQSPSSLSASVGDRVTITCRASQPIGSMLVWYQQKPGKAPKLLIYTPSSLQSGVPSRFSGSGSGTDFTLTISSLQPEDFATYYCQQKYMEPHTFGQGTKVEIKQ

SEQ ID NO: 296 - protein, 108 aa, Homo sapiens
DIQMTQSPSSLSASVGDRVTITCRASQDINRQLVWYQQKPGKAPKLLIYTPSSLQSGVPSRFSGSGSGTDFTLTISSLQPEDYATYYCQQKYRYPFTFGQGTKVEIKR

SEQ ID NO: 297 - protein, 108 aa, Homo sapiens
DIQMTQSPSSLSASVGDRVTITCRASQDISRFLNWYQQKPGKAPKLLIYWTSLLQSGVPSRFSGSGSGTDFTLTISSLQPEDFATYYCQQSRHHPTTFGQGTKVEIKR

SEQ ID NO: 298 - protein, 108 aa, Homo sapiens
DIQMSQSPSSLSASVGDRVTITCRASQDIGNMLAWYQQKPGKAPKPLIYYASYLQSGVPSRFSGSGSGTDFTLTISSLQPEDFATYYCQQLGAKPHTFGQGTKVEIKR

SEQ ID NO: 299 - protein, 108 aa, Homo sapiens
DIQMTQSPSSLSASVGDRVTITCRASQEINNMLVWYQQKPGKAPKLLIYAPSGLQSGVPSRFSGSGSGTDFTLTISSLQPEDFATYYCQQRRYPPFTFGQGTKVEIKR

SEQ ID NO: 300 - protein, 108 aa, Homo sapiens
DIQMTQSPSSLSASVGDRVTITCRASQEIGSHLRWYQQKPGKAPKLLIYQESQLQSGVPSRFSGSGSGTDFTLTISSLQPEDFATYYCQQTWNSPMTFGQGTKVEIKR

SEQ ID NO: 301 - protein, 108 aa, Homo sapiens
DIQMTQSPSSLSASVGDRVTITCRASQGIGRHLRWYQQKPGKAPKLLIYSPSGLQGGVPSRFSGSGSGTDYTLTISSLQPEDYATYYCQQVYSPPFTFGQGTKVEIKR

SEQ ID NO: 302 - protein, 108 aa, Homo sapiens
DIQMTQSPSSLSASVGDRVTITCRASQKIGNMLAWYQQKPGKAPKLLIYKYSKLQSGVPSRFSGSGSGTDFTLTISSLQPEDFATYYCQQLAVPPHTFGQGTKVEIKR

SEQ ID NO: 303 - protein, 108 aa, Homo sapiens
DIQMTQSPSSLSASVGDRVTITCRASQYIQMRLRWYQQKPGKAPRLLIYGASMLQSGVPSRFSGSGSGTDFTLTISSLQPEDFATYYCQQDWTAPHTFGQGTKVEIKR

SEQ ID NO: 304 - protein, 108 aa, Homo sapiens
DIQMTQSPSSLSASVGDRVTITCRASQQIGQLLNWYQQKPGKAPKLLIYAGSRLQSGVPSRFSGSGSGTDFTLTISSLQPEDFATYYCQQMRQTPVTFGQGTKVEIKR

SEQ ID NO: 305 - protein, 108 aa, Homo sapiens
DIQMTQSPSSLSASVGDRVTITCRASQDIGQLLNWYQQKPGKAPKLLIYASSRLQSGVPSRFSGSGSGTDFTLTISSLQPEDFATYYCQQRTYNPSTFGPGTKVEIKR

SEQ ID NO: 306 - protein, 108 aa, Homo sapiens
DIQMTQSPSSLSASVGDRVTITCRASQDIGALLRWYQQKPGKAPKLLIYTPSELQSGVPSRFSGSGSGTDFTLTISSLQPEDFATYYCQQVFRSPYTFGQGTKVEIKR

SEQ ID NO: 307 - protein, 108 aa, Homo sapiens
DIQMTQSPSSLSASVGDRVTITCRASQDIGAQLRWYRQKPGKAPKLLIYAPSALQSGVPSRFSGSGSGTDFTLTISSLQPEDFATYYCQQVALRPYTFGQGTRVEIKR

SEQ ID NO: 308 - protein, 108 aa, Homo sapiens
DIQMTQSPSSLSASVGDRVTITCRASQDIGHKLRWYQQKPGKAPKLLIYTPSTLQSGVPSRFSGSGSGTDFTLTISSLQPEDFATYYCQQTWTPPYTFGQGTKVEIKR

SEQ ID NO: 309 - protein, 108 aa, Homo sapiens
DIQMTQSPSSLSASVGDRVTITCRASQDIDTHLRWYQQKPGKAPKLLIYGSSFLQSGVPSRFSGSGSGTDFTLTISSLQPEDFATYYCQQTWARPMTFGQGTKVEIKR

SEQ ID NO: 310 - protein, 108 aa, Homo sapiens
DIQMTQSPSSLSASVGDRVTITCRASQDIKGMLVWYQQKPGKAPKLLIYTPSRLQSGVPSRFSGSGSGTDFTLTISSLQPEDFATYYCQQTWVSPQTFGQGTKVEIKR

SEQ ID NO: 311 - protein, 108 aa, Homo sapiens
DIQMTQSPSSLSASVGDRVTITCRASQDIKSHLRWYQQKPGKAPKLLIYTPSSLQSGVPSRFSGSGSGTDFTLTISSLQPEDFATYYCQQVSSTPYTFGQGTKVEIKR

SEQ ID NO: 312 - protein, 108 aa, Homo sapiens; identical to SEQ ID NO: 300

SEQ ID NO: 313 - protein, 108 aa, Homo sapiens; identical to SEQ ID NO: 298

SEQ ID NO: 314 - protein, 108 aa, Homo sapiens
DIQMTQSPSSLSASVGDRVTITCRASQEIGGNLVWYQQKPGKAPKLLIYAPSRLQSGVPSRFSSSGSGTDFTLTISSLQPEDFATYYCQQKFSYPFTFGQGTKVEIKR

SEQ ID NO: 315 - protein, 108 aa, Homo sapiens; identical to SEQ ID NO: 299

SEQ ID NO: 316 - protein, 108 aa, Homo sapiens
DIQMTQSPSSLSASVGDRVTITCRASQWIGNHLRWYQQKPGKAPTLLIYGSSRLQSGVPSRFSGSGSGTDFTLTISSLQPEDFATYYCQQTWNSPMTFGQGTKVEIKR

SEQ ID NO: 317 - protein, 108 aa, Homo sapiens
DIQMTQSPSSLSASVGDRVTITCRASQAIDIHLRWYQQKPGKAPKLLIYHASSLQSGVPSRFSGSGSGTDFTLTISSLQPEDFATYYCQQTYRSPMTFGQGTKVEIKR

SEQ ID NO: 318 - protein, 108 aa, Homo sapiens
  VARIANT 50: Xaa = Any Amino Acid
DIQMTQSPSSLSASVGDRVTITCRASQAIGQSLRWYQQKPGKAPTLLIYXSSNLQSGVPSRFSGSGSGTDFTLTISSLQPEDFATYYCQQTWVSPMTFGQGTKVEIKR

SEQ ID NO: 319 - protein, 108 aa, Homo sapiens
DIQMTQSPSSLSASVGDRVTITCRASQYIGGSLRWYQQKPGKAPKLLIYSGSTLQSGVPSRFSGSGSGTDFTLTISSLQPEDFATYYCQQTWVSPMTFGQGTKVEIKR

SEQ ID NO: 320 - protein, 108 aa, Homo sapiens
  VARIANT 38: Xaa = Any Amino Acid
DIQMTQSPSSLSASVGDRVTITCRASQYINAHLRWYQXKPGKAPKLLIYMSSYLQSGVPSRFSGSGSGTDFTLTISSLQPEDFATYYCQQTWSSPMTFGQGTKVEIKR

SEQ ID NO: 321 - protein, 108 aa, Homo sapiens
DIQMTQSPSSLSASVGDRVTITCRASQGIMYHLRWYQQKPGKAPRLLIYHGSTLQSGVPARFSGSGSGTDFTLTISSLQPEDFATYYCQQTWNAPLTFGQGTKVEIKR

SEQ ID NO: 322 - protein, 108 aa, Homo sapiens
DIQMTQSPSSLSASVGDRVTITCRASQGIGNSLRWYQQKPGKAPKLLIYYSSHLQSGVPSRFSGSGSGTDFTLTISSLQPEDFATYYCQQIRTKPFTFGQGTKVEIKR

SEQ ID NO: 323 - protein, 108 aa, Homo sapiens
DIQMTQSPSSLSASVGDRVTITCRASQKIMTHLRWYQQKPGKAPKLLIYGGSHLQSGVPSRFSGSGSGTDFTLTISSLQPEDFATYYCQQTWVSPMTFGQGTKVEIRR

SEQ ID NO: 324 - protein, 108 aa, Homo sapiens
DIQMTQSPSSLSASVGDRVTITCRASQVIGNALRWYQQKPGKAPKLLIYYGSYLQSGVPSRFSGSGSGTDFTLTISSLQPEDFATYYCQQIHFKPFTFGQGTKVEIKR

SEQ ID NO: 325 - protein, 108 aa, Homo sapiens
DIQMTQSPSSLSASVGDRVTITCRASQRIGHHLRWYQQKPGKAPKLLIYSASALQSGVPSRFSGSGSGTDFTLTISSLQPEDFATYYCQQTWNAPMTFGQGTKVEIKR

SEQ ID NO: 326 - protein, 108 aa, Homo sapiens
DIQMTQSPSSLSASVGDRVTITCRASQRIGLMLNWYQQKPGKAPRLLIYAASRLQSGVPSRFSGSGSGTDFTLTISSLQPEDFATYYCQQMLHPPVTFGQGTKVEIKR

SEQ ID NO: 327 - protein, 108 aa, Homo sapiens
  VARIANT 3, 38: Xaa = Any Amino Acid
DIXMTQSPSSLSASVGDRVTITCRASQRIEGKLLWYQXKPGKAPKLLIYCPSNLQSGVPSRFSCSGSGTDFTLTISSLQPEDFATYYCQQKFREPSTFGQGTKVEIKR

SEQ ID NO: 328 - protein, 108 aa, Homo sapiens; identical to SEQ ID NO: 289

SEQ ID NO: 329 - protein, 108 aa, Homo sapiens
DIQMTQSPSSLSASVGDRVTITCRASQSIGTLLNWYQQKPGKAPKLLIYASSRLQSGVPSRFSGSGSGTDFTLTISSLQPEDFATYYCQQMNRVPITFGQGTKVEIKR

SEQ ID NO: 330 - protein, 108 aa, Homo sapiens
DIQMTQSPSSLSASVGDRVTITCRASQSIGMLLSWYQQKPGKAPKLLIYAVSRLQSGVPSRFSGSGSGTDFTLTISSLQPEDYATYYCQQMQRPPITFGQGTKVEIKR

SEQ ID NO: 331 - protein, 108 aa, Homo sapiens
DIQMTQSPSSLSASVGDRVTITCRASQSIGVNLLWYQQIPGKAPRLLIYGASYLQSGVPSRFSGSGSGTDFTLTISSLQPEDFATYYCQQYFFAPLTFGQGTKVEIKR

SEQ ID NO: 332 - protein, 108 aa, Homo sapiens
DIQMTQSPSSLSASVGDRVTITCRASQSIGHNLVWYQQKPGKAPKLLIYTPSPLQSGVPSRFSGSGSGTDFTLTISSLQPEDFATYYCQQKYTPPYTFGQGTKVEIKR

SEQ ID NO: 333 - protein, 108 aa, Homo sapiens
  VARIANT 17: Xaa = Any Amino Acid
DIQMTQSPSSLSASVGXRVTITCRASQSIGVQLRWYQQKPGKAPKLLIYHGSQLQSGVPSRFSGSGSGTDFTLTISSLQPEDFATYYCQQNWARPITFGQGTKVEIKR

SEQ ID NO: 334 - protein, 108 aa, Homo sapiens
DIQMTQSPSSLSASVGDRVTITCRASQSIATSLRWYQQKPGKAPKLLIYHSSVLQSGVPSRFSGSGSGTDFTLTISSLQPEDFATYYCQQTWVVPMTFGQGTKVEIKR

SEQ ID NO: 335 - protein, 108 aa, Homo sapiens; identical to SEQ ID NO: 319

SEQ ID NO: 336 - protein, 108 aa, Homo sapiens
DIQMTQSPSSLSASVGDRVTITCRASQSIKGHLVWYQQKPGKAPMLLIYSPSSLRSGVPSRFSGSGSGTDFTLTISSLQPEDFATYYCQQVYEKPFTFGQGTKVEIKR

SEQ ID NO: 337 - protein, 108 aa, Homo sapiens
  VARIANT 93: Xaa = Any Amino Acid
DIQMTQSPSSLSASVGDRVTITCRASQPIHGALRWYQQKPGKAPMLLIYTPSQLQSGVPSRFSGSGSGTDFTLTISSLQPEDFATYYCQQVGXKPYTFGQGTKVEIKR

SEQ ID NO: 338 - protein, 108 aa, Homo sapiens
DIQMTQSPSSLSASVGDRVTITCQASQPIDGRLVWYQQKPGKASKLLIYVPSGLQSGVPSRFSGSGSGTDFTLTISSLQPEDFATYYCQQRHTPPYTFGQGTKVEIKR

SEQ ID NO: 339 - protein, 108 aa, Homo sapiens
DIQMTQSPSSLSASVGDRVTITCRASQPINNWLNWYQQKPGKAPKLLIYATSRLQSGVPSRFSGSGSGTDFTLTISSLQPEDFATYYCQPSWTPPPTFGQGTKVEIKR

SEQ ID NO: 340 - protein, 108 aa, Homo sapiens
DIQMTQSPSSLSASVGDRVTITCRASQPIKMMLSWYQQKPGKAPKLLIYNNSTLQSGVPSRFSGSGSGTDFTLTISSLQPEDFATYYCQQYRRWPYTFSQGTKVEIKR

SEQ ID NO: 341 - protein, 108 aa, Homo sapiens; identical to SEQ ID NO: 295

SEQ ID NO: 342 - protein, 108 aa, Homo sapiens; identical to SEQ ID NO: 304

SEQ ID NO: 343 - protein, 108 aa, Homo sapiens
  VARIANT 82: Xaa = Any Amino Acid
DIQMTQSPSSLSASVGDRATITCRASQQIGAHLRWYQQKPGKAPKLLIYQSSQLQSGVPSRFSGSGSGTDFTLTISSLQPEXFATYYCQQTWASPMTFGQGTKVEIKR

SEQ ID NO: 344 - protein, 108 aa, Homo sapiens; identical to SEQ ID NO: 281

SEQ ID NO: 345 - protein, 108 aa, Homo sapiens
  VARIANT 27: Xaa = Any Amino Acid
DIQMTQSPSSLSASVGDRVTITCRASXNIGGRLVWYQQKPGKAPKLLIYTPSPLQSGVPSRFSGSGSGTDFTLTISSLQPEDFATYYCQQRSVSPYTFGQGTKVEIKR

SEQ ID NO: 346 - protein, 108 aa, Homo sapiens
  VARIANT 28, 30, 32, 34, 36, 93: Xaa = Any Amino Acid
DIQMTQSPSSLSASVGDRVTITCRASQXIXTXLXWXQQKPGKAPTLLIYNSSQLQSGVPSRFSGSGSGTDFTLTISSLQPEDFATYYCQQTWXRPMTFGQGTKVEIKR

SEQ ID NO: 347 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRHYPPFTFGQGTKVEIKR

SEQ ID NO: 348 - protein, 108 aa, Homo sapiens
  VARIANT 3: Xaa = Any Amino Acid
DIXMTQSPSSLSASVGDRVTITCRASQNIGGRLVWYQQKPGKAPKLLIYTPSPLQSGVPSRFSGSGSGTDFTLTISSLQPEDFATYYCQQRHTSPYTFGQGTKVEIKR

SEQ ID NO: 349 - protein, 108 aa, Homo sapiens
  VARIANT 38: Xaa = Any Amino Acid
DIQMTQSPSSLSASVGDRVTITCRASQNIGGRLVWYQXKPGKAPKLLIYTPSPLQSGVPSRFSGSGSGTDFTLTISSLQPEDFATYYCQQRHSEPWTFGQGTKVEIKR

SEQ ID NO: 350 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRSKLPYTFGQGTKVEIKR

SEQ ID NO: 351 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQKFKQPYTFGQGTKVEIKR

SEQ ID NO: 352 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRFSSPFTFGQGTKVEIKR

SEQ ID NO: 353 - protein, 108 aa, Homo sapiens
  VARIANT 3, 56, 82: Xaa = Any Amino Acid
DIXMTQSPSSLSASVGDRVTITCRASQNIGGRLVWYQQKPGKAPKLLIYTPSPLQXGVPSRFSGSGSGTDFTLTISSLQPEXFATYYCQQRAVTPFTFGQGTKVEIKR

SEQ ID NO: 354 - protein, 108 aa, Homo sapiens
  VARIANT 27: Xaa = Any Amino Acid
DIQMTQSPSSLSASVGDRVTITCRASXNIGGRLVWYQQKPGKAPKLLIYTPSPLQSGVPSRFSGSGSGTDFTLTISSLQPEDFATYYCQQRATQPYTFGQGTKVEIKR

SEQ ID NO: 355 - protein, 108 aa, Homo sapiens
DIQMTQSPSSLSASVGDRVTITCRASQNIGGRLVWYQQKPGKAPKLLIYTPSPLQSGVPSRFSGSGSGTDFTLTISSLQPEDFATYYCQQRKAPPYTFGQGTKVEIKR

SEQ ID NO: 356 - protein, 108 aa, Homo sapiens
DIQMTQSPSSLSASVGDRVTITCQASQNIGVLLNWYQQKPGKAPKLLIYASSRLQSGVPSRFSGSGSGTDFTLTISSLQPEDFATYYCQQRNFPPPTFGQGTKVEIKR

SEQ ID NO: 357 - protein, 108 aa, Homo sapiens; identical to SEQ ID NO: 283

SEQ ID NO: 358 - protein, 108 aa, Homo sapiens
  VARIANT 3: Xaa = Any Amino Acid
DIXMTQSPSSLSASVGDRVTITCRASQNIHGMLRWYQQKPGKAPKLLIYTPSPSQSGVPSRFSGSGSGTDFTLTISSLQPEDFATYYCQQTATWPFTFGQGTKVEIKR

SEQ ID NO: 359 - protein, 108 aa, Homo sapiens; identical to SEQ ID NO: 294

SEQ ID NO: 360 - protein, 108 aa, Homo sapiens
DIQMTQSPSSLSASVGDRVTITCRASQDIGNMLAWYQQKPGKAPKPLIYYASYLQSGVPSRFSGSGSGTDFTLTISSLQPEDFATYYCQQMRDYPVTFGQGTKVEIKR

SEQ ID NO: 361 - protein, 108 aa, Homo sapiens
DIQMTQSPSSLSASVGDRVTITCRASQDIGNMLAWYQQKPGKAPKPLIYYASYLQSGVPSRFSGSGSGTDFTLTISSLQPEDFATYYCQQMRNLPRTFGQGTKVEIKR

SEQ ID NO: 362 - protein, 108 aa, Homo sapiens; identical to SEQ ID NO: 298

SEQ ID NO: 363 - protein, 108 aa, Homo sapiens
DIQMTQSPSSLSASVGDRVTITCRASQSIGPWLSWYQQKPGKAPKLLFYQVSRLQSGVPSRFSGSGSGTDFTLTIISLQPEDFATYYCQQNLAPPYTFGQGTKVEIKR

SEQ ID NO: 364 - protein, 108 aa, Homo sapiens
DIQMTQSPSSLSASVGDRVTITCRASQSIGPWLSWYQQKPGKAPKLLFYQVSRLPSGVPSRFSGSGSGTDFTVTISSLQPEDFATYYCQQNLAPPYTFGQGTKVEIKR

SEQ ID NO: 365 - protein, 108 aa, Homo sapiens
DIQMTQSPSSLSASVGDRVTITCRASQSIGPWLSWYQQKPGKAPKLLFYQVSRLRSGVPSRFSGSGSGTDFTLTISSLQPEDFASYYCQQNLAPPYTFGQGTKVEIKR

SEQ ID NO: 366 - protein, 108 aa, Homo sapiens
DIQMTQSPSSLSASVGDRVTITCRASQSIGPWLSWYQQKPGKAPKLLIYQVSRLQSGVPSRFSVSGSGTDFTLTISSLQPEDFATYYCQQNLAPPYTFGQGTKVEIKR

SEQ ID NO: 367 - protein, 108 aa, Homo sapiens
DIQMTQSPSSLSASVGDRVTITCRASQPIGSLLEWYQQKPGKAPKLLIYNVSRLQSGVPSRFSGSGSGTDFTLTISSLQPEDFATYYCQQRRFAPRTFGQGTKVEIKR

SEQ ID NO: 368 - protein, 108 aa, Homo sapiens
DIQMTQSPSSLSASVGDRVTITCRASQNIDLELSWYQQKPGKAPKLLIYFTSVLQSGVPSRFSGSGSGTDFTLTISSLQPEDYATYYCQQRIRRPYTFGQGTKVEIKR

SEQ ID NO: 369 - protein, 108 aa, Homo sapiens; identical to SEQ ID NO: 15

SEQ ID NO: 370 - protein, 108 aa, Homo sapiens
DIQMTQSPSSLSASVGDRVTITCRASQNIIDYLNWYQQKPGKAPKLLIYWGSLLQSGVPSRFSGSGSGTDFTLTISSLQPEDFATYYCQQTYRRPFTFGQGTKVEIKR

SEQ ID NO: 371 - protein, 108 aa, Homo sapiens
DIQMTQSPSSLSASVGDRVTITCRASQPIDEWLVWYQQKPGKAPKLLIYRGSLLQSGVPSRFSGSGSGTDFTLTISSLQPEDFATYYCQQYRQMPATFGQGTKVEIKR

SEQ ID NO: 372 - protein, 108 aa, Homo sapiens
DIQMTQSPSSLSASVGDRVTITCRASQPIASRLLWYQQKPGKAPKLLIYYGSVLQSGVPSRFSGSGSGTDFTLTISSLQPEDFATYYCQQTWAHPITFGQGTKVEIKR

SEQ ID NO: 373 - protein, 108 aa, Homo sapiens
DIQMTQSPSSLSASVGDRVTITCRASQPIYKMLRWYQQKPGEAPKLLIYQASNLQSGVPSRFSGSGSGTDFTLTISGLQPEDLATYYCQQFAKWPYTFGQGTKVEIKR

SEQ ID NO: 374 - protein, 108 aa, Homo sapiens
DIQMTQSPSSLSASVGDRVTITCRASQPINTSLNWYQQKPGKAPKLLIYGGSWLQSGVPSRFSGSGSGTDFTLTISSLQPEDFATYYCQQYLYSPSTFGQGTKVEIKR

SEQ ID NO: 375 - protein, 108 aa, Homo sapiens
DIQMTQSPSSLSASVGDRVTITCRASQPIHENLDWYQQKPGKAPKLLIYGASMLQSGVPSRFSGSGSGTDFTLTISSLQPEDSATYYCQQGWVYPQTFGQGTKVEIKR

SEQ ID NO: 376 - protein, 108 aa, Homo sapiens
DIQMTQSPSSLSASVGDRVTITCRASQPIDTFLRWYQQKPGKAPKLLIYRASQLQSGVPSRFSGSGSGTDFTLTISSLQPEDFATYYCQQWARSPFTFGQGTKVKIKR

SEQ ID NO: 377 - protein, 108 aa, Homo sapiens
DIQMTQSPSSLSASVGDRVTITCRASQFIEWYLAWYQQKPGKAPKLLIYNGSVLQSGVPSRFSGSGSGTDFTLTISSLQPEDFATYYCQQRVARPFTFGQGTKVEIKR

SEQ ID NO: 378 - protein, 108 aa, Homo sapiens
DIQMTQSPSSLSASVGDRVTITCRASQYIGTALDWYQQKPGKAPKLLIYAVSLLQSGVPSRFSGSGSGTDFTLTISSLQPEDLATYYCQQAFAPPMTFGQGTKVEIKR

SEQ ID NO: 379 - protein, 108 aa, Homo sapiens
DIQMTQSPSSLSASVGDRVTITCRASQHITDQLRWYQKKPGKAPKLLIYSASILQSGVPSRFSGSGSGTDFTLTISSLQPEDFATYYCQQIYIRPGTFGQGTKVEIKR

SEQ ID NO: 380 - protein, 108 aa, Homo sapiens
DIQMTQSPSSLSASVGDRVTITCRASQHIGDYLAWYQQKPGKAPKLLIYPSSQLQSGVPSRFSGSGSGTDFTLTISSLQPEDFATYYCQQRRYLPMTFGQGTKVEIKR

SEQ ID NO: 381 - protein, 108 aa, Homo sapiens
DIQMTQSPSSLSASVGDRVTITCRASQDIGEYLQWYQQKPGKAPKLLIYWTSMLQSGVPSRFSGSGSGTDFTLTISSLQPEDFATYYCQQEARTPFTFGQGTKVEIKR

SEQ ID NO: 382 - protein, 108 aa, Homo sapiens
DIQMTQSPSSLSASVGDRVTITCRASQDINDYLSWYQQKPGKAPKLLIYWGSSLQSGVPSRFSGSGSGTDFTLTISSLQPEDFATYYCQQRAYRPYTFGQGTKVEIKR

SEQ ID NO: 383 - protein, 108 aa, Homo sapiens
DIQMTQSPSSLSASVGDRVTITCRASQDIFPFLNWYQQKPGKAPELLIYRASILHSGVPSRFSGSGSGTDFTLTISSLQPEDFATYYCQQIARSPRTFGQGTKVEIKR

SEQ ID NO: 384 - protein, 108 aa, Homo sapiens
DIQMTQSPSSLSASVGDRVTITCRASQDIEDWLAWYQQKPGKAPKLLIYWGSTLQSGVPSRFSGSGSGTDFTLTISSLQPEDFATYYCQQSKGTPYTFGQGTKVEIKR

SEQ ID NO: 385 - protein, 108 aa, Homo sapiens
DIQMTQSPSSLSASVGDRVTITCRASQDIDDWLHWYQQKPGKAPKLLIYWSSSLQSGVPSRFSGSGSGTDFTLTISSLQPEDFATYYCQQEKYRPFTFGQGTKVEIKR

SEQ ID NO: 386 - protein, 108 aa, Homo sapiens
DIQMTQSPSSLSASVGDRVTITCRASQDIQTWLSWYQQKPGKAPKLLIYHSSYLQSGVPSRFSGSGSGTDFTLTISSLQPEDFATYYCQQYDTLPGTFGQGTKVEIKR

SEQ ID NO: 387 - protein, 108 aa, Homo sapiens
  VARIANT 28: Xaa = Any Amino Acid
DIQMTQSPSSLSASVGDRVTITCRASQXISGCLYWYQQKPGKAPKLLIYRGSHLQSGVPSRFSGSGSGTDFTLTISSLQPEDFATYYCQQDCDPPSTFGQGTKVEIKR

SEQ ID NO: 388 - protein, 108 aa, Homo sapiens
DIQMTQSPSSLSASVGDRVTITCRASQSIEKKLVWYQQKPGKAPKLLIYYTSYLQSGVPSRFSGSGSGTDFTLTISSLQPEDFATYYCQQYQGHPLTFGQGTKVEIKR

SEQ ID NO: 389 - protein, 108 aa, Homo sapiens; identical to SEQ ID NO: 379

SEQ ID NO: 390 - protein, 108 aa, Homo sapiens
DIQMTQSPSSLSASVGDRVTITCRASQPIGDMLMWYQQKPGKAPKLLIYGGSNLQSGVPSRFSGSGSGTDFTLTISSLQPEDFATYYCQQRRLAPSTFGQGTKVEIKR

SEQ ID NO: 391 - protein, 108 aa, Homo sapiens
DIQMTQSPSSLSASVGDRVTITCRASQPIDERLNWYQQKPGKAPKLLIYRRSWLQSGVPSRFSGSGSGTDFTLTISSLQPEDFATYYCQQWGHHPSTFGQGTKVEIKR

SEQ ID NO: 392 - protein, 108 aa, Homo sapiens
DIQMTQSPSSLSASVGDRVTITCRASQPIDSRLMWYQQKPGKAPKLLIYFASYLQSGVPSRFSGSGSGTDFTLTISSLQPEDFATYYCQQYLMHPLTFGQGTKVEIKR

SEQ ID NO: 393 - protein, 108 aa, Homo sapiens
DIQMTQSPSSLSASVGDRVTITCRASQPIHYALDWYQQKPGKAPKLLIYSTSILQSGVPSRFSGSGSGTDFTLTISSLQPEDFATYYCQQWFRWPTTFGQGTKVEIKR

SEQ ID NO: 394 - protein, 108 aa, Homo sapiens
DIQMTQSPSSLSASVGDRVTITCRASQPIGDFLLWYQQKPGKAPKLLIYGASTLQSGVPSRFSGSGSGTDFTLTISSLQPEDFATYYCQQRRFFPSTFGQGTKVEIKR

SEQ ID NO: 395 - protein, 108 aa, Homo sapiens
DIQMTQSPSSLSASVGDRVTITCRASQHIGQNLNWYQQKPGKAPKLLIYWGSDLQSGVPSRFSGSGSGTDFTLTISSLQPEDFATYYCQQLRFPPLTFGQGTKVEIKR

SEQ ID NO: 396 - protein, 108 aa, Homo sapiens
DIQMTQSPSSLSASVGDRVTITCRASQDIGEYLYWYQQKPGKAPKLLIYMISNLQSGVPSRFSGSGSGTDFTLTISSLQPEDFATYYCQQLVAWPYTFGQGTKVEIKR

SEQ ID NO: 397 - protein, 108 aa, Homo sapiens
DIQMTQSPSSLSASVGDRVTITCRASQDIYGELSWYQQKPGKAPKLLIYFSSILQSGVPSRFSGSGSGTDFTLTISSLQPEDFATYYCQQRSVRPYTFGQGTKVEIKR

SEQ ID NO: 398 - protein, 108 aa, Homo sapiens
DIQMTQSPSSLSASVGDRVTITCRASQDIHGYLDWYQQKPGKAPKLLIYYASYLQSGVPSRFSGSGSGTDFTLTISSLQPEDFATYYCQQRYQHPVTFGQGTKVEIKR

SEQ ID NO: 399 - protein, 108 aa, Homo sapiens
DIQMTQSPSSLSASVGDRVTITCRASQEIGQWLNWYQQKPGKAPKLLIYWGSELQSGVPSRFSGSGSGTDFTLTISSLQPEDFATYYCQQTSRRPFTFGQGTKVEIKR

SEQ ID NO: 400 - protein, 108 aa, Homo sapiens
DIQMTQSPSSLSASVGDRVTITCRASQDINSRLSWYQQKPGKAPKLLIYYASYLRSGVPSRFSGSGSGTDFTLTISSLQPEDFATYYCQQWWSHPITFGQGTKVEIKR

SEQ ID NO: 401 - protein, 108 aa, Homo sapiens
DIQLTQSPSSLSASVGDRVTITCRASQDIGDHLLWYQQKPGKAPKLLIYGASQLQSGVPSRFSGSGSGTDFTLTISSLQPEDFATYYCQQVRIYPRTFGQGTKVEIKR

SEQ ID NO: 402 - protein, 108 aa, Homo sapiens
DIQMTQSPSSLSASVGDRVTITCRASQDIDRWLRWYQQKPGKAPRLLIYWTSELQSGVPSRFSGSGSGTDFTLTISSLQPEDFATYYCQQEFRMPVTFGQGTKVEIKR

SEQ ID NO: 403 - protein, 108 aa, Homo sapiens
DIQMTQSPSSLSASVGDRVTITCRASQDIGDHLLWYQQKPGKAPKLLIYGSSALQSGVPSRFSGSGSGTDFTLTISSLQPEDFATYYCQQVRGFPSTFGQGTKVEIKR

SEQ ID NO: 404 - protein, 108 aa, Homo sapiens
DIQMTQSPSSLSASVGDRVTITCRASQDISDYLSWYQQKPGKAPKLLIYWTSMLQSGVPSRFSGSRSGTDFTLTISSLQPEDFATYYCQQRYRRPFTFGQGTKVEIKR

SEQ ID NO: 405 - protein, 108 aa, Homo sapiens
DIQMTQSPSSLSASVGDRVTITCRASQSIGKHLAWYQQKPGKAPKLLIYRASLLQSGVPSRFSGSGSGTDFTLTISSLQPEDFATYYCQQHSRSPRTFGQGTKVEIKR

SEQ ID NO: 406 - protein, 108 aa, Homo sapiens
DIQMTQSPSSLSASVGDRVTITCRASQFIGLHLVWYQQKPGKASKLLIYNTSDLQSGVPSRFSGSGSGTDFTLTISSLQPEDFATYYCQQMAHYPYTFSQGTKVEIKR

SEQ ID NO: 407 - protein, 108 aa, Homo sapiens
DIQMTQSPSSLSASVGDRVTITCRASQSIGDMLLWYQQKPGKAPKLLIYGSSALQSGVPSRFSGSGSGTDFTLTISSLQPEDFATYYCQQVRTYPSTFGQGTKVEIKR

SEQ ID NO: 408 - protein, 108 aa, Homo sapiens
DIQMTQSPSSLSASVGDRVTITCRASRLIGKHLSWYQQKPGKAPKLLIYRSSVLQSGVPSRFSGSGSGTDFTLTISSLQPEDFATYYCQQHATSPRTFGQGTKVEIKR

SEQ ID NO: 409 - protein, 108 aa, Homo sapiens
DIQMTQSPSSLSASVGDRVTITCRASQYIDKRLLWYQQKPGEAPKLLIYYASYLQSGVPSRFSGSGSGTDFTLTISSLQPEDFATYYCQQQFIHPLTFGQGTKVEIKR

SEQ ID NO: 410 - protein, 108 aa, Homo sapiens
DIQMTQSPSSLSASVGDRVTITCRASQYIGQMLNWYQQKPGKAPKLLIYQASGLQSGVPSRFSGSGSGTDFTLTISSLQPEDFATYYCQQSYVHPYTFGQGTKVEIKR

SEQ ID NO: 411 - protein, 108 aa, Homo sapiens
DIQMTQSPSSLSASVGDRVTITCRASQAIGNWLDWYQQKPGKAPKLLIYWGSELQSGVPSRFSGSGSGTDFTLTISSLQPEDFATYYCQQRSSSPFTFGQGTKVEIKR

SEQ ID NO: 412 - protein, 108 aa, Homo sapiens
DIQMTQSPSSLSASVGDRVTITCRASQAIDMYLTWYQQKPGKAPRLLIYWASISQSGVPSRFSGSGSGTDYTLTISSLQPEDFATYYCQQRKARPYTFGQGTKVEIKR

SEQ ID NO: 413 - protein, 108 aa, Homo sapiens
DIQMTQSPSSLSASVGDRVTITCRASQAIEWYLAWYQQKPGKAPKLLIYNASILQSGVPSRFSGSGSGTDFTLTISSLQPEDFATYYCQQRAFSPLTFGQGTKVEIKR

SEQ ID NO: 414 - protein, 108 aa, Homo sapiens
DIQMTQSPSSLSASVGDRVTITCRASQAIWTYLNWYQQKPGKAPKLLIYGASQLQSGVPSRFSGSGSGTDFTLTISSLQPEDFATYYCQQTESFPVTFGQGTKVEIKR

SEQ ID NO: 415 - protein, 108 aa, Homo sapiens
DIQMTQSPSSLSASVGDRVTITCRASQTITDYLNWYQQKPGKAPKLLIYWGSILQSGVPSRFSGSGSGTDFTLTISSLQPEDFATYYCQQSAHRPYTFGQGTKVEIKR

SEQ ID NO: 416 - protein, 108 aa, Homo sapiens
DIQMTQSPSSLSASVGDRVTITCRASQKIGSHLSWYQQKPGKAPKLLIYRTSQLQSGAPSRFSGSGSGTDFTLTISSLQPEDFATYYCQQQAKSPRTFGQGTKVEIKR

SEQ ID NO: 417 - protein, 108 aa, Homo sapiens
DIQMTQSPSSLSASVGDRVTITCRASQQIDDYLNWYQQKPGKAPKLLIYWTSLLQSGVPSRFSGSGSGTDFTLTISSLQPEDFATYYCQQSAHRPFTFGQGTKVEIKR

SEQ ID NO: 418 - protein, 108 aa, Homo sapiens
DIQMTQSPSSLSASVGDRVTITCRASQQIDDRLSWYQQKPGKAPKLLIYFKSFLQSGVPSRFSGSGSGTDFTLTISSLQPEDFATYYCQQYQAHPLTFGQGTKVEIKR

SEQ ID NO: 419 - protein, 108 aa, Homo sapiens
DIQMTQSPSSLSASVGDRVTITCRASQRIAGCLSWYQQKPGKAPKLLIYRTSLLQSGVPSRFSGSGSGTDFTLTISSLQPEDFATYYCQQDCTFPRTFGQGTKVEIKR

SEQ ID NO: 420 - protein, 108 aa, Homo sapiens
DIQMTQSPSSLSASVGDRVTITCRASQRISGCLYWYQQKPGKAPKLLIYRGSHLQSGVPSRFSGSGSGTDFTLTISSLQPEDFATYYCQQDCDPPSTFGQGTKVEIKR

SEQ ID NO: 421 - protein, 108 aa, Homo sapiens; identical to SEQ ID NO: 410

SEQ ID NO: 422 - protein, 108 aa, Homo sapiens
DIQMTQSPSSLSASVGDRVTITCRASQNISYHLVWYQQKPGKAPKLLIYSSSNLQSGVPSRFSGSGSGTDFTLTISSLQPEDLATYYCQQLASWPHTLGQGTKVEIKR

SEQ ID NO: 423 - protein, 108 aa, Homo sapiens
DIQMTQSPSSLSASVGDRVTITCRASQNISRGLRWYQQKPGKAPKLLIYHASKLQSGVPSRFSGSGSGTDFTLTISSLQPEDFATYYCQQYKVFPGTFGQGTKVEIKR

SEQ ID NO: 424 - protein, 108 aa, Homo sapiens
DIQMTQSPSSLSASVGDRVTITCRASQNIGSHLLWYQQKPGKAPKLLIYGSSSLQSGVPSRFSGSGSGTDFTLTISSLQPEDFATYYCQQVRLAPHTFGQGTKVEIKR

SEQ ID NO: 425 - protein, 108 aa, Homo sapiens
DIQMTQSPSSLSASVGDRVTITCRASQNIGMYLKWYQQKPGKAPKLLIYYSSSLQSGVPSRFSGSGSGTDFTLTISSLQPEDFATYYCQQNRMRPTTFGQGTKVEIKR

SEQ ID NO: 426 - protein, 108 aa, Homo sapiens
DIQMTQSPSSLSASVGDRVTITCRASQGIDWYLSWYQQKPGKAPKLLIYEGSNLQSGVPSRFSGSGSGTDFTLTISSLQPEDSATYYCQQRAAYPFTFGQGTKVEIKR

SEQ ID NO: 427 - protein, 108 aa, Homo sapiens
DIQMTQSPSSLSASVGDRVTITCRASQGIGVALDWYQQKPGKAPKLLIYMASRLQSGVPSRFSGSGSGTDFTLTISSLQPEDFATYYCQQYSELPVTFGQGTKVEIKR

SEQ ID NO: 428 - protein, 108 aa, Homo sapiens
DIQMTQSPSSLSASVGDRVTITCRASQEIGQWLNWYQQKPGKAPKLLIYWGSELQSGVPSRFSGSGSGTDFTLTISSLQPEDFATYYCQQVSRNPFTFGQGTKVEIKR

SEQ ID NO: 429 - protein, 108 aa, Homo sapiens
DIQMTQSPSSLSASVGDRVTITCRASQEISGELTWYQQKPGKAPKLLIYFSSILQSGVPSRFSGSGSGTDFTLTISSLQPEDFATYYCQQRKLRPYTFGQGTKVEIKR

SEQ ID NO: 430 - protein, 108 aa, Homo sapiens
DIQMTQSPSSLSASVGDRVTITCRASQEIGQWLNWYQQKPGKAPKLLIYWGSELQSGVPSRFSGSGSGTDFTLTISSLQPEDFATYYCQQTQLRPSTFGQGTKVEIKR

SEQ ID NO: 431 - protein, 108 aa, Homo sapiens; identical to SEQ ID NO: 428

SEQ ID NO: 432 - protein, 108 aa, Homo sapiens
DIQMTQSPSSLSASVGDRVTITCRASQKIATYLNWYQQKPGKAPKLLIYRSSSLQSAVPSRFSGSGSGTVFTLTISSLQPEDFATYYCQQTYAVPPTFGQGTKVEIKR

SEQ ID NO: 433 - protein, 108 aa, Homo sapiens
DIQMTQSPSSLSASVGDRVTITCRASQWIDTGLAWYQQKPGKAPRLLIYNVSRLQSGVPSRFSGSGSGTDFTLTISSLQPEDFATYYCQQYWGSPTTFGQGTKVEIKR

SEQ ID NO: 434 - protein, 108 aa, Homo sapiens
DIQMTQSPSSLSASVGDRVTITCRASQEIYSWLAWYQQRPGKAPKLLIYNASHLQSGVPSRFSGSGSGTDFTLTISSLQPEDFATYYCQQVIGDPVTFGQGTKVEIKR

SEQ ID NO: 435 - protein, 108 aa, Homo sapiens
DIQMTQSPSSLSASVGDRVTITCRASQSISSYLNWYQQKPGKAPTLLIYRLSVLQSGVPSRFSGSGSGTDFTLTISSLQPEDFATYYCQQTYNVPPTFGQGTKVEIKR

SEQ ID NO: 436 - protein, 108 aa, Homo sapiens
DIQMTQSPSSLSASVGDRVTITCRASQSISSYLNWYQQKPGKAPKLLIYRNSFLQSGVPSRFSGSGSGTDFTLTISSLQPEDFATYYCQQTYTVPPTFGQGTKVEIKQ

SEQ ID NO: 437 - protein, 108 aa, Homo sapiens
DIQMTQSPSSLSASVGDRVTITCRASQSISSYLNWYQQKPGKAPKLLIYRNSQLQSGVPSRFSGSGSGTDFTLTISSLQPEDFATYYCQQTFAVPPTFGQGTKVEIKR

SEQ ID NO: 438 - protein, 108 aa, Homo sapiens
DIQMTQSPSSLSASVGDRVTITCRASQSISSYLNWYQQKPGKAPKLLIYRNSPLQSGVPSRFSGSGSGTDFTLTISSLQPEDFATYYCQQTYRVPPTFGQGTKVEIKR

SEQ ID NO: 439 - protein, 108 aa, Homo sapiens
DIQMTQSPSSLSASVGDRVTITCRASQHIHRELRWYQQKPGKAPKLLIYQASRLQSGVPSRFSGSGSGTDFTLTISSLQPEDFATYYCQQKYLPPYTFGQGTKVEIKR

SEQ ID NO: 440 - protein, 108 aa, Homo sapiens
DIQMTQSPSSLSASVGDRVTITCRASQHIHRELRWYQQKPGKAPKLLIYQASRLQSGVPSRFSGSGSGTDFTLTISSLQPEDFATYYCQQRYRVPYTFGQGTKVEIKR

SEQ ID NO: 441 - protein, 123 aa, Homo sapiens
EVQLLESGGGLVQPGGSLRLSCAASGFTFSKYWMSWVRQAPGKGLEWVSSIDFMGPHTYYADSVKGRFTISRDNSKNTLYLQMNSLRAEDTAVYYCAKGRTSMLPMKGKFDYWGQGTLVTVSS

SEQ ID NO: 442 - protein, 118 aa, Homo sapiens
EVQLLESGGGLVQPGGSLRLSCAASGFTFYDYNMSWVRQAPGKGLEWVSTITHTGGVTYYADSVKGRFTISRDNSKNTLYLQMNSLRAEDTAVYYCAKQNPSYQFDYWGQGTLVTVSS

SEQ ID NO: 443 - protein, 118 aa, Homo sapiens
EVQLLESGGGLVQPGGSLRLSCAASGFTFHRYSMSWVRQAPGKGLEWVSTILPGGDVTYYADSVKGRFTISRDNSKNTLYLQMNSLRAEDTAVYYCAKQTPDYMFDYWGQGTLVTVSS

SEQ ID NO: 444 - protein, 117 aa, Homo sapiens
EVQLLESGGGLVQPGGSLRLSCAASGFTFWKYNMAWVRQAPGKGLEWVSTILGEGNNTYYADSVKGRFTISRDNSKNTLYLQMNSLRAEDTAVYYCAKTMDYKFDYWGQGTLVTVSS

SEQ ID NO: 445 - protein, 118 aa, Homo sapiens
EVQLLESGGGLVQPGGSLRLSCTASGFTFDEYNMSWVRQAPGKGLEWVSTILPHGDRTYYADSVKGRFTISRDNSKNTLYLQMNSLRAEDTAVYYCAKQDPLYRFDYWGQGTLVTVSS

SEQ ID NO: 446 - protein, 120 aa, Homo sapiens
EVQLLESGGGLVQPGGSLRLSCAASGFTFDLYDMSWVRQAPGKGLEWVSSIVNSGVRTYYADSVKGRFTISRDNSKNTLYLQMNSLRAEDTAVYYCAKLNQSYHWDFDYWGQGTLVTVSS

SEQ ID NO: 447 - protein, 118 aa, Homo sapiens
EVQLLESGGGLVQPGGSLRLSCAASGFTFSDYRMSWVRQAPGKGLEWVSTIISNGKFTYYADSVKGRFTISRDNSKNTLYLQMNSLRAEDTAVYYCAKQDWMYMFDYWGQGTLVTVSS

SEQ ID NO: 448 - protein, 115 aa, Camelid
QVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 449 - protein, 115 aa, Camelid
QVQLQESGGGLVQPGNSLRLSCAASGFTFRNFGMSWVRQAPGKEPEWVSSISGSGSNTIYADSVKDRFTISRDNAKSTLYLQMNSLKPEDTAVYYCTIGGSLSRSSQGTQVTVSS

SEQ ID NO: 450 - protein, 114 aa, Camelid
QVQLQESGGGLVQPGGSLRLTCTASGFTFSSFGMSWVRQAPGKGLEWVSAISSDSGTKNYADSVKGRFTISRDNAKKMLFLQMNSLRPEDTAVYYCVIGRGSPSSQGTQVTVSS

SEQ ID NO: 451 - protein, 114 aa, Camelid
QVQLQESGGGLVQPGGSLRLTCTASGFTFRSFGMSWVRQAPGKGLEWVSAISADGSDKRYADSVKGRFTISRDNGKKMLTLDMNSLKPEDTAVYYCVIGRGSPASQGTQVTVSS

SEQ ID NO: 452 - protein, 128 aa, Camelid
AVQLVESGGGLVQAGDSLRLSCVVSGTTFSSAAMGWFRQAPGKEREFVGAIKWSGTSTYYTDSVKGRFTISRDNVKNTVYLQMNNLKPEDTGVYTCAADRDRYRDRMGPMTTTDFRFWGQGTQVTVSS

SEQ ID NO: 453 - protein, 124 aa, Camelid
QVKLEESGGGLVQTGGSLRLSCAASGRTFSSFAMGWFRQAPGREREFVASIGSSGITTNYADSVKGRFTISRDNAKNTVYLQMNSLKPEDTGLCYCAVNRYGIPYRSGTQYQNWGQGTQVTVSS

SEQ ID NO: 454 - protein, 120 aa, Camelid
EVQLEESGGGLVQPGGSLRLSCAASGLTFNDYAMGWYRQAPGKERDMVATISIGGRTYYADSVKGRFTISRDNAKNTVYLQMNSLKPEDTAIYYCVAHRQTVVRGPYLLWGQGTQVTVSS

SEQ ID NO: 455 - protein, 123 aa, Camelid
QVQLVESGGKLVQAGGSLRLSCAASGRTFSNYAMGWFRQAPGKEREFVAGSGRSNSYNYYSDSVKGRFTISRDNAKNTVYLQMNSLKPEDTAVYYCAASTNLWPRDRNLYAYWGQGTQVTVSS

SEQ ID NO: 456 - protein, 125 aa, Camelid
EVQLVESGGGLVQAGDSLRLSCAASGRSLGIYRMGWFRQVPGKEREFVAAISWSGGTTRYLDSVKGRFTISRDSTKNAVYLQMNSLKPEDTAVYYCAVDSSGRLYWTLSTSYDYWGQGTQVTVSS

SEQ ID NO: 457 - protein, 125 aa, Camelid
QVQLVEFGGGLVQAGDSLRLSCAASGRSLGIYKMAWFRQVPGKEREFVAAISWSGGTTRYIDSVKGRFTLSRDNTKNMVYLQMNSLKPDDTAVYYCAVDSSGRLYWTLSTSYDYWGQGTQVTVSS

SEQ ID NO: 458 - protein, 124 aa, Camelid
EVQLVESGGGLVQAGGSLSLSCAASGRTFSPYTMGWFRQAPGKEREFLAGVTWSGSSTFYGDSVKGRFTASRDSAKNTVTLEMNSLNPEDTAVYYCAAAYGGGLYRDPRSYDYWGRGTQVTVSS

SEQ ID NO: 459 - protein, 131 aa, Camelid
AVQLVESGGGLVQAGGSLRLSCAASGFTLDAWPIAWFRQAPGKEREGVSCIRDGTTYYADSVKGRFTISSDNANNTVYLQTNSLKPEDTAVYYCAAPSGPATGSSHTFGIYWNLRDDYDNWGQGTQVTVSS

SEQ ID NO: 460 - protein, 126 aa, Camelid
EVQLVESGGGLVQAGGSLRLSCAASGFTFDHYTIGWFRQVPGKEREGVSCISSSDGSTYYADSVKGRFTISSDNAKNTVYLQMNTLEPDDTAVYYCAAGGLLLRVEELQASDYDYWGQGIQVTVSS

SEQ ID NO: 461 - protein, 128 aa, Camelid
AVQLVDSGGGLVQPGGSLRLSCTASGFTLDYYAIGWFRQAPGKEREGVACISNSDGSTYYGDSVKGRFTISRDNAKTTVYLQMNSLKPEDTAVYYCATADRHYSASHHPFADFAFNSWGQGTQVTVSS

SEQ ID NO: 462 - protein, 120 aa, Camelid
EVQLVESGGGLVQAGGSLRLSCAAYGLTFWRAAMAWFRRAPGKERELVVARNWGDGSTRYADSVKGRFTISRDNAKNTVYLQMNSLKPEDTAVYYCAAVRTYGSATYDIWGQGTQVTVSS

SEQ ID NO: 463 - protein, 123 aa, Camelid
EVQLVESGGGLVQDGGSLRLSCIFSGRTFANYAMGWFRQAPGKEREFVAAINRNGGTTNYADALKGRFTISRDNTKNTAFLQMNSLKPDDTAVYYCAAREWPFSTIPSGWRYWGQGTQVTVSS

SEQ ID NO: 464 - protein, 125 aa, Camelid
DVQLVESGGGWVQPGGSLRLSCAASGPTASSHAIGWFRQAPGKEREFVVGINRGGVTRDYADSVKGRFAVSRDNVKNTVYLQMNRLKPEDSAIYICAARPEYSFTAMSKGDMDYWGKGTLVTVSS

SEQ ID NO: 465 - protein, 119 aa, Homo sapiens
EVQLLESGGGLVQPGGSLRLSCAASGFTFIGYNMYWVRQAPGKGLEWVSFISPSGRETYYADSVKGRFTISRDNSKNTLYLQMNSLRAEDTAVYYCAKTLSADGRFDYWGQGTLVTVSS

SEQ ID NO: 466 - protein, 120 aa, Homo sapiens
EVQLLESGGGLVQPGGSLRLSCAASGFTFGSYDMGWVRQAPGKGLEWVSFIDVSGTLTYYADSVKGRFTISRDNSKNTLYLQMNSLRAEDTAVYYCAKTVELDGLDFDYWGQGTLVTVSS

SEQ ID NO: 467 - protein, 120 aa, Homo sapiens
EVQLLESGGGLVQPGGSLRLSCAASGFTFADYDMGWVRQAPGKGLEWVSFIDSSGSRTYYADSVKGRFTISRDNSKNTLYLQMNSLRAEDTAVYYCAKTAEIVNSRFDYWGQGTLVTVSS

SEQ ID NO: 468 - protein, 118 aa, Homo sapiens
EVQLLESGGGLVQPGGSLRLSCAASGFTFRYGMVWVRQAPGKGLEWVSAISGSGGSTYYADSVKGRFTISRDNSKNTLYLQMNSLRAEDTAVYYCAKRHSSEARQFDYWGQGTLVTVS

SEQ ID NO: 469 - DNA, 357 bp, Homo sapiens
gaggtgcagctgttggagtctgggggaggcttggtacagcctggggggtccctgcgtctctcctgtgcagcctccggattcaccttttagcggtatgggatggtttgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaacggcatagttctgaggctaggcagtttgactactggggccagggaaccctggtcaccgtctcg

SEQ ID NO: 470 - DNA, 357 bp, Homo sapiens
cgagacggtgaccagggttccctggccccagtagtcaaactgcctagcctcagaactatgccgtttcgcacagtaatataccgcggtgtcctcggcacgcaggctgttcatttgcagatacagcgtgttcttggaattgtcgcgggagatggtgaaccggcccttcacggagtctgcgtagtatgtgctaccaccactaccactaatagctgagacccactctagacccttccctggagcctggcggacccaaaccatcccataccgctaaaaggtgaatccggaggctgcacaggagagacgcagggaccccccaggctgtaccaagcctcccccagactccaacagctgcacctc

SEQ ID NO: 471 - DNA, 39 bp, Artificial Sequence
  primers
tggagcgcgtcgacggacatccagatgacccagtctcca

SEQ ID NO: 472 - DNA, 39 bp, Artificial Sequence
  primers
ttagcagccggatccttattagcaccgtttgatttccac

SEQ ID NO: 473 - protein, 5 aa, Artificial Sequence
  CDR1
XXXLX

SEQ ID NO: 474 - protein, 7 aa, Artificial Sequence
  CDR2
XASXLQS

SEQ ID NO: 475 - protein, 9 aa, Artificial Sequence
  CDR3
QQXXXXPXT

SEQ ID NO: 476 - protein, 5 aa, Artificial Sequence
  CDR1
SSYLN

SEQ ID NO: 477 - protein, 7 aa, Artificial Sequence
  CDR2
RASPLQS

SEQ ID NO: 478 - protein, 9 aa, Artificial Sequence
  CDR3
QQTYSVPPT

SEQ ID NO: 479 - protein, 9 aa, Artificial Sequence
  CDR3
QQTYRIPPT

SEQ ID NO: 480 - protein, 5 aa, Artificial Sequence
  CDR1
FKSLK

SEQ ID NO: 481 - protein, 7 aa, Artificial Sequence
  CDR2
NASYLQS

SEQ ID NO: 482 - protein, 9 aa, Artificial Sequence
  CDR3
QQVVYWPVT

SEQ ID NO: 483 - protein, 5 aa, Artificial Sequence
  CDR1
YYHLK

SEQ ID NO: 484 - protein, 7 aa, Artificial Sequence
  CDR2
KASTLQS

SEQ ID NO: 485 - protein, 9 aa, Artificial Sequence
  CDR3
QQVRKVPRT

SEQ ID NO: 486 - protein, 5 aa, Artificial Sequence
  CDR1
RRYLK

SEQ ID NO: 487 - protein, 7 aa, Artificial Sequence
  CDR2
QASVLQS

SEQ ID NO: 488 - protein, 9 aa, Artificial Sequence
  CDR3
QQGLYPPIT

SEQ ID NO: 489 - protein, 5 aa, Artificial Sequence
  CDR1
YNWLK

SEQ ID NO: 490 - protein, 7 aa, Artificial Sequence
  CDR2
RASSLQS

SEQ ID NO: 491 - protein, 9 aa, Artificial Sequence
  CDR3
QQNVVIPRT

SEQ ID NO: 492 - protein, 5 aa, Artificial Sequence
  CDR1
LWHLR

SEQ ID NO: 493 - protein, 7 aa, Artificial Sequence
  CDR2
HASLLQS

SEQ ID NO: 494 - protein, 9 aa, Artificial Sequence
  CDR3
QQSAVYPKT

SEQ ID NO: 495 - protein, 5 aa, Artificial Sequence
  CDR1
FRYLA

SEQ ID NO: 496 - protein, 7 aa, Artificial Sequence
  CDR2
HASHLQS

SEQ ID NO: 497 - protein, 9 aa, Artificial Sequence
  CDR3
QQRLLYPKT

SEQ ID NO: 498 - protein, 5 aa, Artificial Sequence
  CDR1
FYHLA

SEQ ID NO: 499 - protein, 7 aa, Artificial Sequence
  CDR2
PASKLQS

SEQ ID NO: 500 - protein, 9 aa, Artificial Sequence
  CDR3
QQRARWPRT

SEQ ID NO: 501 - protein, 5 aa, Artificial Sequence
  CDR1
IWHLN

SEQ ID NO: 502 - protein, 7 aa, Artificial Sequence
  CDR2
RASRLQS

SEQ ID NO: 503 - protein, 9 aa, Artificial Sequence
  CDR3
QQVARVPRT

SEQ ID NO: 504 - protein, 5 aa, Artificial Sequence
  CDR1
YRYLR

SEQ ID NO: 505 - protein, 7 aa, Artificial Sequence
  CDR2
KASSLQS

SEQ ID NO: 506 - protein, 9 aa, Artificial Sequence
  CDR3
QQYVGYPRT

SEQ ID NO: 507 - protein, 5 aa, Artificial Sequence
  CDR1
LKYLK

SEQ ID NO: 508 - protein, 7 aa, Artificial Sequence
  CDR2
NASHLQS

SEQ ID NO: 509 - protein, 9 aa, Artificial Sequence
  CDR3
QQTTYYPIT

SEQ ID NO: 510 - protein, 5 aa, Artificial Sequence
  CDR1
LRYLR

SEQ ID NO: 511 - protein, 7 aa, Artificial Sequence
  CDR2
KASWLQS

SEQ ID NO: 512 - protein, 9 aa, Artificial Sequence
  CDR3
QQVLYYPQT

SEQ ID NO: 513 - protein, 5 aa, Artificial Sequence
  CDR1
LRSLK

SEQ ID NO: 514 - protein, 7 aa, Artificial Sequence
  CDR2
AASRLQS

SEQ ID NO: 515 - protein, 9 aa, Artificial Sequence
  CDR3
QQVVYWPAT

SEQ ID NO: 516 - protein, 5 aa, Artificial Sequence
  CDR1
FRHLK

SEQ ID NO: 517 - protein, 9 aa, Artificial Sequence
  CDR3
QQVALYPKT

SEQ ID NO: 518 - protein, 5 aa, Artificial Sequence
  CDR1
RKYLR

SEQ ID NO: 519 - protein, 7 aa, Artificial Sequence
  CDR2
TASSLQS

SEQ ID NO: 520 - protein, 9 aa, Artificial Sequence
  CDR3
QQNLFWPRT

SEQ ID NO: 521 - protein, 5 aa, Artificial Sequence
  CDR1
RRYLN

SEQ ID NO: 522 - protein, 7 aa, Artificial Sequence
  CDR2
AASSLQS

SEQ ID NO: 523 - protein, 9 aa, Artificial Sequence
  CDR3
QQMLFYPKT

SEQ ID NO: 524 - protein, 5 aa, Artificial Sequence
  CDR1
IKHLK

SEQ ID NO: 525 - protein, 7 aa, Artificial Sequence
  CDR2
GASRLQS

SEQ ID NO: 526 - protein, 9 aa, Artificial Sequence
  CDR3
QQGARWPQT

SEQ ID NO: 527 - protein, 5 aa, Artificial Sequence
  CDR1
YKHLK

SEQ ID NO: 528 - protein, 9 aa, Artificial Sequence
  CDR3
QQVGRYPKT

SEQ ID NO: 529 - protein, 6 aa, Artificial Sequence
  CDR1
XXYXXX

SEQ ID NO: 530 - protein, 17 aa, Artificial Sequence
  CDR2
XIXXXGXXTXYADSVKG

SEQ ID NO: 531 - protein, 11 aa, Artificial Sequence
  CDR3
XXXXXXXXFDY

SEQ ID NO: 532 - protein, 6 aa, Artificial Sequence
  CDR1
WVYQMD

SEQ ID NO: 533 - protein, 17 aa, Artificial Sequence
  CDR2
SISAFGAKTLYADSVKG

SEQ ID NO: 534 - protein, 7 aa, Artificial Sequence
  CDR3
LSGKFDY

SEQ ID NO: 535 - protein, 6 aa, Artificial Sequence
  CDR1
WSYQMT

SEQ ID NO: 536 - protein, 17 aa, Artificial Sequence
  CDR2
SISSFGSSTLYADSVKG

SEQ ID NO: 537 - protein, 11 aa, Artificial Sequence
  CDR3
GRDHNYSLFDY

SEQ ID NO: 538 - protein, 119 aa, Homo sapiens
EVQLLESGGGLVQPGGSLRLSCAASGFTFPEYGMAWVRQAPGKGLEWVSTISHGGEHTYYADSVKGRFTISRDNSKNTLYLQMNSLRAEDTAVYYCAQHPVSHPKFDYWGQGTLVTVSS

SEQ ID NO: 539 - protein, 116 aa, Homo sapiens
EVQLLESGGGLVQPGGSLRLSCAASGFTFDAYNMFWVRQAPGKGLEWVSAISPSGRETYYADSVKGRFTISRDNSKNTLYLQMNSLRAEDTAVYYCAKRYPDFDYWGQGTLVTVSS

SEQ ID NO: 540 - DNA, 36 bp, Artificial Sequence
  Primer
aaggaaaaaagcggccgcaactgtggctgcaccatc

SEQ ID NO: 541 - DNA, 40 bp, Artificial Sequence
  Primer
ccgctcgagtcaacactctcccctgttgaagctctttgtg

SEQ ID NO: 542 - DNA, 40 bp, Artificial Sequence
  Primer
aaggaaaaaagcggccgcctccaccaagggcccatcggtc

SEQ ID NO: 543 - DNA, 43 bp, Artificial Sequence
  Primer
gtgaggtttgtcacaagatttgggctcaactttcttgtccacc

SEQ ID NO: 544 - DNA, 24 bp, Artificial Sequence
  Primer
cccaaatcttgtgacaaacctcac

SEQ ID NO: 545 - DNA, 31 bp, Artificial Sequence
  Primer
ccgctcgagtcatttacccggagacagggag

SEQ ID NO: 546 - DNA, 64 bp, Artificial Sequence
  Primer
ctagccaccatgccgctgctgctactgctgccactgctgtgggcaggagcactggctatggata

SEQ ID NO: 547 - DNA, 64 bp, Artificial Sequence
  Primer
agcttatccatagccagtgctcctgcccacagcagtggcagcagtagcagcagcggcatggtgg

SEQ ID NO: 548 - DNA, 73 bp, Artificial Sequence
  Primer
ctagccaccatggagacagacacactcctgctatgggtactgctgctctgggttccaggttccactggtgaca

SEQ ID NO: 549 - DNA, 73 bp, Artificial Sequence
  Primer
agcttgtcaccagtggaacctggaacccagagcagcagtacccatagcaggagtgtgtctgtctccatggtgg

SEQ ID NO: 550 - DNA, 20 bp, Artificial Sequence
  Primer
taatacgactcactataggg

SEQ ID NO: 551 - DNA, 18 bp, Artificial Sequence
  Primer
tagaaggcacagtcgagg

SEQ ID NO: 552 - protein, 122 aa, Homo sapiens
EVQLLESGGGLVQPGGSLRLSCAASGFTFEWYWMGWVRQAPGKGLEWVSAISGSGGSTYYADSVKGRFTISRDNSKNTLYLQMNSLRAEDAAVYYCAKVKLGGGPNFGYRGQGTLVTVSSAA

SEQ ID NO: 553 - DNA, 368 bp, Homo sapiens
gaggtgcagctgttggagtctgggggaggcttggtacagcctggggggtccctgcgtctctcctgtgcagcctccggattcacctttgagtggtattggatgggttgggtccgccaggctccagggaagggtctagagtgggtctcagctatcagtggtagtggtggtagcacatactacgcagactccgtgaagggccggttcaccatctcccgcgacaattccaagaacacgctgtatctgcaaatgaacagcctgcgtgccgaggacgccgcggtatattactgtgcgaaagttaagttggggggggggcctaattttggctaccggggccagggaaccctggtcaccgtctcgagcgcggccgc